(19) United States
(12) Patent Application Publication
(10) Pub. No.: US 2004/0176324 A1
(43) Pub. Date: Sep. 9, 2004

(54) Fused heterocyclic succinimide compounds and analogs thereof, modulators of nuclear hormone receptor function

(76) Inventors: Mark E. Salvati, Lawrenceville, NJ (US); James Aaron Balog, Lambertville, NJ (US); Dacia A. Pickering, Lawrenceville, NJ (US); Soren Giese, New Hope, PA (US); Aberra Fura, Lawrenceville, NJ (US); Wenying Li, Middletown, CT (US); Ramesh N. Patel, Bridgewater, NJ (US); Ronald L. Hanson, Morris Plains, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 09/885,381

(22) Filed: Jun. 20, 2001

Related U.S. Application Data
(60) Provisional application No. 60/233,519, filed on Sep. 19, 2000.
(60) Provisional application No. 60/284,730, filed on Apr. 18, 2001.
(60) Provisional application No. 60/284,438, filed on Apr. 18, 2001.

(51) Int. Cl. A61K 31/675; A61K 31/655; A61K 31/407; C07D 487/02

(52) U.S. Cl. 514/79; 514/150; 534/787; 514/411; 548/414; 548/428; 548/429

(57) ABSTRACT

Fused cyclic compounds, methods of using such compounds in the treatment of nuclear hormone receptor-associated conditions such as cancer and immune disorders, and pharmaceutical compositions containing such compounds.

This application claims priority from U.S. Application Serial No. 60/233,519, filed Sep. 19, 2000, from U.S. Application Serial No. 60/284,730, filed Apr. 18, 2001, and from U.S. Application Serial No. 60/284,438, filed Apr. 18, 2001, which provisional applications are incorporated herein by reference in their entirety.

### FIELD OF THE INVENTION

The present invention relates to fused cyclic compounds, to methods of using such compounds in the treatment of nuclear hormone receptor-associated conditions such as cancer, and to pharmaceutical compositions containing such compounds.

### BACKGROUND OF THE INVENTION

Nuclear hormone receptors (NHR's) constitute a large super-family of ligand-dependent and sequence-specific transcription factors. Members of this family influence transcription either directly, through specific binding to the promoter target genes (Evans, in Science 240: 889-895 (1988)), or indirectly, via protein-protein interactions with other transcription factors (Jonat et al., Cell 62: 1189-1204 (1990), Schuele et al., Cell 62: 1217-1226 (1990), and Yang-Yen et al., Cell 62: 1205-1215 (1990)). The nuclear hormone receptor super-family (also known in the art as the “steroid/thyroid hormone receptor super-family”) includes receptors for a variety of hydrophobic ligands, including cortisol, aldosterone, estrogen, progesterone, testosterone, vitamine D3, thyroid hormone and retinoic acid (Evans, 1988, supra). In addition to these conventional nuclear hormone receptors, the super-family contains a number of proteins that have no known ligands, termed orphan nuclear hormone receptors (Mangelsdorf et al., Cell 83: 835-839 (1995), O'Malley et al., Mol. Endocrinol. 10: 1293 (1996), Enmark et al., Mol. Endocrinol. 10, 1293-1307 (1996) and Giguere, Eindocrin. Rev. 20, 689-725 (1999)). The conventional nuclear hormone receptors are generally transactivators in the presence of ligand, and can either be active repressors or transcriptionally inert in the absence of ligand. Some of the orphan receptors behave as if they are transcriptionally inert in the absence of ligand. Others, however, behave as either constitutive activators or repressors. These orphan nuclear hormone receptors are either under the control of ubiquitous ligands that have not been identified, or do not need to bind ligand to exert these activities.

In common with other transcription factors, the nuclear hormone receptors have a modular structure, being comprised of three distinct domains: an N-terminal domain of variable size containing a transcriptional activation function AF-1, a highly conserved DNA binding domain and a moderately conserved ligand-binding domain. The ligand-binding domain is not only responsible for binding the specific ligand but also contains a transcriptional activation function called AF-2 and a dimerisation domain (Wurtz et al., Nature Struc. Biol. 3, 87-94 (1996), Parker et al., Nature Struc. Biol. 3, 113-115 (1996) and Kumar et al., Steroids 64, 310-319 (1999)). Although the overall protein sequence of these receptors can vary significantly, all share both a common structural arrangement indicative of divergence from an ancestral archetype, and substantial homology (especially, sequence identity) at the ligand-binding domain.

The steroid binding nuclear hormone receptors (SB-NHR's) comprise a sub-family of nuclear hormone receptors. These receptors are related in that they share a stronger sequence homology to one another, particularly in the ligand binding domain (LBD), than to the other members of the NHR super-family (Evans, 1988, supra) and they all utilize steroid based ligands. Some examples of this sub-family of NHR's are the androgen receptor (AR), the estrogen receptor (ER), the progesterone receptor (PR), the glucocorticoid receptor (GR), the mineralocorticoid receptor (MR), the aldosterone receptor (ALDR) and the steroid and xenobiotic receptor (SXR) (Evans et al., WO 99/35246). Based on the strong sequence homology in the LBD, several orphan receptors may also be members of the SB-NHR sub-family.

Consistent with the high sequence homology found in the LBD for each of the SB-NHR's, the natural ligands for each is derived from a common steroid core. Examples of some of the steroid based ligands utilized by members of the SB-NHR's include cortisol, aldosterone, estrogen, progesterone, testosterone and dihydrotestosterone. Specificity of a particular steroid based ligand for one SB-NHR versus another is obtained by differential substitution about the steroid core. High affinity binding to a particular SB-NHR, coupled with high level specificity for that particular SB-NHR, can be achieved with only minor structural changes about the steroid core (e.g., Waller et al., Toxicol. Appl. Pharmacol. 137, 219-227 (1996) and Mekenyan et al., Environ. Sci. Technol. 31, 3702-3711 (1997), binding affinity for progesterone towards the androgen receptor as compared to testosterone).

Numerous synthetically derived steroidal and non-steroidal agonists and antagonists have been described for the members of the SB-NHR family. Many of these agonist and antagonist ligands are used clinically in man to treat a variety of medical conditions. RU486 is an example of a synthetic agonist of the PR, which is utilized as a birth control agent (Vegeto et al., Cell 69: 703-713 (1992)), and Flutamide is an example of an antagonist of the AR, which is utilized for the treatment of prostate cancer (Neri et al, Endo. 91, 427-437 (1972)). Tamoxifen is an example of a tissues specific modulator of the ER function, that is used in the treatment of breast cancer (Smigel, J. Natl. Cancer Inst. 90, 647-648 (1998)). Tamoxifen can function as an antagonist of the ER in breast tissue while acting as an agonist of the ER in bone (Grese et al., Proc. Natl. Acad. Sci. USA 94, 14105-14110 (1997)). Because of the tissue selective effects seen for Tamoxifen, this agent and agents like it are referred to as “partial-agonist” or partial-antagonist”. In addition to synthetically derived non-endogenous ligands, non-endogenous ligands for NHR's can be obtained from food sources (Regal et al., Proc. Soc. Exp. Biol. Med. 223, 372-378 (2000) and Hempstock et al., J. Med. Food 2, 267-269 (1999)). The flavanoid phytoestrogens are an example of an unnatural ligand for SB-NHR's that are readily obtained from a food source such as soy (Quella et al., J. Clin. Oncol. 18, 1068-1074 (2000) and Banz et al., J. Med. Food 2, 271-273 (1999)). The ability to modulate the transcriptional activity of individual NHR by the addition of a small molecule ligand, makes them ideal targets for the development of pharmaceutical agents for a variety of disease states.

As mentioned above, non-natural ligands can be synthetically engineered to serve as modulators of the function of NHR's. In the case of SB-NHR's, engineering of an unnatural ligand can include the identification of a core structure which mimics the natural steroid core system. This can be achieved by random screening against several SB-NHR's or through directed approaches using the available crystal structures of a variety of NHR ligand binding domains (Bourguet et al., Nature 375, 377-382 (1995), Brzozowski, et al., Nature 389, 753-758 (1997), Shiau et al., Cell 95, 927-937 (1998) and Tanenbaum et al., Proc. Natl. Acad. Sci. USA 95, 5998-6003 (1998)). Differential substitution about such a steroid mimic core can provide agents with selectivity for one receptor versus another. In addition, such modifications can be employed to obtain agents with agonist or antagonist activity for a particular SB-NHR. Differential substitution about the steroid mimic core can result in the formation of a series of high affinity agonists and antagonists with specificity for, for example, ER versus PR versus AR versus GR versus MR. Such an approach of differential substitution has been reported, for example, for quinoline based modulators of steroid NHR in J. Med. Chem., 41, 623 (1999); WO 9749709; U.S. Pat. Nos. 5,696,133; 5,696,130; 5,696,127; 5,693,647; 5,693,646; 5,688,810; 5,688,808 and WO 9619458, all incorporated herein by reference.

The compounds of the present invention comprise a core which serves as a steroid mimic, and are useful as modulators of the function of steroid binding nuclear hormone receptors, as well as other NHR as described following.

### SUMMARY OF THE INVENTION

The present invention provides fused cyclic compounds of the following formula I and salts thereof, which compounds are especially useful as modulators of nuclear hormone receptor function:

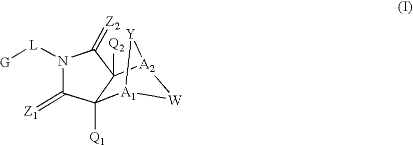

As used in formula I, and throughout the specification, the symbols have the following meanings unless otherwise indicated, and are, for each occurrence, independently selected:

G is an aryl or heterocyclo (e.g., heteroaryl) group, where said group is mono- or polycyclic, and which is optionally substituted at one or more positions, preferably with hydrogen, alkyl or substituted alkyl, alkenyl or substituted alkenyl, alkynyl or substituted alkynyl, halo, cycloalkyl or substituted cycloalkyl, cycloalkenyl or substituted cycloalkenyl, aryl or substituted aryl, heterocyclo or substituted heterocyclo, arylalkyl or substituted arylalkyl, heterocycloalkyl or substituted heterocycloalkyl, CN, R¹OC═O, R¹C═O, R¹C═S, R¹HNC═O, R¹R²NC═O, HOCR³R³′, nitro, R¹OCH₂, R¹O, NH₂, NR⁴R⁵, SR¹, S═OR¹, SO₂R¹, SO₂OR¹, SO₂NR¹R¹′, (R¹O)(R¹40 O)P═O, oxo, (R¹)(R¹′)P═O, or (R¹′)(NHR¹)P═O;

Z, is O, S, NH, or NR⁶;

Z₂ is O, S, NH, or NR⁶;

A₁ is CR⁷ or N;

A₂ is CR⁷ or N;

Y is J—J′—J″ where J is (CR⁷R⁷′)n and n=0-3, J′ is a bond or O, S, S═O, SO₂, NH, NR¹, C═O, OC═O, NR¹C═O, CR⁷R⁷′, C═CR⁸R⁸′, R²P═O, R²P═S, R²OP═O, R²NHP═O, OP═OOR², OP═ONHR², OP═OR², OSO₂, C═NR⁷, NHNH, NHNR⁶, NR⁶NH, N═N, cycloalkyl or substituted cycloalkyl, cycloalkenyl or substituted cycloalkenyl, heterocyclo or substituted heterocyclo or aryl or substituted aryl, and J″ is (CR⁷R⁷′)n and n=0-3, where Y is not a bond;

W is CR⁷R⁷′—CR⁷R⁷′, CR⁸=CR⁸′, CR⁷R⁷′—C═O, NR⁹—CR⁷R⁷′, N═CR⁸, N═N, NR⁹—NR⁹′, S—CR⁷R⁷′, SO—CR⁷R⁷′, SO₂—CR⁷R⁷′, cycloalkyl or substituted cycloalkyl, cycloalkenyl or substituted cycloalkenyl, heterocyclo or substituted heterocyclo, or aryl or substituted aryl, wherein when W is not NR⁹—CR⁷R⁷′, N═CR⁸, N═N, NR⁹—NR⁹′, S—CR⁷R⁷′, SO—CR⁷R⁷′, SO₂—CR⁷R⁷′, or heterocyclo or substituted heterocyclo, then J′ must be O, S, S═O, SO₂, NH, NR⁷, OC═O, NR¹C═O, OP═OOR², OP═ONHR², OSO₂, NHNH, NHNR⁶, NR⁶NH, or N═N;

Q₁ is H, alkyl or substituted alkyl, alkenyl or substituted alkenyl, cycloalkyl or substituted cycloalkyl, cycloalkenyl or substituted cycloalkenyl, heterocycloalkyl or substituted heterocycloalkyl, arylalkyl or substituted arylalkyl, alkynyl or substituted alkynyl, aryl or substituted aryl, heterocyclo (e.g., heteroaryl) or substituted heterocyclo (e.g., substituted heteroaryl), halo, CN, R¹OC═O, R⁴C═O, R⁵R⁶NC═O, HOCR⁷R⁷′, nitro, R¹OCH₂, R¹O, NH₂, C═OSR¹, SO₂R¹ or NR⁴R⁵;

Q₂ is H, alkyl or substituted alkyl, alkenyl or substituted alkenyl, cycloalkyl or substituted cycloalkyl, cycloalkenyl or substituted cycloalkenyl, heterocycloalkyl or substituted heterocycloalkyl, arylalkyl or substituted arylalkyl, alkynyl or substituted alkynyl, aryl or substituted aryl, heterocyclo (e.g., heteroaryl) or substituted heterocyclo (e.g., substituted heteroaryl), halo, CN, R¹OC═O, R⁴C═O, R⁵R⁶NC═O, HOCR⁷R⁷′, nitro, R¹OCH₂, R¹O, NH₂, C═OSR¹, SO₂R¹ or NR⁴R⁵;

L is a bond, (CR⁷R⁷′)n, NH, NR⁵, NH (CR⁷R⁷′)n, or NR⁵(CR⁷R⁷′)n, where n=0-3;

R¹ and R¹′ are each independently H, alkyl or substituted alkyl, cycloalkyl or substituted cycloalkyl, cycloalkenyl or substituted cycloalkenyl, heterocyclo or substituted heterocyclo, cycloalkylalkyl or substituted cycloalkyalkyl, cycloalkenylalkyl or substituted cycloalkenylalkyl, heterocycloalkyl or substituted heterocycloalkyl, aryl or substituted aryl, arylalkyl or substituted arylalkyl;

R² is alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl;

R³ and R³′ are each independently H, alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, halo, CN, hydroxylamine, hydroxamide, alkoxy or substituted alkoxy, amino, NR¹R², thiol, alkylthio or substituted alkylthio;

R⁴ is H, alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, R¹C═O, R¹NHC═O, SO₂OR¹, or SO₂NR¹R¹′;

R⁵ is alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, R¹C═O, R¹NHC═O, SO₂R¹, SO₂OR¹, or SO₂NR¹R¹′;

R⁶ is alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, CN, OH, OR¹, R¹C═O, R¹NHC═O, SO₂R¹, SO₂OR¹, or SO₂NR¹R¹′;

R⁷ and R⁷′ are each independently H, alkyl or substituted alkyl, alkenyl or substituted alkenyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, halo, CN, OR¹, nitro, hydroxylamine, hydroxylamide, amino, NHR⁴, NR²R⁵, NOR¹, thiol, alkylthio or substituted alkylthio, R¹C═O, R¹OC═O, R¹NHC═O, SO₂R¹, SOR¹, PO₃R¹R¹′, R¹R¹′NC═O, C═OSR¹, SO₂R¹, SO₂OR¹, or SO₂NR¹R¹′, or, wherein A₁ or A₂ contains a group R⁷ and W contains a group R⁷, said R⁷ groups of A₁ or A₂ and W together form a heterocyclic ring;

R⁸ and R⁸′ are each independently H, alkyl or substituted alkyl, alkenyl or substituted alkenyl, cycloalkyl or substituted cycloalkyl, cycloalkenyl or substituted cycloalkenyl, heterocyclo or substituted heterocyclo, cycloalkylalkyl or substituted cycloalkyalkyl, cycloalkenylalkyl or substituted cycloalkenylalkyl, heterocycloalkyl or substituted heterocycloalkyl, aryl or substituted aryl, arylalkyl or substituted arylalkyl, nitro, halo, CN, OR¹, amino, NHR⁴, NR²R⁵, NOR¹, alkylthio or substituted alkylthio, C═OSR¹, R¹OC═O, R¹C═O, R¹NHC═O, R¹R¹NC═O, SO₂OR¹, S═OR¹, SO₂R¹, PO₃R¹R¹′, or SO₂NR¹R¹′; and

R⁹ and R⁹′ are each independently H, alkyl or substituted alkyl, alkenyl or substituted alkenyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, CN, OH, OR¹, R¹C═O, R¹OC═O, R¹NHC═O, SO₂R¹, SO₂OR¹, or SO₂NR¹R¹′.

Compounds within formula I are novel, a preferred subgenus of which is the following formula Ia:

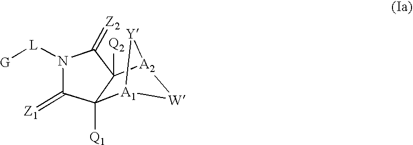

where G, L, Z₁, Z₂, A₁, A₂, Q₁ and Q₂ are as defined above;

Y′ is J—J′—J″ where J is (CR⁷R⁷′)n and n=0-3, J′ is a bond or O, S, S═O, SO₂, NH, NR⁷CR⁷R⁷′, R²P═O, R²P═S, R²OP═O, R²NHP═O, OP═OOR², OP═ONHR², OSO₂, NHNH, NHNR⁶, NR⁶NH, N═N, cycloalkyl or substituted cycloalkyl, cycloalkenyl or substituted cycloalkenyl, or heterocyclo or substituted heterocyclo, and J″ is (CR⁷R⁷′)n and n=0-3, where Y is not a bond; and

W′ is CR⁷R⁷′—CR⁷R⁷′, CR⁷R⁷′C═O, NR⁹—CR⁷R⁷′, N═CR⁸, N═N, NR⁹—N⁹′, cycloalkyl or substituted cycloalkyl, cycloalkenyl or substituted cycloalkenyl, heterocyclo or substituted heterocyclo, or aryl or substituted aryl, wherein, when W′ is not NR⁹—CR⁷R⁷′, N═CR⁸, N═N, NR⁹—NR⁹′, or heterocyclo or substituted heterocyclo, then J′ must be O, S, S═O, SO₂, NH, NR⁷, OP═OOR², OP═ONHR², OSO₂, NHNH, NHNR⁶, NR⁶NH, or N═N; or alternatively,

Y′ is NR⁷13 CR⁷R⁷′ and W′ is CR⁸═CR⁸′; or, alternatively,

Y′ is CR⁷R⁷′—C═O and W′ is NR⁹—CR⁷R⁷′;

where R², R⁶, R⁷, R⁷′, R⁸, R⁹ and R⁹ are as defined above and with the provisos that (1) when Y′ is —O—, Q₁ and Q₂ are hydrogen, Z₁ and Z₂ are O, W′ is —CH₂—CH₂—, and A₁ and A₂ are CH, then G—L is not phenyl, monosubstituted phenyl or phenyl which is substituted with two or more of the following groups: methoxy, halo, NO₂, methyl, CH₃—S—, OH, CO₂H, trifluoromethyl, —C(O)—C₆H₅, NH₂, 4-7-epoxy, hexahydro-1H-isoindole-1,3(2H)dione, or —C(O)—CH₃;

(2) when Y′ is —O—, Q₁ and Q₂ are hydrogen, Z₁ and Z₂ are O, W′ is CH₂—CH₂, and one of A₁ and A₂ is CH and the other is CR⁷, then G—L is not unsubstituted phenyl;

(3) when Y′ is —O—, Q₁ and Q₂ are hydrogen, Z₁ and Z₂ are O, W′ is CH₂—CH₂, and one of A₁ and A₂ is CH and the other is C—CH₃, then G—L is not phenyl substituted with chloro and/or methyl;

(4) when Y′ is —O—, or —S—, Q₁ and Q₂ are hydrogen, Z₁ and Z₂ are O, W′ is CH₂—CH₂, and one of A₁ and A₂ is CH and the other is CH or C-alkyl, then G—L is not N-substituted piperazine-alkyl- or N-substituted imidazolidine-alkyl-;

(5) when Y′ is —O—; Q₁ and Q₂ are hydrogen, Z₁ and Z₂ are O, W′ is CH₂—CH₂, and A₁ and A₂ are CH, then G—L is not oxazole or triazole;

(6) when Y′ is —O—; Q₁ and Q₂ are hydrogen or methyl, Z₁ and Z₂ are O, W′ is CH₂—CH₂, and A₁ and A₂ are CH or C—CH₃, then G—L is not thiazole or substituted thiazole (in addition such compounds where G—L is optionally substituted thiadiazole or partially saturated thiazole are optionally removed by proviso where A₁ and A₂ are both CH);

(7) when Y′ contains a group J′ selected from S, S═O, SO₂, NH, NR⁷, R²P═O, R²P═S, R²OP═O, R²NHP═O, OP═OOR², OP═ONHR², OSO₂, NHNH, NHR⁶, NR⁶NH or N═N, W′ is CR⁷R⁷′—CR⁷R⁷′, and Z₁ and Z₂ are O, then G—L is not unsubstituted phenyl;

(8) when Y′ is NR⁷, W′ is unsubstituted or substituted phenyl, and Q₁ and Q₂ are hydrogen, then Z₁ and Z₂ are not O;

(9) when Y′ is —O—, Q₁ and Q₂ are hydrogen, Z₁ and Z₂ are O, W′ is dihydroisoxazole bearing an optionally substituted phenyl group, and A₁ and A₂ are CH, then G—L is not unsubstituted phenyl or dichlorophenyl;

(10) when Y′ is O, Q₁ and Q₂ are hydrogen, Z₁ and Z₂ are O, W′ is ethylene oxide, and A₁ and A₂ are CH, then G—L is not methylphenyl or chlorophenyl;

(11) when Y′ is NR⁷—CR⁷R⁷′, W′ is CR⁸═CR⁸′, Q₁ and Q₂ are hydrogen, A₁ and A₂ are CH, C—CH₃, C—CH₂—C₆H₅ or C—CH₂—CH₃, and Z₁ and Z₂ are O, then G—L is not unsubstituted phenyl, monosubstituted phenyl or methylpyridinyl;

(12) when Y′ is CR⁷R⁷′—C═O, W′ is NR⁹—CR⁷R⁷′, Q₁ and Q₂ are hydrogen, A₁ and A₂ are CH, and Z₁ and Z₂ are O, then G—L is not unsubstituted phenyl;

(13) when Y′ is CHR⁷—NR⁷ where R⁷′ is unsubstituted phenyl, methoxy or ethoxy and R⁷ is unsubstituted phenyl, methyl or —C(O)—C₆H₅, W′ is dimethoxyphenylene or unsubstituted phenylene, Z₁ and Z₂ are O, Q₁ and Q₂ are hydrogen, and A₁ and A₂ are CH, C—CN, C—C(O)—C₆H₅, or —C(O)—dimethoxyphenyl, then G—L is not unsubstituted phenyl;

(14) the compound of formula Ia is not 6,10-epithio-4H-thieno-[3′,4′:5,6]cyclooct[1,2-ƒ]isoindole-7,9(5H,8H)-dione, 8-(3,5-dichlorophenyl)-6,6a,9a, 10,11,12,-hexahydro-1,3,6,10-tetramethyl-2,2,13-trioxide, (6R,6aR,9aS,10S);

(15) when Y′ is O, W′ is —CH₂—CH₂—, Q₁ and Q₂ are methyl, Z₁ and Z₂ are O, and A₁ and A₂ are CH, then G—L is not unsubstituted phenyl, phenyl substituted with methoxy, phenyl-alkyl-, or morpholine-alkyl, nor is the compound bridged to itself through a group L which is alkylene to form a bis compound;

(16) when Y′ is —O—, Q₁ and Q₂ are hydrogen, Z₁ and Z₂ are O, W′ is CR⁷R⁷′—CR⁷R⁷′, and A₁ and A₂ are CH, then G—L is not an unsubstituted phenyl group; and

(17) when Y′ is —O—, Q₁ and Q₂ are hydrogen, Z₁ and Z₂ are O, W′ is cyclopentyl, cyclohexyl, 3-phenyl-2-isoxazoline or CR⁷R⁷′—CR⁷R⁷′ where R⁷ and R⁷′ are each independently defined as Cl, Br, H and 4-butyrolactone and R⁷ and R⁷′ are not all simultaneously H, and A₁ and A₂ are CH, then G—L is not an unsubstituted naphthyl ring or a monosubstituted phenyl ring, where said substituent is methoxy, Br, Cl, NO₂, methyl, ethyl, CH₂-phenyl, S-phenyl, or O-phenyl.

Preferably, compounds of formula I are monomeric, and are not comprised within other oligomers or polymers.

Another preferred novel subgenus is that of the following formula Ib:

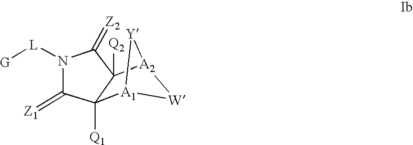

where G, Z₁, Z₂, Q₁ and Q₂ are as defined above;

Y′ is J—J′—J″ where J is (CR⁷R⁷′)n and n=0-3, J′ is a bond or O, S, S═O, SO₂, NH, NR⁷, CR⁷R⁷′, R²P═O, R²P═S, R²OP═O, R²NHP═O, OP═OOR², OP═ONHR², OSO₂, NHNH, NHNR⁶, NR⁶NH, N═N, cycloalkyl or substituted cycloalkyl, cycloalkenyl or substituted cycloalkenyl, or heterocyclo or substituted heterocyclo, and J″ is (CR⁷R⁷′)n and n=0-3, where Y is not a bond; and

W′ is CR⁷R⁷′—CR⁷R⁷′, CR⁷R⁷′—C═O, NR⁹—CR⁷R⁷′, N═CR⁸, N═N, NR⁹—NR⁹′, cycloalkyl or substituted cycloalkyl, cycloalkenyl or substituted cycloalkenyl, heterocyclo or substituted heterocyclo, or aryl or substituted aryl, wherein,

when W′ is not NR⁹—CR⁷R⁷′, N═CR⁸, N═N, NR⁹—NR⁹′, or heterocyclo or substituted heterocyclo, then J′ must be O, S, S═O, SO₂, NH, NR⁷, OP═OOR², OP═ONHR², OSO₂, NHNH, NHNR⁶, NR⁶NH, or N═N; or alternatively,

Y′ is CR⁷R⁷′—C═O and W′ is NR⁹—CR⁷R⁷′;

L is a bond; and

A₁ and A₂ are as defined above, especially where A₁ and/or A₂ are alkyl or optionally substituted alkyl (preferred such optional substituents being one or more groups V¹ defined below), with the proviso that, when Y′═O and W′═—CH₂—CH₂—, then at least one of A₁ or A₂ is not CH;

with the further provisos (2), (3), (6), (7) and (8) above.

The compounds of formula I and salts thereof comprise a core which can serve as a steroid mimic (and do not require the presence of a steroid-type (e.g., cyclo-pentanoperhydrophenanthrene analog) structure).

### FURTHER DESCRIPTION OF THE INVENTION

The following are definitions of terms used in the present specification. The initial definition provided for a group or term herein applies to that group or term throughout the present specification individually or as part of another group, unless otherwise indicated.

The terms “alkyl” and “alk” refers to a straight or branched chain alkane (hydrocarbon) radical containing from 1 to 12 carbon atoms, preferably 1 to 6 carbon atoms. Exemplary such groups include, but are not limited to, methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl, dodecyl, and the like. “Substituted alkyl” refers to an alkyl group substituted with one or more substituents, preferably 1 to 4 substituents, at any available point of attachment. Exemplary substituents include but are not limited to one or more of the following groups: halo (e.g., a single halo substituent or multiple halo substitutents forming, in the latter case, groups such as a perfluoroalkyl group or an alkyl group bearing Cl₃ or CF₃), alkoxy, alkylthio, hydroxy, carboxy (i.e., —COOH), alkoxycarbonyl, alkylcarbonyloxy, amino (i.e., —NH₂), carbamoyl or substituted carbomoyl, carbamate or substituted carbamate, urea or substituted urea, amidinyl or substituted amidinyl, thiol (i.e., —SH), aryl, heterocycle, cycloalkyl, heterocycloalkyl, —S-aryl, —S-heterocycle, —S═O-aryl, —S═O-heterocycle, arylalkyl-O—, —S(O)₂-aryl, —S(O)₂-heterocycle, —NHS(O)₂-aryl, —NHS(O)₂-heterocycle, —NHS(O)₂NH-aryl, —NHS(O)₂NH-heterocycle, —P(O)₂-aryl, —P(O)₂-heterocycle, —NHP(O)₂-aryl, —NHP(O)₂-heterocycle, —NHP(O)₂NH-aryl, —NHP(O)₂NH-heterocycle, —O-aryl, —O-heterocycle, —NH-aryl, —NH-heterocycle, —NHC═O-aryl, —NHC═O-alkyl, —NHC═O-heterocycle, —OC═O-aryl, —OC═O-heterocycle, —NHC═ONH-aryl, —NHC═ONH-heterocycle, —OC═OO-aryl, —OC═OO-heterocycle, —OC═ONH-aryl, —OC═ONH-heterocycle, —NHC═OO-aryl, —NHC═OO-heterocycle, —NHC═OO-alkyl, —C═ONH-aryl, —C═ONH-heterocycle, —C═OO-aryl, —C═OO-heterocycle, —N(alkyl)S(O)₂-aryl, —N(alkyl)S(O)₂-heterocycle, —N(alkyl)S(O)₂NH-aryl, —N(alkyl)S(O)₂NH-heterocycle, —N(alkyl)P(O)₂-aryl, —N(alkyl)P(O)₂-heterocycle, —N(alkyl)P(O)₂NH-aryl, —N(alkyl)P(O)₂NH-heterocycle, —N(alkyl)-aryl, —N(alkyl)-heterocycle, —N(alkyl)C═O-aryl, —N(alkyl)C═O-heterocycle, —N(alkyl)C═ONH-aryl, —N(alkyl)C═ONH-heterocycle, —OC═ON(alkyl)-aryl, —OC═ON(alkyl)-heterocycle, —N(alkyl)C═OO-aryl, —N(alkyl)C═OO-heterocycle, —C═ON(alkyl)-aryl, —C═ON(alkyl)-heterocycle, —NHS(O)₂N(alkyl)-aryl, —NHS(O)₂N(alkyl)-heterocycle, —NHP(O)₂N(alkyl)-aryl, NHP(O)₂N(alkyl)-heterocycle, —NHC═ON(alkyl)-aryl, —NHC═ON(alkyl)-heterocycle, —N(alkyl)S(O)₂N(alkyl)-aryl, —N(alkyl)S(O)₂N(alkyl)-heterocycle, —N(alkyl)P(O)₂N(alkyl)-aryl, —N(alkyl)P(O)₂N(alkyl)-heterocycle, —N(alkyl)C═ON(alkyl)-aryl, and —N(alkyl)C═ON(alkyl)-heterocycle. In the aforementioned exemplary substitutents, in each instance, groups such as “alkyl”, “aryl” and “heterocycle” can themselves be optionally substituted; for example, “alkyl” in the group “NCH═OO-alkyl” recited above can be optionally substituted so that both “NHC═OO-alkyl” and “NHC═OO-substituted alkyl” are exemplary substitutents. Exemplary alkyl substituents also include groups such as “T” and “T-R¹², (which are defined below), especially for substituted alkyl groups within A₁ or A₂.

The term “alkenyl” refers to a straight or branched chain hydrocarbon radical containing from 2 to 12 carbon atoms and at least one carbon-carbon double bond. Exemplary such groups include ethenyl or allyl. “Substituted alkenyl” refers to an alkenyl group substituted with one or more substituents, preferably 1 to 4 substituents, at any available point of attachment. Exemplary substituents include, but are not limited to, alkyl or substituted alkyl, as well as those groups recited above as exemplary alkyl substituents.

The term “alkynyl” refers to a straight or branched chain hydrocarbon radical containing from 2 to 12 carbon atoms and at least one carbon to carbon triple bond. Exemplary such groups include ethynyl. “Substituted alkynyl” refers to an alkynyl group substituted with one or more substituents, preferably 1 to 4 substituents, at any available point of attachment. Exemplary substituents include, but are not limited to, alkyl or substituted alkyl, as well as those groups recited above as exemplary alkyl substituents.

The term “cycloalkyl” refers to a fully saturated cyclic hydrocarbon group containing from 1 to 4 rings and 3 to 8 carbons per ring. Exemplary such groups include cyclopropyl, cyclobutyl, cyclopentyl, cyclohexyl, etc. “Substituted cycloalkyl” refers to a cycloalkyl group substituted with one or more substituents, preferably 1 to 4 substituents, at any available point of attachment. Exemplary substituents include, but are not limited to, nitro, cyano, alkyl or substituted alkyl, as well as those groups recited above as exemplary alkyl substituents, and as previously mentioned as preferred aryl substituents in the definition for G. Exemplary substituents also include spiro-attached or fused cyclic substituents, especially cycloalkenyl or substituted cycloalkenyl.

The term “cycloalkenyl” refers to a partially unsaturated cyclic hydrocarbon group containing 1 to 4 rings and 3 to 8 carbons per ring. Exemplary such groups include cyclobutenyl, cyclopentenyl, cyclohexenyl, etc. “Substituted cycloalkenyl” refers to a cycloalkenyl group substituted with one more substituents, preferably 1 to 4 substituents, at any available point of attachment. Exemplary substituents include but are not limited to nitro, cyano, alkyl or substituted alkyl, as well as those groups recited above as exemplary alkyl substituents, and as previously mentioned as preferred aryl substituents in the definition for G. Exemplary substituents also include spiro-attached or fused cyclic substituents, especially cycloalkyl or substituted cycloalkyl.

The terms “alkoxy” or “alkylthio” refer to an alkyl group as described above bonded through an oxygen linkage (—O—) or a sulfur linkage (—S—), respectively. The terms “substituted alkoxy” or “substituted alkylthio” refer to a substituted alkyl group as described above bonded through an oxygen or sulfur linkage, respectively.

The term “alkoxycarbonyl” refers to an alkoxy group bonded through a carbonyl group.

The term “alkylcarbonyl” refers to an alkyl group bonded through a carbonyl group. The term “alkylcarbonyloxy” refers to an alkylcarbonyl group bonded through an oxygen linkage.

The terms “arylalkyl”, “substituted arylalkyl,” “cycloalkylalkyl,” “substituted cycloalkylalkyl,” “cycloalkenylalkyl”, “substituted cycloalkenylalkyl”, “heterocycloalkyl” and “substituted heterocycloalkyl” refer to aryl, cycloalkyl, cycloalkenyl and heterocyclo groups bonded through an alkyl group, substituted on the aryl, cycloalkyl, cycloalkenyl or heterocyclo and/or the alkyl group where indicated as “substituted.”

The term “aryl” refers to cyclic, aromatic hydrocarbon groups which have 1 to 5 aromatic rings, especially monocyclic or bicyclic groups such as phenyl, biphenyl or naphthyl. Where containing two or more aromatic rings (bicyclic, etc.), the aromatic rings of the aryl group may be joined at a single point (e.g., biphenyl), or fused (e.g., naphthyl, phenanthrenyl and the like). “Substituted aryl” refers to an aryl group substituted by one or more substituents, preferably 1,2,3,4 or 5 substituents, at any point of attachment. Exemplary substituents include, but are not limited to, nitro, cycloalkyl or substituted cycloalkyl, cycloalkenyl or substituted cycloalkenyl, cyano, alkyl-S(O)ₘ-(m=0, 1 or 2), alkyl or substituted alkyl, as well as those groups recited above as exemplary alkyl substituents and as previously mentioned as preferred aryl substituents in the definition for G. Exemplary substituents also include fused cyclic substituents, such as heterocyclo or cycloalkenyl, or substituted heterocyclo or cycloalkenyl, groups (e.g., thereby forming a fluoroenyl, tetrahydronapthalenyl, or dihydroindenyl group).

“Carbamoyl” refers to the group —CONH— which is bonded on one end to the remainder of the molecule and on the other to hydrogen or an organic moiety (such as alkyl, substituted alkyl, aryl, substituted aryl, heterocycle, alkylcarbonyl, hydroxyl and substituted nitrogen). “Carbamate” refers to the group —O—CO—NH— which is bonded on one end to the remainder of the molecule and on the other to hydrogen or an organic moiety (such as those listed above). “Urea” refers to the group —NH—CO—NH— which is bonded on one end to the remainder of the molecule and on the other to hydrogen or an organic moiety (such as those listed above). “Amidinyl” refers to the group —C(═NH)(NH₂). “Substituted carbamoyl,” “substituted carbamate,” “substituted urea” and “substituted amidinyl” refer to carbamoyl, carbamate, urea or amidinyl groups as described above in which one more of the hydrogen groups are replaced by an organic moiety (such as those listed above).

The terms “heterocycle”, heterocyclic” and “heterocyclo” refer to fully saturated, or partially or fully unsaturated, including aromatic (i.e., “heteroaryl”) cyclic groups (for example, 3 to 7 membered monocyclic, 7 to 11 membered bicyclic, or 10 to 16 membered tricyclic ring systems) which have at least one heteroatom in at least one carbon atom-containing ring. Each ring of the heterocyclic group containing a heteroatom may have 1, 2, 3, or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. (The term “heteroarylium” refers to a heteroaryl group bearing a quaternary nitrogen atom and thus a positive charge.) The heterocyclic group may be attached to the remainder of the molecule at any heteroatom or carbon atom of the ring or ring system. Exemplary monocyclic heterocyclic groups include ethylene oxide, azetidinyl, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, hexahydrodiazepinyl, 4-piperidonyl, pyridyl, pyrazinyl, pyrimidinyl, pyridazinyl, triazinyl, triazolyl, tetrazolyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, and the like. Exemplary bicyclic heterocyclic groups include indolyl, isoindolyl, benzothiazolyl, benzodioxolyl, benzoxazolyl, benzoxadiazolyl, benzothienyl, quinuclidinyl, quinolinyl, tetrahydroisoquinolinyl, isoquinolinyl, benzimidazolyl, benzopyranyl, indolizinyl, benzofuryl, benzofurazanyl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydrobenzodioxinyl, dihydrodioxidobenzothiophenyl, dihydroisoindolyl, dihydroindolyl, dihydroquinolinyl, dihydroquinazolinyl (such as 3,4-dihydro-4-oxo-quinazolinyl), triazinylazepinyl, tetrahydroquinolinyl and the like. Exemplary tricyclic heterocyclic groups include carbazolyl, benzidolyl, phenanthrolinyl, dibenzofuranyl, acridinyl, phenanthridinyl, xanthenyl and the like.

“Substituted heterocycle,” “substituted heterocyclic,” and “substituted heterocyclo” (such as “substituted heteroaryl”) refer to heterocycle, heterocyclic or heterocyclo groups substituted with one or more substituents, preferably 1 to 4 substituents, at any available point of attachment. Exemplary substituents include, but are not limited to, cycloalkyl or substituted cycloalkyl, cycloalkenyl or substituted cycloalkenyl, nitro, oxo (i.e., ═O), cyano, alkyl-S(O)ₘ-(m=0, 1 or 2), alkyl or substituted alkyl, as well as those groups recited above as exemplary alkyl substituents, and as previously mentioned as preferred heterocyclo substituents in the definition for G.

The term “quaternary nitrogen” refers to a tetravalent positively charged nitrogen atom including, for example, the positively charged nitrogen in a tetraalkylammonium group (e.g., tetramethylammonium, N-methylpyridinium), the positively charged nitrogen in protonated ammonium species (e.g., trimethyl-hydroammonium, N-hydropyridinium), the positively charged nitrogen in amine N-oxides (e.g., N-methyl-morpholine-N-oxide, pyridine-N-oxide), and the positively charged nitrogen in an N-amino-ammonium group (e.g., N-aminopyridinium).

The terms “halogen” or “halo” refer to chlorine, bromine, fluorine or iodine.

The terms “hydroxylamine” and “hydroxylamide” refer to the groups OH—NH— and OH—NH—CO—, respectively.

When a functional group is termed “protected”, this means that the group is in modified form to mitigate, especially preclude, undesired side reactions at the protected site. Suitable protecting groups for the methods and compounds described herein include, without limitation, those described in standard textbooks, such as Greene, T. W. et al., Protective Groups in Organic Synthesis, Wiley, N.Y. (1991).

When a term such as “(CRR)n” is used, it denotes an optionally substituted alkyl chain existing between the two fragments to which it is bonded, the length of which chain is defined by the range described for the term n. An example of this is n=0-3, implying from zero to three (CRR) units existing between the two fragments, which are attached to the primary and terminal (CRR) units. In the situation where the term n is set to zero (n=0) then a bond exists between the two fragments attached to (CRR).

Unless otherwise indicated, any heteroatom with unsatisfied valences is assumed to have hydrogen atoms sufficient to satisfy the valences.

Divalent groups, such as those in the definition of W (e.g., NR⁹—CR⁷R⁷′), may be bonded in either direction to the remainder of the molecule (e.g,

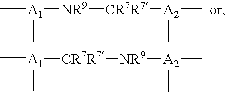

for the aforementioned group within the definition of W).

Carboxylate anion refers to a negatively charged group —COO⁻.

The compounds of formula I form salts which are also within the scope of this invention. Reference to a compound of the formula I herein is understood to include reference to salts thereof, unless otherwise indicated. The term “salt(s)”, as employed herein, denotes acidic and/or basic salts formed with inorganic and/or organic acids and bases. In addition, when a compound of formula I contains both a basic moiety, such as but not limited to a pyridine or imidazole, and an acidic moiety such as but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful, e.g., in isolation or purification steps which may be employed during preparation. Salts of the compounds of the formula I may be formed, for example, by reacting a compound I with an amount of acid or base, such as an equivalent amount, in a medium such as one in which the salt precipitates or in an aqueous medium followed by lyophilization.

The compounds of formula I which contain a basic moiety, such as but not limited to an amine or a pyridine or imidazole ring, may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydroxyethanesulfonates (e.g., 2-hydroxyethanesulfonates), lactates, maleates, methanesulfonates, naphthalenesulfonates (e.g., 2-naphthalenesulfonates), nicotinates, nitrates, oxalates, pectinates, persulfates, phenylpropionates (e.g., 3-phenylpropionates), phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

The compounds of formula I which contain an acidic moiety, such but not limited to a carboxylic acid, may form salts with a variety of organic and inorganic bases. Exemplary basic salts include ammonium salts, alkali metal salts such as sodium, lithium and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glycamides, t-butyl amines, and salts with amino acids such as arginine, lysine and the like. Basic nitrogen-containing groups may be quaternized with agents such as lower alkyl halides (e.g. methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

Prodrugs and solvates of the compounds of the invention are also contemplated herein. The term “prodrug” as employed herein denotes a compound which, upon administration to a subject, undergoes chemical conversion by metabolic or chemical processes to yield a compound of the formula I, or a salt and/or solvate thereof. Solvates of the compounds of formula I include, for example, hydrates.

Compounds of the formula I, and salts thereof, may exist in their tautomeric form (for example, as an amide or imino ether). All such tautomeric forms are contemplated herein as part of the present invention.

All stereoisomers of the present compounds (for example, those which may exist due to asymmetric carbons on various substituents), including enantiomeric forms and diastereomeric forms, are contemplated within the scope of this invention. Individual stereoisomers of the compounds of the invention may, for example, be substantially free of other isomers (e.g., as a pure or substantially pure optical isomer having a specified activity), or may be admixed, for example, as racemates or with all other, or other selected, stereoisomers. The chiral centers of the present invention may have the S or R configuration as defined by the IUPAC 1974 Recommendations. The racemic forms can be resolved by physical methods, such as, for example, fractional crystallization, separation or crystallization of diastereomeric derivatives or separation by chiral column chromatography. The individual optical isomers can be obtained from the racemates by any suitable method, including without limitation, conventional methods, such as, for example, salt formation with an optically active acid followed by crystallization.

All configurational isomers of the compounds of the present invention are contemplated, either in admixture or in pure or substantially pure form. The definition of compounds of the present invention embraces both cis (Z) and trans (E) alkene isomers, as well as cis and trans isomers of cyclic hydrocarbon or heterocyclo rings. In certain cases, for example, the exo or endo conformation can be preferred for the fused ring system bonded to G—L in formula I. For example, for androgen receptor antagonists (or selective androgen receptor modulators), where Y is O or NR⁷, the exo configuration can be preferred, while for most other definitions of Y, the endo configuration can be preferred. As can be appreciated, the preferred configuration can be a function of the particular compound and its preferred activity. Separation of configurational isomers can be achieved by any suitable method, such as column chromatography.

Throughout the specifications, groups and substituents thereof may be chosen to provide stable moieties and compounds.

Embodiments indicated herein as exemplary or preferred are intended to be illustrative and not limiting.

### Methods of Preparation

The compounds of the present invention may be prepared by methods such as those illustrated in the following Schemes I to XI. Solvents, temperatures, pressures, and other reaction conditions may readily be selected by one of ordinary skill in the art. Starting materials are commercially available or readily prepared by one of ordinary skill in the art. Combinatorial techniques may be employed in the preparation of compounds, for example, where the intermediates possess groups suitable for these techniques. See the following which describe other methods which may be employed in the preparation of compounds of the present invention: Li, et al., Eur. J. Org. Chem. 9, 1841-1850 (1998); Li, Y-Q, Synlett. 5, 461-464 (1996); Thiemann, et al., Bull. Chem. Soc. Jpn. 67, 1886-1893 (1994); Tsuge et al., Heterocycles 14, 423-428 (1980); Ward et al., Can J. Chem. 75, 681-693 (1997); Ward et al., Can J. Chem. 69, 1487-1497(1991);Ward etal, Tetrahedron Lett. 31, 845-848 (1990); Fleming et al., J. Org. Chem. 44, 2280-2282 (1979); Jankowski et al., J. Organomet. Chem. 595, 109-113 (2000); Keglevich et al., J. Organomet. Chem. 579, 182-189 (1999); Keglevich et al., J. Organomet. Chem. 570, 49-539 (1998); Jankowski et al., Hetroat. Chem. 7, 369-374 (1996); Jankowski et al., J. Am. Chem. Soc. 113, 7011-7017 (1991); Quin et al., Tetrahedron Lett. 31, 6473-6476 (1990); Quin et al., J. Org. Chem. 59, 120-129 (1994); Quin et al., J. Org. Chem. 58, 6212-6216 (1993); Quin et al., Phosphorous, Sulfur Silicon Relat. Elem. 63, 349-362 (1991); Quin et al., Hetroat. Chem. 2, 359-367 (1991); Hussong et al., Phosphorus Sulfur. 25, 201-212 (1985); Quin et al., J. Org. Chem. 51, 3341-3347 (1986); Myers et al., J. Am. Chem. Soc. 114, 5684-5692(1992); Myers et al., J. Am. Chem. Soc. 113, 6682-6683 (1991); Shen et al., U.S. Pat. No. 5,817,679; Cordone et al., J. Am. Chem. Soc. 111, 5969-5970 (1989); Jung et al., J. Chem. Soc. Commun. 630-632 (1984); Lay et al., J. Am. Chem. Soc. 104, 7658-7659 (1982); Gonzalez et al., J. Am. Chem. Soc. 117, 3405-3421 (1995); Kreher et al., Chem Ber. 125, 183-189 (1992); Simig et al., Synlett. 7, 425-426 (1990); Sha et al., J. Org. Chem. 55, 2446-2450 (1990); Drew et al., J. Chem. Soc., Perkin Trans. 17, 1277-1284 (1985); Kreher et al., Anorg. Chem., Org Chem. 31B, 599-604 (1976); Avalos et al., Tetrahedron Lett. 39, 9301-9304 (1998); Gousse et al., Macromolecules 31, 314-321 (1998); Mikhailyuchenko et al., Khim. Geterotsikl. Soedin. 6, 751-758 (1993); Lubowitz et al., U.S. Pat. No. 4,476,184; Padwa et al., J. Org. Chem. 61, 3706-3714 (1996); Schlessinger et al., J. Org. Chem. 59, 3246-3247 (1994); Buchmeiser et al.,WO Publication No. 9827423; Tanabe et al., Japanese Patent Document JP 07144477; Mochizucki et al., Japanese Patent Document JP 63170383; Hosoda et al., Japanese Patent Document JP 62053963; Onaka et al., Japanese Patent Document JP 62053964; Kato et al., Japanese Patent Document JP 53086035; Kato et al., Japanese Patent Document JP 51088631; Tottori et al., Japanese Patent Document JP 49124225; Augustin et al., German Patent Document DD101271; Title et al., French Patent Document FR 2031538; Gousse et al., Polym. Int. 48, 723-731 (1999); Padwa et al., J. Org. Chem. 62, 4088-4096 (1997); Theurillat-Moritz et al., Tetrahedron: Asymmetry 7, 3163-3168 (1996); Mathews et al., J. Carbohydr. Chem. 14, 287-97 (1995); Srivastava et al., Natl. Acad. Sci. Lett. (India) 15, 41-44 (1992); Mayorga et al., Rev. Cubana Quim. 4, 1-6 (1988); Kondoli et al., J. Chem. Res., Synop. 3, 76 (1987); Primelles et al., Cent. Azucar 7-14 (1985); Solov'eva et al., Khim. Geterotsikl. Soedin. 5, 613-15 (1984); Liu et al., Yaoxue Xuebao 18, 752-759 (1983); Joshi et al., Indian J. Chem, Sect. B. 22B, 131-135 (1983); Amos et al., WO Publication No. 9829495; Odagiri et al., U.S. Pat. No. 4,670,536; Gallucci et al., European Patent Document EP 355435; Redmore, D. U.S. Pat. No. 3,821,232; Nakano et al., Heterocycles 35, 37-40(1993); Tomisawa et al., Chem. Pharm. Bull. 36, 1692-1697(1988); Krow et al., J. Heterocycl. Chem. 22, 131-135 (1985); Krow et al., J. Org. Chem. 47, 1989-1993 (1982); Liu et al., Yaoxue Xuebao 18, 752-759 (1983); Nishikawa et al, Yaoxue Xuebao JP 01061457; and/or Rice et al., J. Med. Chem. 11, 183-185 (1968).

All documents cited in the present specification, such as those cited in this “Methods of Preparation” as well as other sections herein, are incorporated herein by reference in their entirety. Reference to any document herein is not to be construed as an admission that such document is prior art.

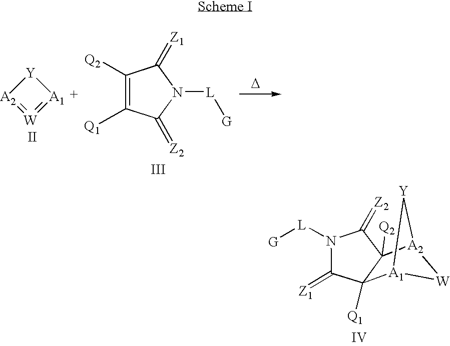

As illustrated in Scheme I, a diene of formula II can be reacted with a dienophile of formula III, under conditions readily selected by one skilled in the art (such as by the addition of heat (“Δ”)), to obtain a compound of formula IV, which is a compound of formula I. An intermediate diene of formula II can be obtained from commercial sources or readily made by one skilled in the art, for example, in accordance with the following literature documents and the references found therein: Hofman et al., J. Agric. Food Chem. 45, 898-906 (1997); Baciocchi et al., J. Chem. Soc., Perkin Trans. 2 8, 821-824 (1975); Wu et al., J. Heterocycles 38, 1507-1518 (1994); Yin et al., Tetrahedron Lett. 38, 5953-5954 (1997); Mic'ovic' et al.,Tetrahedron 20, 2279-2287 (1964); Gorbunova et al., J. Org. Chem. 35, 1557-1566 (1999); Rassu et al., Chem. Soc. Rev. 29, 109-118 (2000); Kaberdin et al., Russ. Chem. Rev. 68, 765-779 (1999); Barluenga et al., Aldrichimica Acta 32, 4-15 (1999); Bogdanowicz-Szwed et al., Pol. Wiad. Chem. 52, 821-842 (1998); Casiraghi et al., Adv. Asymmetric Synth. 3, 113-189 (1998); and/or Baeckvall et al., Chem. Rev. 98, 2291-2312 (1998). An intermediate dieneophile of formula III can be obtained from commercial sources or readily made by one skilled in the art, for example, in accordance with the following literature references and the references found therein: Deshpande et al., Heterocycles 51, 2159-2162 (1999); Seijas et al., J. Chem. Res., Synop. 7, 420-421 (1999); Langer et al., Eur. J. Org. Chem. 7, 1467-1470 (1998); Kita et al., Japanese Patent Document JP 09194458; Lopez-Alvarado et al., J. Org. Chem. 61, 5865-5870 (1996); Condon et al., U.S. Pat. No. 5,523,277; Sasakihara et al., Japanese Patent Document JP 04290868; Igarashi et al., Japanese Patent Document JP 04149173; Aoyama et al., Japanese Patent Document JP 04134063; Aoyama et al., Japanese Patent Document JP 04134062; Pastor et al., J. Org. Chem. 53, 5776-5779 (1988); and/or Takahashi et al., Chem. Lett. 6, 1229-1232 (1987).

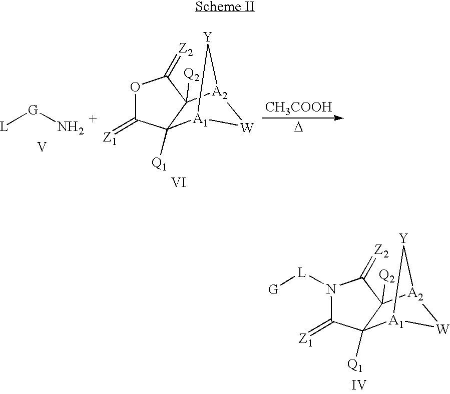

As illustrated in Scheme II, compounds of formula I can be obtained by reaction of a primary amine of formula V with a substituted anhydride-like intermediate of formula VI, for example, in a solvent such as acetic acid with or without heating, to yield a compound of formula IV, which is a compound of formula I. Primary amines of formula V can be obtained from commercial sources or readily synthesized by one skilled in the art. Anhydride-like agents of formula VI can be obtained from commercial sources or readily synthesized by one skilled in the art. The documents listed following describe exemplary approaches for the synthesis of intermediates of formula VI as well as synthetic approaches which can be applied to the synthesis of compounds of formula IV (all incorporated herein by reference in their entirety): Kohler, E. P.; Tishler, M.; Potter, H.; Thompson, H. T. J. Am. Chem. Soc. 1939, 1057-1061; Yur'ev, Y. K.; Zefirov, N. S. J. Gen. Chem. U.S.S.R. (Engl. Transl.) 1961, 31, 772-5; Norman G. Gaylord U.S. Pat. No. 3,995,099; Schueler, P. E.; Rhodes, Y. E. J. Org. Chem. 1974, 39, 2063-9; Ishitobi, H.; Tanida, H; Tsuji, T. Bull. Chem. Soc. Japan 1971, 44, 2993-3000; Stájer, G.; Virag, M.; Szabó, A. E.; Bernath, G.; Sohár, P.; Sillanpää, R. Acta. Chem. Scand. 1996, 50, 922-30; Hart, H.; Ghosh, T. Tetrahedron Lett. 1988,29,881-884; Kato, M.; Yamamoto, S.; Yoshihara, T.; Furuichi, K; Miwa, T. Chem. Lett. 1987, 1823-1826; Kottwitz, J.; Vorbrüggen, H. Synthesis 1995, 636-637; Creary, X. J. Org. Chem. 1975, 40, 3326-3331; Alder, K.; Ache, H.-J.; Flock, F. H. Chem. Ber. 1960, 93, 1888-1895; Toder, B. H.; Branca, S. J.; Dieter, R. K.; Smith, A. B. III Synth. Commun. 1975, 5, 435-439; Sprague, P. W.; Heikes, J. E.; Gougoutas, J. Z.; Malley, M. F.; Harris, D. N.; and/or Greenberg, R. J. Med. Chem. 1985, 28, 1580-1590.

The aforementioned approach(es) can be applied in a combinatorial fashion, for example, by utilizing a multi-well reaction block such as is described in Waldemar Ruediger, Wen-Jeng Li, John W., Allen Jr., and Harold N. Weller III, U.S. Pat. No. 5,961,925, Apparatus for Synthesis of Multiple Organic Compounds With Pinch Valve Block (incorporated herein by reference in its entirety). By utilizing the above-mentioned multi-well reaction block, one can, for example, perform multiples of 96 reactions at a time. Solvent can then be removed from the reaction tubes without removal from the reaction block and the crude products can be precipitated using a base such as sodium bicarbonate. The precipitates can be collected by filtration of the reaction block and then the desired products can be transferred directly to 96 well plates for screening. In this fashion, a large array of compounds of formula I can be synthesized, and tests conducted as desired by an automated approach.

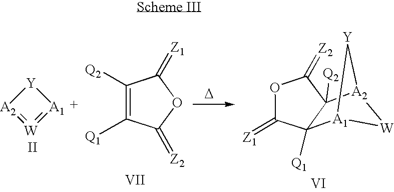

Scheme III describes a method for preparing an intermediate compound of formula VI which can be used to synthesize a compound of formula I, as described in Scheme II. As described in Scheme III, a diene of formula II can be reacted with a dieneophile of formula VII to yield the intermediate of formula VI. The methods applied to obtain such a transformation are analogous to those described in Scheme I.

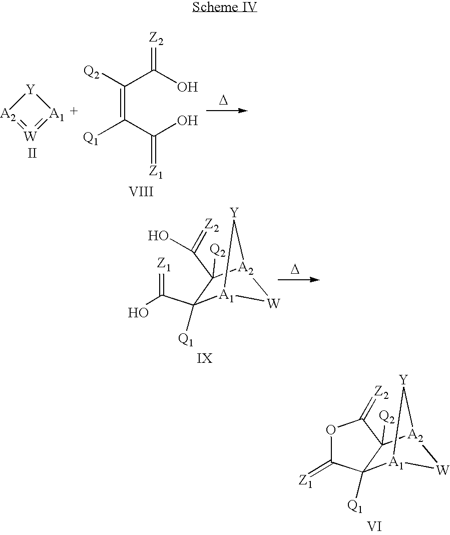

Scheme IV describes a method for preparing an intermediate compound of formula VI which can be used to synthesize a compound of formula I, as described in Scheme II. As shown in Scheme IV, a diene of formula II can be reacted with a dieneophile of formula VIII to yield the intermediate of formula IX. The intermediate of formula IX can be dehydrated to an anhydride-like intermediate of formula VI. Dehydration of the bis-acid intermediate of formula IX to can be achieved by a variety of methods, such as those known to one skilled in the art and described in the following documents and the references embodied therein: Sprague et al., J. Med. Chem. 28, 1580-1590 (1985); and/or Retemi et al., J. Org. Chem. 61, 6296-6301 (1996).

Schemes I to IV describe general methods for the synthesis of compounds of formula I, and intermediates thereof, in which substitution about the ring system is incorporated directly, for example, at the level of the intermediate diene, dienophile, anhydride-like intermediate and amine groups. In addition to these approaches, additional substitution can be incorporated onto an already-prepared compound of formula I by a variety of approaches to prepare other compounds of the formula I. Exemplary methods for further substitution are described in Schemes V to XI.

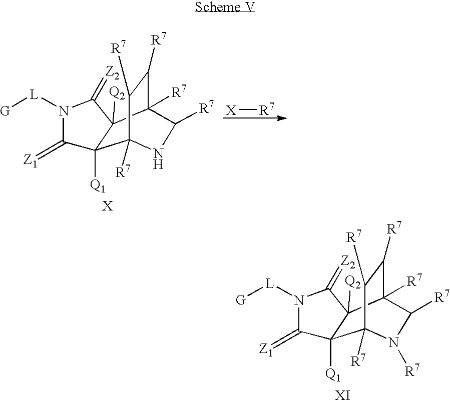

Scheme V describes one such approach to incorporating additional substitution into a structure of formula I. As illustrated in Scheme V, a compound of formula X, which is a compound of formula I where A₁ and A₂ are CR⁷, W is NH—CHR⁷ and Y is CHR⁷—CHR⁷, can be functionalized at the free amine of the group W by reaction with any of a variety of electrophilic agents such as acid halides or alkyl halides in the presence of base, for example, by methods known by one skilled in the art. In Scheme V, X is a leaving group, and a compound of formula XI is a compound of formula I where A₁ and A₂ are CR⁷, W is NR⁷—CHR⁷ and Y is CHR⁷—CHR⁷.

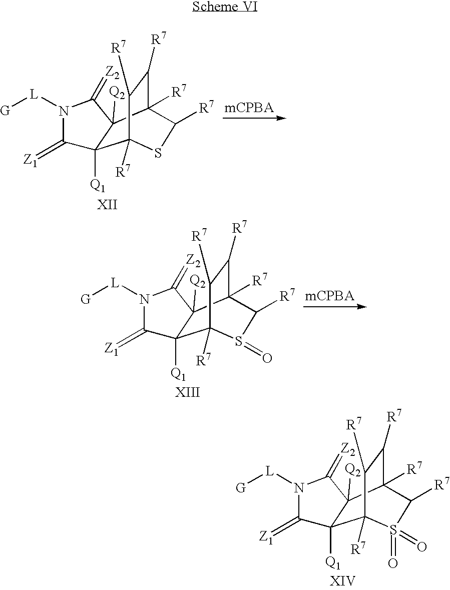

Scheme VI describes an additional approach for further incorporating substitution onto a compound of formula I. As illustrated in Scheme VI, a compound of formula XII, which is a compound of formula I where A₁ and A₂ are CR⁷, W is S—CHR⁷ and Y is CHR ⁷—CHR⁷, can be partially oxidized with an oxidizing agent such as mCPBA or other agents such as those known to one skilled in the art, to give the sulfoxide analog of formula XIII, which is a compound of formula I where A₁ and A₂ are CR⁷, W is SO—CHR⁷ and Y is CHR⁷—CHR⁷. Further treatment of a compound of formula XIII with an oxidizing agent such as mCPBA or other agents such as those known to one skilled in the art, can yield the sulphone analog of formula XIV, which is a compound of formula I where A₁ and A₂ are CR⁷, W is SO₂—CHR⁷ and Y is CHR⁷—CHR⁷. Alternatively, a compound of formula XII can be converted directly to a compound of fonnula XIV by prolonged treatment with an oxidizing agent, such as mCPBA, or with other agents such as those known to one skilled in the art.

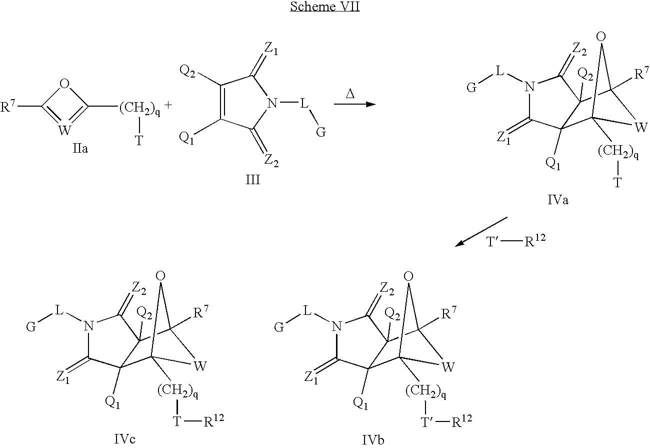

Scheme VII describes another approach to incorporating additional substitution onto a compound of formula I. As illustrated in Scheme VII, a diene of formula IIa can be reacted with a dienophile of formula III, as described in Scheme I, to yield a compound of formula IVa, which is a compound of formula I where Y is O, A₂ is CR⁷ and A₁ is C—(CH₂)_{q}—T. The compound of formula IVa can be reacted with a reagent of formula R¹²—T′ to obtain a compound of formula IVb or IVc which are compounds of formula I where Y is O, A₂ is CR⁷ and A₁ is C—(CH₂)_{q}—T′—R¹² or C—(CH₂)_{q}—T—R¹², respectively. The reagent R¹²—T′ can be obtained from commercial sources or can readily be prepared by one skilled in the art.

In the above Scheme, R¹² has the same definition as R⁷ defined earlier, q is zero or an integer from 0-8, and T is defmed either as (1) a nucleophilic center such as, but not limited, to a nitrogen, oxygen or sulfur-containing group, capable of undergoing a nucleophilic substitution reaction with the leaving group T′ or (2) a leaving group capable undergoing a nucleophilic substitution reaction with a nucleophilic group T′ (such as, but not limited, to a nitrogen, oxygen or sulfur-containing nucleophilic group). T′ has the same definition as T. In the present case, for example, a nucleophilic substitution reaction occurs when the attacking reagent (the nucleophile) brings an electron pair to the substrate, using this pair to form the new bond, and the leaving group (the nucleofuge) comes away with the electron pair, leaving as an anionic intermediate. For a detailed discussion of the mechanism of aliphatic nucleophilic substitutions and a review of specific aliphatic nucleophilic substitution reactions see Advanced Organic Chemistry, Reactions, Mechanisms, and Structure, 4^{th} Addition. Jerry March (Ed.), John Wiley & Sons, New York (1992) 293-500 and the references therein. Compounds of the formulae IVa, IVb, or IVc may, of course, be employed in the methods described herein (especially, in the treatment of nuclear hormone receptor-associated conditions) without undergoing further reaction of T or T′.

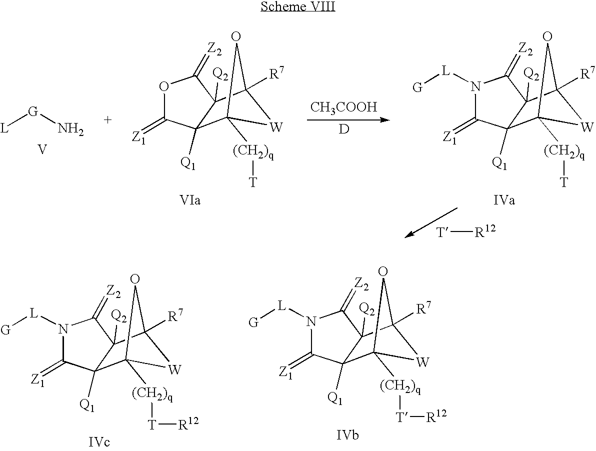

An alternate approach to compounds of formula IVa, IVb and IVc is illustrated in Scheme VIII. For this approach, techniques such as those described in Schemes II, III and IV can be applied to the preparation of an intermediate of formula VIa, where T and q are as defined in Scheme VII. The intermediate of formula VIa can be reacted with a substitited amine of formula V, as described in Scheme II, to yield the compound of formula IVa, which is a compound of formula I where Y is O, A₂ is CR⁷ and A₁ is C—(CH₂)_{q}—T. The compound of formula IVa can be treated in the manner described in Scheme VII to obtain compounds of formula IVb or IVc which are compounds of formula I where Y is O, A₂ is CR⁷ and A₁ is C—(CH₂)_{q}—T′—R¹² or C—(CH₂)_{q}—T—R¹², respectively.

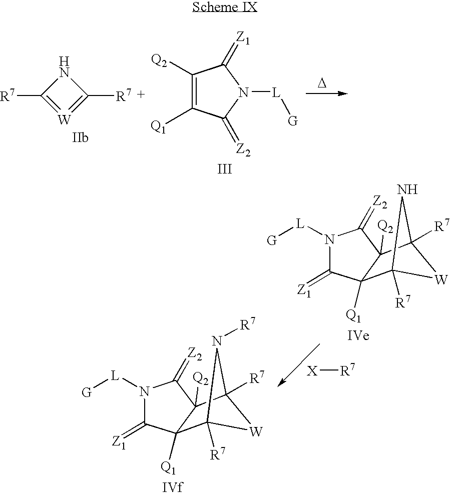

Scheme IX describes another approach to incorporating further substitution onto a compound of formula I. As illustrated in Scheme IX (where X is a leaving group), a diene of formula IIb can be reacted with a dienophile of formula III, as described in Scheme I, to yield a compound of formula IVe, which is a compound of fornula I where Y is NH, and A₁ and A₂ are CR⁷. The compound of formula IVe can be functionalized at the free amine by reacting with a variety of electrophilic agents such as acid halides or alkyl halides in the presence of base, for example by methods known by one skilled in the art and described in Scheme V, to yield a compound of formula IVf, which is a compound of formula I where Y is NR⁷ and A₁ and A₂ are CR⁷.

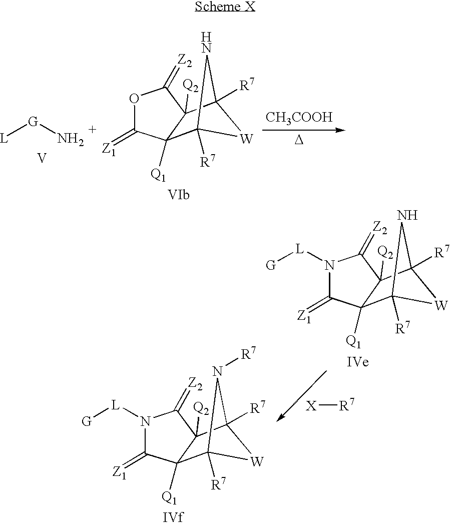

An alternate approach to compounds of formula IVe and IVf is illustrated in Scheme X. For this approach, techniques as described in Schemes II, III and IV can be applied to the preparation of an intermediate of formula VIb. The intermediate of formula VIb can be reacted with a substituted amine of formula V, as described in Scheme II, to yield a compound of formula IVc, which is a compound of formula I where Y is NH, and A₁ and A₂ are CR⁷. The latter intermediate can be treated in the manner described in Scheme V to obtain a compound of formula IVf, which is a compound of formula I where Y is NR⁷, and A₁ and A₂ are CR⁷.

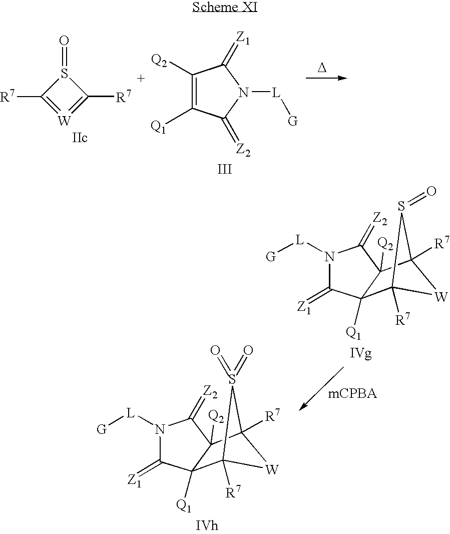

Scheme XI describes another approach to incorporating additional substitution onto a compound of formula I. As illustrated in Scheme XI, a diene of formula IIc can be reacted with a dienophile of formula III, as described in Scheme I, to yield a compound of formula IVg, which is a compound of formula I where Y is SO and A₁ and A₂ are CR⁷. A compound of formula IVg can be treated with an oxidizing agent such as mCPBA, as described in Scheme VI, to yield a compound of formula IVh, which is a compound of formula I where Y is SO₂ and A₁ and A₂ are CR⁷.

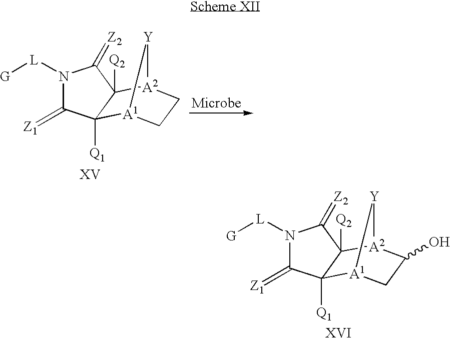

Scheme XII describes another approach to incorporating additional substitution onto a compound of formula I. As illustrated in Scheme XII, a compound of formula XV, which can be prepared in accordance with the above Schemes, can be incubated in the presence of a suitable enzyme or microorganism resulting in the formation of a hydroxylated analog of formula XVI. Such a process can be employed to yield regiospecific as well as enantiospecific incorporation of a hydroxyl group into a molecule of formula XV by a specific microorganism or by a series of different microorganisms. Such microorganisms can, for example, be bacterial, yeast or fungal in nature and can be obtained from distributors such as ATCC or identified for use in this method such as by methods known to one skilled in the art. Compound XVI is a compound of formula I where Y is as described above and A₁ and A₂ are preferably CR⁷.

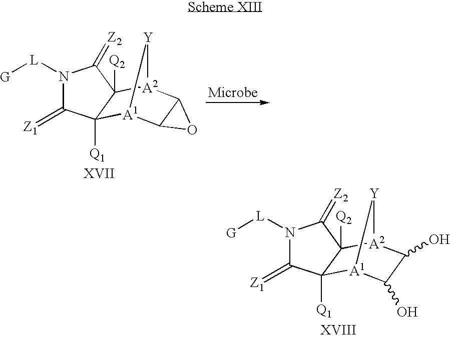

Scheme XIII describes another approach to incorporating additional substitution onto a compound of formula I. As illustrated in Scheme XIII, a compound of formula XVII, which can be prepared in accordance with the above Schemes, can be incubated in the presence of a suitable enzyme or microorganism resulting in the formation of a diol analog of formula XVIII. Such a process can be employed to yield regiospecific as well as enantiospecific transformation of a compound of formula XVII to a 1-2 diol of formula XVIII by a specific microorganism or by a series of different microorganisms. Such microorganisms can, for example, be bacterial, yeast or fungal in nature and can be obtained from distributors such as ATCC or identified for use in this method such as by methods known to one skilled in the art. Compound XVIII is a compound of formula I where Y is as described above and A₁ and A₂ are preferably CR⁷.

The present invention also provides the methods of Schemes XII and XIII.

Thus, in one embodiment, the present invention provides a method for preparation of a compound of the following formula XVI, or salt thereof:

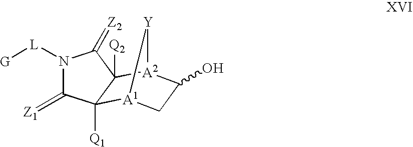

where the symbols are as defined herein,

comprising the steps of contacting a compound of the following formula XV, or salt thereof:

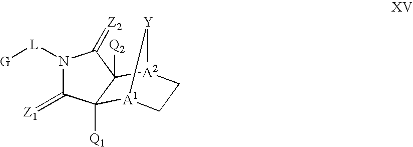

where the symbols are as defined above;

with an enzyme or microorganism capable of catalyzing the hydroxylation of said compound XV to form said compound XVI, and effecting said hydroxylation.

In another preferred embodiment, the present invention provides a method for preparation of a compound of the following formula XVIII, or salt thereof:

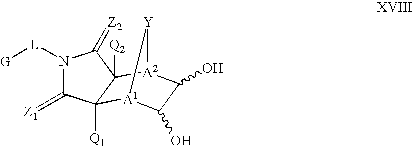

where the symbols are as defined herein,

comprising the steps of contacting a compound of the following formula XVII, or salt thereof:

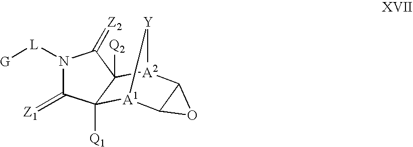

where the symbols are as defined above;

with an enzyme or microorganism capable of catalyzing the opening of the epoxide ring of compound XVII to form the diol of said compound XVIII, and effecting said ring opening and diol formation.

All stereoconfigurations of the unspecified chiral centers of the compounds of the formulae XV, XVI, XVII and XVIII are contemplated in the methods of the present invention, either alone (that is, substantially free of other stereoisomers) or in admixture with other stereoisomeric forms. Conversion of one isomer selectively (e.g., hydroxylation of the exo isomer preferentially to hydroxylation of the endo isomer) when contacting an isomeric mixture is a preferred embodiment of the invention. Conversion to one isomer selectively (e.g., hydroxylation on the exo face “exo isomer” preferentially to the endo face “endo isomer” or regioselective opening of an epoxide to form only one of two possible regioisomers of a trans diol) is a preferred embodiment of the invention. Hydroxylation of an achiral intermediate to form a single optical isomer of the hydroxylated product is also a preferred embodiment of the invention. Resolution of a recemic mixture of an intermediate by selective hydroxylation, or epoxide ring opening and diol formation, to generate one of the two possible optical isomers is also a preferred embodiment of the invention. The term “resolution” as used herein denotes partial, as well as, preferably, complete resolution.

The terms “enzymatic process” or “enzymatic method”, as used herein, denote a process or method of the present invention employing an enzyme or microorganism. The term “hydroxylation”, as used herein, denotes the addition of a hydroxyl group to a methylene group as described above. Hydroxylation can be achieved, for example, by contact with molecular oxygen according to the methods of the present invention. Diol formation can be achieved, for example, by contact with water according to the methods of the present invention. Use of “an enzyme or microorganism” in the present methods includes use of two or more, as well as a single, enzyme or microorganism.

The enzyme or microorganism employed in the present invention can be any enzyme or microorganism capable of catalyzing the enzymatic conversions described herein. The enyzmatic or microbial materials, regardless of origin or purity, can be employed in the free state or immobilized on a support such as by physical adsorption or entrapment. Microorganisms or enzymes suitable for use in the present invention can be selected by screening for the desired activity, for example, by contacting a candidate microorganism or enzyme with a starting compound XV or XVII or salt thereof, and noting conversion to the corresponding compound XVI or XVIII or salt thereof. The enzyme may, for example, be in the form of animal or plant enzymes or mixtures thereof, cells of microorganisms, crushed cells, extracts of cells, or of synthetic origin.

Exemplary microorganisms include those within the genera: Streptomyces or Amycolatopsis. Particularly preferred microorganisms are those within the species Streptomyces griseus, especially Streptomyces griseus ATCC 10137, and Amycolatopsis orientalis such as ATCC 14930, ATCC 21425, ATCC 35165, ATCC 39444, ATCC 43333, ATCC 43490, ATCC 53550, ATCC 53630, and especially ATCC 43491. The term “ATCC” as used herein refers to the accession number of the American Type Culture Collection, 10801 University Blvd., Manassas Va. 20110-2209, the depository for the organism referred to. It should be understood that mutants of these organisms are also contemplated by the present invention, for use in the methods described herein, such as those modified by the use of chemical, physical (for example, X-rays) or biological means (for example, by molecular biology techniques).

Preferred enzymes include those derived from microorganisms, particularly those microorganisms described above. Enzymes may be isolated, for example, by extraction and purification methods such as by methods known to those of ordinary skill in the art. An enzyme may, for example, be used in its free state or in immobilized form. One embodiment of the invention is that where an enzyme is adsorbed onto a suitable carrier, e.g., diatomaceous earth (porous Celite Hyflo Supercel), microporous polypropylene (Enka Accurel(V polypropylene powder), or a nonionic polymeric adsorbent such as Amberlite® XAD-2 (polystyrene) or XAD-7 (polyacrylate) from Rohm and Haas Co. When employed to immobilize an enzyme, a carrier may control the enzyme particle size and prevent aggregation of the enzyme particles when used in an organic solvent. Immobilization can be accomplished, for example, by precipitating an aqueous solution of the enzyme with cold acetone in the presence of the Celite Hyflo Supercel followed by vacuum drying, or in the case of a nonionic polymeric adsorbent, incubating enzyme solutions with adsorbent on a shaker, removing excess solution and drying enzyme-adsorbent resins under vacuum. While it is desirable to use the least amount of enzyme possible, the amount of enzyme required will vary depending upon the specific activity of the enzyme used.

Hydroxylation as described above can occur in vivo. For example, liver enzyme can selectively, relative to the endo isomer, hydroxylate the exo isomer of a compound of the present invention. In conducting the methods of the present invention outside the body, liver microsomal hydroxylase can be employed as the enzyme for catalysis.

These processes may also be carried out using microbial cells containing an enzyme having the ability to catalyze the conversions. When using a microorganism to perform the conversion, these procedures are conveniently carried out by adding the cells and the starting material to the desired reaction medium.

Where microorganisms are employed, the cells may be used in the form of intact wet cells or dried cells such as lyophilized, spray-dried or heat-dried cells, or in the form of treated cell material such as ruptured cells or cell extracts. Cell extracts immobilized on Celite® or Accurel® polypropylene as described earlier may also be employed. The use of genetically engineered organisms is also contemplated. The host cell may be any cell, e.g. Escherichia coli, modified to contain a gene or genes for expressing one or more enzymes capable of catalysis as described herein.

Where one or more microorganisms are employed, the enzymatic methods of the present invention may be carried out subsequent to the fermentation of the microorganism (two-stage fermentation and conversion), or concurrently therewith, that is, in the latter case, by in situ fermentation and conversion (single-stage fermentation and conversion).

Growth of the microorganisms can be achieved by one of ordinary skill in the art by the use of an appropriate medium. Appropriate media for growing microorganisms include those which provide nutrients necessary for the growth of the microbial cells. A typical medium for growth includes necessary carbon sources, nitrogen sources, and elements (e.g. in trace amounts). Inducers may also be added. The term “inducer”, as used herein, includes any compound enhancing formation of the desired enzymatic activity within the microbial cell.

Carbon sources can include sugars such as maltose, lactose, glucose, fructose, glycerol, sorbitol, sucrose, starch, mannitol, propylene glycol, and the like; organic acids such as sodium acetate, sodium citrate, and the like; and alcohols such as ethanol, propanol and the like.

Nitrogen sources can include N-Z amine A, corn steep liquor, soy bean meal, beef extracts, yeast extracts, molasses, baker's yeast, tryptone, nutrisoy, peptone, yeastamin, amino acids such as sodium glutamate and the like, sodium nitrate, ammonium sulfate and the like.

Trace elements can include magnesium, manganese, calcium, cobalt, nickel, iron, sodium and potassium salts. Phosphates may also be added in trace or, preferably, greater than trace amounts.

The medium employed can include more than one carbon or nitrogen source or other nutrient.

Preferred media for growth include aqueous media.

The agitation and aeration of the reaction mixture affects the amount of oxygen available during the conversion process when conducted, for example, in shake-flask cultures or fermentor tanks during growth of microorganisms.

Incubation of the reaction medium is preferably at a temperature between about 4 and about 60° C. The reaction time can be appropriately varied depending upon the amount of enzyme used and its specific activity. Reaction times may be reduced by increasing the reaction temperature and/or increasing the amount of enzyme added to the reaction solution.

It is also preferred to employ an aqueous liquid as the reaction medium, although an organic liquid, or a miscible or immiscible (biphasic) organic/aqueous liquid mixture, may also be employed. The amount of enzyme or microorganism employed relative to the starting material is selected to allow catalysis of the enzymatic conversions of the present invention.

Solvents for the organic phase of a biphasic solvent system may be any organic solvent immiscible in water, such as toluene, cyclohexane, xylene, trichlorotrifluoroethane and the like. The aqueous phase is conveniently of water, preferably deionized water, or a suitable aqueous buffer solution, especially a phosphate buffer solution. The biphasic solvent system preferably comprises between about 10 to 90 percent by volume of organic phase and between about 90 to 10 percent by volume of aqueous phase, and most preferably contains at or about 20 percent by volume of organic phase and at or about 80 percent by volume of the aqueous phase.

An exemplary embodiment of such processes starts with preparation of an aqueous solution of the enzyme(s) or microbes to be used. For example, the preferred enzyme(s) or microbes can be added to a suitable amount of an aqueous solvent, such as phosphate buffer or the like. This mixture is preferably adjusted to and maintained at a desired pH.

The compounds XVI and XVIII produced by the processes of the present invention can be isolated and purified, for example, by methods such as extraction, distillation, crystallization, and column chromatography.

### Preferred Compounds

A preferred subgenus of the compounds of the present invention includes compounds of the formula I or salts thereof wherein one or more, preferably all, of the following substituents are as defined below:

G is an aryl or heterocyclo (e.g., heteroaryl) group, where said group is mono- or polycyclic, and which is optionally substituted at one or more positions, preferably with hydrogen, alkyl or substituted alkyl, alkenyl or substituted alkenyl, alkynyl or substituted alkynyl, halo, cycloalkyl or substituted cycloalkyl, cycloalkenyl or substituted cycloalkenyl, aryl or substituted aryl, heterocyclo or substituted heterocyclo, arylalkyl or substituted arylalkyl, heterocycloalkyl or substituted heterocycloalkyl, CN, R¹OC═O, R¹C═O, R¹HNC═O, R¹R²NC═O, HOCR³R³′, nitro, R¹OCH₂, R¹O, NH₂, NR⁴R⁵, S═OR¹, SO₂R¹, SO₂NR¹R′, (R¹)(R¹′)P═O, or (R¹′)(NHR¹)P═O;

Z₁ is O, S, NH, or NR⁶;

Z₂ is O, S, NH, or NR⁶;

A₁ is CR⁷ or N;

A₂ is CR⁷ or N;

Y is J—J′—J″ where J is (CR⁷R⁷′)n and n=0-3, J′ is a bond or O, S, S═O, SO₂, NH, OC═O, C═O, NR⁷, CR⁷R⁷′, R²P═O, R²P═S, R²OP═O, R²NHP═O, OP═OOR², OP═ONHR², OP═OR², OSO₂, NHNH, NHNR⁶, NR⁶NH, N═N, cycloalkyl or substituted cycloalkyl, cycloalkenyl or substituted cycloalkenyl, or heterocyclo or substituted heterocyclo, and J″ is (CR⁷R⁷′)n and n=0-3, where Y is not a bond;

W is CR⁷R⁷′—CR⁷R⁷′, CR⁷R⁷′—C═O, NR⁹—CR⁷R⁷′, N═CR⁸, N═N, NR⁹—NR⁹—cycloalkyl or substituted cycloalkyl, cycloalkenyl or substituted cycloalkenyl, heterocyclo or substituted heterocyclo, or aryl or substituted aryl, wherein, when W is not NR⁹—CR⁷R⁷′, N═CR⁸, N═N, NR⁹—NR⁹′, or heterocyclo or substituted heterocyclo, then J′ must be O, S, S═O, SO₂, NH, NR⁷, OP═OOR², OP═ONHR², OSO₂, NHNH, NHNR⁶, NR⁶NH, or N═N;

Q₁ is H, alkyl or substituted alkyl, alkenyl or substituted alkenyl, cycloalkyl or substituted cycloalkyl, cycloalkenyl or substituted cycloalkenyl, heterocycloalkyl or substituted heterocycloalkyl, arylalkyl or substituted arylalkyl, alkynyl or substituted alkynyl, aryl or substituted aryl, heterocyclo (e.g., heteroaryl) or substituted heterocyclo (e.g., substituted heteroaryl), halo, CN, R¹OC═O, R⁴C═O, R⁵R⁶NC═O, HOCR⁷R⁷′, nitro, R¹OCH₂, R¹OCH, NH₂, or NR⁴R⁵;

Q₂ is H, alkyl or substituted alkyl, alkenyl or substituted alkenyl, cycloalkyl or substituted cycloalkyl, cycloalkenyl or substituted cycloalkenyl, heterocycloalkyl or substituted heterocycloalkyl, arylalkyl or substituted arylalkyl, alkynyl or substituted alkynyl, aryl or substituted aryl, heterocyclo (e.g., heteroaryl) or substituted heterocyclo (e.g., substituted heteroaryl), halo, CN, R¹OC═O, R⁴C═O, R⁵R⁶NC═O, HOCR⁷R⁷′, nitro, R¹OCH₂, R¹O, NH₂, or NR⁴R⁵;

L is a bond, (CR⁷R⁷′)n, NH, NR⁵ or NR⁵(CR⁷R⁷′)n, where n=0-3;

R¹ and R¹′ are each independently H, alkyl or substituted alkyl, cycloalkyl or substituted cycloalkyl, cycloalkenyl or substituted cycloalkenyl, heterocyclo or substituted heterocyclo, cycloalkylalkyl or substituted cycloalkyalkyl, cycloalkenylalkyl or substituted cycloalkenylalkyl, heterocycloalkyl or substituted heterocycloalkyl, aryl or substituted aryl, arylalkyl or substituted arylalkyl;

R² is alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl;

R³ and R³′ are each independently H, alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, halo, CN, hydroxylamine, hydroxamide, alkoxy or substituted alkoxy, amino, NR¹R², thiol, alkylthio or substituted alkylthio;

R⁴ is H, alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, R¹C═O, R¹NHC═O, or SO₂NR¹R¹′;

R⁵ is alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, R¹C═O, R¹NHC═O, SO₂R¹, or SO₂NR¹R¹′;

R⁶ is alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, CN, OH, OR¹, R¹C═O, R¹NHC═O, SO₂R¹, or SO₂NR¹R¹′;

R⁷ and R⁷′ are each independently H, alkyl or substituted alkyl, alkenyl or substituted alkenyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, halo, CN, OR¹, nitro, hydroxylamine, hydroxylamide, amino, NHR⁴, NR²R⁵, NOR¹, thiol, alkylthio or substituted alkylthio, R¹C═O, R¹OC═O, R¹NHC═O, SOR¹, PO₃R¹R¹′, R¹R¹′NC═O, C═OSR¹, SO₂R¹, or SO₂NR¹R¹′;

R⁸ and R⁸′ are each independently H, alkyl or substituted alkyl, alkenyl or substituted alkenyl, cycloalkyl or substituted cycloalkyl, cycloalkenyl or substituted cycloalkenyl, heterocyclo or substituted heterocyclo, cycloalkylalkyl or substituted cycloalkyalkyl, cycloalkenylalkyl or substituted cycloalkenylalkyl, heterocycloalkyl or substituted heterocycloalkyl, aryl or substituted aryl, arylalkyl or substituted arylalkyl, nitro, halo, CN, OR¹, amino, NHR⁴, NR²R⁵, NOR¹, alkylthio or substituted alkylthio, C═OSR¹, R¹OC═O, R¹C═O, R¹NHC═O, R¹R¹′NC═O, S═OR¹, SO₂R¹, PO₃R¹R¹′, or SO₂NR¹R¹′;

R⁹ and R⁹′ are each independently H, alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, CN, OH, OR¹, R¹C═O, R¹OC═O, R¹NHC═O, or SO₂NR¹R¹′;

especially where the groups W and Y of this preferred subgenus are also within the definitions of W′ and Y′ of formula Ia, with the provisos (1) to (14) of said formula Ia where appropriate to this subgenus, and most preferably where (i) when Y′ is —O— and W′ is CR⁷R⁷′—CR⁷R⁷′, A₁ and A₂ are not simultaneously CH; and (ii) when L is a bond, G is not an unsubstituted phenyl group.

Another, more preferred subgenus of the compounds of the invention includes compounds of the formula I or salts thereof wherein one or more, preferably all, of the following substituents are as defined below:

G is an aryl or heterocyclo (e.g., heteroaryl) group, where said group is mono- or polycyclic, and which is optionally substituted at one or more positions, preferably with hydrogen, alkyl or substituted alkyl, alkenyl or substituted alkenyl, alkynyl or substituted alkynyl, halo, cycloalkyl or substituted cycloalkyl, cycloalkenyl or substituted cycloalkenyl, aryl or substituted aryl, heterocyclo or substituted heterocyclo, arylalkyl or substituted arylalkyl, heterocycloalkyl or substituted heterocycloalkyl, CN, R¹C═O, R¹HNC═O, R¹R²NC═O, HOCR³R³′, nitro, R¹OCH₂, R¹O, NH₂, NR⁴R⁵, SO₂R¹, or SO₂NR¹R¹′;

Z₁ is O ;

Z₂ is O ;

A₁ is CR⁷;

A₂ is CR⁷;

Y is J—J′—J″ where J is (CR⁷R⁷′)n and n=0-3, J′ is a bond or O, S, S═O, SO₂, NH, NR⁷, CR⁷R⁷′, R²P═O, R²P═S, R²OP═O, R²NHP═O, OP═OOR, OP═ONHR², OP═OR², OSO₂, NHNH, NHNR⁶, NR⁶NH, N═N, cycloalkyl or substituted cycloalkyl, cycloalkenyl or substituted cycloalkenyl, or heterocyclo or substituted heterocyclo, and J″ is (CR⁷R⁷′)n and n=0-3, where Y is not a bond;

W is CR⁷R⁷′—CR⁷R⁷′, CR⁷R⁷′—C═O, NR⁹—CR⁷R⁷′, N═CR⁸, N═N, NR⁹—NR⁹′, cycloalkyl or substituted cycloalkyl, cycloalkenyl or substituted cycloalkenyl, heterocyclo or substituted heterocyclo, or aryl or substituted aryl, wherein, when W is not NR⁹—CR⁷R⁷′, N═CR⁸, N═N, NR⁹—NR⁹′, or heterocyclo or substituted heterocyclo, then J′ must be O, S, S═O, SO₂, NH, NR⁷, OP═OOR², OP═ONHR², OSO₂, NHNH, NHNR⁶, NR⁶NH, or N═N;

Q₁ is H, alkyl or substituted alkyl, alkenyl or substituted alkenyl, cycloalkyl or substituted cycloalkyl, cycloalkenyl or substituted cycloalkenyl, heterocycloalkyl or substituted heterocycloalkyl, arylalkyl or substituted arylalkyl, alkynyl or substituted alkynyl, aryl or substituted aryl, heterocyclo (e.g., heteroaryl) or substituted heterocyclo (e.g., substituted heteroaryl), halo, CN, R⁴C═O, R⁵R⁶NC═O, HOCR⁷R⁷′, nitro, R¹OCH₂, R¹O, NH₂, or NR⁴R⁵;

Q₂ is H, alkyl or substituted alkyl, alkenyl or substituted alkenyl, cycloalkyl or substituted cycloalkyl, cycloalkenyl or substituted cycloalkenyl, heterocycloalkyl or substituted heterocycloalkyl, arylalkyl or substituted arylalkyl, alkynyl or substituted alkynyl, aryl or substituted aryl, heterocyclo (e.g., heteroaryl) or substituted heterocyclo (e.g., substituted heteroaryl), halo, CN, R⁴C═O, R⁵R⁶NC═O, HOCR⁷R⁷′, nitro, R¹OCH₂, R¹O, NH₂, or NR⁴R⁵;

L is a bond;

R¹ and R¹′ are each independently H, alkyl or substituted alkyl, cycloalkyl or substituted cycloalkyl, cycloalkenyl or substituted cycloalkenyl, heterocyclo or substituted heterocyclo, cycloalkylalkyl or substituted cycloalkyalkyl, cycloalkenylalkyl or substituted cycloalkenylalkyl, heterocycloalkyl or substituted heterocycloalkyl, aryl or substituted aryl, arylalkyl or substituted arylalkyl;

R² is alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl;

R³ and R³′ are each independently H, alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, halo, CN, alkoxy or substituted alkoxy, amino, NR¹R², alkylthio or substituted alkylthio;

R⁴ is H, alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, R¹C═O, R¹NHC═O, or SO₂NR¹R¹′;

R⁵ is alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, R¹C═O, R¹NHC═O, SO₂R¹, or SO₂NR¹R¹′;

R⁶ is alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, CN, OH, OR¹, R¹C═O, R¹NHC═O, SO₂R¹, or SO₂NR¹R¹′;

R⁷ and R⁷′ are each independently H, alkyl or substituted alkyl, alkenyl or substituted alkenyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, halo, CN, OR¹, nitro, amino, NHR⁴, NR²R⁵, alkylthio or substituted alkylthio, R¹C═O, R¹NHC═O, SO₂R¹, R¹R¹′NC═O, or SO₂NR¹R¹′;

R⁸ and R⁸′ are each independently H, alkyl or substituted alkyl, alkenyl or substituted alkenyl, cycloalkyl or substituted cycloalkyl, cycloalkenyl or substituted cycloalkenyl, heterocyclo or substituted heterocyclo, cycloalkylalkyl or substituted cycloalkyalkyl, cycloalkenylalkyl or substituted cycloalkenylalkyl, heterocycloalkyl or substituted heterocycloalkyl, aryl or substituted aryl, arylalkyl or substituted arylalkyl, nitro, halo, CN, OR¹, amino, NHR⁴, NR²R⁵, alkylthio or substituted alkylthio, R¹C═O, R¹NHC═O, R¹R¹′NC═O, SO₂R¹, or SO₂NR¹R¹′; and

R⁹ and R⁹′ are each independently H, alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, CN, OH, OR¹, R¹C═O, R¹NHC═O, or SO₂NR¹R¹′;

especially where the groups W and Y of this preferred subgenus are also within the definitions of W′ and Y′ of formula Ia, with the provisos (1) to (14) of said formula Ia where appropriate to this subgenus, and most preferably where (i) when Y′ is —O— and W′ is CR⁷R⁷′—CR⁷R⁷′, A₁ and A₂ are not simultaneously CH; and (ii) when L is a bond, G is not an unsubstituted phenyl group.

A particularly preferred subgenus of the compounds of the invention includes compounds of the formula I or salts thereof wherein one or more, preferably all, of the substituents are as defined below:

G is an aryl (especially, phenyl or naphthyl) or heterocyclo (especially those heterocyclo groups G of the compounds of the Examples herein) group, where said group is mono- or polycyclic, and which is optionally substituted at one or more positions, preferably with substituents as exemplified in any of the compounds of the Examples herein;

L is a bond, (CR⁷R⁷′)n (where n is 1 and R⁷ and R⁷′ are each independently H, alkyl or substituted alkyl), or —CH2—NH—;

A₁ and A₂ are each independently CR⁷ where R⁷ (i) is hydrogen, alkyl or substituted alkyl, arylalkyl or substituted arylalkyl, alkenyl or substituted alkenyl (for example, alkenyl substituted with aryl (especially, phenyl or naphthyl) or substituted aryl, or alkenyl substituted with heterocyclo or substituted heterocyclo), aryl or substituted aryl, heterocyclo or substituted heterocyclo, heterocycloalkyl or substituted heterocycloalkyl, where, for each, preferred substituents are one or more groups selected from V¹ (especially A₁ and A₂ groups of the formula CR⁷ where R⁷ for each of A₁ and/or A₂ is independently selected from C₁₋₄alkyl which alkyl is substituted by one or more groups V¹), or (ii) forms, together with R⁷ of a group W (especially where W is CR⁷R⁷′—CR⁷R⁷′), a heterocyclic ring;

V¹ is OH, CN, halo, —O-aryl, —O-substituted aryl, —O-heterocyclo, —O-substituted heterocyclo, —O—CO-alkyl, —O—CO-substituted alkyl, —O-(alkylsilyl), —O-arylalkyl, —O-substituted arylalkyl, —O—CO-alkyl, —O—CO-substituted alkyl, —O-CO-arylalkyl, —O-CO-substituted arylalkyl, —O—CO-aryl, —O-CO-substituted aryl, —O—CO-heterocyclo, —O—CO-substituted heterocyclo, —S-(optionally substituted aryl)-NH—CO-(optionally substituted alkyl), —SO-(optionally substituted aryl)-NH—CO-(optionally substituted alkyl), —SO2-(optionally substituted aryl)-NH—CO-(optionally substituted alkyl), —NH—SO2-aryl, —NH—SO2-substituted aryl, —NH—CO—O-(optionally substituted arylalkyl), —NH—CO—O-alkyl, —NH-CO—O-substituted alkyl, —NH—CO-alkyl, —NH—CO-substituted alkyl, —NH—CO-aryl, —NH—CO-substituted aryl, —NH—CO-(optionally substituted arylalkyl), —NH—CO-(optionally substituted alkyl)-O-(optionally substituted aryl), —N(optionally substituted alkyl)(optionally substituted aryl), —N(optionally substituted alkyl)(optionally substituted arylalkyl), —COH, —COOH, —CO—O-alkyl, —CO—O-substituted alkyl, —CO—O-optionally substituted arylalkyl, —CO-aryl, —CO-substituted aryl, —O—CO—NH-aryl, —O—CO—NH-substituted aryl, —CO—NH-aryl, —CO—NH-substituted aryl, —CO—NH-arylalkyl, —CO—NH-substituted arylalkyl, —O-(optionally substituted aryl)-NH—CO-(optionally substituted alkyl);

Y is —O—, —SO—, —N(V²)-, —CH₂—N(V²)-, —CO—N(alkyl)-, —CH₂—S , —CH₂—SO₂—;

V² is hydrogen, alkyl, arylalkyl, —CO-alkyl, —CO—O-aryl, —CO—O-arylalkyl;

W is CR⁷R⁷′ CR⁷R⁷′ (where R⁷ and R⁷′ are each independently selected from H, OH, alkyl or substituted alkyl (such as hydroxyalkyl), or where R⁷ forms a heterocyclic ring together with R⁷ of A₁ or A₂), CR⁸═CR⁸′ (where R⁸ and R⁸′ are each independently selected from H, alkyl or substituted alkyl (such as hydroxyalkyl)), CR⁷R⁷′—C═O (where R⁷ and R⁷′ are each hydrogen, or where R⁷ forms a heterocyclic ring together with R⁷ of A₁ or A₂), N═CR⁸ (where R⁸ is alkyl), cycloalkyl or substituted cyclalkyl, or heterocyclo or substituted heterocyclo;

Z₁ and Z₂ are O ; and

Q₁ and Q₂ are H.

Preferred G—L groups are optionally substituted naphthyl and optionally substituted fused bicyclic heterocyclic groups such as optionally substituted benzo-fused heterocyclic groups (e.g., bonded to the remainder of the molecule through the benzene portion), especially such groups wherein the heterocyclic ring bonded to benzene has 5 members exemplified by benzoxazole, benzothiazole, benzothiadiazole, benzoxadiazole or benzothiophene, for example:

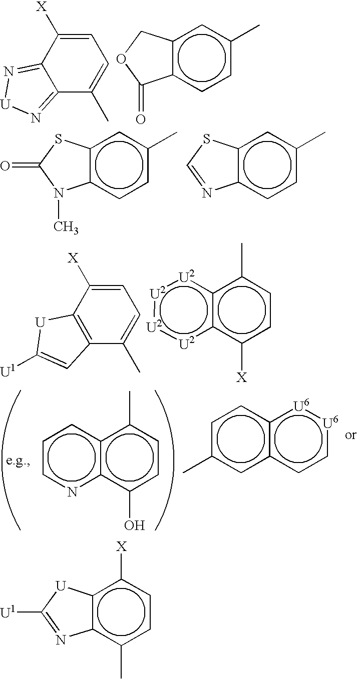

where

X=halo (esp F), OH, CN, NO₂ or

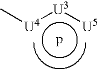

(e.g.,

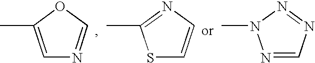

);

U is O or S (where S can optionally be oxygenated, e.g., to SO);

U¹ is CH₃ or CF₃;

each U² is independently N, CH or CF;

U³ is N, O or S;

U⁴ and U⁵, together with the atoms to which they are bonded, form an optionally substituted 5-membered heterocyclic ring which can be partially unsaturated or aromatic and which contains 1 to 3 ring heteroatoms;

each U⁶ is independently CH or N; and

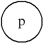

denotes optional double bond(s) within the ring formed by U³, U⁴ and U⁵.

An especially preferred subgenus includes compounds of the formula I having the following structure, or salts thereof:

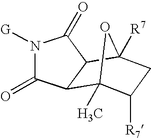

where G is an optionally substituted naphthyl or benzo-fused bicyclic heterocyclic group, R⁷ is CH₃ or C₁₋₄alkyl substituted by V¹ and R⁷′ is H or hydroxyl.

Compounds where R⁷′ is hydroxyl can provide enhanced water solubility and metabolic stability, relative to the corresponding compounds where R⁷′ is H, in addition to having good permeability and high systemic blood levels. These hydroxyl-bearing compounds can be obtained in vivo by metabolism of the corresponding compound where R⁷′ is H, as well as by synthetic preparative methods such as those described herein.

### Use and Utility

The compounds of the present invention modulate the function of nuclear hormone receptors (NHR), and include compounds which are, for example, agonists, partial agonists, antagonists or partial antagonists of the androgen receptor (AR), the estrogen receptor (ER), the progesterone receptor (PR), the glucocorticoid receptor (GR), the mineralocorticoid receptor (MR), the steroid and xenobiotic receptor (SXR), other steroid binding NHR's, the Orphan receptors or other NHR's. Selective modulation of one such NHR relative to others within the NHR family is preferred. “Modulation” includes, for example, activation (e.g., agonist activity such as selective androgen receptor agonist activity) or inhibition (e.g., antagonist activity).

The present compounds are thus useful in the treatment of NHR-associated conditions. A “NHR-associated condition”, as used herein, denotes a condition or disorder which can be treated by modulating the function of a NHR in a subject, wherein treatment comprises prevention (e.g., prophylactic treatment), partial alleviation or cure of the condition or disorder. Modulation may occur locally, for example, within certain tissues of the subject, or more extensively throughout a subject being treated for such a condition disorder.

The compounds of the present invention are useful for the treatment of a variety of conditions and disorders including, but not limited to, those described following:

Compounds of formula I can be applied as agonists, partial agonists, antagonists, or partial antagonists of the estrogen receptor, preferably selectively to that receptor, in an array of medical conditions which involve modulation of the estrogen receptor pathway. Applications of said compounds include but are not limited to: osteoporosis, hot flushes, vaginal dryness, prostate cancer, breast cancer, endometrial cancer, cancers expressing the estrogen receptor such as the aforementioned cancers and others, contraception, pregnancy termination, menopause, amennoreahea, and dysmennoreahea.

Compounds of formula I can be applied as agonists, partial agonists, antagonists or partial antagonists of the progesterone receptor, preferably selectively to that receptor, in an array of medical conditions which involve modulation of the progesterone receptor pathway. Applications of said compounds include but are not limited to: breast cancer, other cancers containing the progesterone receptor, endometriosis, cachexia, contraception, menopause, cyclesynchrony, meniginoma, dysmennoreahea, fibroids, pregnancy termination, labor induction and osteoporosis.

Compounds of formula I can be applied as agonists, partial agonists, antagonists or partial antagonists of the glucocorticoid receptor, preferably selectively to that receptor, in an array of medical conditions which involve modulation of the glucocorticoid receptor pathway. Applications of said compounds include but are not limited to: inflammatory diseases, autoimmune diseases, prostate cancer, breast cancer, Alzheimer's disease, psychotic disorders, drug dependence, non-insulin dependent Diabetes Mellitus, and as dopamine receptor blocking agents or otherwise as agents for the treatment of dopamine receptor mediated disorders.

Compounds of formula I can be applied as agonists, partial agonists, antagonists or partial antagonists of the mineralocorticoid receptor, preferably selectively to that receptor, in an array of medical conditions which involve modulation of the mineralocorticoid receptor pathway. Applications of said compounds include but are not limited to: drug withdrawal syndrome and inflammatory diseases.

Compounds of formula I can be applied as agonists, partial agonists, antagonists or partial antagonists of the aldosterone receptor, preferably selectively to that receptor, in an array of medical conditions which involve modulation of the aldosterone receptor pathway. One application of said compounds includes but is not limited to: congestive heart failure.

Compounds of formula I can be applied as agonists, partial agonists, antagonists or partial antagonists of the androgen receptor, preferably selectively to that receptor, in an array of medical conditions which involve modulation of the androgen receptor pathway. Applications of said compounds include but are not limited to: hirsutism, acne, seborrhea, Alzheimer's disease, androgenic alopecia, hypogonadism, hyperpilosity, benign prostate hypertrophia, adenomas and neoplasies of the prostate (such as advanced metastatic prostate cancer), treatment of benign or malignant tumor cells containing the androgen receptor such as is the case for breast, brain, skin, ovarian, bladder, lymphatic, liver and kidney cancers, pancreatic cancers modulation of VCAM expression and applications therein for the treatment of heart disease, inflammation and immune modulations, modulation of VEGF expression and the applications therein for use as antiangiogenic agents, osteoporosis, suppressing spermatogenesis, libido, cachexia, endometriosis, polycystic ovary syndrome, anorexia, androgen supplement for age related decreased testosterone levels in men, male menopause, male hormone replacement, male and female sexual dysfunction, and inhibition of muscular atrophy in ambulatory patients. For example, pan AR modulation is contemplated, with prostate selective AR modulation (“SARM”) being particularly preferred, such as for the treatment of early stage prostate cancers.

Compounds of formula I can be applied as (preferably, selective) antagonists of the mutated androgen receptor, for example, found in many tumor lines. Examples of such mutants are those found in representative prostate tumor cell lines such as LNCap, (T877A mutation, Biophys. Acta, 187, 1052 (1990)), PCa2b, (L701H & T877A mutations, J. Urol., 162, 2192 (1999)) and CVWR22, (H874Y mutation, Mol. Endo., 11, 450 (1997)). Applications of said compounds include but are not limited to: adenomas and neoplasies of the prostate, breast cancer and endometrial cancer.

Compounds of formula I can be applied as agonists, partial agonists, antagonists or partial antagonists of the steroid and xenobiotic receptor, preferably selectively to that receptor, in an array of medical conditions which involve modulation of the steroid and xenobiotic receptor pathway. Applications of said compounds include but are not limited to: treatment of disregulation of cholesterol homeostasis, attenuation of metabolism of pharmaceutical agents by co-administration of an agent (compound of the present invention) which modulates the P450 regulator effects of SXR.

Along with the aforementioned NHR, there also exist a number of NHR for which the activating or deactivating ligands may not be characterized. These proteins are classified as NHR due to strong sequence homology to other NHR, and are known as the Orphan receptors. Because the Orphan receptors demonstrate strong sequence homology to other NHR, compounds of formula I include those which serve as modulators of the function of the Orphan NHR. Orphan receptors which are modulated by NHR modulators such as compounds within the scope of formula I are exemplified, but not limited to, those listed in Table 1. Exemplary therapeutic applications of modulators of said Orphan receptors are also listed in Table 1, but are not limited to the examples therein.

TABLE 1
Exemplary Orphan nuclear hormone receptors, form
(M = monomeric, D = heterodimeric,
H = homodimeric), tissue expression and target therapeutic
applications. (CNS = central nervous system)

| | | | Target |
|---|---|---|---|
| Receptor | Form | Tissue Expression | Therapeutic Application |
| NURR1 | M/D | Dopaminergic Neurons | Parkinson's Disease |
| RZRβ | M | Brain (Pituitary), Muscle | Sleep Disorders |
| RORα | M | Cerebellum, Purkinje | Arthritis, Cerebellar |
| | | Cells | Ataxia |
| NOR-1 | M | Brain, Muscle, Heart, | CNS Disorders, |
| | | Adrenal, Thymus | Cancer |
| NGFI-Bβ | M/D | Brain | CNS Disorders |
| COUP-Tfα | H | Brain | CNS Disorders |
| COUP-TFβ | H | Brain | CNS Disorders |
| COUP-TFγχ | H | Brain | CNS Disorders |
| Nur77 | H | Brain, Thymus, Adrenals | CNS Disorders |
| Rev-ErbAα | H | Muscle, Brain | Obesity |
| | | (Ubiquitous) | |
| HNF4α | H | Liver, Kidney, Intestine | Diabetes |
| SF-1 | M | Gonads, Pituitary | Metabolic Disorders |
| LXRα,β | D | Kidney (Ubiquitous) | Metabolic Disorders |
| GCNF | M/H | Testes, Ovary | Infertility |
| ERRα,β | M | Placenta, Bone | Infertility, |
| | | | Osteoporosis |
| FXR | D | Liver, Kidney | Metabolic Disorders |
| CARα | H | Liver, Kidney | Metabolic Disorders |
| PXR | H | Liver, Intestine | Metabolic Disorders |

The present invention thus provides methods for the treatment of NHR-associated conditions, comprising the step of administering to a subject in need thereof at least one compound of formula I in an amount effective therefor. Other therapeutic agents such as those described below may be employed with the inventive compounds in the present methods (for example, separately, or formulated together as a fixed dose). In the methods of the present invention, such other therapeutic agent(s) can be administered prior to, simultaneously with or following the administration of the compound(s) of the present invention.

The present invention also provides pharmaceutical compositions comprising at least one of the compounds of the formula I capable of treating a NHR-associated condition in an amount effective therefor, and a pharmaceutically acceptable carrier (vehicle or diluent). The compositions of the present invention can contain other therapeutic agents as described below, and can be formulated, for example, by employing conventional solid or liquid vehicles or diluents, as well as pharmaceutical additives of a type appropriate to the mode of desired administration (for example, excipients, binders, preservatives, stabilizers, flavors, etc.) according to techniques such as those well known in the art of pharmaceutical formulation.

It should be noted that the compounds of the present invention are, without limitation as to their mechanism of action, useful in treating any of the conditions or disorders listed or described herein such as inflammatory diseases or cancers, or other proliferate diseases, and in compositions for treating such conditions or disorders. Such conditions and disorders include, without limitation, any of those described previously, as well as those described following such as: maintenance of muscle strength and function (e.g., in the elderly); reversal or prevention of frailty or age-related functional decline (“ARFD”) in the elderly (e.g., sarcopenia); treatment of catabolic side effects of glucocorticoids; prevention and/or treatment of reduced bone mass, density or growth (e.g., osteoporosis and osteopenia); treatment of chronic fatigue syndrome (CFS); chronic malagia; treatment of acute fatigue syndrome and muscle loss following elective surgery (e.g., post-surgical rehabilitation); acceleration of wound healing; accelerating bone fracture repair (such as accelerating the recovery of hip fracture patients); accelerating healing of complicated fractures, e.g. distraction osteogenesis; in joint replacement; prevention of post-surgical adhesion formation; acceleration of tooth repair or growth; maintenance of sensory function (e.g., hearing, sight, olefaction and taste); treatment of periodontal disease; treatment of wasting secondary to fractures and wasting in connection with chronic obstructive pulmonary disease (COPD), chronic liver disease, AIDS, weightlessness, cancer cachexia, bum and trauma recovery, chronic catabolic state (e.g., coma), eating disorders (e.g., anorexia) and chemotherapy; treatment of cardiomyopathy; treatment of thrombocytopenia; treatment of growth retardation in connection with Crohn's disease; treatment of short bowel syndrome; treatment of irritable bowel syndrome; treatment of inflammatory bowel disease; treatment of Crohn's disease and ulcerative colits; treatment of complications associated with transplantation; treatment of physiological short stature including growth hormone deficient children and short stature associated with chronic illness; treatment of obesity and growth retardation associated with obesity; treatment of anorexia (e.g., associated with cachexia or aging); treatment of hypercortisolism and Cushing's syndrome; Paget's disease; treatment of osteoarthritis; induction of pulsatile growth hormone release; treatment of osteochondrodysplasias; treatment of depression, nervousness, irritability and stress; treatment of reduced mental energy and low self-esteem (e.g., motivation/assertiveness); improvement of cognitive function (e.g., the treatment of dementia, including Alzheimer's disease and short term memory loss); treatment of catabolism in connection with pulmonary dysfunction and ventilator dependency; treatment of cardiac dysfunction (e.g., associated with valvular disease, myocardial infarction, cardiac hypertrophy or congestive heart failure); lowering blood pressure; protection against ventricular dysfunction or prevention of reperfusion events; treatment of adults in chronic dialysis; reversal or slowing of the catabolic state of aging; attenuation or reversal of protein catabolic responses following trauma (e.g., reversal of the catabolic state associated with surgery, congestive heart failure, cardiac myopathy, burns, cancer, COPD etc.); reducing cachexia and protein loss due to chronic illness such as cancer or AIDS; treatment of hyperinsulinemia including nesidioblastosis; treatment of immunosuppressed patients; treatment of wasting in connection with multiple sclerosis or other neurodegenerative disorders; promotion of myelin repair; maintenance of skin thickness; treatment of metabolic homeostasis and renal homeostasis (e.g., in the frail elderly); stimulation of osteoblasts, bone remodeling and cartilage growth; regulation of food intake; treatment of insulin resistance, including NIDDM, in mammals (e.g., humans); treatment of insulin resistance in the heart; improvement of sleep quality and correction of the relative hyposomatotropism of senescence due to high increase in REM sleep and a decrease in REM latency; treatment of hypothermia; treatment of congestive heart failure; treatment of lipodystrophy (e.g., in patients taking HIV or AIDS therapies such as protease inhibitors); treatment of muscular atrophy (e.g., due to physical inactivity, bed rest or reduced weight-bearing conditions); treatment of musculoskeletal impairment (e.g., in the elderly); improvement of the overall pulmonary function; treatment of sleep disorders; and the treatment of the catabolic state of prolonged critical illness; treatment of hirsutism, acne, seborrhea, androgenic alopecia, anemia, hyperpilosity, benign prostate hypertrophy, adenomas and neoplasies of the prostate (e.g., advanced metastatic prostate cancer) and malignant tumor cells containing the androgen receptor, such as is the case for breast, brain, skin, ovarian, bladder, lymphatic, liver and kidney cancers; cancers of the skin, pancreas, endometrium, lung and colon; osteosarcoma; hypercalcemia of malignancy; metastatic bone disease; treatment of spermatogenesis, endometriosis and polycystic ovary syndrome; conteracting preeclampsia, eclampsia of pregnancy and preterm labor; treatment of premenstrual syndrome; treatment of vaginal dryness; age related decreased testosterone levels in men, male menopause, hypogonadism, male hormone replacement, male and female sexual dysfunction (e.g., erectile dysfunction, decreased sex drive, sexual well-being, decreased libido), male and female contraception, hair loss, Reaven's Syndrome and the enhancement of bone and muscle performance/strength; and the conditions, diseases, and maladies collectively referenced to as “Syndrome X” or Metabolic Syndrome as detailed in Johannsson J. Clin. Endocrinol. Metab., 82, 727-34 (1997).

The present compounds have therapeutic utility in the modulation of immune cell activation/proliferation, e.g., as competitive inhibitors of intercellular ligand/receptor binding reactions involving CAMs (Cellular Adhesion Molecules) and Leukointegrins. For example, the present compounds modulate LFA-ICAM 1, and are particularly useful as LFA-ICAM 1 antagonists, and in the treatment of all conditions associated with LFA-ICAM 1 such as immunological disorders. Preferred utilities for the present compounds include, but are not limited to: inflammatory conditions such as those resulting from a response of the non-specific immune system in a mammal (e.g., adult respiratory distress syndrome, shock, oxygen toxicity, multiple organ injury syndrome secondary to septicemia, multiple organ injury syndrome secondary to trauma, reperfusion injury of tissue due to cardiopulmonary bypass, myocardial infarction or use with thrombolysis agents, acute glomerulonephritis, vasculitis, reactive arthritis, dermatosis with acute inflammatory components, stroke, thermal injury, hemodialysis, leukapheresis, ulcerative colitis, necrotizing enterocolitis and granulocyte transfusion associated syndrome) and conditions resulting from a response of the specific immune system in a mammal (e.g., psoriasis, organ/tissue transplant rejection, graft vs. host reactions and autoimmune diseases including Raynaud's syndrome, autoimmune thyroiditis, dermatitis, multiple sclerosis, rheumatoid arthritis, insulin-dependent diabetes mellitus, uveitis, inflammatory bowel disease including Crohn's disease and ulcerative colitis, and systemic lupus erythematosus). The present compounds can be used in treating asthma or as an adjunct to minimize toxicity with cytokine therapy in the treatment of cancers. The present compounds can be employed in the treatment of all diseases currently treatable through steroid therapy. The present compounds may be employed for the treatment of these and other disorders alone or with other immunosuppressive or antiinflammatory agents. In accordance with the invention, a compound of the formula I can be administered prior to the onset of inflammation (so as to suppress an anticipated inflammation) or after the initiation of inflammation. When provided prophylactically, the immunosupressive compound(s) are preferably provided in advance of any inflammatory response or symptom (for example, prior to, at, or shortly after the time of an organ or tissue transplant but in advance of any symptoms or organ rejection). The prophylactic administration of a compound of the formula I prevents or attenuates any subsequent inflammatory response (such as, for example, rejection of a transplanted organ or tissue, etc.) Administration of a compound of the formula I attenuates any actual inflammation (such as, for example, the rejection of a transplanted organ or tissue).

The compounds of the formula I can be administered for any of the uses described herein by any suitable means, for example, orally, such as in the form of tablets, capsules, granules or powders; sublingually; bucally; parenterally, such as by subcutaneous, intravenous, intramuscular, or intrasternal injection or infusion techniques (e.g., as sterile injectable aqueous or non-aqueous solutions or suspensions); nasally, including administration to the nasal membranes, such as by inhalation spray; topically, such as in the form of a cream or ointment; or rectally such as in the form of suppositories; in dosage unit formulations containing non-toxic, pharmaceutically acceptable vehicles or diluents. The present compounds can, for example, be administered in a form suitable for immediate release or extended release. Immediate release or extended release can be achieved by the use of suitable pharmaceutical compositions comprising the present compounds, or, particularly in the case of extended release, by the use of devices such as subcutaneous implants or osmotic pumps. The present compounds can also be administered liposomally.

Exemplary compositions for oral administration include suspensions which can contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which can contain, for example, microcrystalline cellulose, dicalcium phosphate, starch, magnesium stearate and/or lactose and/or other excipients, binders, extenders, disintegrants, diluents and lubricants such as those known in the art. The compounds of formula I can also be delivered through the oral cavity by sublingual and/or buccal administration. Molded tablets, compressed tablets or freeze-dried tablets are exemplary forms which may be used. Exemplary compositions include those formulating the present compound(s) with fast dissolving diluents such as mannitol, lactose, sucrose and/or cyclodextrins. Also included in such formulations may be high molecular weight excipients such as celluloses (avicel) or polyethylene glycols (PEG). Such formulations can also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g. Carbopol 934). Lubricants, glidants, flavors, coloring agents and stabilizers may also be added for ease of fabrication and use.

Exemplary compositions for nasal aerosol or inhalation administration include solutions in saline which can contain, for example, benzyl alcohol or other suitable preservatives, absorption promoters to enhance bioavailability, and/or other solubilizing or dispersing agents such as those known in the art.

Exemplary compositions for parenteral administration include injectable solutions or suspensions which can contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor.

Exemplary compositions for rectal administration include suppositories which can contain, for example, a suitable non-irritating excipient, such as cocoa butter, synthetic glyceride esters or polyethylene glycols, which are solid at ordinary temperatures, but liquify and/or dissolve in the rectal cavity to release the drug.

Exemplary compositions for topical administration include a topical carrier such as Plastibase (mineral oil gelled with polyethylene).

The effective amount of a compound of the present invention can be determined by one of ordinary skill in the art, and includes exemplary dosage amounts for a adult human of from about 1 to 100 (for example, 15) mg/kg of body weight of active compound per day, which can be administered in a single dose or in the form of individual divided doses, such as from 1 to 4 times per day. It will be understood that the specific dose level and frequency of dosage for any particular subject can be varied and will depend upon a variety of factors including the activity of the specific compound employed, the metabolic stability and length of action of that compound, the species, age, body weight, general health, sex and diet of the subject, the mode and time of administration, rate of excretion, drug combination, and severity of the particular condition. Preferred subjects for treatment include animals, most preferably mammalian species such as humans, and domestic animals such as dogs, cats and the like, subject to NHR-associated conditions.

As mentioned above, the compounds of the present invention can be employed alone or in combination with each other and/or other suitable therapeutic agents useful in the treatment of NHR-associated conditions, e.g., an antibiotic or other pharmaceutically active material.

For example, the compounds of the present invention can be combined with growth promoting agents, such as, but not limited to, TRH, diethylstilbesterol, theophylline, enkephalins, E series prostaglandins, compounds disclosed in U.S. Pat. No. 3,239,345, e.g., zeranol, and compounds disclosed in U.S. Pat. No. 4,036,979, e.g., sulbenox or peptides disclosed in U.S. Pat. No. 4,411,890.

The compounds of the invention can also be used in combination with growth hormone secretagogues such as GHRP-6, GHRP-1 (as described in U.S. Pat. No. 4,411,890 and publications WO 89/07110 and WO 89/07111), GHRP-2 (as described in WO 93/04081), NN703 (Novo Nordisk), LY444711 (Lilly), MK-677 (Merck), CP424391 (Pfizer) and B-HT920, or with growth hormone releasing factor and its analogs or growth hormone and its analogs or somatomedins including IGF-1 and IGF-2, or with alpha-adrenergic agonists, such as clonidine or serotinin 5-HT_{D} agonists, such as sumatriptan, or agents which inhibit somatostatin or its release, such as physostigmine and pyridostigmine. A still further use of the disclosed compounds of the invention is in combination with parathyroid hormone, PTH(1-34) or bisphosphonates, such as MK-217 (alendronate).

A still further use of the compounds of the invention is in combination with estrogen, testosterone, a selective estrogen receptor modulator, such as tamoxifen or raloxifene, or other androgen receptor modulators, such as those disclosed in Edwards, J. P. et al., Bio. Med. Chem. Let., 9, 1003-1008 (1999) and Hamann, L. G. et al., J. Med. Chem., 42, 210-212 (1999).

A further use of the compounds of this invention is in combination with progesterone receptor agonists (“PRA”), such as levonorgestrel, medroxyprogesterone acetate (MPA).

The compounds of the present invention can be employed alone or in combination with each other and/or other modulators of nuclear hormone receptors or other suitable therapeutic agents useful in the treatment of the aforementioned disorders including: anti-diabetic agents; anti-osteoporosis agents; anti-obesity agents; anti-inflammatory agents; anti-anxiety agents; anti-depressants; anti-hypertensive agents; anti-platelet agents; anti-thrombotic and thrombolytic agents; cardiac glycosides; cholesterol/lipid lowering agents; mineralocorticoid receptor antagonists; phospodiesterase inhibitors; protein tyrosine kinase inhibitors; thyroid mimetics (including thyroid receptor agonists); anabolic agents; HIV or AIDS therapies; therapies useful in the treatment of Alzheimer's disease and other cognitive disorders; therapies useful in the treatment of sleeping disorders; anti-proliferative agents; and anti-tumor agents.

Examples of suitable anti-diabetic agents for use in combination with the compounds of the present invention include biguanides (e.g., metformin), glucosidase inhibitors (e.g,. acarbose), insulins (including insulin secretagogues or insulin sensitizers), meglitinides (e.g., repaglinide), sulfonylureas (e.g., glimepiride, glyburide and glipizide), biguanide/glyburide combinations (e.g., Glucovance®), thiazolidinediones (e.g., troglitazone, rosiglitazone and pioglitazone), PPAR-alpha agonists, PPAR-gamma agonists, PPAR alpha/gamma dual agonists, SGLT2 inhibitors, glycogen phosphorylase inhibitors, inhibitors of fatty acid binding protein (aP2) such as those disclosed in U.S. Ser. No. 09/519,079 filed Mar. 6, 2000, glucagon-like peptide-1 (GLP-1), and dipeptidyl peptidase IV (DP4) inhibitors.

Examples of suitable anti-osteoporosis agents for use in combination with the compounds of the present invention include alendronate, risedronate, PTH, PTH fragment, raloxifene, calcitonin, steroidal or non-steroidal progesterone receptor agonists, RANK ligand antagonists, calcium sensing receptor antagonists, TRAP inhibitors, selective estrogen receptor modulators (SERM), estrogen and AP-1 inhibitors.

Examples of suitable anti-obesity agents for use in combination with the compounds of the present invention include aP2 inhibitors, such as those disclosed in U.S. Ser. No. 09/519,079 filed Mar. 6, 2000, PPAR gamma antagonists, PPAR delta agonists, beta 3 adrenergic agonists, such as AJ9677 (Takeda/Dainippon), L750355 (Merck), or CP331648 (Pfizer) or other known beta 3 agonists as disclosed in U.S. Pat. Nos. 5,541,204, 5,770,615, 5,491,134, 5,776,983 and 5,488,064, a lipase inhibitor, such as or list at or ATL-962 (Alizyme), a serotonin (and dopamine) reuptake inhibitor, such as sibutramine, topiramate (Johnson & Johnson) or axokine (Regeneron), a thyroid receptor beta drug, such as a thyroid receptor ligand as disclosed in WO 97/21993 (U. Cal SF), WO 99/00353 (KaroBio) and GB98/284425 (KaroBio), and/or an anorectic agent, such as dexamphetamine, phentermine, phenylpropanolamine or mazindol.

Examples of suitable anti-inflammatory agents for use in combination with the compounds of the present invention include prednisone, dexamethasone, Enbrel®, cyclooxygenase inhibitors (i.e., COX-1 and/or COX-2 inhibitors such as NSAIDs, aspirin, indomethacin, ibuprofen, piroxicam, Naproxen®, Celebrex®, Vioxx®), CTLA4-Ig agonists/antagonists, CD40 ligand antagonists, IMPDH inhibitors, such as mycophenolate (CellCept®) integrin antagonists, alpha-4 beta-7 integrin antagonists, cell adhesion inhibitors, interferon gamma antagonists, ICAM-1, tumor necrosis factor (TNF) antagonists (e.g., infliximab, OR1384), prostaglandin synthesis inhibitors, budesonide, clofazimine, CNI-1493, CD4 antagonists (e.g., priliximab), p38 mitogen-activated protein kinase inhibitors, protein tyrosine kinase (PTK) inhibitors, IKK inhibitors, and therapies for the treatment of irritable bowel syndrome (e.g., Zelmac® and Maxi-K® openers such as those disclosed in U.S. Pat. No. 6,184,231 B1).

Example of suitable anti-anxiety agents for use in combination with the compounds of the present invention include diazepam, lorazepam, buspirone, oxazepam, and hydroxyzine pamoate.

Examples of suitable anti-depressants for use in combination with the compounds of the present invention include citalopram, fluoxetine, nefazodone, sertraline, and paroxetine.

Examples of suitable anti-hypertensive agents for use in combination with the compounds of the present invention include beta adrenergic blockers, calcium channel blockers (L-type and T-type; e.g. diltiazem, verapamil, nifedipine, amlodipine and mybefradil), diuretics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, furosemide, musolimine, bumetanide, triamtrenene, amiloride, spironolactone), renin inhibitors, ACE inhibitors (e.g., captopril, zofenopril, fosinopril, enalapril, ceranopril, cilazopril, delapril, pentopril, quinapril, ramipril, lisinopril), AT-1 receptor antagonists (e.g., losartan, irbesartan, valsartan), ET receptor antagonists (e.g., sitaxsentan, atrsentan and compounds disclosed in U.S. Pat. Nos. 5,612,359 and 6,043,265), Dual ET/AII antagonist (e.g., compounds disclosed in WO 00/01389), neutral endopeptidase (NEP) inhibitors, vasopepsidase inhibitors (dual NEP-ACE inhibitors) (e.g., omapatrilat and gemopatrilat), and nitrates.

Examples of suitable anti-platelet agents for use in combination with the compounds of the present invention include GPIIb/IIIa blockers (e.g., abciximab, eptifibatide, tirofiban), P2Y12 antagonists (e.g., clopidogrel, ticlopidine, CS-747), thromboxane receptor antagonists (e.g., ifetroban), aspirin, and PDE-III inhibitors (e.g., dipyridamole) with or without aspirin.

Examples of suitable cardiac glycosides for use in combination with the compounds of the present invention include digitalis and ouabain. Examples of suitable cholesterol/lipid lowering agents for use in combination with the compounds of the present invention include HMG-CoA reductase inhibitors (e.g., pravastatin, lovastatin, atorvastatin, simvastatin, NK-104 (a.k.a. itavastatin, or nisvastatin or nisbastatin) and ZD-4522 (a.k.a. rosuvastatin, or atavastatin or visastatin)), squalene synthetase inhibitors, fibrates, bile acid sequestrants, ACAT inhibitors, MTP inhibitors, lipooxygenase inhibitors, cholesterol absorption inhibitors, and cholesterol ester transfer protein inhibitors (e.g., CP-529414).

Examples of suitable mineralocorticoid receptor antagonists for use in combination with the compounds of the present invention include spironolactone and eplerinone.

Examples of suitable phospodiesterase inhibitors for use in combination with the compounds of the present invention include PDEIII inhibitors such as cilostazol, and PDE V inhibitors such as sildenafil.

Examples of suitable thyroid mimetics for use in combination with the compounds of the present invention include thyrotropin, polythyroid, KB-130015, and dronedarone.

Examples of suitable anabolic agents for use in combination with the compounds of the present invention include testosterone, TRH diethylstilbesterol, estrogens, β-agonists, theophylline, anabolic steroids, dehydroepiandrosterone, enkephalins, E-series prostagladins, retinoic acid and compounds as disclosed in U.S. Pat. No. 3,239,345, e.g., Zeranol®; U.S. Pat. No. 4,036,979, e.g., Sulbenox® or peptides as disclosed in U.S. Pat. No. 4,411,890.

Examples of suitable HIV or AIDS therapies for use in combination with the compounds of the present invention include indinavir sulfate, saquinavir, saquinavir mesylate, ritonavir, lamivudine, zidovudine, lamivudine/zidovudine combinations, zalcitabine, didanosine, stavudine, and megestrol acetate.

Examples of suitable therapies for treatment of Alzheimer's disease and cognitive disorders for use in combination with the compounds of the present invention include donepezil, tacrine, revastigmine, 5HT6, gamma secretase inhibitors, beta secretase inhibitors, SK channel blockers, Maxi-K blockers, and KCNQs blockers.

Examples of suitable therapies for treatment of sleeping disorders for use in combination with the compounds of the present invention include melatonin analogs, melatonin receptor antagonists, ML1B agonists, and GABA/NMDA receptor antagonists.

Examples of suitable anti-proliferative agents for use in combination with the compounds of the present invention include cyclosporin A, paclitaxel, FK 506, and adriamycin.

Examples of suitable anti-tumor agents for use in combination with the compounds of the present invention include paclitaxel, adriamycin, epothilones, cisplatin and carboplatin.

Compounds of the present invention can further be used in combination with nutritional supplements such as those described in U.S. Pat. No. 5,179,080, especially in combination with whey protein or casin, amino acids (such as leucine, branched amino acids and hydroxymethylbutyrate), triglycerides, vitamins (e.g., A, B6, B12, folate, C, D and E), minerals (e.g., selenium, magnesium, zinc, chromium, calcium and potassium), carnitine, lipoic acid, creatine, and coenzyme Q-10.

In addition, compoumds of the present invention can be used in combination with therapeutic agents used in the treatment of sexual dysfunction, including but not limited to PDE5 inhibitors, such as sildenafil or IC-351; with an antiresorptive agent, hormone replacement therapies, vitamin D analogues, calcitonins, elemental calcium and calcium supplements, cathepsin K inhibitors, MMP inhibitors, vitronectin receptor antagonists, Src SH₂ antagonists, vacular —H⁺-ATPase inhibitors, progesterone receptor agonists, ipriflavone, fluoride, RANK antagonists, PTH and its analogues and fragments, Tibolone, HMG-CoA reductase inhibitors, SERM's, p38 inhibitors, prostanoids, 17-beta hydroxysteroid dehydrogenase inhibitors and Src kinase inhibitors.

Compounds of the present invention can be used in combination with male contraceptives, such as nonoxynol 9 or therapeutic agents for the treatment of hair loss, such as minoxidil and finasteride or chemotherapeutic agents, such as with LHRH agonists.

For their preferred anticancer or antiangiogenic use, the compounds of the present invention can be administered either alone or in combination with other anti-cancer and cytotoxic agents and treatments useful in the treatment of cancer or other proliferative diseases, for example, where the second drug has the same or different mechanism of action than the present compounds of formula I. Examples of classes of anti-cancer and cytotoxic agents useful in combination with the present compounds include but are not limited to: alkylating agents such as nitrogen mustards, alkyl sulfonates, nitrosoureas, ethylenimines, and triazenes; antimetabolites such as folate antagonists, purine analogues, and pyrimidine analogues; antibiotics such as anthracyclines, bleomycins, mitomycin, dactinomycin, and plicamycin; enzymes such as L-asparaginase; famesyl-protein transferase inhibitors; 5α reductase inhibitors; inhibitors of 17β-hydroxy steroid dehydrogenase type 3; hormonal agents such as glucocorticoids, estrogens/antiestrogens, androgens/antiandrogens, progestins, and luteinizing hormone-releasing hormone antagonists, octreotide acetate; microtubule-disruptor agents, such as ecteinascidins or their analogs and derivatives; microtubule-stabilizing agents such as taxanes, for example, paclitaxel (Taxol®), docetaxel (Taxotere®), and their analogs, and epothilones, such as epothilones A-F and their analogs; plant-derived products, such as vinca alkaloids, epipodophyllotoxins, taxanes; and topiosomerase inhibitors; prenyl-protein transferase inhibitors; and miscellaneous agents such as hydroxyurea, procarbazine, mitotane, hexamethylmelamine, platinum coordination complexes such as cisplatin and carboplatin; and other agents used as anti-cancer and cytotoxic agents such as biological response modifiers, growth factors; immune modulators and monoclonal antibodies. The compounds of the invention may also be used in conjunction with radiation therapy.

Representative examples of these classes of anti-cancer and cytotoxic agents include but are not limited to mechlorethamine hydrochloride, cyclophosphamide, chlorambucil, melphalan, ifosfamide, busulfan, carmustin, lomustine, semustine, streptozocin, thiotepa, dacarbazine, methotrexate, thioguanine, mercaptopurine, fludarabine, pentastatin, cladribin, cytarabine, fluorouracil, doxorubicin hydrochloride, daunorubicin, idarubicin, bleomycin sulfate, mitomycin C, actinomycin D, safracins, saframycins, quinocarcins, discodermolides, vincristine, vinblastine, vinorelbine tartrate, etoposide, etoposide phosphate, teniposide, paclitaxel, tamoxifen, estramustine, estramustine phosphate sodium, flutamide, buserelin, leuprolide, pteridines, diyneses, levamisole, aflacon, interferon, interleukins, aldesleukin, filgrastim, sargramostim, rituximab, BCG, tretinoin, irinotecan hydrochloride, betamethosone, gemcitabine hydrochloride, altretamine, and topoteca and any analogs or derivatives thereof.

Preferred member of these classes include, but are not limited to, paclitaxel, cisplatin, carboplatin, doxorubicin, carminomycin, daunorubicin, aminopterin, methotrexate, methopterin, mitomycin C, ecteinascidin 743, or porfiromycin, 5-fluorouracil, 6-mercaptopurine, gemcitabine, cytosine arabinoside, podophyllotoxin or podophyllotoxin derivatives such as etoposide, etoposide phosphate or teniposide, melphalan, vinblastine, vincristine, leurosidine, vindesine and leurosine.

Examples of anticancer and other cytotoxic agents include the following: epothilone derivatives as found in German Patent No. 4138042.8; WO 97/19086, WO 98/22461, WO 98/25929, WO 98/38192, WO 99/01124, WO 99/02224, WO 99/02514, WO 99/03848, WO 99/07692, WO 99/27890, WO 99/28324, WO 99/43653, WO 99/54330, WO 99/54318, WO 99/54319, WO 99/65913, WO 99/67252, WO 99/67253 and WO 00/00485; cyclin dependent kinase inhibitors as found in WO 99/24416 (see also U.S. Pat. No. 6,040,321); and prenyl-protein transferase inhibitors as found in WO 97/30992 and WO 98/54966; and agents such as those described generically and specifically in U.S. Pat. No. 6,011,029 (the compounds of which U.S. Patent can be employed together with any NHR modulators (including, but not limited to, those of present invention) such as AR modulators, ER modulators, with LHRH modulators, or with surgical castration, especially in the treatment of cancer).

The combinations of the present invention can also be formulated or co-administered with other therapeutic agents that are selected for their particular usefulness in administering therapies associated with the aforementioned conditions.

For example, the compounds of the invention may be formulated with agents to prevent nausea, hypersensitivity and gastric irritation, such as antiemetics, and H₁ and H₂ antihistaminics.

As it pertains to the treatment of cancer, the compounds of this invention are most preferably used alone or in combination with anti-cancer treatments such as radiation therapy and/or with cytostatic and/or cytotoxic agents, such as, but not limited to, DNA interactive agents, such as cisplatin or doxorubicin; inhibitors of famesyl protein transferase, such as those described in U.S. Pat. No. 6,011,029; topoisomerase II inhibitors, such as etoposide; topoisomerase I inhibitors, such as CPT-11 or topotecan; tubulin stabilizing agents, such as paclitaxel, docetaxel, other taxanes, or epothilones; hormonal agents, such as tamoxifen; thymidilate synthase inhibitors, such as 5-fluorouracil; antimetabolites, such as methoxtrexate; antiangiogenic agents, such as angiostatin, ZD6474, ZD6126 and comberstatin A2; kinase inhibitors, such as her2 specific antibodies, Iressa and CDK inhibitors; histone deacetylase inhibitors, such as CI-994 and MS-27-275. Such compounds may also be combined with agents which suppress the production of circulating testosterone such as LHRH agonists or antagonists or with surgical castration.

For example, known therapies for advanced metastatic prostate cancer include “complete androgen ablation therapy” wherein tumor growth is inhibited by controlling the supply of androgen to the prostate tissues via chemical castration (castration serves to inhibit the production of circulating testosterone (T) and dihydrotestosterone (DHT)) followed by the administration of androgen receptor (AR) antagonists (which inhibit the function T/DHT derived from the conversion of circulating androgen precursors to T/DHT by the prostate tissue). The compounds of the present invention can be employed as AR antagonists in complete ablation therapy, alone or in combination with other AR antagonists such as Flutamide, Casodex, Nilutamide, or Cyproterone acetate.

The compounds of the present invention may be employed adjuvant to surgery.

Another application of the present compounds is in combination with antibody therapy such as but not limited to antibody therapy against PSCA. An additional application is in concert with vaccine/immune modulating agents for the treatment of cancer.

Compounds of the present invention can be employed in accordance with the methods described in U.S. Provisional Patent Application Serial No. 60/284,438, entitled “Selective Androgen Receptor Modulators and Methods for Their Identification, Design and Use” filed Apr. 18, 2001 by Mark E. Salvati et al. (Attorney Docket No. LD0250(PSP)), which Provisional Patent Application is incorporated herein by reference in its entirety (including, but not limited to, reference to all specific compounds within formula I of the present invention), and U.S. patent application Ser. No. ______ (unassigned), entitled “Selective Androgen Receptor Modulators and Methods for Their Identification, Design and Use” filed Jun. 20, 2001 by Mark E. Salvati et al. (Attorney Docket No. LD0250(NP)), which Patent Application is incorporated herein by reference in its entirety (including, but not limited to, reference to all specific compounds within formula I of the present invention).

For racemates of compounds of the present invention, one enantiomer can, for example be a full AR antagonist while the other can be an AR antagonist in tumor tissue while having no activity or agonist activity in nontumor tissue containing the androgen receptor.

The above other therapeutic agents, when employed in combination with the compounds of the present invention, can be used, for example, in those amounts indicated in the Physicians' Desk Reference (PDR) or as otherwise determined by one of ordinary skill in the art.

The following assays can be employed in ascertaining the activity of a compound as a NHR modulator. Preferred are those compounds with an activity greater than 20 μm for binding or transactivation in any of these assays. Various compounds of the present invention were determined to have AR modulator activity utilizing the transactivation assay, and standard AR binding assays as described following.

Transactivation Assays:

AR Specific Assay:

Compounds of the present invention were tested in transactivation assays of a transfected reporter construct and using the endogenous androgen receptor of the host cells. The transactivation assay provides a method for identifying functional agonists and partial agonists that mimic, or antagonists that inhibit, the effect of native hormones, in this case, dihydrotestosterone (DHT). This assay can be used to predict in vivo activity as there is a good correlation in both series of data. See, e.g. T. Berger et al., J. Steroid Biochem. Molec. Biol. 773 (1992), the disclosure of which is herein incorporated by reference.

For the transactivation assay a reporter plasmid is introduced by transfection (a procedure to induce cells to take foreign genes) into the respective cells. This reporter plasmid, comprising the cDNA for a reporter protein, such as secreted alkaline phosphatase (SEAP), controlled by prostate specific antigen (PSA) upstream sequences containing androgen response elements (AREs). This reporter plasmid functions as a reporter for the transcription-modulating activity of the AR. Thus, the reporter acts as a surrogate for the products (niRNA then protein) normally expressed by a gene under control of the AR and its native hormone. In order to detect antagonists, the transactivation assay is carried out in the presence of constant concentration of the natural AR hormone (DHT) known to induce a defined reporter signal. Increasing concentrations of a suspected antagonist will decrease the reporter signal (e.g., SEAP production). On the other hand, exposing the transfected cells to increasing concentrations of a suspected agonist will increase the production of the reporter signal.

For this assay, LNCaP and MDA 453 cells were obtained from the American Type Culture Collection (Rockville, Md.), and maintained in RPMI 1640 or DMEM medium supplemented with 10% fetal bovine serum (FBS; Gibco) respectively. The respective cells were transiently transfected by electroporation according to the optimized procedure described by Heiser, 130 Methods Mol. Biol., 117 (2000), with the pSEAP2/PSA540/Enhancer reporter plasmid. The reporter plasmid, was constructed as follows: commercial human placental genomic DNA was used to generate by Polymerase Cycle Reaction (PCR) a fragment containing the BglII site (position 5284) and the Hind III site at position 5831 of the human prostate specific antigen promoter (Accession # U37672), Schuur, et al., J. Biol. Chem., 271 (12): 7043-51 (1996). This fragment was subdloned into the pSEAP2/basic (Clontech) previously digested with BglII and HindIII to generate the pSEAP2/PSA540 construct. Then a fragment bearing the fragment of human PSA upstream sequence between positions-5322 and -3873 was amplified by PCR from human placental genomic DNA. A XhoI and a BglII sites were introduced with the primers. The resulting fragment was subcloned into pSEAP2/PSA540 digested with XhoI and BglII respectively, to generate the pSEAP2/PSA540/Enhancer construct. LNCaP and MDA 453 cells were collected in media containing 10% charcoal stripped FBS. Each cell suspension was distributed into two Gene Pulser Cuvetts (Bio-Rad) which then received 8 μg of the reporter construct, and electoporated using a Bio-Rad Gene Pulser at 210 volts and 960 μFaraday. Following the transfections the cells were washed and incubated with media containing charcoal stripped fetal bovine serum in the absence (blank) or presence (control) of 1 nM dihydrotestosterone (DHT; Sigma Chemical) and in the presence or absence of the standard anti-androgen bicalutamide or compounds of the present invention in concentrations ranging from 10-10 to 10-5 M (sample). Duplicates were used for each sample. The compound dilutions were performed on a Biomek 2000 laboratory workstation. After 48 hours, a fraction of the supernatant was assayed for SEAP activity using the Phospha-Light Chemiluminescent Reporter Gene Assay System (Tropix, Inc). Viability of the remaining cells was determined using the CellTiter 96 Aqueous Non-Radioactive Cell Proliferation Assay (MTS Assay, Promega). Briefly, a mix of a tetrazolium compound (3-(4,5-dimethylthiazol-2-yl)-5-(3-carboxymethoxyphenyl)-2-(4-sulfophenyl)-2H-tetrazolium, inner salt; MTS) and an electron coupling reagent (phenazine methosulfate; PMS) are added to the cells. MTS (Owen's reagent) is bioreduced by cells into a formazan that is soluble in tissue culture medium, and therefore its absorbance at 490 nm can be measured directly from 96 well assay plates without additional processing. The quantity of formazan product as measured by the amount of 490 nm absorbance is directly proportional to the number of living cells in culture. For each replicate the SEAP reading was normalized by the Abs490 value derived from the MTS assay. For the antagonist mode, the % Inhibition was calculated as:

% Inhibition=100×(1−[average control−average blank/average sample−average blank])

Data was plotted and the concentration of compound that inhibited 50% of the normalized SEAP was quantified (IC₅₀).

For the agonist mode % Control was referred as the effect of the tested compound compared to the maximal effect observed with the natural hormone, in this case DHT, and was calculated as:

% Control=100×average sample−average blank/average control−average blank

Data was plotted and the concentration of compound that activates to levels 50% of the normalized SEAP for the control was quantified (EC₅₀).

GR Specificity Assay:

The reporter plasmid utilized was comprised of the cDNA for the reporter SEAP protein, as described for the AR specific transactivation assay. Expression of the reporter SEAP protein was controlled by the mouse mammary tumor virus long terminal repeat (MMTV LTR) sequences that contains three hormone response elements (HREs) that can be regulated by both GR and PR see, e.g. G. Chalepakis et al., Cell, 53(3), 371 (1988). This plasmid was transfected into A549 cells, which expresses endogenous GR, to obtain a GR specific transactivation assay. A549 cells were obtained from the American Type Culture Collection (Rockville, Md.), and maintained in RPMI 1640 supplemented with 10% fetal bovine serum (FBS; Gibco). Determination of the GR specific antagonist activity of the compounds of the present invention was identical to that described for the AR specific transactivation assay, except that the DHT was replaced with 5 nM dexamethasone (Sigma Chemicals), a specific agonist for GR. Determination of the GR specific agonist activity of the compounds of the present invention was performed as described for the AR transactivation assay, wherein one measures the activation of the GR specific reporter system by the addition of a test compound, in the absence of a known GR specific agonists ligand.

PR Specific Assay:

The reporter plasmid utilized was comprised of the cDNA for the reporter SEAP protein, as described for the AR specific transactivation assay. Expression of the reporter SEAP protein was controlled by the mouse mammary tumor virus long terminal repeat (MMTV LTR) sequences that contains three hormone response elements (HREs) that can be regulated by both GR and PR. This plasmid was transfected into T47D, which expresses endogenous PR, to obtain a PR specific transactivation assay. T47D cells were obtained from the American Type Culture Collection (Rockville, Md.), and maintained in DMEM medium supplemented with 10% fetal bovine serum (FBS; Gibco). Determination of the PR specific antagonist activity of the compounds of the present invention was identical to that described for the AR specific transactivation assay, except that the DHT was replaced with 1 nM Promegastone (NEN), a specific agonist for PR. Determination of the PR specific agonist activity of the compounds of the present invention was performed as described for the AR transactivation assay, wherein one measures the activation of the PR specific reporter system by the addition of a test compound, in the absence of a known PR specific agonists ligand.

AR Binding Assay:

For the whole cell binding assay, human LNCaP cells (T877A mutant AR) or MDA 453 (wild type AR) in 96-well microtiter plates containing RPMI 1640 or DMEM supplemented with 10% charcoal stripped CA-FBS (Cocaleco Biologicals) respectively, were incubated at 37° C. to remove any endogenous ligand that might be complexed with the receptor in the cells. After 48 hours, either a saturation analysis to determine the K_{d} for tritiated dihydrotestosterone, [³H]-DHT, or a competitive binding assay to evaluate the ability of test compounds to compete with [³H]-DHT were performed. For the saturation analysis, media (RPMI 1640 or DMEM-0.2% CA-FBS) containing [³H]-DHT (in concentrations ranging from 0.1 nM to 16 nNM) in the absence (total binding) or presence (non-specific binding) of a 500-fold molar excess of unlabeled DHT were added to the cells. After 4 hours at 37° C., an aliquot of the total binding media at each concentration of [³H]-DHT was removed to estimate the amount of free [³H]-DHT. The remaining media was removed, cells were washed three times with PBS and harvested onto UniFilter GF/B plates (Packard), Microscint (Packard) was added and plates counted in a Top-Counter (Packard) to evaluate the amount of bound [³H]-DHT.

For the saturation analysis, the difference between the total binding and the non-specific binding, was defined as specific binding. The specific binding was evaluated by Scatchard analysis to determine the K_{d} for [³H]-DHT. See e.g. D. Rodbard, Mathematics and statistics of ligand assays: an illustrated guide: In: J. Langon and J. J. Clapp, eds., Ligand Assay, Masson Publishing U.S.A., Inc., New York, pp. 45-99, (1981), the disclosure of which is herein incorporated by reference.

For the competition studies, media containing 1 nM [³H]-DHT and compounds of the invention (“test compounds”) in concentrations ranging from 10⁻¹⁰ to 10⁻⁵ M were added to the cells. Two replicates were used for each sample. After 4 hours at 37° C., cells were washed, harvested and counted as described above. The data was plotted as the amount of [³H]-DHT (% of control in the absence of test compound) remaining over the range of the dose response curve for a given compound. The concentration of test compound that inhibited 50% of the amount of [³H]-DHT bound in the absence of competing ligand was quantified (IC₅₀) after log-logit transformation. The K_{I} values were determined by application of the Cheng-Prusoff equation to the IC₅₀ values, where:

$${K}_{I} = \frac{{IC}_{50}}{( 1 + ( {}^{3} \text{H-DHT} ) / {K}_{d} for {}^{3} \text{H-DHT} )} .$$

After correcting for non-specific binding, IC₅₀ values were determined. The IC₅₀ is defined as the concentration of competing ligand needed to reduce specific binding by 50%. The K_{d}s for [³H]-DHT for MDA 453 and LNCaP were 0.7 and 0.2 nM respectively.

Human Prostate Cell Proliferation Assay:

Compounds of the present invention were tested (“test compounds”) on the proliferation of human prostate cancer cell lines. For that, MDA PCa2b cells, a cell line derived from the metastasis of a patient that failed castration, Navone et al., Clin. Cancer Res., 3, 2493-500 (1997), were incubated with or without the test compounds for 72 hours and the amount of [³H]-thymidine incorporated into DNA was quantified as a way to assess number of cells and therefore proliferation. The MDA PCa2b cell line was maintained in BRFF-HPC1 media (Biological Research Faculty & Facility Inc., MD) supplemented with 10% FBS. For the assay, cells were plated in Biocoated 96-well microplates and incubated at 37° C. in 10% FBS (charcoal-stripped)/BRFF-BMZERO (without androgens). After 24 hours, the cells were treated in the absence (blank) or presence of 1 nM DHT (control) or with test compounds (sample) of the present invention in concentrations ranging from 10⁻¹⁰ to 10⁻⁵ M. Duplicates were used for each sample. The compound dilutions were performed on a Biomek 2000 laboratory work station. Seventy two hours later 0.44 uCi. of [³H]-Thymidine (Amersham) was added per well and incubated for another 24 h followed by tripsinization, harvesting of the cells onto GF/B filters. Micro-scint PS were added to the filters before counting them on a Beckman TopCount.

The % Inhibition was calculated as:

% Inhibition=100×(1−[average_{control}−average_{blank}/averageₛₐₘₚₗₑ−average_{blank}])

Data was plotted and the concentration of compound that inhibited 50% of the [³H]-Thymidine incorporation was quantified (IC₅₀).

C2C12 Mouse Myoblast Transactivation Assay:

Two functional transactivation assays were developed to assess the efficacy of androgen agonists in a muscle cell background using a luciferase reporter. The first assay (ARTA Stable 1) uses a cell line, Stable 1 (clone #72), which stably expresses the full length rat androgen receptor but requires the transient transfection of an enhancer/reporter. This cell line was derived from C2C12 mouse moyoblast cells. The second assay (ARTA Stable 2) uses a cell line, Stable 2 (clone #133), derived from Stable 1 which stably expresses both rAR and the enhancer/luciferase reporter.

The enhancer/reporter construct used in this system is pGL3/2×DR-1/luciferase. 2×DR-1 was reported to be an AR specific response element in CV-1 cells, Brown et. al. The Journal ofBiological Chemistry 272, 8227-8235, (1997). It was developed by random mutagenesis of an AR/GR consensus enhancer sequence.

ARTA Stable 1:

1. Stable 1 cells are plated in 96 well format at 6,000 cells/well in high glucose DMEM without phenol red (Gibco BRL, Cat. No.: 21063-029) containing 10% charcoal and dextran treated FBS (HyClone Cat. No.: SH30068.02), 50 mM HEPES Buffer (Gibco BRL, Cat. No.: 15630-080), 1×MEM Na Pyruvate (Gibco BRL, Cat. No.: 11360-070), 0.5×Antibiotic-Antimycotic, and 800 μg/ml Geneticin (Gibco BRL, Cat. No.: 10131-035).

2. 48 hours later, cells are transfected with pGL3/2×DR-1/luciferase using LipofectAMINE Plus™ Reagent (Gibco BRL, Cat. No.: 10964-013). Specifically, 5 ng/well pGL3/2×DR-1/luciferase DNA and 50 ng/well Salmon Sperm DNA (as carrier) are diluted with 5 μl/well Opti-MEMem media (Gibco BRL, Cat. No.: 31985-070). To this, 0.5 μl/well Plus reagent is added. This mixture is incubated for 15 minutes at room temperature. In a separate vessel, 0.385 μl/well LipofectAMINE reagent is diluted with 5 μl/well Opti-MEM. The DNA mixture is then combined with the LipofectAMINE mixture and incubated for an additional 15 minutes at room temperature. During this time, the media from the cells is removed and replaced with 60 μl/well of Opti-MEM. To this is added 10 μl/well of the DNA/LipofectAMINE transfection mixture. The cells are incubated for 4 hours.

3. The transfection mixture is removed from the cells and replaced with 90 μl of media as in #1 above.

4. 10 μl/well of appropriate drug dilution is placed in each well.

5. 24 hours later, the Steady-Glo™Luciferase Assay System is used to detect activity according to the manufacturer's instructions (Promega, Cat. No.: E2520).

ARTA stable 2

1. Stable 2 cells are plated in 96 well format at 6,000 cells/well in high glucose DMEM without phenol red (Gibco BRL, Cat. No.: 21063-029) containing 10% charcoal and dextran treated FBS (HyClone Cat. No.: SH30068.02), 50 mM HEPES Buffer (Gibco BRL, Cat. No.: 15630-080), 1×MEM Na Pyruvate (Gibco BRL, Cat. No.: 11360-070), 0.5×Antibiotic-Antimycotic, 800 μg/ml Geneticin (Gibco BRL, Cat. No.: 10131-035) and 800 μg/ml Hygromycin β (Gibco BRL, Cat. No.: 10687-010).

2. 48 hours later, the media on the cells is removed and replaced with 90 μl fresh. 10 μl/well of appropriate drug dilution is placed in each well.

3. 24 hours later, the Steady-GloTM Luciferase Assay System is used to detect activity according to the manufacturer's instructions (Promega, Cat. No.: E2520).

See U.S. patent application Ser. No. ______ (unassigned), entitled “Cell Lines and Cell-BasedAssays for Identification of Androgen Receptor Modulators” filed Jun. 20, 2001 by Jacek Ostrowski et al. (Attorney Docket No. D0177), which Patent Application is incorporated herein by reference in its entirety.

Proliferation Assays

Murine Breast Cell Proliferation Assay:

The ability of compounds of the present invention (“test compounds”) to modulate the function of the AR was determined by testing said compounds in a proliferation assay using the androgen responsive murine breast cell line derived from the Shionogi tumor, Hiraoka et al., Cancer Res., 47, 6560-6564 (1987). Stable AR dependent clones of the parental Shionogi line were established by passing tumor fragments under the general procedures originally described in Tetuo, et. al., Cancer Research 25, 1168-1175 (1965). From the above procedure, one stable line, SC114, was isolated, characterized and utilized for the testing of example compounds. SC114 cells were incubated with or without the test compounds for 72 hours and the amount of [3H]-thymidine incorporated into DNA was quantified as a surrogate endpoint to assess the number of cells and therefore the proliferation rate as described in Suzuki et. al., J. Steroid Biochem. Mol. Biol. 37, 559-567 (1990). The SC114 cell line was maintained in MEM containing 10⁻⁸ M testosterone and 2% DCC-treated FCS. For the assay, cells were plated in 96-well microplates in the maintenance media and incubated at 37° C. On the following day, the medium was changed to serum free medium [Ham's F-12:MEM (1;1, v/v) containing 0.1% BSA] with (antagonist mode) or without (agonist mode) 10⁻⁸ M testosterone and the test compounds of the present invention in concentrations ranging from 10⁻¹⁰ to 10⁻⁵ M. Duplicates were used for each sample. The compound dilutions were performed on a Biomek 2000 laboratory work station. Seventy two hours later 0.44 uCi of [3H]-Thymidine (Amersham) was added per well and incubated for another 2 hr followed by tripsinization, and harvesting of the cells onto GF/B filters. Micro-scint PS were added to the filters before counting them on a Beckman TopCount.

For the antagonist mode, the % Inhibition was calculated as:

Inhibition 100×(1−[averageₛₐₘₚₗₑ−average_{blank}/average_{control}−average_{blank}])

Data was plotted and the concentration of compound that inhibited 50% of the [³H]-Thymidine incorporation was quantified (IC₅₀).

For the agonist mode % Control was referred as the effect of the tested compound compared to the maximal effect observed with the natural hormone, in this case DHT, and was calculated as:

% Control=100×(averageₛₐₘₚₗₑ−average_{blank})/(average_{control}−average_{blank})

Data was plotted and the concentration of compound that inhibited 50% of the [³H]-Thymidine incorporation was quantified (EC₅₀).

In Vitro Assay to Measure GR Induced AP-1 Transrepression:

The AP-1 assay is a cell based luciferase reporter assay. A549 cells, which contain endogenous glucocorticoid receptor, were stably transfected with an AP-1 DNA binding site attached to the luciferase gene. Cells are then grown in RPMI+10% fetal calf serum (charcoal-treated)+Penicillin/Streptomycin with 0.5 mg/ml geneticin. Cells are plated the day before the assay at approximately 40000 cells/well. On assay day, the media is removed by aspiration and 20 μl assay buffer (RPMI without phenol red+10% FCS (charcoal-treated)+Pen/Strep) is added to each well. At this point either 20 μl assay buffer (control experiments), the compounds of the present invention (“test compounds”) (dissolved in DMSO and added at varying concentrations) or dexamethasome (100 nM in DMSO, positive control) are added to each well. The plates are then pre-incubated for 15 minutes at 37° C, followed by stimulation of the cells with 10 ng/ml PMA. The plates are then incubated for 7 hrs at 37° C. after which 40 μl luciferase substrate reagent is added to each well. Activity is measured by analysis in a luminometer as compared to control experiments treated with buffer or dexamethasome. Activity is designated as % inhibition of the reporter system as compared to the buffer control with 10 ng/ml PMA alone. The control, dexamethasone, at a concentration of ≦10 μM typically suppresses activity by 65%. Test compounds which demonstrate an inhibition of PMA induction of 50% or greater at a concentration of test compound of <10 μM are deemed active.

Wet Prostate Weight Assay AR Antagonist Assay:

The activity of compounds of the present invention as AR antagonists was investigated in an immature male rat model, a standard, recognized test of antiandrogen activity of a given compound, as described in L. G. Hershberger et al., Proc. Soc. Expt. Biol. Med., 83, 175 (1953); P. C. Walsh and R. F. Gittes, “Inhibition of extratesticular stimuli to prostate growth in the castrated rat by antiandrogens”, Endocrinology, 86, 624 (1970); and B. J. Furr et al., “ICI 176,334: A novel non-steriod, peripherally selective antiandrogen”, J. Endocrinol., 113, R7-9 (1987), the disclosures of which are herein incorporated by reference.

The basis of this assay is the fact that male sexual accessory organs, such as the prostate and seminal vesicles, play an important role in reproductive function. These glands are stimulated to grow and are maintained in size and secretory function by the continued presence of serum testosterone (T), which is the major serum androgen (>95%) produced by the Leydig cells in the testis under the control of the pituitary luteinizing hormone (LH) and follicle stimulating hormone (FSH). Testosterone is converted to the more active form, dihydrotestosterone, (DHT), within the prostate by 5α-reductase. Adrenal androgens also contribute about 20% of total DHT in the rat prostate, compared to 40% of that in 65-year-old men. F. Labrie et al. Clin. Invest. Med., 16, 475-492 (1993). However, this is not a major pathway, since in both animals and humans, castration leads to almost complete involution of the prostate and seminal vesicles without concomitant adrenalectomy. Therefore, under normal conditions, the adrenals do not support significant growth of prostate tissues. M. C. Luke and D. S. Coffey, “The Physiology of Reproduction” ed. By E. Knobil and J. D. Neill, 1, 1435-1487 (1994). Since the male sex organs are the tissues most responsive to modulation of the androgen activity, this model is used to determine the androgen dependent growth of the sex accessory organs in immature castrated rats.

Male immature rats (19-20 days old Sprague-Dawley, Harlan Sprague-Dawely) were castrated under metofane ansestesia. Five days after surgery these castrated rats (60-70 g, 23-25 day-old) were dosed for 3 days. Animals were dosed sub-cutaneously (s.c.) 1 mg/kg with Testosterone Proprionate (TP) in arachis oil vehicle and anti-androgen test compounds (compounds of the present invention) were dosed orally by gavage (p.o.) in dissolved/suspensions of 80% PEG 400 and 20% Tween 80 (PEGTW). Animals were dosed (v/w) at 0.5 ml of vehicle/100 g body weight. Experimental groups were as follows:

1. Control vehicle

2. Testosterone Propionate (TP) (3 mg/rat/day, subcutaneous)

3. TP plus Casodex (administered p.o. in PEGTW, QD), a recognized antiandrogen, as a reference compound.

4. To demonstrate antagonist activity, a compound of the present invention (“test compound”) was administered (p.o. in PEGTW, QD) with TP (s.c. as administered in group 2) in a range of doses.

5. To demonstrate agonist activity a compound of the present invention (“test compound”) was administered alone (p.o. in PEGTW, QD) in a range of doses.

At the end of the 3-day treatment, the animals were sacrificed, and the ventral prostate weighed. To compare data from different experiments, the sexual organs weights were first standardized as mg per 100 g of body weight, and the increase in organ weight induced by TP was considered as the maximum increase (100%). ANOVA followed by one-tailed Student or Fischer's exact test was used for statistical analysis.

The gain and loss of sexual organ weight reflect the changes of the cell number (DNA content) and cell mass (protein content), depending upon the serum androgen concentration. See Y. Okuda et al., J. Urol., 145, 188-191 (1991), the disclosure of which is herein incorporated by reference. Therefore, measurement of organ wet weight is sufficient to indicate the bioactivity of androgens and androgen antagonist. In immature castrated rats, replacement of exogenous androgens increases seminal vesicles (SV) and the ventral prostate (VP) in a dose dependent manner.

The maximum increase in organ weight was 4 to 5-fold when dosing 3 mg/rat/day of testosterone (T) or 1 mg/rat/day of testosterone propionate (TP) for 3 days. The EC₅₀ of T and TP were about 1 mg and 0.03 mg, respectively. The increase in the weight of the VP and SV also correlated with the increase in the serum T and DHT concentration. Although administration of T showed 5-times higher serum concentrations of T and DHT at 2 hours after subcutaneous injection than that of TP, thereafter, these high levels declined very rapidly. In contrast, the serum concentrations of T and DHT in TP-treated animals were fairly consistent during the 24 hours, and therefore, TP showed about 10-30-fold higher potency than free T.

In this immature castrated rat model, a known AR antagonist (Casodex) was 30 also administered simultaneously with 0.1 mg of TP (ED₈₀), inhibiting the testosterone-mediated increase in the weights of the VP and SV in a dose dependent manner. The antagonist effects were similar when dosing orally or subcutaneously.

Compounds of the invention also exhibited AR antagonist activity by suppressing the testosterone-mediated increase in the weights of VP and SV.

Levator Ani & Wet Prostate Weight Assay AR Agonist Assay:

The activity of compounds of the present invention as AR agonists was investigated in an immature male rat model, a recognized test of anabolic effects in muscle and sustaining effects in sex organs for a given compound, as described in L. G. Hershberger et al., Proc. Soc. Expt. Biol. Med., 83, 175 (1953); B. L. Beyler et al, “Methods for evaluating anabolic and catabolic agents in laboratory animals”, J. Amer. Med. Women's Ass., 23, 708 (1968); H. Fukuda et al., “Investigations of the levator ani muscle as an anabolic steroid assay”, Nago Dai. Yak. Ken. Nem. 14, 84 (1966) the disclosures of which are herein incorporated by reference.

The basis of this assay lies in the well-defined action of androgenic agents on the maintenance and growth of muscle tissues and sexual accessory organs in animals and man. Androgenic steroids, such as testosterone (T), have been well characterized for their ability to maintain muscle mass. Treatment of animals or humans after castrations with an exogenous source of T results in a reversal of muscular atrophy. The effects of T on muscular atrophy in the rat levator ani muscle have been well characterized. M. Masuoka et al., “Constant cell population in normal, testosterone deprived and testosterone stimulated levator ani muscles” Am. J. Anat. 119, 263 (1966); Z. Gori et al., “Testosterone hypertrophy of levator ani muscle of castrated rats. I. Quantitative data” Boll.-Soc. Ital. Biol. Sper. 42, 1596 (1966); Z. Gori et al., “Testosterone hypertrophy of levator ani muscle of castrated rats. II. Electron-microscopic observations” Boll.-Soc. Ital. Biol. Sper. 42, 1600 (1966); A. Boris et al., Steroids 15, 61 (1970). As described above, the effects of androgens on maintenance of male sexual accessory organs, such as the prostate and seminal vesicles, is well described. Castration results in rapid involution and atrophy of the prostate and seminal vesicles. This effect can be reversed by exogenous addition of androgens. Since both the levator ani muscle and the male sex organs are the tissues most responsive to the effects of androgenic agents, this model is used to determine the androgen dependent reversal of atrophy in the levator ani muscle and the sex accessory organs in immature castrated rats. Sexually mature rats (200-250 g, 6-8 weeks-old, Sprague-Dawley, Harlan) were acquired castrated from the vendor (Taconic). The rats were divided into groups and treated daily for 7 to 14 days with one of the following:

1. Control vehicle

2. Testosterone Propionate (TP) (3 mg/rat/day, subcutaneous)

3. TP plus Casodex (administered p.o. in PEGTW, QD), a recognized antiandrogen, as a reference compound.

4. To demonstrate antagonist activity, a compound of the present invention (“test compound”) was administered (p.o. in PEGTW, QD) with TP (s.c. as administered in group 2) in a range of doses.

5. To demonstrate agonist activity a compound of the present invention (“test compound”) was administered alone (p.o. in PEGTW, QD) in a range of doses.

At the end of the 7-14-day treatment, the animals were sacrificed by carbon dioxide, and the levator ani, seminal vesicle and ventral prostate weighed. To compare data from different experiments, the levator ani muscle and sexual organ weights were first standardized as mg per 100 g of body weight, and the increase in organ weight induced by TP was considered as the maximum increase (100%). Super-anova (one factor) was used for statistical analysis.

The gain and loss of sexual organ weight reflect the changes of the cell number (DNA content) and cell mass (protein content), depending upon the serum androgen concentration. See Y. Okuda et al., J. Urol., 145, 188-191 (1991), the disclosure of which is herein incorporated by reference. Therefore, measurement of organ wet weight is sufficient to indicate the bioactivity of androgens and androgen antagonist. In immature castrated rats, replacement of exogenous androgens increases levator ani, seminal vesicles (SV) and prostate in a dose dependent manner.

The maximum increase in organ weight was 4 to 5-fold when dosing 3 mg/rat/day of testosterone (T) or 1 mg/rat/day of testosterone propionate (TP) for 3 days. The EC₅₀ of T and TP were about 1 mg and 0.03 mg, respectively. The increase in the weight of the VP and SV also correlated with the increase in the serum T and DHT concentration. Although administration of T showed 5-times higher serum concentrations of T and DHT at 2 hours after subcutaneous injection than that of TP, thereafter, these high levels declined very rapidly. In contrast, the serum concentrations of T and DHT in TP-treated animals were fairly consistent during the 24 hours, and therefore, TP showed about 10-30-fold higher potency than free T.

MDA PCa2b Human Prostate Zenograft Assay:

In Vivo Antitumor Testing: MDA-PCa-2b human prostate tumors were maintained in Balb/c nu/nu nude mice. Tumors were propagated as subcutaneous transplants in adult male nude mice (4-6 weeks old) using tumor fragments obtained from donor mice. Tumor passage occurred every 5-6 weeks.

For antitumor efficacy trial, the required number of animals needed to detect a meaningful response were pooled at the start of the experiment and each was given a subcutaneous implant of a tumor fragment (˜50 mg) with a 13-gauge trocar. Tumors were allowed to grow to approx. 100-200 mg (tumors outside the range were excluded) and animals were evenly distributed to various treatment and control groups. Treatment of each animal was based on individual body weight. Treated animals were checked daily for treatment related toxicity/mortality. Each group of animals was weighed before the initiation of treatment (Wt1) and then again following the last treatment dose (Wt2). The difference in body weight (Wt2−Wt1) provides a measure of treatment-related toxicity.

Tumor response was determined by measurement of tumors with a caliper twice a week, until the tumors reach a predetermined “target” size of 0.5 gm. Tumor weights (mg) were estimated from the formula: Tumor weight=(length×width2)÷2

Tumor response end-point was expressed in terms of tumor growth inhibition (% T/C), defined as the ratio of median tumor weights of the treated tumors (T) to that of the control group (C).

To estimate tumor cell kill, the tumor volume doubling time was first calculated with the formula:

TVDT=Median time (days) for control tumors to reach target size−Median time (days) for control tumors to reach half the target size s

And,

Log cell kill=(T−C)÷(3.32×TVDT)

Statistical evaluations of data were performed using Gehan's generalized Wilcoxon test.

Dunning Prostate Tumor:

Dunning R3327H prostate tumor is a spontaneously derived, well differentiated androgen responsive adenocarcinoma of the prostate (Smolev J K, Heston W D, Scott W W, and Coffey D S, Cancer Treat Rep. 61, 273-287 (1977)). The growth of the R3327H subline has been selected for its highly androgen-dependent and reproducible growth in intact male rats. Therefore, this model and other sublines of this tumor have been widely used to evaluate in vivo antitumor activities of antiandrogens such as flutamide and bacilutamide/Casodex (Maucher A., and von Angerer, J. Cancer Res. Clin. Oncol., 119, 669-674 (1993), Furr B. J. A. Euro. URL. 18 (suppl. 3), 2-9 (1990), Shain S. A. and Huot RI. J. Steriod Biochem. 31, 711-718 (1988)).

At the beginning of the study, the Dunning tumor pieces (about 4×4 mm) are transplanted subcutaneously to the flank of mature male Copenhagen rats (6-7 weeks old, Harlan—Sprague Dawley, Indianapolis, Md.). About 6 weeks after the implantation, the animals with tumors of measurable size (about 80-120 mm²) are randomized into treatment groups (8-10 rats/group) and the treatments are initiated. One group of the rats are castrated to serve as the negative control of tumor growth. Animals are treated daily with compounds of the current invention, standard antiandrogens such as bacilutamide or vehicle (control) for an average of 10 to 14 weeks. Test compounds are dissolved in a vehicle of (2.5 ml/kg of body weight) 10% polyethylene glycol and 0.05% Tween-80 in 1% carboxymethyl cellulose, PEG/CMC, (Sigma, St Louis, Mo.). Typical therapeutic experiments would include three groups of three escalating doses for each standard or test compound (in a range of 300-3 mg/kg).

Tumors in the vehicle (control) group reach a size of 1500 to 2500 mm³, whereas the castrated animal group typically shows tumor stasis over the 14 weeks of observation. Animals treated orally with 20 mg/kg of bicalutamide or flutamide would be expected to show a 40% reduction in tumor volumes compared to control after 14 weeks of treatment. The size of tumors are measured weekly by vernier caliper (Froboz, Switzerland), taking perpendicular measurements of length and width. Tumor volumes are measured in mm³ using the formula: Length×Width×Height=Volume. Statistical differences between treatment groups and control are evaluated using multiple ANOVA analysis followed by one tail non-parametric Student t test.

Mature Rat Prostate Weight Assay:

The activity of compounds of the present invention were investigated in a mature male rat model, which is a variation of the Levator ani & wet prostate weight assay described above. The above in vivo assays are recognized assays for determining the anabolic effects in muscle and sustaining effects in sex organs for a given compound, as described in L. G. Hershberger et al., 83 Proc. Soc. Expt. Biol. Med., 175 (1953); B. L. Beyler et al, “Methods for evaluating anabolic and catabolic agents in laboratory animals”, 23 J. Amer. Med. Women's Ass., 708 (1968); H. Fukuda et al., “Investigations of the levator ani muscle as an anabolic steroid assay”, 14 Nago Dai. Yak. Ken. Nem. 84 (1966) the disclosures of which are herein incorporated by reference. The basis of this assay lies in the well-defined action of androgenic agents on the maintenance and growth of muscle tissues and sexual accessory organs in animals and man.

The male sexual accessory organs, such as the prostate and seminal vesicles, play an important role in reproductive function. These glands are stimulated to grow and are maintained in size and secretory function by the continued presence of serum testosterone (T), which is the major serum androgen (>95%) produced by the Leydig cells in the testis under the control of the pituitary luteinizing hormone (LH) and follicle stimulating hormone (FSH). Testosterone is converted to the more active form, dihydrotestosterone, (DHT), within the prostate by 5α-reductase. Adrenal androgens also contribute about 20% of total DHT in the rat prostate, compared to 40% of that in 65-year-old men. F. Labrie et. al. 16 Clin. Invest. Med., 475-492 (1993). However, this is not a major pathway, since in both animals and humans, castration leads to almost complete involution of the prostate and seminal vesicles without concomitant adrenalectomy. Therefore, under normal conditions, the adrenals do not support significant growth of prostate tissues, M. C. Luke and D. S. Coffey, “The Physiology of Reproduction” ed. By E. Knobil and J. D. Neill, 1, 1435-1487 (1994). Since the male sex organs and the levator ani are the tissues most responsive to modulation of the androgen activity, this model is used to determine the activity of compounds that modulate the androgen receptor pathway in mature rats.

Along with its mitogenic activity on tissues such as prostate, seminal vesicle and muscle, testosterone also serves as a negative regulator for its own biosynthesis. Testosterone production in the Leydig cells of the testis is controlled by the level of circulating LH released from the pituitary gland. LH levels are themselves controlled by the level of LHRH produced in the hypothalmic region. Testosterone levels in the blood serve to inhibit the secretion of LHRH and subsequently reduce levels of LH and ultimately the levels of circulating testosterone levels. By measuring blood levels of LH as they are effected by compounds of the present invention (“test compounds”), it is possible to determine the level of agonist or antagonist activity of said compounds at the hypothalamic axis of this endocrine cycle.

Matched sets of Harlan Sprague-Dawely rats (40-42 days old, 180-220 g), were dosed orally by gavage (p.o.) with the test compounds in dissolved/suspensions of 80% PEG 400 and 20% Tween 20 (PEGTW) for 14 days. Two control groups, one intact and one castrated were dose orally only with the PEGTW vehicle. Animals were dosed (v/w) at 0.5 ml of vehicle/100 g body weight. Experimental groups were as follows:

1. Intact vehicle (p.o., PEGTW, QD)

2. Control vehicle (p.o., PEGTW, QD)

3. Bicalutamide (Casodex, a recognized antiandrogen, as a reference compound) or a compound of the present invention, p.o. in PEGTW QD. (in a range of doses). At the end of the 14-day treatment, the animals were sacrificed, and the ventral prostate, the seminal vesicles, and the levator ani were removed surgically and weighed. To compare data from different experiments, the organs weights were first standardized as mg per 100 g of body weight, and expressed as a percentage of the value of the respective organ in the intact group.

Rat luteinizing hormone (rLH) is quantitatively determined with the Biotrak [125 I] kit (Amersham Pharmacia Biotek), following the manufacturer directions. The assay is based on the competition by the LH present in the serum of the binding of [¹²⁵I] rLH to an Amerlex-M bead/antibody suspension. The radioactivity that remains after incubation with the serum and subsequent washes is extrapolated into a standard curve to obtain a reading in ng/ml.

The gain and loss of sexual organ and levator ani weight reflect the changes of the cell number (DNA content) and cell mass (protein content), depending upon the serum androgen concentration, see Y. Okuda et al., J. Urol., 145, 188-191 (1991), the disclosure of which in herein incorporated by reference. Therefore, measurement of organ wet weight is sufficient to indicate the bioactivity of androgens and androgen antagonist. In the mature rats assay, active agonist agents will have no effect or will increase the weight of one or more of the androgen responsive organs (levator ani, prostate, seminal vessicle) and will have no effect or a suppressive effect on LH secretion. Compounds with antagonist activity will decrease the weight of one or more of the androgen responsive organs (levator ani, prostate, seminal vesicle) and will have no effect or a reduced suppressive effect on LH secretion.

CWR22 Human Prostate Zenograft Assay:

In Vivo Antitumor Testing: CWR22 human prostate tumors were maintained in Balbic nu/nu nude mice. Tumors were propagated as subcutaneous transplants in adult male nude mice (4-6 weeks old) using tumor fragments obtained from donor mice. Tumor passage occurred every 5-6 weeks.

For antitumor efficacy trial, the required number of animals needed to detect a meaningful response were pooled at the start of the experiment and each was given a subcutaneous implant of a tumor fragment (˜50 mg) with a 13-gauge trocar. Tumors were allowed to grow to approx. 100-200 mg (tumors outside the range were excluded) and animals were evenly distributed to various treatment and control groups. Treatment of each animal was based on individual body weight. Treated animals were checked daily for treatment related toxicity/mortality. Each group of animals was weighed before the initiation of treatment (Wt1) and then again following the last treatment dose (Wt2). The difference in body weight (Wt2−Wt1) provides a measure of treatment-related toxicity.

Tumor response was determined by measurement of tumors with a caliper twice a week, until the tumors reach a predetermined “target” size of 0.5 gm. Tumor weights (mg) were estimated from the formula: Tumor weight=(length×width2)÷2.

Tumor response end-point was expressed in terms of tumor growth inhibition (% T/C), defined as the ratio of median tumor weights of the treated tumors (T) to that of the control group (C).

To estimate tumor cell kill, the tumor volume doubling time was first calculated with the formula:

TVDT=Median time (days) for control tumors to reach target size−Median time (days) for control tumors to reach half the target size

And,

Log cell kill=(T−C)÷(3.32×TVDT)

Statistical evaluations of data were performed using Gehan's generalized Wilcoxon test.

The following Examples illustrate embodiments of the present invention, and are not intended to limit the scope of the claims.

Abbreviations

The following abbreviations are used herein:

DBU=1,8-diazabicyclo[5.4.0]undec-7-ene

4-DMAP=4-dimethylaminopyridine

ee=enantiomeric excess

DMF=dimethylformamide

EtOAc=ethyl acetate

LDA=lithium diisopropylamide

Hünig's Base=N,N-diisopropylethylamine

Me=methyl

RT=retention time

TFA=trifluoroacetic acid

THF=tetrahydrofuran

TLC=thin layer chromatography

TMS=trimethylsilyl

pTSA=para-toluenesulfonic acid

Δ=heat

t-Bu=tert-butyl

PhCH₃=toluene

Pd/C=palladium on activated charcoal

TsCl=tosyl chloride

TBSOTf=tert-butyldimethylsilyl trifluoromethane sulfonate

TBS=tert-butyldimethylsilane

MeI=methyl iodide

(BOC)₂O=di-tert-butyl dicarbonate

TEA=triethylamine

n-BuLi=n-butyllithium

rt=room temperature

LC=liquid chromatography

Ts=tosyl

Ph=phenyl

EtOH=ethanol

DCE=dichloroethane

DMSO=dimethylsulfoxide

Ra—Ni=Raney Nickel

MS=molecular sieves

MS(ES)=Electro—Spray Mass Spectrometry

mCPBA=m-chloroperoxybenzoic acid

sat=saturated

AcOH=acetic acid

MeOH=methanol

Et₂O=diethyl ether

Ac=acetyl

DEAD=diethyl azodicarboxylate

h=hours

Et=ethyl

WSDCC=water soluble dicarbonyl diimide, 1-(3-dimethylaminopropyl)-3-ethylcarbodiimide hydrochloride

TBAF=tetrabutylammonium fluoride

DBAD=di-terbutylazodicarboxylate

DCC=Dicyclohexylcarbodiimide

Wilkinson's catalyst=RhCl(PPh₃)₃

ADDP=1,1-[azodicarbonyl]dipiperidine

DMA=dimethylacetamide

DME=1,2-dimethoxyethane

BOP=benzotriazol-1-yloxytris(dimethylamino)-phosphonium hexafluorophosphate

### EXAMPLE 1

### (3aα,4α,7α,7aα-2-(4-Bromo-3-methylphenyl)tetrahydro-4,7-ethanothiopyrano[3,4-c]pyrrole-1,3,8(2H,4H)-trione (1C)

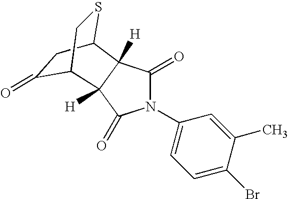

A. 4-(tert-Butyldimethylsiloxy)-2H-thiopyran (1A)

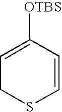

2,3-Dihydro-4H-thiopyran-4-one (1.50 g, 13.14 mol, synthesized as described in Richards et al., J. Org. Chem. 46, 4836-4842 (1981)) was dissolved in CH₂Cl₂ (130 mL) and triethylamine (5.47 mL, 39.41 mmol) was added. tert-Butyldimethylsilyl trifluoromethanesulfonate (3.62 mL, 15.77 mmol) was then added. After 10 minutes, the volatiles were removed by rotary evaporator at 25° C. The resulting yellow oil was passed through a short column of SiO₂ eluting with 3% TEA in hexanes to yield 1.82 g of compound 1A as an orange oil.

B. 1-[4-bromo-3-methylphenyl]-1H-pyrrole-2,5-dione (1B)

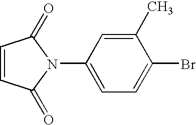

4-Bromo-3-methylaniline (1.55 g, 8.33 mmol) and maleic anhydride (0.898 g, 9.16 mmol) were dissolved in acetic acid (10 mL) and heated at 115° C. for 12 h. The reaction was then cooled to 25° C. and the acetic acid was removed in vacuo. The resulting residue was suspended in 5% K₂CO₃ (100 mL), stirred for 25 minutes and followed by filtering and rinsing with water. The material was then dried in vacuo to give compound 1B as a light brown solid (1.65 g). HPLC: 100% at 2.96 min (retention time) (YMC S5 ODS column 4.6×50 mm eluting with 10-90% aqueous methanol over 4 minutes containing 0.1% TFA, 4 mL/min, monitoring at 220 nm).

C. (3aα,4α,7α,7aα)-2-(4-Bromo-3-methylphenyl)tetrahydro-4,7-ethanothiopyrano[3,4-c]pyrrole-1,3,8(2H,4H)-trione (1C)

Compound 1A (0.313 g, 1.41 mmol) and compound 1B (0.250 g, 0.94 mmol) were dissolved in toluene and heated to reflux for 5 h. The toluene was then removed by passing a stream of argon through the reaction flask. The residue was then purified by flash chromatography on SiO₂ eluting with 20% hexane in chloroform. This gave 0.168 g of the enol ether intermediate as a yellow solid. The enol ether intermediate was dissolved in dichloroethane (2.0 mL) and TFA (0.25 mL) was added. After 0.5 h, the reaction was quenched with saturated aqueous NaHCO₃ and extracted with CH₂Cl₂ (2×30 mL). The organics were dried over anhydrous sodium sulfate and evaporated to give 0.079 g of compound 1C as a white solid. HPLC: 99% at 3.010 min (retention time) (YMC S5 ODS column 4.6×50 mm eluting with 10-90% aqueous methanol over 4 minutes containing 0.1% TFA, 4 mL/min, monitoring at 220 nm), MS (ES): m/z 396.9 [M+NH₄]⁺.

### EXAMPLE 2

### (3aα,4α,7α,7aα)-2-(4-Bromo-3-methylphenyl)tetrahydro-4,7-ethanothiopyrano[3,4-c]pyrrole-1,3,8(2H,4H)-trione 5,5-dioxide (2)

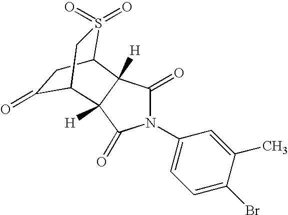

Compound 1C (0.040 g, 0.105 mmol) was dissolved in CH₂Cl₂ (4.0 mL) and cooled to 0° C. m-CPBA (60% purity, 0.061 g, 0.210 mmol) was added and the reaction was then warmed to 25° C. After 1 h, a 1:1 mixture of saturated NaHCO₃ and saturated sodium sulfite (20 mL) was added with vigorous stirring. After 15 minutes, the mixture was extracted with CH₂Cl₂ (2×30 mL) and the organics were dried over anhydrous sodium sulfate to yield 0.031 g of compound 2 as a white solid. No purification was necessary. HPLC: 78% at 2.290 min (retention time) (YMC S5 ODS column 4.6×50 mm eluting with 10-90% aqueous methanol over 4 minutes containing 0.1% TFA, 4 mL/min, monitoring at 220 nm), MS (ES): m/z 429.8 [M+NH₄]⁺.

### EXAMPLE 3

### (3aα,4β,7β,7aα)-2-(3-Chlorophenyl)hexahydro-4-methyl-4,7-epoxy-1H-isoindole-1,3(2H)-dione (3)

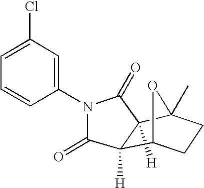

3-Chloroaniline (0.100 g, 0.787 mmol) and 3,6-endoxo-3-methylhexahydrophthalic anhydride (0.172 g, 0.945 mmol) were dissolved in AcOH (2.0 mL) and heated to 110° C. for 11 h. The reaction was then cooled to 25° C. and poured into cold saturated aq K₂CO₃ and stirred vigorously for 10 min. The solution was then filtered and rinsed with water. The resulting filtrate was dried in vacuo to give 0.118 g of compound 3 as a white solid. No further purification was needed. HPLC: 99% at 2.510 min (retention time) (YMC S5 ODS column 4.6×50 mm eluting with 10-90% aqueous methanol over 4 minutes containing 0.1% TFA, 4 mL/min, monitoring at 220 nm), MS (ES): m/z 292.32 [M+H]⁺.

### EXAMPLE 4

### (3aα,4α,7α,7aα)- and (3aα,4β,7β,7aα)-4-[(Acetyloxy)methyl]-3a,4,7,7a-tetrahydro-2-[3-(trifluoromethyl)phenyl]-4,7-epoxy-1H-isoindole-1,3(2H)-dione (4i and 4ii, respectively)

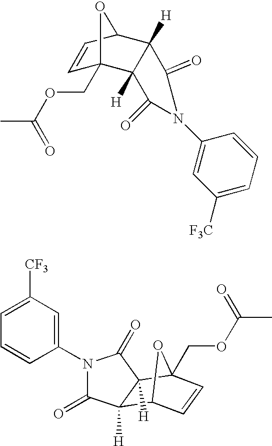

2-Acetoxymethylfuran (0.599 mL, 4.78 mmol) and 1-[3-(trifluoromethyl)-phenyl]-1H-pyrrole-2,5-dione (0.500 g, 2.39 mmol) were dissolved in methylene chloride (3.0 mL) at 25° C. After 22 h, the volatiles were removed in vacuo and the resulting residue was purified by flash chromatography on SiO₂ eluting with 0-15% acetone in methylene chloride to give 0.438 g of a yellow oil, as a 2:1 mixture of compound 4i and compound 4ii, which was not separated. HPLC: 100% at 3.093 min (retention time) (YMC S5 ODS column 4.6×50 mm eluting with 10-90% aqueous methanol over 4 minutes containing 0.1% TFA, 4 mL/min, monitoring at 220 nm), MS (ES): m/z 398.9 [M+NH₄]⁺.

### EXAMPLE 5

### (3aα,4α,7α,7aα)- and (3aα,4β,7β,7aα)-4-[(Acetyloxy)methyl]-Hexahydro-2-[3(trifluoromethyl)phenyl]-4,7-epoxy-1H-isoindole-1,3(2H)-dione (5i and 5ii, respectively)

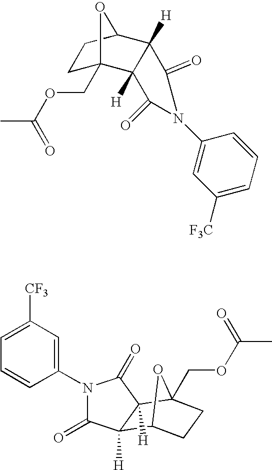

The 2:1 mixture of compounds 4i and 4ii (0.361 g, from Example 4) was dissolved in ethyl acetate (25 mL) and Pd/C (10% Pd, 0.2 g) was added. Hydrogen was introduced via a balloon and the reaction was stirred at 25° C. for 4 h; followed by filtration through celite and rinsed with ethyl acetate. Concentration in vacuo gave a yellow oil that was determined to be a 2:1 mixture of compound 5i and compound 5ii (0.348 g), which was not separated. HPLC: 100% at 2.900 min (retention time) (YMC S5 ODS column 4.6×50 mm eluting with 10-90% aqueous methanol over 4 minutes containing 0.1% TFA, 4 mL/min, monitoring at 220 nm), MS (ES): m/z 401.0 [M+NH₄]⁺.

### EXAMPLE 6

### (3aα,4α,7α,7aα)- and (3aα,4β,7β,7aα)-3a,4,7,7a-Tetrahydro-5-(hydroxymethyl)-2-[3-(trifluoromethyl)phenyl]-4,7-epoxy-1H-isoindole-1,3(2H)-dione (6i and 6ii, respectively)

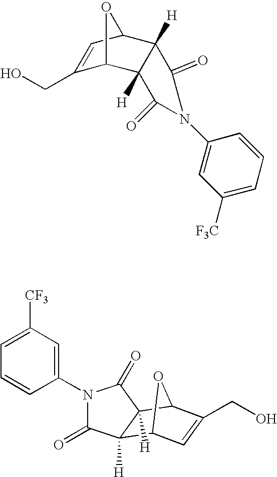

1-[3-(Trifluoromethyl)phenyl]-1H-pyrrole-2,5-dione (0.500 g, 2.39 mmol) and 3-furanmethanol (0.412 mL, 4.78 mmol) were dissolved in methylene chloride (3.0 mL) and stirred at 25° C. for 20 h. The volatiles were then removed in vacuo and the resulting material purified by flash chromatography on SiO₂ eluting with chloroform/acetone to give 0.379 g of compound 6i and 0.220 g of compound 6ii, both as white solids. Compound 6i: HPLC: 100% at 2.197 min (retention time) (YMC S5 ODS column 4.6×50 mm eluting with 10-90% aqueous methanol over 4 minutes containing 0.1% TFA, 4 mL/min, monitoring at 220 nm), MS (ES): m/z 338.0 [M−H]⁻. Compound 6ii: HPLC: 100% at 2.477 min (retention time) (YMC S5 ODS column 4.6×50 mm eluting with 10-90% aqueous methanol over 4 minutes containing 0.1% TFA, 4 mL/min, monitoring at 220 nm), MS (ES): m/z 338.0 [M−H]⁻.

### EXAMPLE 7

### (3aα,4α,7α,7aα)-3a,4,7,7a-Tetrahydro-5-(hydroxymethyl)-4-methyl-2-[3-(trifluoromethyl)phenyl]-4,7-epoxy-1H-isoindole-1,3(2H)-dione (7)

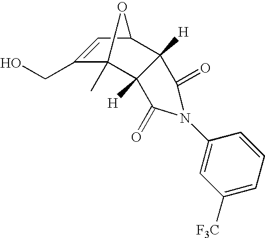

2-Methyl-3-furanmethanol (0.537 g, 4.78 mmol) and 1-[3-(trifluoromethyl)-phenyl]-1H-pyrrole-2,5-dione (0.500 g, 2.39 mmol) were dissolved in dichloroethane (2.0 mL) and stirred at 25° C. for 20 h. The reaction was then concentrated in vacuo and purified by flash chromatography in SiO₂ eluting with ethyl acetate/methylene chloride to give 0.317 g of compound 7 as a white solid. No other possible isomer was isolated after chromatography. HPLC: 100% at 2.197 min (retention time) (YMC S5 ODS column 4.6×50 mm eluting with 10-90% aqueous methanol over 4 minutes containing 0.1% TFA, 4 mL/min, monitoring at 220 nm), MS (ES): m/z 351.9[M−H]⁻.

### EXAMPLE 8

### (3aα,4β,7β,7aα)-2-[3,5-Bis(trifluoromethyl)phenyl]hexahydro-4,7-epoxy-1H-isoindole-1,3(2H)-dione (8)

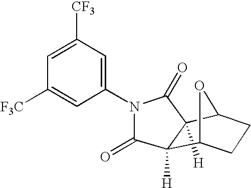

3,5-Bis-(trifluoromethyl)-aniline (0.017 g, 0.0075 mmol) was dissolved in acetic acid (0.300 mL) and transferred to a 1.5 mL conical vial with a septa cap. Stock solutions of an additional 95 amines were prepared as described above. To each of the above vials was added 0.4 mL (0.12 mmol) of a stock solution of exo-7-oxabicyclo[2.2.1]heptane-2,3-dicarboxylic anhydride in acetic acid. The vials were then sealed and heated at 110° C. for 11 h. Upon cooling to 25° C., the caps were removed from the vials and the acetic acid was removed in vacuo. To each vial was added 1 mL of 2:1 acetone/methylene chloride and the vials were heated at 40° C. for 1 h. Once all products were in solution, they were transferred via robot to filter tubes with coarse frits pre-wetted with 0.2 mL of water. Nitrogen was blown through each tube until the volatile organics were removed. 1.5 mL of 10% aq K₂CO₃ was then added to each tube followed by vigorous shaking at 25° C. for 15 min. The tubes were then drained, resealed and 1.0 mL of water was added to each tube followed by shaking. The tubes were drained again and washed with water a second time. The resulting residues in each tube was then dried in vacuo for 48 h. After drying, 1.0 mL of 20% TFA in methylene chloride was added to each tube and the racks were shaken for 30 min. The tubes were then drained into a 96-well plate with pre-tared custom micro-tubes present. Each tube was assayed for product purity (analytical LC) and identity (LC-MS). The tubes were then concentrated in vacuo and weighed for yields. The tube containing the reaction of 3,5-bistrifluoromethylaniline and exo-7-oxabicyclo[2.2.1]heptane-2,3-dicarboxylic anhydride, yielded 0.022 g of compound 8 as a white solid. HPLC: 94% at 4.03 min (retention time) (YMC S5 ODS column 4.6×50 mm eluting with 10-90% aqueous methanol over 4 minutes containing 0.1% TFA, 4 mL/min, monitoring at 220 nm), MS (ES): m/z 434.2 [M+Na+MeOH]⁺. Of the remaining 95 additional reactions run, a total of 80 final compounds were obtained in >70% purity and >5 mg yield. Several samples needed further purification which was performed by short SiO₂ column eluting with methylene chloride/acetone. See Table 2 below.

### EXAMPLE 9

### (3aα,4α,7α,7aα)-2-(4-Bromophenyl)octahydro-1,3-dioxo-4,7-etheno-5H-pyrrolo[3,4-c]pyridine-5-carboxylic acid phenyl ester (9)

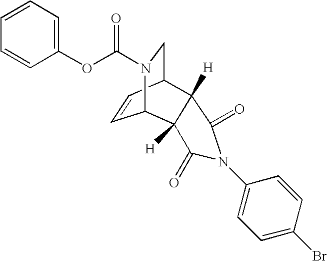

1-[4-Bromophenyl]-1H-pyrrole-2,5-dione (0.250 g, 0.992 mmol, synthesized as described in Example 1B) and 1 (2H)-pyridinecarboxylic acid phenylmethyl ester (0.299 g, 1.49 mmol, synthesized as described in Richard et. al., J. Org. Chem. 46, 4836-4842 (1981)) were dissolved in toluene and heated to 85° C. for 1 h. Upon cooling to 25° C., the toluene was removed in vacuo. The resulting residue was dissolved in a minimum amount of chloroform and the product was precipitated by addition of hexanes. After 1 h at 25° C., the product was filtered and rinsed with cold 20% hexanes in chloroform giving compound 9 as a white solid (0.243 g) as a single isomer. HPLC: 100% at 3.393 min (retention time) (YMC S5 ODS column 4.6×50 mm eluting with 10-90% aqueous methanol over 4 minutes containing 0.1% TFA, 4 mL/min, monitoring at 220 nm), MS (ES): m/z 454.98 [M+H]⁺.

### EXAMPLE 10

### (3aα,4α,7α,7aα)-2-(4-Bromophenyl)octahydro-1,3-dioxo-4,7-etheno-5H-pyrrolo[3,4-c]pyridine-5-carboxylic acid phenylmethyl ester (10)

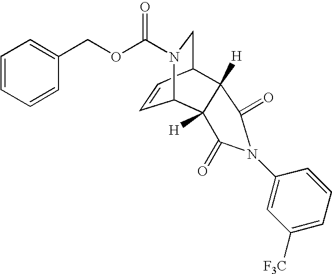

1-[3-(Trifluoromethyl)phenyl]-1H-pyrrole-2,5-dione (3.78 g, 15.7 mmol) and 1(2H)-pyridinecarboxylic acid phenylmethyl ester (4.0 g, 18.8 mmol, synthesized as described in Richard et. al., J. Org. Chem. 46, 4836-4842 (1981)) were dissolved in toluene and heated at 80° C. for 3 h. After cooling to 25° C., the toluene was removed in vacuo and the resulting residue was purified by flash chromatography on SiO₂ eluting with methanol/methylene chloride to give 3.2 g of compound 10 as a yellow oil. HPLC: 95% at 3.510 min (retention time) (YMC S5 ODS column 4.6×50 mm eluting with 10-90% aqueous methanol over 4 minutes containing 0.1% TFA, 4 mL/min, monitoring at 220 nm), MS (ES): m/z 457.2 [M+H]⁺.

### EXAMPLE 11

### (3aα,4α,7α,7aα)-Hexahydro-2-[3-(trifluoromethyl)phenyl]-4,7-ethano-1H-pyrrolo[3,4-c]pyridine-1,3(2H)-dione trifluoroacetate (11)

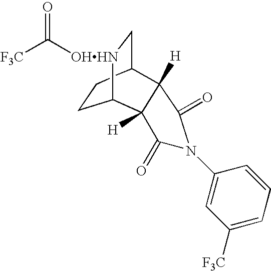

Compound 10 (3.2 g) was dissolved in 100 ml of MeOH and 10% Pd/C DeGussa catalyst (2 g) was added. Hydrogen was then introduced via a balloon.

After 1 h, the reaction was filtered through celite and rinsed with MeOH. The volatiles were removed in vacuo and the resulting crude material was purified by reverse phase preparative HPLC to yield 2.5 g of compound 11 as the TFA salt (white solid). HPLC: 99% at 1.843 min (retention time) (YMC S5 ODS column 4.6×50 mm eluting with 10-90% aqueous methanol over 4 minutes containing 0.1% TFA, 4 mL/min, monitoring at 220 nm), MS (ES): m/z 325.12 [M+H]⁺.

### EXAMPLE 12

### (3aα,4α,7α,7aα)-5-Acetylhexahydro-2-[3-(trifluoromethylphenyl-4,7-ethano-1H-pyrrolo[3,4-c]pyridine-1,3(2H)-dione (12)

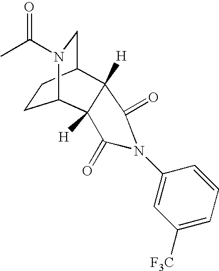

Compound 11 (0.100 g, 0.23 mmol) was suspended in THF (5.0 mL) and TEA (0.097 mL, 0.46 mmol) was added resulting in a homogeneous solution. Acetyl chloride (0.033 mL, 0.46 mmol) was then added. After 2 h, the reaction was quenched with saturated aqueous NaHCO₃ and extracted with methylene chloride (3×15 mL). The crude material was purified by preparative-TLC eluting with chloroform/acetone to give 0.099 g of compound 12 as a colorless oil. HPLC: 99% at 2.66 min (retention time) (YMC S5 ODS column 4.6×50 mm eluting with 10-90% aqueous methanol over 4 minutes containing 0.1% TFA, 4 mL/min, monitoring at 220 nm), MS (ES): m/z 367.0 [M+H]⁺.

### EXAMPLE 13

### (3aα,4α,7α,7aα)-5-Benzoylhexahydro-2-[3-(trifluoromethyl)phenyl]-4,7-ethano-1H-pyrrolo[3,4-c]pyridine-1,3(2H)-dione (13)

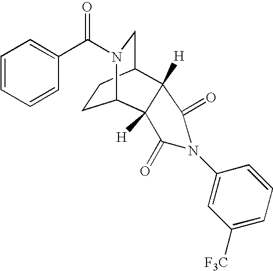

Compound 11 (0.100 g, 0.23 mmol) was suspended in THF (5.0 mL) and TEA (0.097 mL, 0.46 mmol) was added resulting in a homogeneous solution. Benzoyl chloride (0.05433 mL, 0.46 mmol) was then added. After 2 h, the reaction was quenched with saturated aqueous NaHCO₃ and extracted with methylene chloride (3×15 mL). The crude material was purified by reverse phase preparative-HPLC to give 0.020 g of compound 13 as a white foam. HPLC: 99% at 3.183 min (retention time) (YMC S5 ODS column 4.6×50 mm eluting with 10-90% aqueous methanol over 4 minutes containing 0.1% TFA, 4 mL/min, monitoring at 220 nm), MS (ES): m/z 429.1 [M+H]⁺.

### EXAMPLE 14

### (3aα,4α,7α,7aα)-Hexahydro-5-methyl-2-[3-(trifluoromethyl)phenyl]-4,7-ethano-1H-pyrrolo[3,4-c]pyridine-1,3(2H)-dione (14)

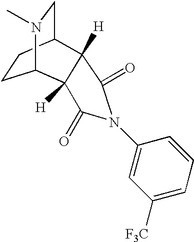

Compound 11 (0.100 g, 0.23 mmol) was suspended in THF (5.0 mL) and TEA (0.097 mL, 0.46 mmol) was added resulting in a homogeneous solution. Dimethyl sulfate (0.043 mL, 0.46 mmol) was added and the reaction stirred at 25° C. After 14 h, the reaction was concentrated in vacuo and the crude material was purified by preparative-TLC eluting with 10% MeOH in methylene chloride to give 0.030 g of compound 14 as a white solid. HPLC: 100% at 1.797 min (retention time) (YMC S5 ODS column 4.6×50 mm eluting with 10-90% aqueous methanol over 4 minutes containing 0.1% TFA, 4 mL/min, monitoring at 220 nm), MS (ES): m/z 339.21 [M+H]⁺.

### EXAMPLE 15

### (3aα,4α,7α,7aα)-Hexahydro-5-(phenylmethyl)-2-[3-(trifluoromethylphenyl]-4,7-ethano-1H-pyrrolo[3,4-c]pyridine-1,3(2H)-dione trifluoroacetate (15)

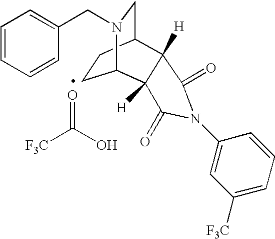

Compound 11 (0.100 g, 0.23 mmol) was dissolved in DMF (5.0 mL) and K₂CO₃ (0.063 g, 0.46 mmol) was added. Benzyl bromide (0.041 mL, 0.35 mmol) was then added. The reaction was stirred at 25° C. for 1 h, and then filtered and concentrated. The crude material was purified by reverse phase preparative-HPLC to give 0.055 g of compound 15 as a white solid. HPLC: 100% at 2.31 min (retention time) (YMC S5 ODS column 4.6×50 mm eluting with 10-90% aqueous methanol over 4 minutes containing 0.1% TFA, 4 mL/min, monitoring at 220 nm), MS (ES): m/z 415.36 [M+H]⁺.

### EXAMPLE 16

### (3aα4α,7α,7aα)-Hexahydro-5-propyl-2-[3-(trifluoromethyl)phenyl]-4,7-ethano-1H-pyrrolo[3,4-c]pyridine-1,3(2H)-dione trifluoroacetate (16)

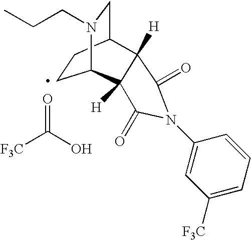

Compound 11 (0.100 g, 0.23 mmol) was dissolved in DMF (5.0 mL) and K₂CO₃ (0.079 g, 0.57 mmol) was added, followed by 1-bromopropane (0.031 mL, 0.34 mmol). The reaction was stirred at 25° C. for 6 h, and then filtered and concentrated. The crude material was purified by reverse phase preparative-HPLC to give 0.070 g of compound 16 as a white solid. HPLC: 100% at 1.907 min (retention time) (YMC S5 ODS column 4.6×50 mm eluting with 10-90% aqueous methanol over 4 minutes containing 0.1% TFA, 4 mL/min, monitoring at 220 nm), MS (ES): m/z 340.22 [M+H]⁺.

### EXAMPLE 17

### (3aα,4α,4aβ,5aβ,6α,6aα)-2-[4-Cyano-3-(trifluoromethyl)phenyl]decahydro-1,3-dioxo-4,6-(iminomethano)cycloprop[f]isoindole-7-carboxylic acid phenylmethyl ester (17)

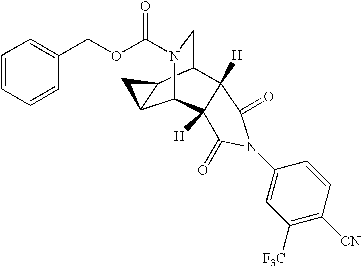

1-Methyl-3-nitro-1-nitrosoguanidine (2.5 g, 17 mmol) was added portion-wise to a solution of 40% KOH/H₂O (15 mL) and Et₂O (25 mL) at 0° C. The ether layer turned yellow once addition was complete. After 30 min at 0° C., the ether layer was poured into a solution of (3aα,4α,7α,7aα)-2-[4-cyano-3-(trifluoromethyl)phenyl]-octahydro-1,3-dioxo-4,7-etheno-5H-pyrrolo[3,4-c]pyridine-5-carboxylic acid phenylmethyl ester (0.50 g, 1.09 mmol, prepared as described in Example 10) and Pd(OAc)₂ (0.010 g) in THF (10 mL) at 0° C. The reaction was then warmed slowly to 25° C. and stirred for 24 h and then filtered through celite, rinsing with THF. The crude material was then purified by flash chromatography on SiO₂ eluting with MeOH/CH₂Cl₂ to give 0.34 g of compound 17 as a white solid and a single isomer. HPLC: 100% at 3.61 min (retention time) (YMC S5 ODS column 4.6×50 mm eluting with 10-90% aqueous methanol over 4 minutes containing 0.1% TFA, 4 mL/min, monitoring at 220 nm), MS (ES): m/z 496.25 [M+H]⁺.

### EXAMPLE 18

### (3aα,4α,4aβ,5aβ,6α,6aα)-4-[Decahydro-1,3-dioxo-4,6-(iminomethano)cycloprop[f]isoindol-2-yl]-2-(trifluoromethyl)benzonitrile (18)

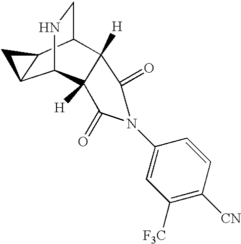

Compound 17 (0.200 g, 0.404 mmol) was dissolved in MeOH (20 mL) and 5% Pd/C (0.200 g) was added. Hydrogen was then introduced via balloon. After 3 h, the reaction was filtered through celite, rinsed with MeOH and the volatiles were removed in vacuo to yield compound 18 (0.130 g) as a white solid. HPLC: 100% at 1.80 min (retention time) (YMC S5 ODS column 4.6×50 mm eluting with 10-90% aqueous methanol over 4 minutes containing 0.1% TFA, 4 mL/min, monitoring at 220 nm), MS (ES): m/z 362.09 [M+H]⁺.

### EXAMPLE 19

### (3aα,4α,4aβ,5aβ,6α,6aα)-4-[Decahydro-7-methyl-1,3-dioxo-4,6-(iminomethano)cycloprop[f]isoindol-2-yl]-2-(trifluoromethyl)benzonitrile (19)

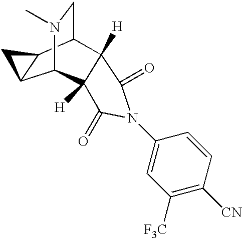

Compound 17 (0.100 g, 0.277 mmol) was dissolved in CH₃CN (2.0 mL). TEA (0.19 mL, 1.39 mmol) and MeI (0.052 mL, 0.83 mmol) were then added and the reaction was stirred at 25° C. for 14 h. The reaction was concentrated and the crude material was dissolved in CH₂Cl₂/water and extracted with CH₂Cl₂ (3×15 mL). The combined organics were dried over anhydrous Na₂SO₄. The crude material was purified by flash chromatography eluting with 3% MeOH/CH₂Cl₂ to give 0.030 g of compound 19 as a light yellow solid. HPLC: 100% at 1.720 min (retention time) (YMC S5 ODS column 4.6×50 mm eluting with 10-90% aqueous methanol over 4 minutes containing 0.1% TFA, 4 mL/min, monitoring at 220 nm), MS (ES): m/z 376.11 [M+H]⁺.

### EXAMPLE 20

### (3aα,4β,7β,7aα)-4-(Octahydro-4,7-dimethyl-1,3-dioxo-4,7-epoxy-2H-isoindol-2-yl)-2-(trifluoromethyl)benzonitrile (20B)

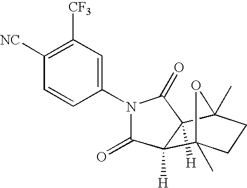

A. (3aα,4β,7β,7aα)-Hexahydro-4,7-epoxyisobenzofuran-1,3-dione (20A)

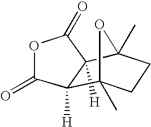

Freshly distilled dimethyl furan (1.60 mL, 15.3 mmol) was dissolved in CH₂Cl₂ (2.0 mL) and maleic anhydride (1.0 g, 10.2 mmol) was added. The reaction was stirred at 25° C. for 16 h and was then concentrated in vacuo to give a yellow solid. This solid was dissolved in ethyl acetate (30 mL) and Pd/C (10% Pd, 0.200 g) was added. Hydrogen was then introduced by a balloon and the reaction stirred for 24 h. The Pd was removed by filtration through celite rinsing with EtOAc followed by concentration in vacuo to give the compound 20A (1.69 g) as a white solid. 2-Dimensional NOE experiments confirmed the structural assignment to be that of compound 20A.

B. (3aα,4β,7β,7aα)-4-(Octahydro-4,7-dimethyl-1,3-dioxo-4,7-epoxy-2H-isoindol-2-yl)-2-(trifluoromethyl)benzonitrile (20B)

A solution of compound 20A (603 mg, 3.21 mmol, 1 eq), 5-amino-2-cyanobenzotrifluoride (640 mg, 3.44 mmol, 1.07 eq) and TsOH (10 mg, cat amount) in toluene (5 mL) was heated in a sealed tube for 2 days. The reaction mixture was cooled to room temperature and then concentrated under reduced pressure. Purification by flash chromatography on silica gel eluting with 50% EtOAc/hexanes gave 400 mg (1.10 mmol, 34%) of compound 20B as a white solid. HPLC: 99% at 3.04 min (retention time) (YMC S5 ODS column 4.6×50 mm, 10-90% aqueous methanol over 4 minutes containing 0.2% phosphoric acid, 4 mL/min, monitoring at 220 nm), MS (ESI): m/z 382.2 [M+NH₄]⁺.

### EXAMPLE 21

### (3aα,4β,7β,7aα)-N-[4-[[2-[2-[4-Cyano-3-(trifluoromethyl)phenyl]octahydro-7-methyl-1,3-dioxo-4,7-epoxy-4H-isoindol-4-yl]ethyl]thio]phenyl]acetamide (21E)

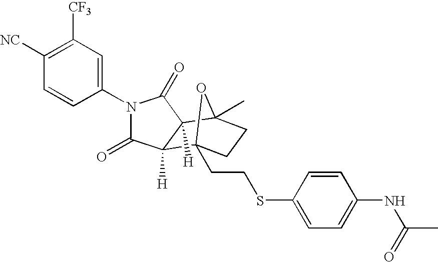

A. 5-Methyl-2-furanethanol (21A)

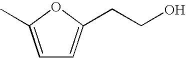

A solution of n-BuLi (83 mL, 133.0 mmol, 1.2 eq, 1.6 M in hexanes) was added to a stirred solution of 2-methylfuran (10 mL, 110.8 mmol, 1 eq) in THF (85 mL) at 0° C. under inert atmosphere. The reaction mixture was stirred for 4 h at room temperature then cooled to 0° C. Ethylene oxide (8.3 mL, 166.3 mmol, 1.5 eq) was added dropwise and the reaction mixture was allowed to warm to room temperature overnight. After quenching with saturated aqueous NH₄Cl, the resulting layers were separated and the aqueous layer was extracted with Et₂O (2×). The combined organic layers were dried over Na₂SO₄ and concentrated under reduced pressure. Distillation at atmospheric pressure (170-185° C.) gave 10.13 g (80.3 mmol, 72%) of compound 21A as a light yellow oil.

B. 2-(2-Bromoethyl)-5-methylfuran (21B)

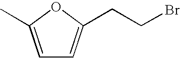

Ph₃Br₂ (3.68 g, 8.72 mmol, 1.1 eq) was added to a solution of compound 21A (1 g, 7.93 mmol, 1 eq) in DMF (8 mL) and the reaction mixture was stirred at room temperature for 1 h. The reaction mixture was added to H₂O and extracted with EtOAc (3×). The combined organic layers were washed with H₂O (2×), dried over Na₂SO₄ and concentrated under reduced pressure. Purification by flash chromatography on silica gel eluting with 10% EtOAc/hexanes gave 0.507 g (2.68 mmol, 34%) of compound 21B.

C. N-[4-[[2-(5-Methyl-2-furanyl)ethyl]thio]phenyl]acetamide (21C)

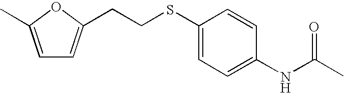

To a solution of 4-acetamidothiophenol (442 mg, 2.64 mmol, 1 eq) in THF (1 mL) at 0° C. under inert atmosphere was added a solution of n-BuLi (2 mL, 3.17 mmol, 1.2 eq, 1.6 M in hexanes) in THF (1 mL). The reaction solution was stirred at room temperature for 10 mins and a solution of compound 21B (0.5 g, 2.64 mmol, 1 eq) in THF (3 mL) was added. After all the starting material was consumed as determined by TLC, the reaction was quenched with H₂O and the mixture was extracted with EtOAc (2×), dried over Na₂SO₄ and concentrated under reduced pressure. Purification by flash chromatography on silica gel eluting with 50% EtOAc/hexanes gave 0.644 g (2.34 mmol, 88%) of compound 21C. MS (ESI): m/z 276.09 [M+H]⁺.

D. (3aα,4β,7β,7aα)-N-[4-[[2-[2-[4-Cyano-3-(trifluoromethyl)phenyl]-1,2,3,3a,7,7a-hexahydro-7-methyl-1,3-dioxo-4,7-epoxy-4H-isoindol-4-yl]ethyl]thio]phenyl]acetamide (21D)

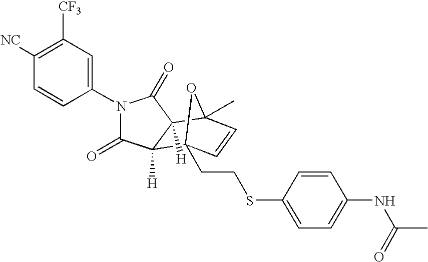

A solution of compound 21C (195 mg, 0.708 mmol, 1 eq) and 4-(2,5-dihydro-2,5-dioxo-1H-pyrrol-1-yl)-2-trifluoromethylbenzonitrile (377 mg, 1.416 mmol, 2 eq, prepared as described for Example 1B) in CH₂Cl₂ (1.5 mL) was stirred at room temperature for two days. The reaction mixture was concentrated under reduced pressure to yield compound 21D as determined by NMR analysis. Compound 21D was used directly in th/e next step without purification.

E. (3aα,4β,7β,7aα)-N-[4-[[2-[2-[4-Cyano-3-(trifluoromethyl)phenyl]-octahydro-7-methyl-1,3-dioxo-4,7-epoxy-4H-isoindol-4-yl]ethyl]thio]-phenyl]acetamide (21E)

A solution of crude compound 21D (0.708 mmol) and 10% Pd/C (200 mg) in MeOH (20 mL) was stirred under a hydrogen atmosphere over night. Purification by preparative chromatography [HPLC at 34.4 min (retention time) (YMC S5 ODS column 20×250 mm, 0-100% aqueous methanol over 30 minutes containing 0.1% TFA, 10 mL/min, monitoring at 220 nm)] followed by flash chromatography on silica gel eluting with 1% MeOH/CH₂Cl₂ gave 29 mg (0.053 mmol, 7.5%) of compound 21E as a yellow powder. HPLC: 99% at 3.44 min (retention time) (YMC S5 ODS column 4.6×50 mm, 10-90% aqueous methanol over 4 minutes containing 0.2% phosphoric acid, 4 mL/min, monitoring at 220 nm), MS (ESI): m/z 544.01 [M+H]⁺.

### EXAMPLE 22

### (3aα,4β,7β,7aα)-N-[4-[[2-2-[4-Cyano-3-(trifluoromethyl)phenyl]octahydro-7-methyl-1,3-dioxo-4,7-epoxy-4H-isoindol-4-yl]ethyl]sulfinyl]phenyl]acetamide (22)

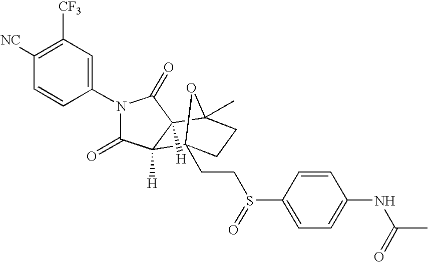

mCPBA (12 mg, 0.05 mmol) was added portion-wise to a solution of crude compound 21E (65 mg, 0.12 mmol, 1 eq) in CH₂Cl₂ (6 mL) until the starting material was consumed. Purification by preparative chromatography [HPLC at 30.5 min (retention time) (YMC S5 ODS column 30×250 mm, 0-100% aqueous methanol over 30 minutes containing 0.1% TFA, 25 mL/min, monitoring at 220 nm)] gave 27.5 mg (0.049 mmol, 41%) of compound 22 as a tan solid (˜1:1 mixture of diastereomers). HPLC: 96% at 2.88 min (retention time) (YMC S5 ODS column 4.6×50 mm eluting with 10-90% aqueous methanol over 4 minutes containing 0.1% TFA, 4 mL/min, monitoring at 220 nm), MS (ESI): m/z 559.97 [M+H]⁺.

### EXAMPLE 23

### (3aα,4β,7β,7aα)-N-[4-[[2-[2-[4-Cyano-3-(trifluoromethyl)phenyl]octahydro-7-methyl-1,3-dioxo-4,7-epoxy-4H-isoindol-4-yl]ethyl]sulfonyl]phenyl]acetamide (23)

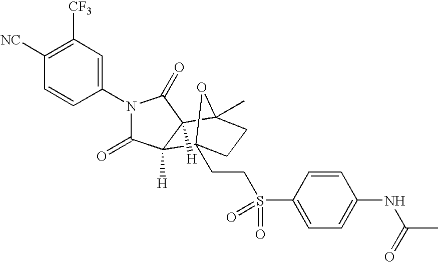

mCPBA (26 mg, 0.105 mmol, 3 eq) was added to a solution of compound 21E (19 mg, 0.035 mmol, 1 eq) in CH₂Cl₂ (6 mL) and the reaction was stirred at rt until starting material and the intermediate sulfoxide (compound 22) were consumed as was apparent by TLC. Purification by preparative chromatography [HPLC at 53.3 min (retention time) (YMC S5 ODS column 30×250 mm, 0-70% aqueous methanol over 45 minutes containing 0.1% TFA, 25 mL/min, monitoring at 220 nm)] gave 27.5 mg (0.049 mmol, 40%) of compound 23 as a white solid. HPLC: 99% at 2.94 min (retention time) (YMC 5S ODS column 4.6×50 mm, 10-90% aqueous methanol over 4 minutes containing 0.2% phosphoric acid, 4 mL/min, monitoring at 220 nm), MS (ESI): m/z 575.95 [M+H]⁺.

### EXAMPLE 24

### (3aα,4β,7β,7aα)- and (3aα,4α,7α,7aα)-N-[2-[2-[4-Cyano-3-(trifluoromethyl)phenyl]octahydro-7-methyl-1,3-dioxo-4,7-epoxy-4H-isoindol-4-yl]ethyl]benzenesulfonamide (24Ci and 24Cii, respectively)

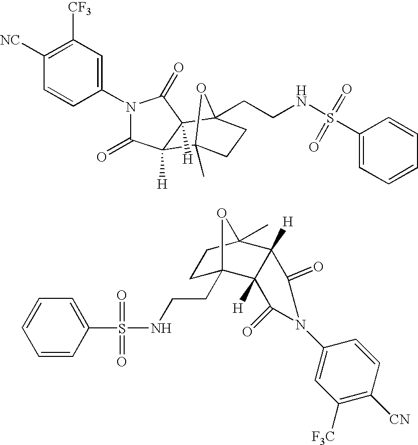

A. 5-Methyl-2-furanethanol 4-methylbenzenesulfonate (24A)

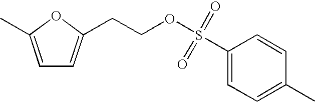

4-Methylbenzenesulfonyl chloride (907 mg, 4.76 mmol) was added to a solution of compound 21A (500 mg, 3.96 mmol) in 6 ml of dry pyridine. The reaction was stirred at room temperature for 4 h, then quenched with ice. The reaction mixture was extracted with CH₂Cl₂ and the combined organic layers were washed with saturated aqueous sodium bicarbonate and water, dried and concentrated under reduced pressure to give 900 mg (81%) of compound 24A as a yellow oil.

B. N-[2-(5-Methyl-2-furanyl)ethyl]benzenesulfonamide (24B)

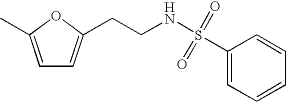

Benzenesulfonamide (157 mg, 1 mmol) was added to a 10% aqueous solution of sodium hydroxide (0.4 ml, 1 mmol). A solution of compound 24A (280 mg, 1 mmol) in acetone (1 mL) was then added. The reaction mixture was heated at 90° C. for 8 h then cooled to room temperature. Ice was added and the mixture was extracted with CH₂Cl₂. The combined organic layers were washed with water, dried and concentrated under reduced pressure. Purification by flash chromatography on silica gel, eluting with CH₂Cl₂ gave 60 mg (23%) of compound 24B as yellow oil.

C. (3aα,4β,7β,7aα)- and (3aα,4α,7α,7aα)-N-[2-[2-[4-Cyano-3-(trifluoromethyl)phenyl]octahydro-7-methyl-1,3-dioxo-4,7-epoxy-4H-isoindol-4-yl]ethyl]benzenesulfonamide (24Ci and 24Cii, respectively)

4-(2,5-Dihydro-2,5-dioxo-1H-pyrrol-1-yl)-2-trifluoromethylbenzonitrile (129 mg, 0.45 mmol, prepared as described in Example 1B) was added to a solution of compound 24B (60 mg, 0.23 mmol) in CH₂Cl₂ (2 mL). The reaction mixture was stirred at room temperature for 2 days, concentrated under reduced pressure and purified by flash chromatography on silica gel, eluting with 70% EtOAc/hexanes, to give 20 mg (16%) of the unsaturated Diels-Alder product. The unsaturated product (20 mg) was immediately dissolved in 2 ml of ethanol and 10 mg of 10% Pd/C was added. The solution was stirred at room temperature overnight under a hydrogen atmosphere. The mixture was filtered and the filtrate was concentrated under reduced pressure. Purification by preparative reverse phase HPLC gave 7 mg of compound 24Ci and 2 mg of compound 24Cii. Compound 24Ci: HPLC: 96% at 3.17 min (retention time) (YMC ODSA S5 C18 4.6×50 mm, 10%-90% aqueous methanol over 4 min gradient with 0.1% TFA, detected at 220 nm), MS (ES): m/z: 533.99 [M+H]⁺. Compound 24Cii: HPLC: 99% at 38.95 min (retention time) (YMC ODS S5 20×250 mm, 10%-90% aqueous methanol over 40 min gradient with 0.1% TFA, detected at 220 nm), MS (ES): m/z 533.99 [M+H]⁺.

### EXAMPLE 25

### (3aα,4β,7β,7aα)-4-[Octahydro-4-(2-hydroxyethyl)-7-methyl-1,3-dioxo-4,7-epoxy-2H-isoindol-2-yl]-2-(trifluoromethyl)benzonitrile (25B)

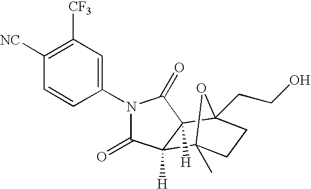

A. (3aα,4β,7β,7aα)- and (3aα,4α,7α,7aα)-4-[1,3,3a,4,7,7a-Hexahydro-4-(2-hydroxyethyl)-7-methyl-1,3-dioxo-4,7-epoxy-2H-isoindol-2-yl]-2-(trifluoromethyl)benzonitrile (25Ai and 25Aii, respectively)

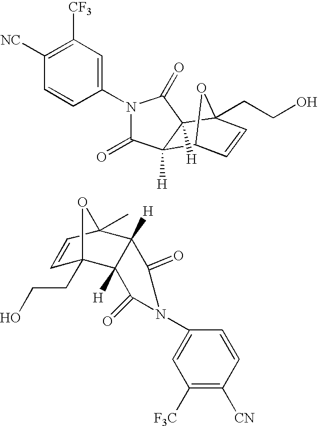

A solution of compound 21A (252 mg, 2 mmol, 1 eq) and 4-(2,5-dihydro-2,5-dioxo-1H-pyrrol-1-yl)-2-trifluoromethylbenzonitrile (798 mg, 3 mmol, 1.5 eq) in CH₂Cl₂ (10 mL) was stirred at room temperature for 2 days. The reaction mixture was concentrated under reduced pressure. Purification by flash chromatography on silica gel eluting with 65% EtOAc/hexanes gave 217 mg of pure compound 25Ai, 73 mg of pure compound 25Aii and 310 mg of a mixture of both compound 25Ai and 25Aii. All three fractions were isolated as white solids with a total isolated yield of 600 mg (1.53 mmol, 76.5%). Compound 25Ai: HPLC 90% at 2.56 min (retention time) (YMC S5 ODS column 4.6×50 mm, 10-90% aqueous methanol over 4 minutes containing 0.2% phosphoric acid, 4 mL/min, monitoring at 220 nm). Compound 25Aii: HPLC 90% at 2.56 min (retention time) (YMC S5 ODS column 4.6×50 mm, 10-90% aqueous methanol over 4 minutes containing 0.2% phosphoric acid, 4 mL/min, monitoring at 220 nm).

B. (3aα,4β,7β,7aα)-4-[Octahydro-4-(2-hydroxyethyl)-7-methyl-1,3-dioxo-4,7-epoxy-2H-isoindol-2-yl]-2-(trifluoromethyl)benzonitrile (25B)

A solution of compound 25Ai (0.2 g, 0.51 mmol, 1 eq) and 10% Pd/C (43 mg, cat.) in EtOH (12 mL) was stirred under a hydrogen atmosphere at room temperature for 2 h. The reaction mixture was filtered through celite and concentrated under reduced pressure to give 0.2 g (0.51 mmol, 100%) of compound 25B as a white solid. HPLC: 95% at 2.59 min (retention time) (YMC S5 ODS column 4.6×50 mm eluting with 10-90% aqueous methanol over 4 minutes containing 0.1% TFA, 4 mL/min, monitoring at 220 nm), MS (ESI): m/z 394.97 [M+H]⁺.

### EXAMPLE 26

### (3aα,4α,7α,7aα)- and (3aα,4β,7β,7aα)-N-[4-[2-[2-[4-Cyano-3-(trifluoromethyl)phenyl]octahydro-7-methyl-1,3-dioxo-4,7-epoxy-4H-isoindol-4-yl]ethoxy]phenyl]acetamide (26Ci and 26Cii, respectively)

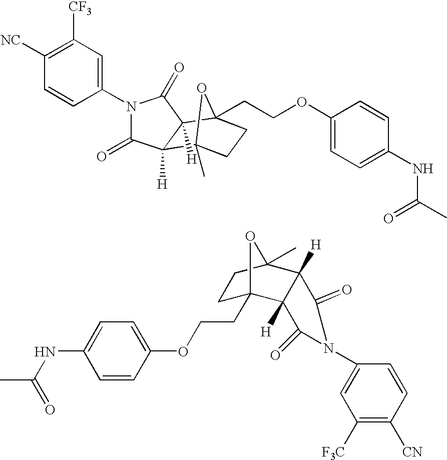

A. 2-[4-[2-(5-Methyl-2-furanyl)ethoxy]phenyl]acetamide (26A)

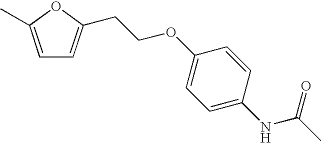

Triphenylphosphine (681 mg, 2.6 mmol, 1.3 eq) was added to a solution of compound 21A (252 mg, 2 mmol, 1 eq) and 4-acetamidophenol (302 mg, 2 mmol, 1 eq) in CH₂Cl₂ (4 mL). THF (5 mL) was added to make the reaction mixture homogeneous and the mixture was then cooled to 0° C. DEAD (0.41 mL, 2.6 mmol, 1.3 eq) was added dropwise and the reaction mixture was stirred at room temperature overnight, then concentrated under reduced pressure. Purification by flash chromatography on silica gel eluting with 60% EtOAc/hexanes followed by preparative reverse phase HPLC gave 270 mg (52%, 1.04 mmol) of compound 26A as a light brown solid. MS (ESI): m/z 260.09 [M+H]⁺.

B. (3aα,4α,7α,7aα)- and (3aα,4β,7β,7aα)-N-[4-[2-[2-[4-Cyano-3-(trifluoromethyl)phenyl]-1,2,3,3a,7,7a-hexahydro-7-methyl-1,3-dioxo-4,7-epoxy-4H-isoindol-4-yl]ethoxy]phenyl]acetamide (26Bi and 26Bii, respectively)

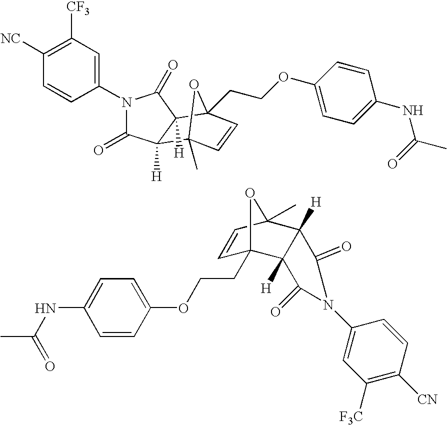

A solution of compound 26A (40 mg, 0.154 mmol, 1 eq) and 4-(2,5-dihydro-2,5-dioxo-1H-pyrrol-1-yl)-2-trifluoromethylbenzonitrile (88 mg, 0.31 mmol, 2 eq) in CH₂Cl₂ (2 mL) was stirred at room temperature for 2 days. The reaction mixture was concentrated under reduced pressure. Purification by flash chromatography on silica gel eluting with 75% EtOAc/hexanes gave 55 mg (0.105 mmol, 68%) of a 5 to 1 mixture of compounds 26Bi and 26Bii as a white solid, which was used directly in the next step. HPLC 90% at 3.28 min (retention time) (YMC S5 ODS column 4.6×50 mm, 10-90% aqueous methanol over 4 minutes containing 0.2% phosphoric acid, 4 mL/min, monitoring at 220 nm).

C. (3aα,4α,7α,7aα)- and (3aα,4β,7β,7aα)-N-[4-[2-[2-[4-Cyano-3(trifluoromethyl)phenyl]octahydro-7-methyl-1,3-dioxo-4,7-epoxy-4H-isoindol-4-yl]ethoxylphenyl]acetamide (26Ci and 26Cii, respectively)

A solution of a mixture of compounds 26Bi and 26Bii (55 mg, 0.105 mmol, 1 eq) and 10% Pd/C (12 mg, cat.) in EtOH (3 mL) was stirred under a hydrogen atmosphere at room temperature overnight. The reaction mixture was filtered through celite and concentrated under reduced pressure to give 50 mg of crude product.

Purification by flash chromatography on silica gel eluting with 70% EtOAc/hexanes gave 18 mg (0.034 mmol, 32%) of compound 26Ci [HPLC: 96% at 3.33 min (retention time) (YMC S5 ODS column 4.6×50 mm, 10-90% aqueous methanol over 4 minutes containing 0.2% phosphoric acid, 4 mL/min, monitoring at 220 nm). MS (ES): m/z 528.01 [M+H]⁺]; and 2.3 mg (0.004 mmols, 4%, 85:15-endo:exo) of an 85:15 mixture of compound (by ¹H NMR) 26Cii and compound 26Ci respectively [HPLC: 90% at 3.35 min (retention time) (YMC S5 ODS column 4.6×50 mm, 10-90% aqueous methanol over 4 minutes containing 0.2% phosphoric acid, 4 mL/min, monitoring at 220 nm), MS (ESI): m/z 528.12 [M+H]+].

### EXAMPLE 27

C. (3aα,4α,7α,7aα)-Hexahydro-2-(2-naphthalenyl)-4,7-epoxy-1H-isoindole-1,3(2H)-dione (27D)

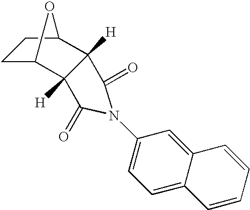

A. (endo, endo)-7-Oxabicyclo[2.2.1]hept-5-ene-2,3-dicarboxylic acid (27A)

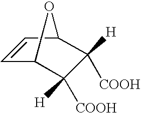

Compounds 27A, 27B and 27C were synthesized in accordance with the approaches described in Sprague et al., J. Med. Chem. 28, 1580-1590 (1985). A mixture of furan (100 mL, 1.38 mol, 1 eq) and maleic acid (159.6 g, 1.38 mol, 1 eq) in H₂O (340 mL) was stirred at room temperature for 5 days. The mixture was placed in a separatory funnel and the aqueous layer was separated from the layer containing the unreacted furan. The aqueous layer was treated with charcoal, filtered through celite and placed in the refrigerator. The desired product crystallized from solution upon seeding, was filtered, washed with cold water and dried over P₂0₅ to give 70 g (0.38 mol, 28%) of compound 27A as a white solid.

B. (endo, endo)-7-Oxabicyclo[2.2.1]heptane-2,3-dicarboxylic acid (27B)

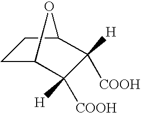

To a solution of compound 27A (69 g, 0.375 mol, 1 eq) in EtOH (700 mL) was added 10% Pd/C (4.5 g, cat.) and the mixture was shaken under a hydrogen atmosphere at 55 psi until gas uptake ceased. The mixture was filtered through celite and concentrated in vacuo to give 66 g (0.355 mol, 95%) of compound 27B as a white solid.

C. (3aα,4α,7α,7aα)-Hexahydro-4,7-epoxyisobenzofuran-1,3-dione (27C)

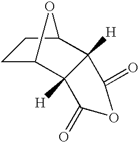

A solution of compound 27B (66 g, 355 mol) in acetyl chloride (300 mL) was refluxed for 1 h. The reaction solution was concentrated in vacuo and the resulting residue was recrystallized from benzene to give 49.2 g (0.292 mol, 82%) of compound 27C as a white solid (>99% endo by ¹H NMR).

D. (3aα,4α,7α,7aα)-Hexahydro-2-(2-naphthalenyl)-4,7-epoxy-1H-isoindole-1,3(2H)-dione (27D)

Compound 27C (45 mg, 0.30 mmol, 1 eq) was combined with 2-naphthalenamine (47 mg, 0.33 mmol, 1.1 eq) in acetic acid (1 mL) and heated at 115° C. overnight. After the reaction was cooled to rt, a drop of water was added, and the resulting precipitate was filtered. The material was washed with methanol and dried to provide 65.7 mg (74.5%) of compound 27D as a white crystalline solid. HPLC: 99% at 2.68 min (retention time) (YMC S5 ODS column 4.6×50 mm, 10-90% aqueous methanol over 4 minutes containing 0.2% phosphoric acid, 4 mL/min, monitoring at 220 nm), MS (ESI): m/z 294.0 [M+H]⁺.

### EXAMPLE 28

### (1aα,2β,2aα,5aα,6β,6aα)-Hexahydro-4-(2-naphthalenyl)-2,6-epoxy-3H-oxireno[f]isoindole-3,5(4H)-dione (28B)

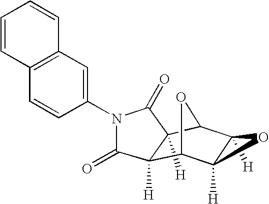

A. (1aα,2β,2aα,5aα,6β,6aα)-Tetrahydro-2,6-epoxyoxireno[f]isobenzofuran-3,5(2aH,5aH)-dione (28A)

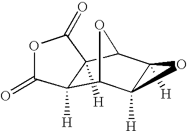

As described in Yur'ev, et al., J. Gen. Chem. U.S.S.R. (Engl. Transl.) 31, 772-775 (1961), a solution of exo-7-oxabicyclo[2.2.1]heptane-2,3-dicarboxylic anhydride (5 g, 30.09 mmol), formic acid (10 mL) and hydrogen peroxide (6 mL) was stirred at room temperature. After 30 min, the reaction was placed in an ice bath (it became exothermic along with gas evolution) and was allowed to warm to room temperature slowly. After stirring overnight, the resulting precipitate was collected by filtration and washed with glacial acetic acid and dried to yield 3.02 g of a white powder. The crude solid was boiled in acetyl chloride (100 mL) for 10 hours and the mixture was concentrated to 20 mL under reduced pressure. The resulting precipitate was filtered, washed with dioxanes and dried to give 2.37 g of compound 28A as a white powder.

B. (1aα,2β,2aα,5aα,6β,6aα)-Hexahydro-4-(2-naphthalenyl)-2,6-epoxy-3H-oxireno[f]isoindole-3,5(4H)-dione (28B)

Compound 28A (100 mg, 0.520 mmol, 1.2 eq) was combined with 2-naphthalenamine (0.434 mmol, 1 eq) in acetic acid (2 mL) and heated at 115° C. overnight. After the reaction was allowed to cool to rt, water was added, and the resulting precipitate was filtered. The material was washed sequentially with aqueous K₂CO₃ and water and then dried in a vacuum oven to provide 113.7 mg (85.3%) of compound 28B as an off-white crystalline solid. HPLC: 99% at 1.76 min (retention time) (YMC S5 ODS column 4.6×50 mm, 10-90% aqueous methanol over 4 minutes containing 0.2% phosphoric acid, 4 mL/min, monitoring at 220 nm), MS (ESI): m/z 308.0 [M+H]⁺.

### EXAMPLE 29

### (3aα,4α,7α,7aα)-2-[4-Bromo-3-(trifluoromethyl)phenyl]-3a,4,7,7a-tetrahydro-4,7-dimethyl-4,7-epithio-1H-isoindole-1,3(2H)-dione 8-oxide (29)

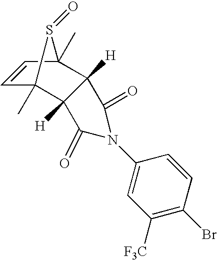

2,5-Dimethylthiophene (0.048 mL, 0.42 mmol) and 4-(2,5-dihydro-2,5-dioxo-1H-pyrrol-1-yl)-2-trifluoromethylbenzonitrile (0.290 g, 0.625 mmol) were dissolved in CH₂Cl₂ (8.0 mL) and cooled to −20° C. BF₃.Et₂O (0.412 mL, 3.36 mmol) was added slowly followed by addition of mCPBA (˜50%, 0.290 g, 0.84 mmol). After 2 h at −20° C., the reaction was poured into saturated aq NaHCO₃ and extracted with CH₂Cl₂ (3×20 mL) and the organics dried over anhydrous Na₂SO₄. The crude product was purified by flash chromatography on SiO₂ eluting with 5%-10%-20% EtOAc in CH₂Cl₂ to give 0.119 g of compound 29 as a white solid. HPLC: 91% at 3.303 min (retention time) (YMC S5 ODS column 4.6×50 mm, 10-90% aqueous methanol over 4 minutes containing 0.2% phosphoric acid, 4 mL/min, monitoring at 220 nm). MS (ESI): m/z 480.2 [M+H]⁺.

### EXAMPLE 30

### (3aα,4α,7α,7aα)-2-[4-Bromo-3-(trifluoromethyl)phenyl]-3a,4,7,7a-tetrahydro-4,7-epithio-1H-isoindole-1,3(2H)-dione 8-oxide (30)

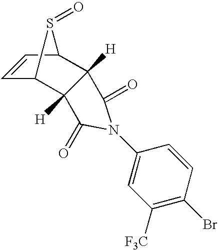

Thiophene (0.375 mL, 4.69 mmol) and 4-(2,5-dihydro-2,5-dioxo-1H-pyrrol-1-yl)-2-trifluoromethylbenzonitrile (0.100 g, 0.313 mmol) were dissolved in CH₂Cl₂ (50 20 mL) and mCPBA (˜50%, 1.62 g, 4.69 mmol) was added and the stirred at 25° C. for 3 h. Triphenylphosphine (2.0 g) was then added. After 15 min, the volatiles were removed in vacuo and the resulting residue was dissolved in CH₂Cl₂ (200 mL) and washed with saturated aq NaHCO₃ (3×50 mL) and dried over Na₂SO₄. The crude material was then purified by flash chromatography on SiO₂ eluting with 1%-3%-5% methanol in CH₂Cl₂ to give compound 30 as a white powder (0.059 g). NMR spectroscopy and LC analysis showed a single diastereomer. HPLC: 100% at 3.437 min (retention time) (YMC S5 ODS column 4.6×50 mm, 10-90% aqueous methanol over 4 minutes containing 0.2% phosphoric acid, 4 mL/min, monitoring at 220 nm), MS (ESI): m/z 443.2 [M+H]⁺.

### EXAMPLE 31

### (3aα,4α,7α,7aα)-Hexahydro-2-[3-(trifluoromethyl)phenyl]-4,7-imino-1H-isoindole-1,3(2H)-dione (31D)

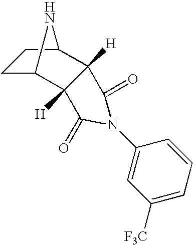

A. 7-Azabicyclo[2.2.1]hepta-2,5-diene-2,3,7-tricarboxylic acid 2,3-dimethyl 7-(1,1-dimethylethyl) ester (31A)

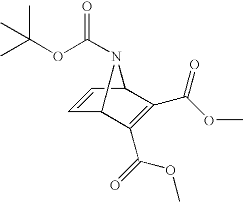

The freshly distilled acetylenedicarboxylic acid dimethyl ester (6.7 mL, 54.0 mmol) and N-(tert-butyloxycarbonyl)-1H-pyrrole (9.0 mL, 54.0 mmol) were combined and heated at 120° C. for 3 h. Purification by flash chromatography on SiO₂ eluting with EtOAc/CH₂Cl₂ gave 8.3 g of compound 31A as a yellow solid.

B. (exo,endo)-7-Azabicyclo[2.2.1]hept-2,5-diene-2,3,7-tricarboxylic acid 7-(1,1-dimethylethyl) ester (31B)

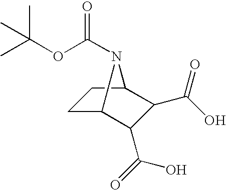

Compound 31A (1.0 g, 3.5 mmol) was dissolved in MeOH (2.0 mL) and aq KOH (1 g in 5 mL H2O) was added. The reaction was heated to 50° C. for 1 h. The reaction was then cooled to 25° C. and Pd/C (0.5 g, 10% Pd) was added and the mixture was placed in a Parr apparatus for 14 h at 25° C. The reaction was then filtered through celite rinsing with water. The aqueous solution was acidified to pH 2 by addition of 1 N HCl and then extracted with EtOAc (2×100 mL). Concentration of the organics gave the compound 31B as a pale yellow solid.

C. (3aα,4α,7α,7aα)-Hexahydro-1,3-dioxo-4,7-iminoisobenzofuran-8-carboxylic acid 1,1-dimethylethyl ester (31C)

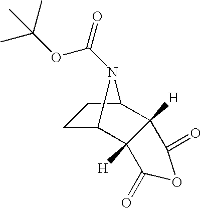

The crude compound 31B was heated to 120° C. in vacuo in a sublimation chamber, resulting in sublimation of compound 31C as a white solid (0.051 g), which was collected directly and used in the next step without further purification.

D. (3aα,4α, 7α,7aα)-Hexahydro-2-[3-(trifluoromethyl)phenyl]-4,7-imino-1H-isoindole-1,3(2H)-dione (31D)

Compound 31C (0.050 g, 0.187 mmol) and the 1-amino-3-(trifluoromethyl)benzene (0.030 g, 0. 187 mmol) were dissolved in AcOH (2.5 mL) and heated to 115° C. for 4.5 h. The reaction was quenched by addition of saturated aqueous NaHCO₃ and the extracted with methylene chloride (3×15 mL). The crude material was purified by preparative reverse phase HPLC to give 0.030 g of compound 31D as a white solid. HPLC: 99% at 2.33 min (retention time) (YMC S5 ODS column 4.6×50 mm, 10-90% aqueous methanol over 4 minutes containing 0.2% phosphoric acid, 4 mL/min, monitoring at 220 nm), MS (ESI): m/z 311.15 [M+H]⁺.

### EXAMPLE 32

### (3aα,4β,7β,7aα)- and (3aα,4α,7α,7aα)-3a4,7,7a-Tetrahydro-4,7-dimethyl-2-[3-(trifluoromethyl)phenyl]-4,7-epoxy-1H-isoindole-1,3(2H)-dione (32i and 32ii, respectively)

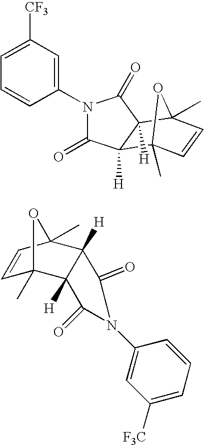

Freshly distilled 2,5-dimethylfuran (0.32 mL, 2.6 mmol) was dissolved in CH₂Cl₂ (2.0 mL) and 1-[3-(trifluoromethyl)phenyl]-1H-pyrrole-2,5-dione (0.5 g, 2.5 mmol) was added. The reaction was stirred at 25° C. for 16 h and was then concentrated under reduced pressure. Purification by flash chromatography on silica gel eluting with 0.5% MeOH/CH₂Cl₂ gave 50 mg of compound 32i and 250 mg of compound 32ii, as white solids. Compound 32i: HPLC: 92% at 3.047 min (retention time) (YMC S5 ODS column 4.6×50 mm, 10-90% aqueous methanol over 4 minutes containing 0.2% phosphoric acid, 4 mL/min, monitoring at 220 nm), MS (ES): m/z: 338.15 [M+H]⁺; Compound 32ii: HPLC: 98% at 3.08 min (retention time) (YMC S5 ODS column 4.6×50 mm, 10-90% aqueous methanol over 4 minutes containing 0.2% phosphoric acid, 4 mL/min, monitoring at 220 nm), MS (ES): m/z 338.30 [M+H]⁺.

### EXAMPLE 33

### (3aα,4α,7α,7aα)-Hexahydro-4,7-dimethyl-2-[3-(trifluoromethyl)]phenyl]-4,7-epoxy-1H-isoindole-1,3(2H)-dione (33)

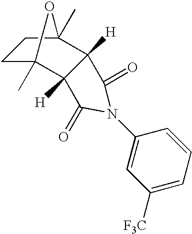

Compound 32ii (0.080 g, 0.237 mmol) was dissolved in EtOAc (2 mL) and EtOH (1 ml) and Pd/C (10% Pd, 0.050 g) was added. Hydrogen was then introduced by a balloon and the reaction was stirred for 24 h. The mixture was filtered through celite, rinsed with EtOAc and concentrated in vacuo to give compound 33 (0.075 g) as a white solid. No further purification was needed. HPLC: 90% at 3.233 min (retention time) (YMC S5 ODS column 4.6×50 mm, 10-90% aqueous methanol over 4 minutes containing 0.2% phosphoric acid, 4 mL/min, monitoring at 220 nm), MS (ES): m/z 340.40 [M+H]⁺.

### EXAMPLE 34

### (3aα,4α,7α,7aα)-Tetrahydro-5-methyl-2-(4-nitro-1-naphthalenyl)-4,7-etheno-1H-pyrrolo[3,4-c]pyridine-1,3,6(2H,5H)-trione (34B)

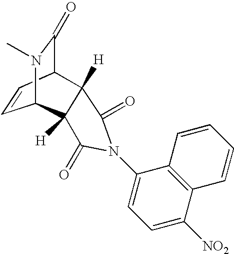

A. 4,5,7,7a-Tetrahydro-5-methyl-4,7-ethenofuro [3,4-c]pyridine-1,3,6(3aH)-trione (34A)

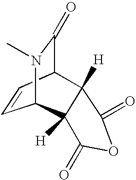

Compound 34A was synthesized by a modification of the methods described in Tomisawa et al., Heterocycles 6, 1765-1766 (1977) and Tetrahedron Lett. 29, 2465-2468 (1969). Maleic anhydride and 1-methyl-2-pyridone were suspended in 30 ml of anhydrous tolulene. The reaction vessel was fitted with a Dean Stark trap and refluxed for 48 hours. The dark colored solution was allowed to cool to rt and then the volatiles were removed in vacuo. The resulting brown paste was dissolved in 10 ml of boiling toluene and the hot solution was filtered under a nitrogen flow to remove particulates. On standing at 25° C. the desired product precipitated from solution. The solid was isolated by filtration and washed with cold toluene to give compound 34A, which was used without further purification.

B. (3aα,4α,7α,7aα)-Tetrahydro-5-methyl-2-(4-nitro-1-naphthalenyl)-4,7-etheno-1H-pyrrolo[3,4-c]pyridine-1,3,6(2H,5H)-trione (34B)

1-Amino-4-nitronaphthalene (0.094 g, 0.5 mmol) and compound 34A (0.130 g, 0.63 mmol) were dissolved in AcOH (2.0 mL) and heated to 110° C. for 11 h. The reaction was then cooled to 25° C. and poured into cold saturated aqueous K₂CO₃ and stirred vigorously for 10 min. The solution was filtered and rinsed with water. The resulting filtrate was dried in vacuo and purified by silica gel chromatography using a solvent system of 4:6 EtOAc/Hexane, to give 0.172 g of compound 34B as a white solid. HPLC: 92% at 2.472 min (retention time) (YMC S5 ODS column 4.6×50 mm eluting with 10-90% aqueous methanol over 4 minutes containing 0.1% TFA, 4 mL/min, monitoring at 220 nm), MS (ES): m/z 378.29 [M+H]⁺.

### EXAMPLE 35

### (3aα,4β,7β,7aα)-4-[4-[2-(4-Fluorophenoxy)ethyl]octahydro-7-methyl-1,3-dioxo-4,7-epoxy-2H-isoindol-2-yl]-2-(trifluoromethyl)benzonitrile (35)

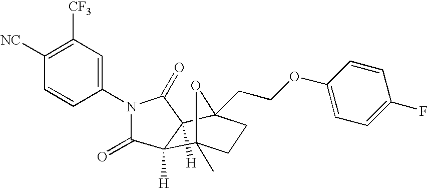

DEAD (0.06 mL, 0.380 mmol, 1.5 eq) was added to a solution of triphenylphosphine (100 mg, 0.380 mmol, 1.5 eq) in THF (1.3 mL) at room temperature under an inert atmosphere. After stirring for 10 mins, 4-fluorophenol (43 mg, 0.380 mmol, 1.5 eq) was added in one portion. The reaction mixture was stirred for 5 mins, compound 25B (100 mg, 0.254 mmol, 1 eq) was added and stirring was continued for 3.5 h. Purification by flash chromatography on silica gel eluting with 50% EtOAc/Hexanes followed by preparative chromatography [HPLC: 11.93 min (retention time) (YMC S5 ODS column 20×100 mm, 0-100% aqueous methanol over 10 minutes containing 0.1% TFA, 20 mL/min, monitoring at 220 nm)] gave 72 mg (58%) of compound 35 as a solid. HPLC: 99% at 3.74 min (retention time) (YMC S5 ODS column 4.6×50 mm, 10-90% aqueous methanol over 4 minutes containing 0.2% phosphoric acid, 4 mL/min, monitoring at 220 nm), MS (ESI): m/z 487.1 [M−H]⁻.

### EXAMPLE 36

### (3aα,4β,7β,7aα)-4-[4-(2-Bromoethyl]octahydro-7-methyl-1,3-dioxo-4,7-epoxy-2H-isoindol-2-yl]-2-(trifluoromethyl)benzonitrile (36)

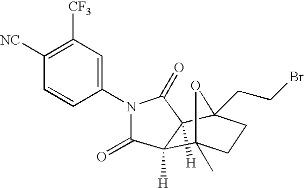

A solution of 25B (495 mg, 1.26 mmol, 1 eq) and pyridine (0.1 ml, 1.26 mmol, 1 eq) in CH₂Cl₂ (2 ml) was added to a solution of Ph₃PBr₂ (636 mg, 1.51 mmol, 1.2 eq) in CH₂Cl₂ (2 ml) at 0° C. The reaction mixture was stirred at room temperature for 3 hr, then the solvent was removed under reduced pressure. The resulting residue was washed 2× with 10 ml portions of EtOAc-hexane (6:4) and the combined washings were purified by flash chromatography on silica gel eluting with 60% EtOAc/hexane to give 390 mg (0.85 mmol, 67.7%) of compound 36 as a white solid. HPLC: 99% at 3.51 min (retention time) (YMC S5 ODS column 4.6×50 mm, 10-90% aqueous methanol over 4 minutes containing 0.2% phosphoric acid, 4 mL/min, monitoring at 220 nm). MS (ESI): m/z 456.7 [M−H]⁻.

### EXAMPLE 37

### (3aα,4β,7β,7aα)-Hexahydro-4,7-dimethyl-2-(3-methyl-4-nitrophenyl)-4,7-epoxy-1H-isoindole-1,3(2H)-dione (37)

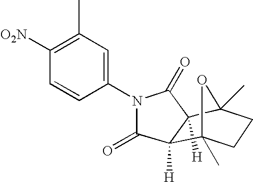

A combination of 4-nitro-3-methylaniline (0.050 g, 0.33 mmol), compound 20A (0.083 g, 0.43 mmol), TEA (0.2 mL), MgSO₄ (0.075 g) and toluene (0.8 mL) were combined in a sealed tube and the mixture was heated to 120° C. for 14 h. After cooling to 25° C., the reaction was filtered, rinsed with CH₂Cl₂ and concentrated. The crude product was purified by preparative-TLC on SiO₂ eluting with CH₂Cl₂ to give 0.075 g of compound 37 as a pale yellow solid. HPLC: 100% at 2.733 min (retention time) (YMC S5 ODS column, 4.6×50 mm; 10-90% MeOH/H₂O gradient,+0.1% TFA; 4 mL/min, 220 nM detection), MS (ES): m/z 348.2 [M+NH₄]⁺.

### EXAMPLES 38 to 121

Additional compounds of the present invention were prepared by procedures analogous to those described above. The compounds of Examples 38 to 121 have the following structure (L is a bond):

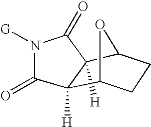

where G, the compound name, retention time, molecular mass, and the procedure employed, are set forth in Table 2. The chromatography techniques used to determine the compound retention times of Table 2 are as follows: LCMS=YMC S5 ODS column, 4.6×50 mm eluting with 10-90% MeOH/H₂O over 4 minutes containing 0.1% TFA; 4 mL/min, monitoring at 220 nm. The molecular mass of the compounds listed in Table 2, where provided, were determined by MS (ES) by the formula m/z.

TABLE 2

| | | | Retention | |
|---|---|---|---|---|
| | | | Time | |
| Ex. | | Compound | (Min.)/ | Pro. |
| No. | G | Name | Molecular Mass | of Ex. |
| 38 | 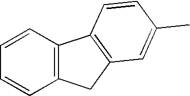 | (3aα,4β,7β,7aα)-2-(2- Fluorenyl)hexahydro-4,7-epoxy-1H- isoindole-1,3(2H)-dione | 3.72 LCMS/ 332.20 [M + H]⁺ | 8 |
| 39 | 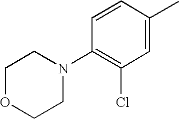 | (3aα,4β,7β,7aα)-2-[3-Chloro-4-(4- morpholinyl)phenyl]hexahydro-4,7- epoxy-1H-isoindole-1,3(2H)-dione | 3.20 LCMS/ 363.20 [M + H]⁺ | 8 |
| 40 | 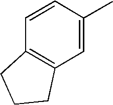 | (3aα,4β,7β,7aα)-2-(2,3-Dihydro-1H- inden-5-yl)hexahydro-4,7-epoxy- 1H-isoindole-1,3(2H)-dione | 3.26 LCMS/ 284.22 [M + H]⁺ | 8 |
| 41 | 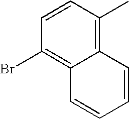 | (3aα,4β,7β,7aα)-2-(4-Bromo-1- naphthalenyl)hexahydro-4,7-epoxy- 1H-isoindole-1,3(2H)-dione | 3.73 LCMS/ 404.11 [M + CH₃OH + H]⁺ | 8 |
| 42 | 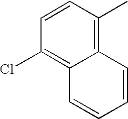 | (3aα,4β,7β,7aα)-2-(4-Chloro-1- naphthalenyl)hexahydro-4,7-epoxy- 1H-isoindole-1,3(2H)-dione | 3.63 LCMS/ 328.14 [M + H]⁺ | 8 |
| 43 | 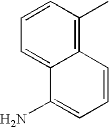 | (3aα,4β,7β,7aα)-2-(5-Amino-1- naphthalenyl)hexahydro-4,7-epoxy- 1H-isoindole-1,3(2H)-dione | 1.64 LCMS/ | 8 |
| 44 | 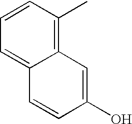 | (3aα,4β,7β,7aα)-Hexahydro-2-(7- hydroxy-1-naphthalenyl)-4,7-epoxy- 1H-isoindole-1,3(2H)-dione | 2.54 LCMS/ 308.23 [M − H]⁻ | 8 |
| 45 | 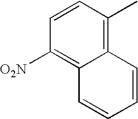 | (3aα,4β,7β,7aα)-Hexahydro-2-(4- nitro-1-naphthalenyl)-4,7-epoxy-1H- isoindole-1,3(2H)-dione | 3.117 LCMS/ 404.11 [M + CH₃OH + H]⁺ | 8 |
| 46 | 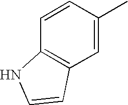 | (3aα,4β,7β,7aα)-Hexahydro-2-(1H- indol-5-yl)-4,7-epoxy-1H-isoindole- 1,3(2H)-dione | 2.39 LCMS/ 283.23 [M + H]⁺ | 8 |
| 47 | 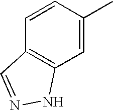 | (3aα,4β,7β,7aα)-Hexahydro-2-(1H- indazol-6-yl)-4,7-epoxy-1H- isoindole-1,3(2H)-dione | 2.35 LCMS/ 282.23 [M − H]⁻ | 8 |
| 48 | 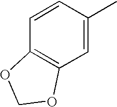 | (3aα,4β,7β,7aα)-2-(1,3- Benzodioxol-5-yl)hexahydro-4,7- epoxy-1H-isoindole-1,3(2H)-dione | 2.47 LCMS/ 288.20 [M + H]⁺ | 8 |
| 49 | 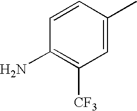 | (3aα,4β,7β,7aα)-2-[4-Amino-3- (trifluoromethyl)phenyl]hexahydro- 4,7-epoxy-1H-isoindole-1,3(2H)-dione | 2.71 LCMS/ 327.20 [M + H]⁺ | 8 |
| 50 | 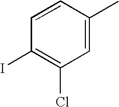 | (3aα,4β,7β,7aα)-2-(3-Chloro-4- iodophenyl)hexahydro-4,7-epoxy- 1H-isoindole-1,3(2H)-dione | 3.70 LCMS/ 435.2 [M + CH₃OH]⁺ | 8 |
| 51 | 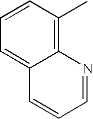 | (3aα,4β,7β,7aα)-Hexahydro-2-(8- quinolinyl)-4,7-epoxy-1H-isoindole- 1,3(2H)-dione | 2.28 LCMS/ 295.22 [M + H]⁺ | 8 |
| 52 | 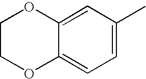 | (3aα,4β,7β,7aα)-2-(2,3-Dihydro-1,4- benzodioxin-6-yl)hexahydro-4,7- epoxy-1H-isoindole-1,3(2H)-dione | 2.55 LCMS/ 302.23 [M + H]⁺ | 8 |
| 53 | 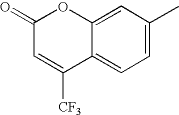 | (3aα,4β,7β,7aα)-Hexahydro-2-[2- oxo-4-(trifluoromethyl)-2H-1- benzopyran-7-yl]-4,7-epoxy-1H- isoindole-1,3(2H)-dione | 3.38 LCMS/ 412.17 [M + CH₃OH + H]⁺ | 8 |
| 54 | 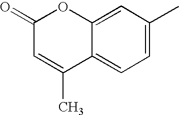 | (3aα,4β,7β,7aα)-Hexahydro-2-(4- methyl-2-oxo-2H-1-benzopyran-7- yl)-4,7-epoxy-1H-isoindole-1,3(2H)- dione | 2.74 LCMS/ 326.20 [M + H]⁺ | 8 |
| 55 | 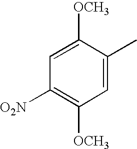 | (3aα,4β,7β,7aα)-2-(2,5-Dimethoxy- 4-nitropheny)hexahydro-4,7-epoxy- 1H-isoindole-1,3(2H)-dione | 2.70 LCMS/ 349.23 [M + H]⁺ | 8 |
| 56 | 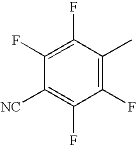 | (3aα,4β,7β,7aα)-2,3,5,6- Tetrafluoro-4-(octahydro-1,3-dioxo- 4,7-epoxy-2H-isoindol-2- yl)benzonitrile | 2.97 LCMS | 8 |
| 57 | 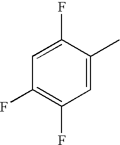 | (3aα,4β,7β,7aα)-Hexahydro-2- (2,4,5-trifluorophenyl)-4,7-epoxy- 1H-isoindole-1,3(2H)-dione | 2.90 LCMS | 8 |
| 58 | 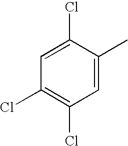 | (3aα,4β,7β,7aα)-Hexahydro-2- (2,4,5-trichlorophenyl)-4,7-epoxy- 1H-isoindole-1,3(2H)-dione | 3.64 LCMS/ 346.39 [M]^{+.} | 8 |
| 59 | 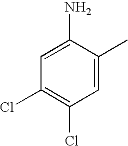 | (3aα,4β,7β,7aα)-2-(2-Amino-4,5- dichlorophenyl)hexahydro-4,7- epoxy-1H-isoindole-1,3(2H)-dione | 3.23 LCMS | 8 |
| 60 | 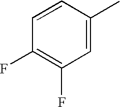 | (3aα,4β,7β,7aα)-2-(3,4- Difluorophenyl)hexahydro-4,7- epoxy-1H-isoindole-1,3(2H)-dione | 2.91 LCMS/ 280.23 [M + H]⁺ | 8 |
| 61 | 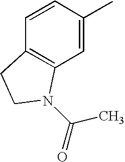 | (3aα,4β,7β,7aα)-1-Acetyl-2,3- dihydro-6-(octahydro-1,3-dioxo-4,7- epoxy-2H-isoindol-2-yl)-1H-indole | 2.43 LCMS/ 359.26 [M + CH₃OH + H]⁺ | 8 |
| 62 | 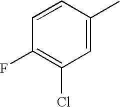 | (3aα,4β,7β,7aα)-2-(3-Chloro-4- fluorophenyl)hexahydro-4,7-epoxy- 1H-isoindole-1,3(2H)-dione | 3.21 LCMS/ 328.14 [M + CH₃OH + H]⁺ | 8 |
| 63 | 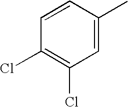 | (3aα,4β,7β,7aα)-2-(3,4- Dichlorophenyl)hexahydro-4,7- epoxy-1H-isoindole-1,3(2H)-dione | 3.54 LCMS/ 311.79 [M − H]⁻ | 8 |
| 64 | 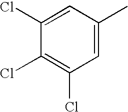 | (3aα,4β,7β,7aα)-Hexahydro-2- (3,4,5-trichlorophenyl)-4,7-epoxy- 1H-isoindole-1,3(2H)-dione | 4.05 LCMS/ 378.10 [M + CH₃OH + H]⁺ | 8 |
| 65 | 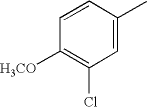 | (3aα,4β,7β,7aα)-2-(3-Chloro-4- methoxyphenyl)hexahydro-4,7- epoxy-1H-isoindole-1,3(2H)-dione | 2.99 LCMS/ 308.11 [M + H]⁺ | 8 |
| 66 | 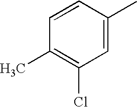 | (3aα,4β,7β,7aα)-2-(3-Chloro-4- methylphenyl)hexahydro-4,7-epoxy- 1H-isoindole-1,3(2H)-dione | 3.39 LCMS/ 292.20 [M + H]⁺ | 8 |
| 67 | 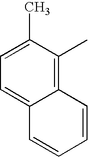 | (3aα,4β,7β,7aα)-Hexahydro-2-(2- methyl-1-naphthalenyl)-4,7-epoxy- 1H-isoindole-1,3(2H)-dione | 3.28 LCMS/ 308.23 [M + H]⁺ | 8 |
| 68 | 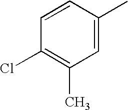 | (3aα,4β,7β,7aα)-2-(4-Chloro-3- methylphenyl)hexahydro-4,7-epoxy- 1H-isoindole-1,3(2H)-dione | 3.40 LCMS/ 292.20 [M + H]⁺ | 8 |
| 69 | 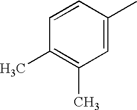 | (3aα,4β,7β,7aα)-2-(3,4- Dimethylphenyl)hexahydro-4,7- epoxy-1H-isoindole-1,3(2H)-dione | 3.11 LCMS/ 272.23 [M + H]⁺ | 8 |
| 70 | 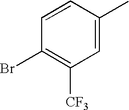 | (3aα,4β,7β,7aα)-2-[4-Bromo-3- (trifluoromethyl)phenyl]hexahydro- 4,7-epoxy-1H-isoindole-1,3(2H)- dione | 3.76 LCMS/ 421.98 [M + CH₃OH + H]⁺ | 8 |
| 71 | 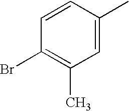 | (3aα,4β,7β,7aα)-2-(4-Bromo-3- methylphenyl)hexahydro-4,7-epoxy- 1H-isoindole-1,3(2H)-dione | 3.50 LCMS/ 336.05 [M + H]⁺ | 8 |
| 72 | 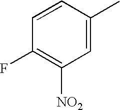 | (3aα,4β,7β,7aα)-2-(4-Fluoro-3- nitrophenyl)hexahydro-4,7-epoxy- 1H-isoindole-1,3(2H)-dione | 2.80 LCMS/ 305.25 [M − H]⁻ | 8 |
| 73 | 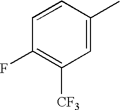 | (3aα,4β,7β,7aα)-2-[4-Fluoro-3- (trifluoromethyl)phenyl]hexahydro- 4,7-epoxy-1H-isoindole-1,3(2H)- dione | 3.45 LCMS/ 362.26 [M + CH₃OH + H]⁺ | 8 |
| 74 | 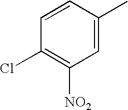 | (3aα,4β,7β,7aα)-2-(4-Chloro-3- nitrophenyl)hexahydro-4,7-epoxy- 1H-isoindole-1,3(2H)-dione | 3.19 LCMS/ 322.86 [M]^{+.} | 8 |
| 75 | 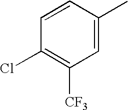 | (3aα,4β,7β,7aα)-2-[4-Chloro-3- (trifluoromethyl)phenyl]hexahydro- 4,7-epoxy-1H-isoindole-1,3(2H)- dione | 3.68 LCMS/ 345.83 [M]^{+.} | 8 |
| 76 | 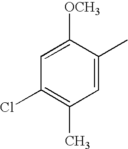 | (3aα,4β,7β,7aα)-2-(4-Chloro-2- methoxy-5- methylphenyl)hexahydro-4,7-epoxy- 1H-isoindole-1,3(2H)-dione | 3.31 LCMS/ 322.20 [M + H]⁺ | 8 |
| 77 | 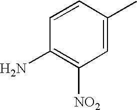 | (3aα,4β,7β,7aα)-2-(4-Amino-3- nitrophenyl)hexahydro-4,7-epoxy- 1H-isoindole-1,3(2H)-dione | 2.34 LCMS/ 302.27 [M − H]⁻ | 8 |
| 78 | 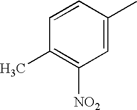 | (3aα,4β,7β,7aα)-Hexahydro-2-(4- methyl-3-nitrophenyl)-4,7-epoxy- 1H-isoindole-1,3(2H)-dione | 3.02 LCMS/ 335.20 [M + CH₃OH + H]⁺ | 8 |
| 79 | 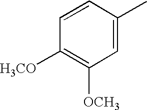 | (3aα,4β,7β,7aα)-2-(3,4- Dimethoxyphenyl)hexahydro-4,7- epoxy-1H-isoindole-1,3(2H)-dione | 2.35 LCMS/ 304.25 [M + H]⁺ | 8 |
| 80 | 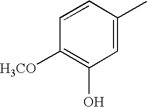 | (3aα,4β,7β,7aα)-Hexahydro-2-(3- hydroxy-4-methoxyphenyl)-4,7- epoxy-1H-isoindole-1,3(2H)-dione | 0.98 LCMS/ 321.19 [M + CH₃OH]⁺ | 8 |
| 81 | 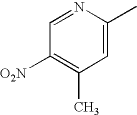 | (3aα,4β,7β,7aα)-Hexahydro-2-(4- methyl-5-nitro-2-pyridinyl)-4,7- epoxy-1H-isoindole-1,3(2H)-dione | 0.54 LCMS/ 304.20 [M + H]⁺ | 8 |
| 82 | 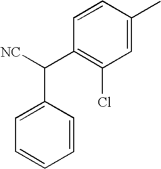 | (3aα,4β,7β,7aα)-2-Chloro-4- (octahydro-1,3-dioxo-4,7-epoxy-2H- isoindol-2-yl)-α- phenylbenzeneacetonitrile | 3.67 LCMS/ 423.8 [M + CH₃OH]^{+.} | 8 |
| 83 | 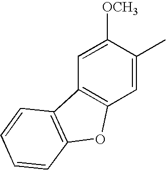 | (3aα,4β,7β,7aα)-Hexahydro-2-(2- methoxy-3-dibenzofuranyl)-4,7- epoxy-1H-isoindole-1,3(2H)-dione | 3.66 LCMS/ 364.25 [M + H]⁺ | 8 |
| 84 | 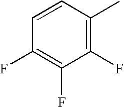 | (3aα,4β,7β,7aα)-Hexahydro-2- (2,3,4-trifluorophenyl)-4,7-epoxy- 1H-isoindole-1,3(2H)-dione | 3.06 LCMS/ 298.14 [M + H]⁺ | 8 |
| 85 | 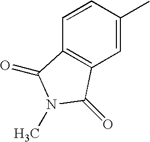 | (3aα,4β,7β,7aα)-2-(2,3-Dihydro-2- methyl-1,3-dioxo-1H-isoindol-5- yl)hexahydro-4,7-epoxy-1H- isoindole-1,3(2H)-dione | 2.70 LCMS/ 359.22 [M + CH₃OH + H]⁺ | 8 |
| 86 | 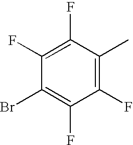 | (3aα,4β,7β,7aα)-2-(4-Bromo-2,3,5,6- tetrafluorophenyl)hexahydro-4,7- epoxy-1H-isoindole-1,3(2H)-dione | 3.72 LCMS/ 426.07 [M + CH₃OH + H]⁺ | 8 |
| 87 | 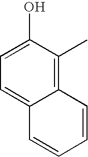 | (3aα,4β,7β,7aα)-Hexahydro-2-(2- hydroxy-1-naphthalenyl)-4,7-epoxy- 1H-isoindole-1,3(2H)-dione | 2.52 LCMS/ 308.26 [M − H]⁻ | 8 |
| 88 | 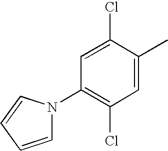 | (3aα,4β,7β,7aα)-2-[2,5-Dichloro-4- (1H-pyrrol-1-yl)phenyl]hexahydro- 4,7-epoxy-1H-isoindole-1,3(2H)- dione | 3.70 LCMS/ 376.64 [M − H]⁻ | 8 |
| 89 | 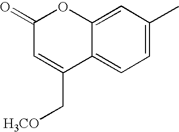 | (3aα,4β,7β,7aα)-Hexahydro-2-[4- (methoxymethyl)-2-oxo-2H-1- benzopyran-7-yl]-4,7-epoxy-1H- isoindole-1,3(2H)-dione | 2.79 LCMS/ 356.26 [M + H]⁺ | 8 |
| 90 | 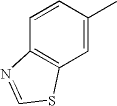 | (3aα,4β,7β,7aα)-2-(6- Benzothiazolyl)hexahydro-4,7- epoxy-1H-isoindole-1,3(2H)-dione | 2.46 LCMS/ 301.19 [M + H]⁺ | 8 |
| 91 | 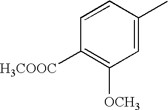 | (3aα,4β,7β,7aα)-2-Methoxy-4- (octahydro-1,3-dioxo-4,7-epoxy-2H- isoindol-2-yl)benzoic acid methyl ester | 2.75 LCMS/ 332.25 [M + H]⁺ | 8 |
| 92 | 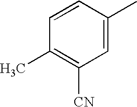 | (3aα,4β,7β,7aα)-2-Methyl-5- (octahydro-1,3-dioxo-4,7-epoxy-2H- isoindol-2-yl)benzonitrile | 2.80 LCMS/ 315.26 [M + CH₃OH + H]⁺ | 8 |
| 93 | 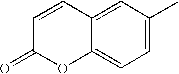 | (3aα,4β,7β,7aα)-Hexahydro-2-(2- oxo-2H-1-benzopyran-6-yl)-4,7- epoxy-1H-isoindole-1,3(2H)-dione | 2.45 LCMS/ 312.20 [M + H]⁺ | 8 |
| 94 | 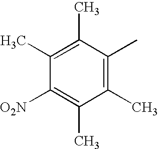 | (3aα,4β,7β,7aα)-Hexahydro-2- (2,3,5,6-tetramethyl-4-nitrophenyl)- 4,7-epoxy-1H-isoindole-1,3(2H)- dione | 3.59 LCMS/ 377.25 [M + CH₃OH + H]⁺ | 8 |
| 95 | 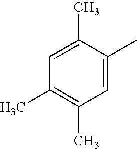 | (3aα,4β,7β,7aα)-Hexahydro-2- (2,4,5,-trimethylphenyl)-4,7-epoxy- 1H-isoindole-1,3(2H)-dione | 3.33 LCMS/ 286,30 [M + H]⁺ | 8 |
| 96 | 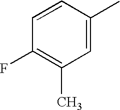 | (3aα,4β,7β,7aα)-2-(4-Fluoro-3- methylphenyl)hexahydro-4,7-epoxy- 1H-isoindole-1,3(2H)-dione | 3.00 LCMS/ 276.23 [M + H]⁺ | 8 |
| 97 | 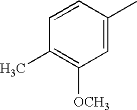 | (3aα,4β,7β,7aα)-Hexahydro-2-(3- methoxy-4-methylphenyl)-4,7- epoxy-1H-isoindole-1,3(2H)-dione | 3.05 LCMS/ 288.23 [M + H]⁺ | 8 |
| 98 | 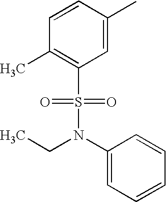 | (3aα,4β,7β,7aα)-N-Ethyl-2-methyl- 5-(octahydro-1,3-dioxo-4,7-epoxy- 2H-isoindol-2-yl)-N- phenylbenzenesulfonamide | 3.56 LCMS/ 441.26 [M + H]⁺ | 8 |
| 99 | 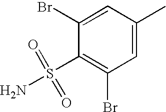 | (3aα,4β,7β,7aα)-2,6-Dibromo-4- (octahydro-1,3-dioxo-4,7-epoxy-2H- isoindol-2-yl)benzenesulfonamide | 2.25 LCMS | 8 |
| 100 | 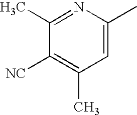 | (3aα,4β,7β,7aα)-2,4-Dimethyl-6- (octahydro-1,3-dioxo-4,7-epoxy-2H- isoindol-2-yl)-3-pyridinecarbonitrile | 2.75 LCMS/ 298.23 [M + H]⁺ | 8 |
| 101 | 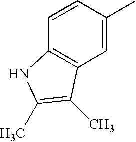 | (3aα,4β,7β,7aα)-2-(2,3-Dimethyl- 1H-indol-5-yl)hexahydro-4,7-epoxy- 1H-isoindole-1,3(2H)-dione | 3.00 LCMS/ 311.26 [M + H]⁺ | 8 |
| 102 | 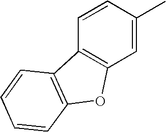 | (3aα,4β,7β,7aα)-2-(3- Dibenzofuranyl)hexahydro-4,7- epoxy-1H-isoindole-1,3(2H)-dione | 3.72 LCMS/ 366.23 [M + CH₃OH + H]⁺ | 8 |
| 103 | 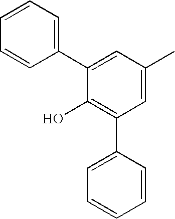 | (3aα,4β,7β,7aα)-Hexahydro-2-(2′- hydroxy[1,1′:3′,1″-terphenyl]-5′-yl)- 4,7-epoxy-1H-isoindole-1,3(2H)- dione | 3.70 LCMS/ 412.23 [M + H]⁺ | 8 |
| 104 | 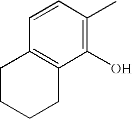 | (3aα,4β,7β,7aα)-Hexahydro-2- (5,6,7,8-tetrahydro-3-hydroxy-2- naphthalenyl)-4,7-epoxy-1H- isoindole-1,3(2H)-dione | 3.24 LCMS/ 312.32 [M + H]⁺ | 8 |
| 105 | 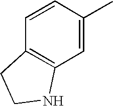 | (3aα,4β,7β,7aα)-2-(2,3-Dihydro-1H- indol-6-yl)hexahydro-4,7-epoxy-1H- isoindole-1,3(2H)-dione | 2.42 LCMS/ 285.29 [M + H]⁺ | 8 |
| 106 | 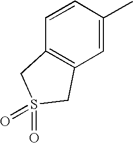 | (3aα,4β,7β,7aα)-2-(1,3-Dihydro-2,2- dioxidobenzo[c]thiophen-5- yl)hexahydro-4,7-epoxy-1H- isoindole-1,3(2H)-dione | 1.99 LCMS/ 366.26 [M + CH₃OH + H]⁺ | 8 |
| 107 | 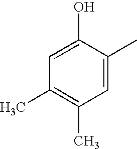 | (3aα,4β,7β,7aα)-Hexahydro-2-(2- hydroxy-4,5-dimethylphenyl)-4,7- epoxy-1H-isoindole-1,3(2H)-dione | 2.78 LCMS/ 286.32 [M − H]⁻ | 8 |
| 108 | 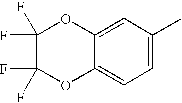 | (3aα,4β,7β,7aα)-2-(2,3-Dihydro- 2,2,3,3-tetrafluoro-1,4-benzodioxin- 6-yl)hexahydro-4,7-epoxy-1H- isoindole-1,3(2H)-dione | 3.82 LCMS/ 406.19 [M + CH₃OH + H]⁺ | 8 |
| 109 | 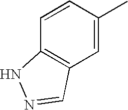 | (3aα,4β,7β,7aα)-Hexahydro-2-(1H- indazol-5-yl)-4,7-epoxy-1H- isoindole-1,3(2H)-dione | 2.13 LCMS/ 284.23 [M + H]⁺ | 8 |
| 110 | 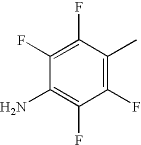 | (3aα,4β,7β,7aα)-2-(4-Amino- 2,3,5,6-tetrafluorophenyl)- hexahydro-4,7-epoxy-1H-isoindole- 1,3(2H)-dione | 2.60 LCMS/ 363.22 [M + CH₃OH + H]⁺ | 8 |
| 111 | 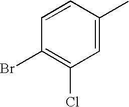 | (3aα,4β,7β,7aα)-2-(4-Bromo-3- chlorophenyl)hexahydro-4,7-epoxy- 1H-isoindole-1,3(2H)-dione | 3.64 LCMS/ 389.64 [M + CH₃OH + H]⁺ | 8 |
| 112 | 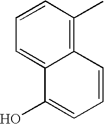 | (3aα,4β,7β,7aα)-Hexahydro-2-(5- hydroxy-1-naphthalenyl)-4,7-epoxy- 1H-isoindole-1,3(2H)-dione | 2.48 LCMS/ 308.27 [M − H]⁻ | 8 |
| 113 | 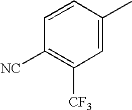 | (3aα,4β,7β,7aα)-4-(Octahydro-1,3- dioxo-4,7-epoxy-2H-isoindol-2-yl)-2- (trifluoromethyl)benzonitrile | 3.28 LCMS/ 337.16 [M + H]⁺ | 8 |
| 114 | 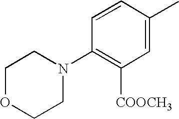 | (3aα,4β,7β,7aα)-2-(4-Morpholinyl)- 5-(octahydro-1,3-dioxo-4,7-epoxy- 2H-isoindol-2-yl)benzoic acid methyl ester | 2.72 LCMS/ 387.17 [M + H]⁺ | 8 |
| 115 | 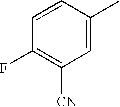 | (3aα,4β,7β,7aα)-2-Fluoro-5- (octahydro-1,3-dioxo-4,7-epoxy-2H- isoindol-2-yl)benzonitrile | 2.69 LCMS/ 319.26 [M + CH₃OH + H]⁺ | 8 |
| 116 | 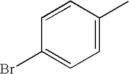 | (3aα,4β,7β,7aα)-2-(4- Bromophenyl)hexahydro-4,7-epoxy- 1H-isoindole-1,3(2H)-dione | 5.80 LCMS/ 393.0 [M + H]⁺ | 8 |
| 117 | 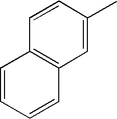 | (3aα,4β,7β,7aα)-Hexahydro-2-(2- naphthalenyl)-4,7-epoxy-1H- isoindole-1,3(2H)-dione | 6.92 LCMS/ 333.7 [M + H]⁺ | 8 |
| 118 | 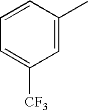 | (3aα,4β,7β,7aα)-Hexahydro-2-[3- trifluoromethyl)phenyl]-4,7-epoxy- 1H-isoindole-1,3(2H)-dione | 3.27 LCMS/ 312.2 [M + H]⁺ | 8 |
| 119 | 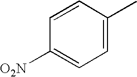 | (3aα,4β,7β,7aα)-Hexahydro-2-(4- nitrophenyl)-4,7-epoxy-1H- isoindole-1,3(2H)-dione | 2.88 LCMS/ 343.2 [M + H]⁺ | 8 |
| 120 | 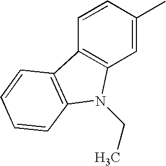 | (3aα,4β,7β,7aα)-2-(9-Ethyl-9H- carbazol-3-yl)hexahydro-4,7-epoxy- 1H-isoindole-1,3(2H)-dione | 3.73 LCMS/ 360.1 [M + H]⁺ | 8 |
| 121 | 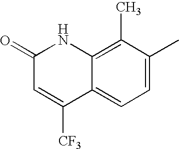 | (3aα,4β,7β,7aα)-2-[1,2-Dihydro-8- methyl-2-oxo-4-(trifluoromethyl)-7- quinolinyl]hexahydro-4,7-epoxy-1H- isoindole-1,3(2H)-dione | 3.11 LCMS/ 393.0 [M + H]⁺ | 8 |

### EXAMPLES 122 to 164

Further compounds of the present invention were prepared by procedures analogous to those described above. Table 3 provides the compound name and structure, retention time, as well as the Example number of the procedure on which the preparation of Table 3 was based, for the compounds of Examples 122 to 164. The chromatography techniques used to determine the compound retention times of Table 3 are as follows:

LCMS=YMC S5 ODS column, 4.6×50 mm eluting with 10-90% MeOH/H₂O over 4 minutes containing 0.1% TFA; 4 mL/min, monitoring at 220 nm.

LC=YMC S5 ODS column 4.6×50 mm eluting with 10-90% MeOH/H₂O over 4 minutes containing 0.2% phosphoric acid, 4 mL/min, monitoring at 220 nm

TABLE 3

| | | | Retention | |
|---|---|---|---|---|
| | | | Time | |
| | | | Min./ | |
| Ex. | Compound | Compound | Molecular | Pro. |
| No. | Structure | Name | Mass | of Ex. |
| 122 | 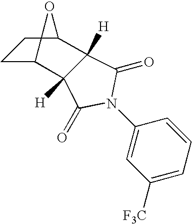 | (3aα,4α,7α,7aα)-Hexahydro- 2-[3-(trifluoromethyl)phenyl]- 4,7-epoxy-1H-isoindole- 1,3(2H)-dione | 2.66 LCMS | 27 |
| 123 | 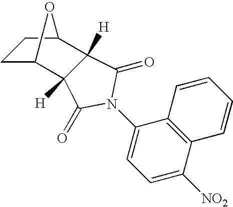 | (3aα,4α,7α,7aα)-Hexahydro- 2-(4-nitro-1-naphthalenyl- 4,7-epoxy-1H-isoindole- 1,3(2H)-dione | 2.76 LCMS | 27 |
| 124 | 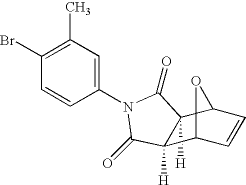 | (3aα,4β,7β,7aα)-2-(4- Bromo-3-methylphenyl)- 3a,4,7,7a-tetrahydro-4,7- epoxy-1H-isoindole-1,3(2H)- dione | 6.36 LCMS | 8 |
| 125 | 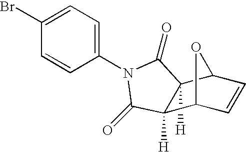 | (3aα,4β,7β,7aα)-2-(4- Bromophenyl)-3a,4,7,7a- tetrahydro-4,7-epoxy-1H- isoindole-1,3(2H)-dione | 5.72 LCMS | 8 |
| 126 | 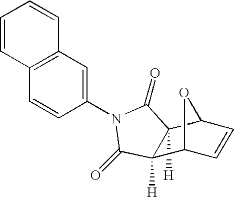 | (3aα,4β,7β,7aα)-3a,4,7,7a- Tetrahydro-2-(2- naphthalenyl)-4,7-epoxy-1H- isoindole-1,3(2H)-dione | 5.92 LCMS | 8 |
| 127 | 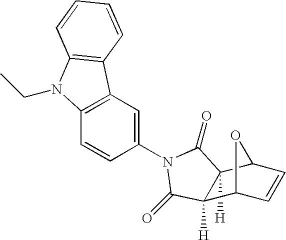 | (3aα,4β,7β,7aα)-2-(9-Ethyl- 9H-carbazol-3-yl)-3a,4,7,7a- tetrahydro-4,7-epoxy-1H- isoindole-1,3(2H)-dione | 3.73 LCMS | 8 |
| 128 | 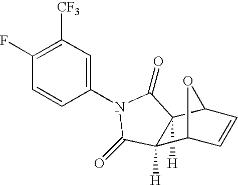 | (3aα,4β,7β,7aα)-2-[4-Fluoro- 3-(trifluoromethyl)phenyl]- 3a,4,7,7a-tetrahydro-4,7- epoxy-1H-isoindole-1,3(2H)- dione | 3.40 LCMS | 8 |
| 129 | 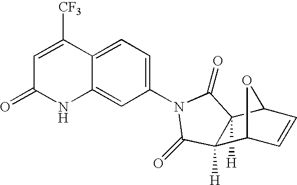 | (3aα,4β,7β,7aα)-2-[1,2- Dihydro-8-methyl-2-oxo-4- (trifluoromethyl)-7- quinolinyl]-3a,4,7,7a- tetrahydro-4,7-epoxy-1H- isoindole-1,3(2H)-dione | 3.14 LCMS | 8 |
| 130 | 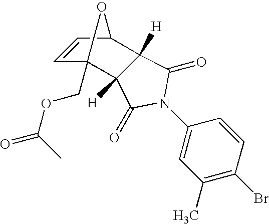 | (3aα,4α,7α,7aα)-4- [(Acetyloxy)methyl]-2-(4- bromo-3-methylphenyl)- 3a,4,7,7a-tetrahydro-4,7- epoxy-1H-isoindole-1,3(2H)- dione | 2.95 LC | 4 |
| 131 | 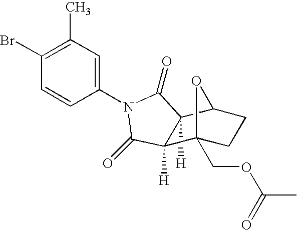 | (3aα,4β,7β,7aα)-4- [(Acetyloxy)methyl]-2-(4- bromo-3-methylphenyl)- 3a,4,7,7a-tetrahydro-4,7- epoxy-1H-isoindole-1,3(2H)- dione | 2.97 LC | 4 |
| 132 | 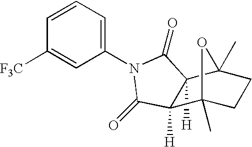 | (3aα,4β,7β,7aα)-Hexahydro- 4,7-dimethyl-2-[3- (trifluoromethyl)phenyl]-4,7- epoxy-1H-isoindole-1,3(2H)- dione | 3.08 LC | 20 |
| 133 | 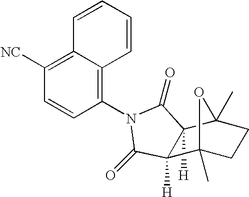 | (3aα,4β,7β,7aα)-4- (Octahydro-4,7-dimethyl-1,3- dioxo-4,7-epoxy-2H-isoindol-2- yl)-1-naphthalenecarbonitrile | 3.00 LC | 20 |
| 134 | 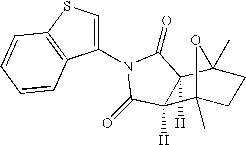 | (3aα,4β,7β,7aα)- (Benzo[b]thiophen-3- yl)hexahydro-4,7-dimethyl- 4,7-epoxy-1H-isoindole- 1,3(2H)-dione | 3.61 LC | 20 |
| 135 | 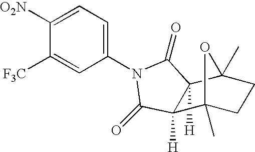 | (3aα,4β,7β,7aα)-Hexahydro- 4,7-dimethyl-2-[4-nitro-3- (trifluoromethyl)phenyl]-4,7- epoxy-1H-isoindole-1,3(2H)- dione | 3.21 LC | 20 |
| 136 | 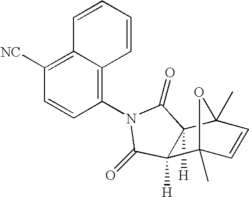 | (3aα,4β,7β,7aα)-4- (1,3,3a,4,7,7a-Hexahydro- 4,7-dimethyl-1,3-dioxo-4,7- epoxy-2H-isoindol-2-yl)-1- naphthalenecarbonitrile | 2.94 LC | 32 |
| 137 | 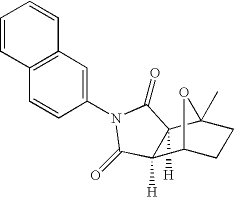 | (3aα,4α,7α,7aα)-Hexahydro- 4-methyl-2-(2-naphthalenyl)- 4,7-epoxy-1H-isoindole- 1,3(2H)-dione | 2.88 LC | 3 |
| 138 | 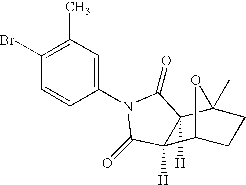 | (3aα,4β,7β,7aα)-2-(4- Bromo-3- methylphenyl)hexahydro-4- methyl-4,7-epoxy-1H- isoindole-1,3(2H)-dione | 3.11 LC | 3 |
| 139 | 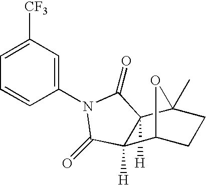 | (3aα,4β,7β,7aα)-Hexahydro- 4-methyl-2-[3- (trifluoromethyl)phenyl]-4,7- epoxy-1H-isoindole-1,3(2H)- dione | 2.90 LC | 3 |
| 140 | 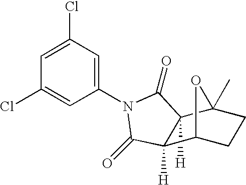 | (3aα,4β,7β,7aα)-2-(3,5- Dichlorophenyl)hexahydro-4- methyl-4,7-epoxy-1H- isoindole-1,3(2H)-dione | 3.31 LC | 3 |
| 141 | 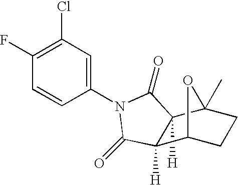 | (3aα,4β,7β,7aα)-2-(3- Chloro-4-fluorophenyl)- hexahydro-4-methyl-4,7- epoxy-1H-isoindole-1,3(2H)- dione | 2.72 LC | 3 |
| 142 | 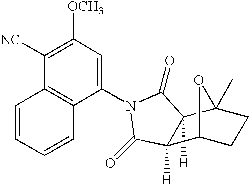 | (3aα,4β,7β,7aα)-2-Methoxy- 4-(octahydro-1,3-dioxo-4- methyl-4,7-epoxy-2H- naphthalenecarbonitrile | 2.72 LC | 3 |
| 143 | 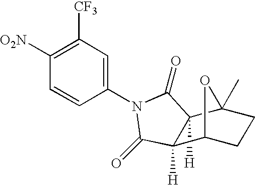 | (3aα,4β,7β,7aα)-Hexahydro- 4-methyl-2-[4-nitro-3- (trifluoromethyl)phenyl]-4,7- epoxy-1H-isoindole-1,3(2H)- dione | 3.10 LC | 3 |
| 144 | 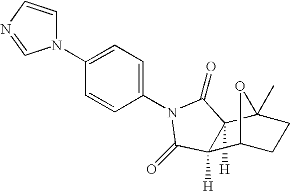 | (3aα,4β,7β,7aα)-Hexahydro- 2-[4-(1H-imidazol-1- yl)phenyl]-4-methyl-4,7- epoxy-1H-isoindole-1,3(2H)- dione | 1.16 LC | 3 |
| 145 | 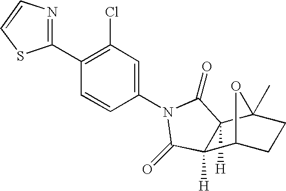 | (3aα,4β,7β,7aα)-2-[3-Chloro-4- (2-thiazolyl)phenyl]hexahydro- 4-methyl-4,7-epoxy-1H- isoindole-1,3(2H)-dione | 2.81 LC | 3 |
| 146 | 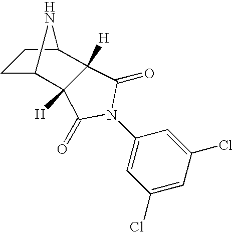 | (3aα,4α,7α,7aα)-2-(3,5- Dichlorophenyl)hexahydro- 4,7-imino-1H-isoindole- 1,3(2H)-dione | 2.72 LC | 31 |
| 147 | 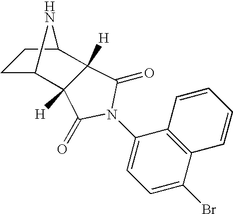 | (3aα,4α,7α,7aα)-2-(4-Bromo- 1-naphthalenyl)hexahydro-4,7- imino-1H-isoindole-1,3(2H)- dione | 2.95 LC | 31 |
| 148 | 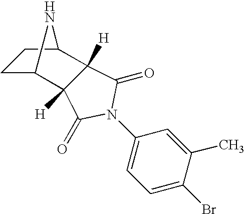 | (3aα,4α,7α,7aα)-2-(4-Bromo- 3-methylphenyl)hexahydro- 4,7-imino-1H-isoindole- 1,3(2H)-dione | 2.65 LC | 31 |
| 149 | 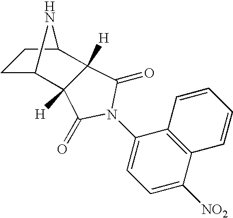 | (3aα,4α,7α,7aα)-Hexahydro- 2-(4-nitro-1-naphthalenyl)- 4,7-imino-1H-isoindole- 1,3(2H)-dione | 2.46 LC | 31 |
| 150 | 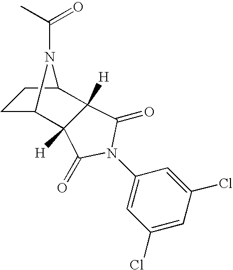 | (3aα,4α,7α,7aα)-8-Acetyl-2- (3,5-dichlorophenyl)hexahydro- 4,7-imino-1H-isoindole- 1,3(2H)-dione | 3.53 LC | 31 |
| 151 | 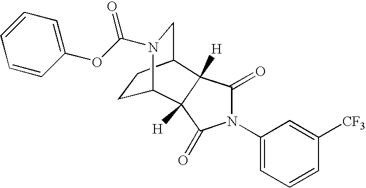 | (3aα,4α,7α,7aα)-Octahydro- 1,3-dioxo-2-[3- (trifluoromethyl)phenyl]-4,7- ethano-5H-pyrrolo[3,4- c]pyridine-5-carboxylic acid phenyl ester | 3.397 LC | 9 |
| 152 | 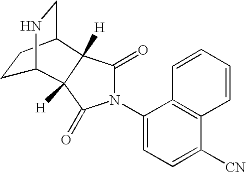 | (3aα,4α,7α,7aα)-4- (Octahydro-1,3-dioxo-4,7- ethano-2H-pyrrolo[3,4- c]pyridin-2-yl)-1- naphthalenecarbonitrile | 1.74 LC | 11 |
| 153 | 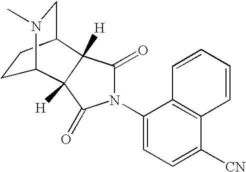 | (3aα,4α,7α,7aα)-4- (Octahydro-5-methyl-1,3- dioxo-4,7-ethano-2H- pyrrolo[3,4-c]pyridin-2-yl)-1- naphthalenecarbonitrile | 1.71 LC | 14 |
| 154 | 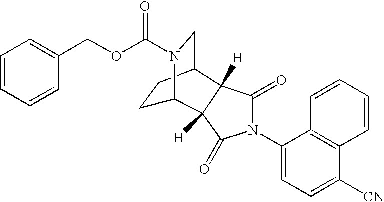 | (3aα,4α,7α,7aα)-2-(4-Cyano- 1-naphthalenyl)octahydro-1,3- dioxo-4,7-etheno-5H- pyrrolo[3,4-c]pyridine-5- carboxylic acid phenylmethyl ester | 3.40 LC | 10 |
| 155 | 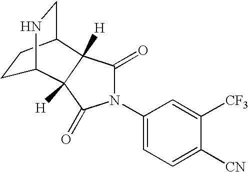 | (3aα,4α,7α,7aα)-4- (Octahydro-1,3-dioxo-4,7- ethano-2H-pyrrolo[3,4- c]pyridin-2-yl)-2- (trifluoromethyl)benzonitrile | 1.74 LC | 11 |
| 156 | 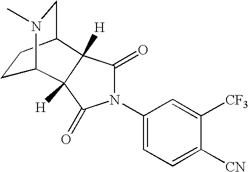 | (3aα,4α,7α,7aα)-4- (Octahydro-5-methyl-1,3- dioxo-4,7-ethano-2H- pyrrolo[3,4-c]pyridin-2-yl)-2- (trifluoromethyl)benzonitrile | 1.65 LC | 14 |
| 157 | 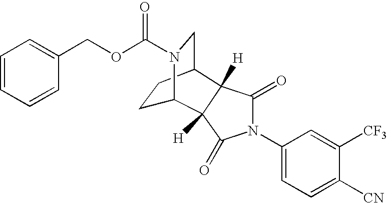 | (3aα,4α,7α,7aα)-2-[4-Cyano- 3-(trifluoromethyl)phenyl]octa- hydro-1,3-dioxo-4,7-etheno- 5H-pyrrolo[3,4-c]pyridine-5- carboxylic acid phenylmethyl ester | 3.53 LC | 10 |
| 158 | 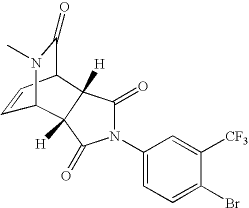 | (3aα,4α,7α,7aα)-2-[4-Bromo- 3-(trifluoromethyl)phenyl]tetra- hydro-5-methyl-4,7-etheno- 1H-pyrrolo[3,4-c]pyridine- 1,3,6(2H,5H)-trione | 2.95 LCMS | 34 |
| 159 | 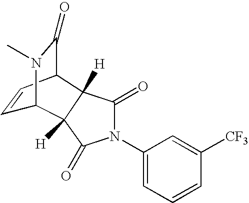 | (3aα,4α,7α,7aα)-Tetrahydro- 5-methyl-2-[3- (trifluoromethyl)phenyl]-4,7- etheno-1H-pyrrolo[3,4- c]pyridine-1,3,6(2H,5H)- trione | 2.53 LCMS | 34 |
| 160 | 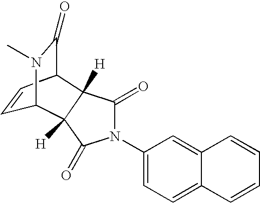 | (3aα,4α,7α,7aα)-Tetrahydro- 5-methyl-2-(2-naphthalenyl)- 4,7-etheno-1H-pyrrolo[3,4- c]pyridine-1,3,6(2H,5H)- trione | 2.58 LCMS | 34 |
| 161 | 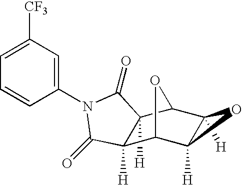 | (1aα,2β,2aα,5aα,6β,6aα)- Hexahydro-4-[3- (trifluoromethyl)phenyl]-2,6- epoxy-3H-oxireno[f]iso- indole-3,5(4H)-dione | 1.80 LCMS | 28 |
| 162 | 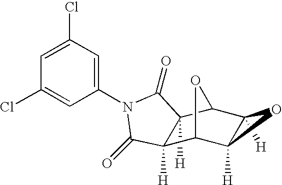 | (1aα,2β,2aα,5aα,6β,6aα)-4- (3,5-Dichlorophenyl)- hexahydro-2,6-epoxy-3H- oxireno[f]isoindole-3,5(4H)- dione | 1.45 LCMS | 28 |
| 163 | 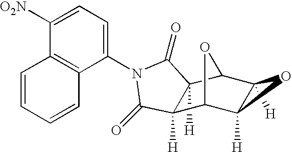 | (1aα,2β,2aα,5aα,6β,6aα)- Hexahydro-4-(4-nitro-1- naphthalenyl)-2,6-epoxy-3H- oxireno[f]isoindole-3,5(4H)- dione | 1.52 LCMS | 28 |
| 164 | 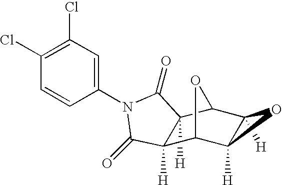 | (1aα,2β,2aα,5aα,6β,6aα)-4- (3,4-Dichlorophenyl)- hexahydro-2,6-epoxy-3H- oxireno[f]isoindole-3,5(4H)- dione | 3.21 LCMS | 28 |

### EXAMPLES 165 to 203

Additional compounds of the present invention were prepared and are described further below in Table 4. Table 4 sets forth the compound name and structure, as well as the Example number of the procedure on which the preparation of Table 4 was based, for the compounds of Examples 165 to 203.

TABLE 4

| Ex. | Compound | Compound | Pro. |
|---|---|---|---|
| No. | Structure | Name | of Ex. |
| 165 | 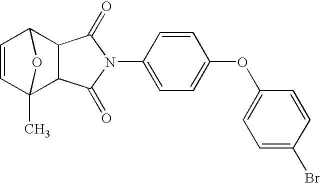 | 2-[4-(4-Bromo- phenoxy)phenyl]- 3a,4,7,7a-tetrahydro- 4,7-dimethyl-4,7- epoxy-1H-isoindole- 1,3(2H)-dione | 32 |
| 166 | 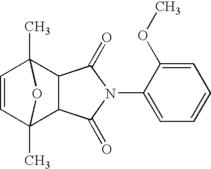 | 3a,4,7,7a-Tetrahydro- 2-(2-methoxyphenyl)- 4,7-dimethyl-4,7- epoxy-1H-isoindole- 1,3(2H)-dione | 32 |
| 167 | 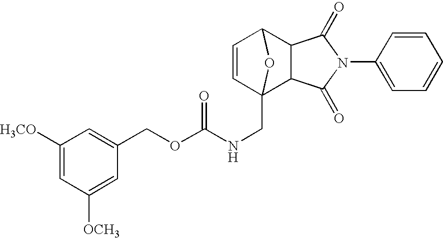 | [(1,2,3,3a,7,7a- Hexahydro-2-phenyl- 4,7-epoxy-4H- isoindol-4- yl)methyl]carbamic acid (3,5- dimethoxyphenyl)methyl ester | 21-26 |
| 168 | 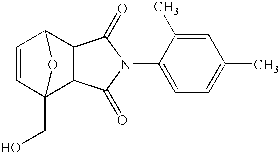 | 2-(2,4- Dimethylphenyl)- 3a,4,7,7a-tetrahydro- 4-(hydroxymethyl)- 4,7-epoxy-1H- isoindole-1,3(2H)- dione | 21-26 |
| 169 | 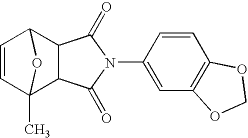 | 2-(1,3-Benzodioxol-5- yl)-3a,4,7,7a- tetrahydro-4-methyl- 4,7-epoxy-1H- isoindole-1,3(2H)- dione | 32 |
| 170 | 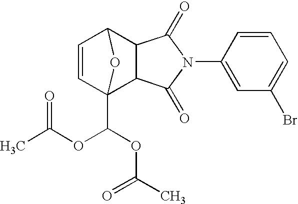 | 4-[Bis(acetyloxy)methyl]- 2-(3-bromophenyl)- 3a,4,7,7a-tetrahydro- 4,7-epoxy-1H- isoindole-1,3(2H)- dione | 21-26 |
| 171 | 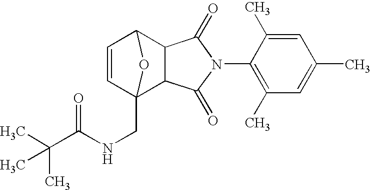 | N-[[1,2,3,3a,7,7a- Hexahydro-2-(2,4,6- trimethylphenyl)-4,7- epoxy-4H-isoindol-4- yl]methyl]-2,2- dimethylpropanamide | 21-26 |
| 172 | 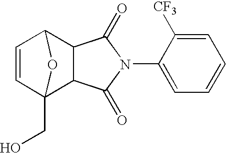 | 3a,4,7,7a-Tetrahydro- 4-(hydroxymethyl)-2- [2-(trifluoromethyl)phenyl]- 4,7-epoxy-1H- isoindole-1,3(2H)- dione | 21—26 |
| 173 | 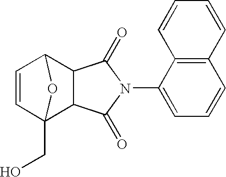 | 3a,4,7,7a-Tetrahydro- 4-(hydroxymethyl)-2- (1-naphthalenyl)-4,7- epoxy-1H-isoindole- 1,3(2H)-dione | 21-26 |
| 174 | 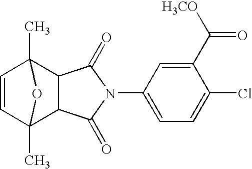 | 2-Chloro-5- (1,3,3a,4,7,7a- hexahydro-4,7- dimethyl-4,7-epoxy- 2H-isoindol-2- yl)benzoic acid methyl ester | 32 |
| 175 | 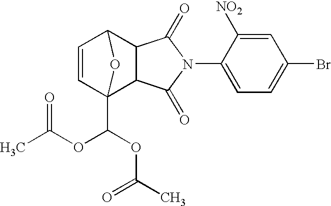 | 4-[Bis(acetyloxy)methyl]- 2-(4-bromo-2- nitrophenyl)- 3a,4,7,7a-tetrahydro- 4,7-epoxy-1H- isoindole-1,3(2H)- dione | 21-26 |
| 176 | 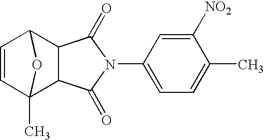 | 3a,4,7,7a-Tetrahydro- 4-methyl-2-(4-methyl- 3-nitrophenyl)-4,7- epoxy-1H-isoindole- 1,3(2H)-dione | 32 |
| 177 | 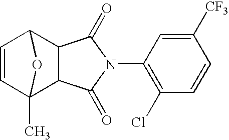 | 2-[2-Chloro-5- (trifluoromethyl)phenyl]- 3a,4,7,7a- tetrahydro-4-methyl- 4,7-epoxy-1H- isoindole-1,3(2H)- dione | 32 |
| 178 | 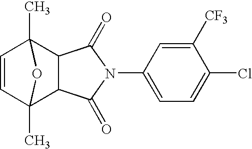 | 2-[4-Chloro-3- (trifluoromethyl)phenyl]- 3a,4,7,7a- tetrahydro-4,7- dimethyl-4,7-epoxy- 1H-isoindole-1,3(2H)- dione | 32 |
| 179 | 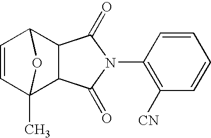 | 2-(1,3,3a,4,7,7a- Hexahydro-4-methyl- 4,7-epoxy-2H- isoindol-2- yl)benzonitrile | 32 |
| 180 | 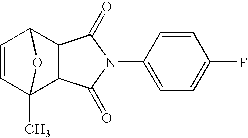 | 2-(4-Fluorophenyl)- 3a,4,7,7a-tetrahydro- 4-methyl-4,7-epoxy- 1H-isoindole-1,3(2H)- dione | 32 |
| 181 | 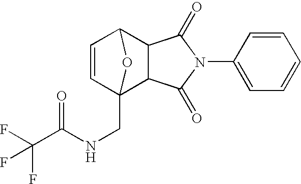 | 2,2,2-Trifluoro-N- [(1,2,3,3a,7,7a- hexahydro-2-phenyl- 4,7-epoxy-4H- isoindol-4- yl)methyl]acetamide | 21-26 |
| 182 | 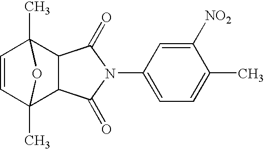 | 3a,4,7,7a-Tetrahydro- 4,7-dimethyl-2-(4- methyl-3-nitrophenyl)- 4,7-epoxy-1H- isoindole-1,3(2H)- dione | 32 |
| 183 | 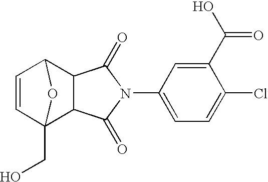 | 2-Chloro-5- [1,3,3a,4,7,7a- hexahydro-4- (hydroxymethyl)-4,7- epoxy-2H-isoindol-2- yl]benzoic acid | 21-26 |
| 184 | 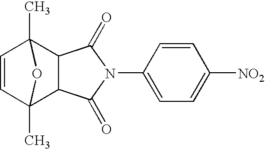 | 3a,4,7,7a-Tetrahydro- 4,7-dimethyl-2-(4- nitrophenyl)-4,7- epoxy-1H-isoindole- 1,3(2H)-dione | 32 |
| 185 | 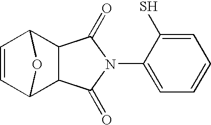 | 3a,4,7,7a-Tetrahydro- 2-(2-mercaptophenyl)-4,7- epoxy-1H-isoindole- 1,3(2H)-dione | 32 |
| 186 | 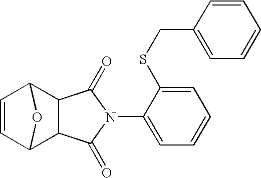 | 3a,4,7,7a-Tetrahydro- 2-[2-[(phenyl- methyl)thio]phenyl]-4,7- epoxy-1H-isoindole- 1,3(2H)-dione | 32 |
| 187 | 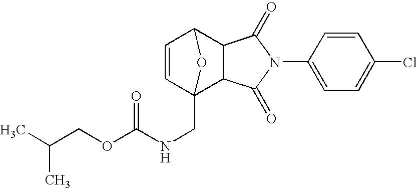 | [[2-(4-Chlorophenyl)- 1,2,3,3a,7,7a- hexahydro-4,7-epoxy- 4H-isoindol-4- yl]methyl]carbamic acid 2-methylpropyl ester | 21-26 |
| 188 | 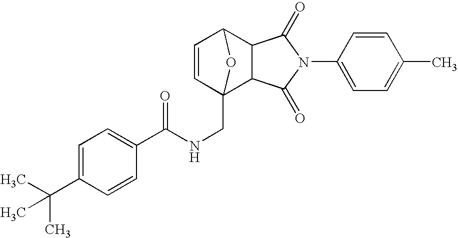 | 4-(1,1-Dimethylethyl)- N-[[1,2,3,3a,7,7a- hexahydro-2-(4- methylphenyl)-4,7- epoxy-4H-isoindol-4- yl]methyl]benzamide | 21-26 |
| 189 | 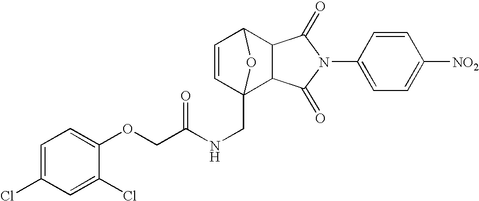 | 2,4-Dichloro-N- [[1,2,3,3a,7,7a- hexahydro-2-(4- nitrophenyl)-4,7- epoxy-4H-isoindol-4- yl]methyl]benzamide | 21-26 |
| 190 | 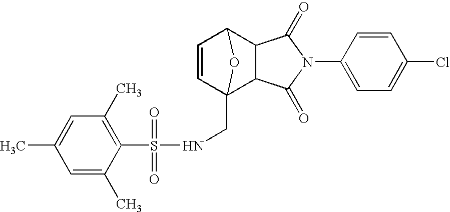 | N-[[2-(4- Chlorophenyl)- 1,2,3,3a,7,7a- hexahydro-4,7-epoxy- 4H-isoindol-4- yl]methyl]-2,4,6- trimethylbenzene- sulfonamide | 21-26 |
| 191 | 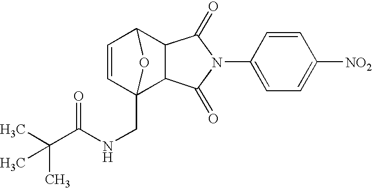 | N-[[1,2,3,3a,7,7a- Hexahydro-2-(4- nitrophenyl)-4,7- epoxy-4H-isoindol-4- yl]methyl]-2,2- dimethylpropanamide | 21-26 |
| 192 | 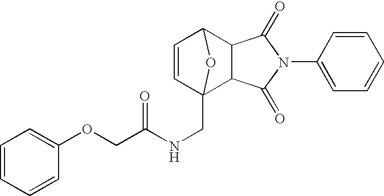 | N-[(1,2,3,3a,7,7a- Hexahydro-2-phenyl- 4,7-epoxy-4H- isoindol-4-yl)methyl]- 2-phenoxyacetamide | 21-26 |
| 193 | 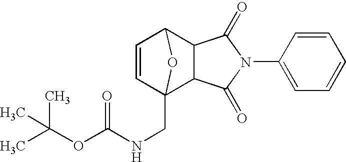 | [(1,2,3,3a,7,7a- Hexahydro-2-phenyl- 4,7-epoxy-4H- isoindol-4- yl)methyl]carbamic acid 1,1-dimethylethyl ester | 21-26 |
| 194 | 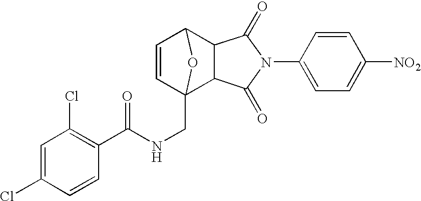 | 2-(2,4- Dichlorophenoxy)-N- [[1,2,3,3a,7,7a- hexahydro-2-(4- nitrophenyl)-4,7- epoxy-4H-isoindol-4- yl]methyl]acetamide | 21-26 |
| 195 | 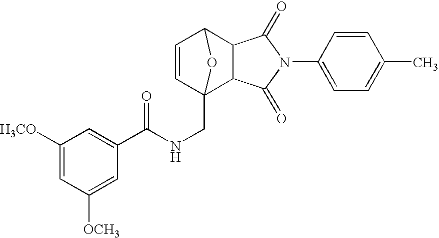 | N-[[1,2,3,3a,7,7a- Hexahydro-2-(4- methylphenyl)-4,7- epoxy-4H-isoindol-4- yl]methyl]-3,5- dimethoxybenzamide | 21-26 |
| 196 | 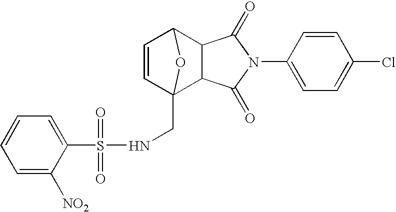 | N-[[2-(4-Chlorophenyl)- 1,2,3,3a,7,7a- hexahydro-4,7-epoxy- 4H-isoindol-4- yl]methyl]-2- nitrobenzenesulfonamide | 21-26 |
| 197 | 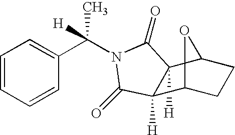 | (3aα,4β,7β,7aα)- Hexahydro-2-[(1S)-1- phenylethyl]-4,7- epoxy-1H-isoindole- 1,3(2H)-dione. | 8 |
| 198 | 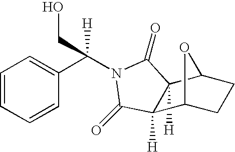 | (3aα,4β,7β,7aα)- Hexahydro-2-[(1S)-2- hydroxy-1- phenylethyl]-4,7- epoxy-1H-isoindole- 1,3(2H)-dione. | 8 |
| 199 | 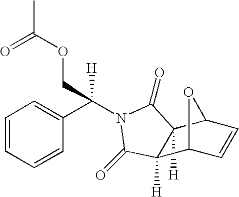 | (3aα,4β,7β,7aα)-2- [(1S)-2-(Acetyloxy)-1- phenylethyl]- 3a,4,7,7a-tetrahydro- 4,7-epoxy-1H- isoindole-1,3(2H)- dione. | 8 |
| 200 | 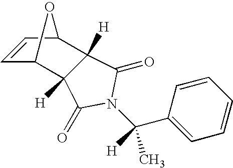 | (3aα,4α,7α,7aα)- 3a,4,7,7a-Tetrahydro- 2-[(1S)-1-phenylethyl]-4,7- epoxy-1H-isoindole- 1,3(2H)-dione. | 8 |
| 201 | 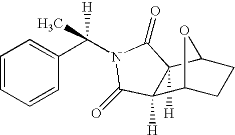 | (3aα,4β,7β,7aα)- Hexahydro-2-[(1R)-1- phenylethyl]-4,7- epoxy-1H-isoindole- 1,3(2H)-dione. | 8 |
| 202 | 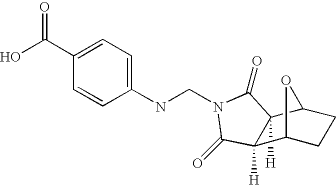 | (3aα,4β,7β,7aα)- [[[(Octahydro-1,3- dioxo-4,7-epoxy-2H- isoindol-2- yl)methyl]amino]benzoic acid. | 8 |
| 203 | 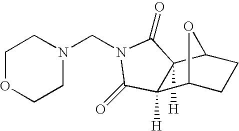 | (3aα,4β,7β,7aα)- Hexahydro-2-(4- morpholinylmethyl)- 4,7-epoxy-1H- isoindole-1,3(2H)- dione. | 8 |

### EXAMPLE 204

### (3aα,4β,7β,7aα)-4-[Octahydro-4-(2-hydroxyethyl)-7-methyl-1,3-dioxo-4,7-epoxy-2H-isoindol-2-yl]-2-(trifluoromethyl)benzonitrile (204D/25B)

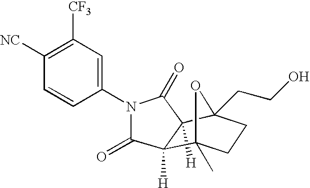

A. 2-[2-[[(1,1-Dimethylethyl)dimethylsilyl]oxy]ethyl]-5-methylfuran (204A)

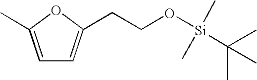

To a solution of compound 21A (2.00 g, 15.9 mmol) in DMF (50 mL) was added imidazole (1.62 g, 23.9 mmol), followed by tert-butyldimethylsilyl chloride (2.63 g, 17.5 mmol). After 2 h at 25° C., the reaction was poured into diethyl ether (300 mL) and washed with water (1×100 mL), 1N HCl (1×100 mL), water (1×100 mL), brine (1×50 mL) and dried over anhydrous MgSO₄. Crude compound 204A was analyzed by LCMS and NMR and determined to be pure enough to be carried on directly to the next step. HPLC: 100% at 4.347 min (retention time) (YMC S5 ODS column 4.6×50 mm eluting with 10-90% aqueous methanol over 4 minutes containing 0.1% TFA, 4 mL/min, monitoring at 220 nm).

B. (3aα,4β,7β,7aα)-4-[2-[[(1,1-Dimethylethyl)dimethylsilyl]-oxy]ethyl]hexahydro-7-methyl-4,7-epoxy-1H-isobenzofuran-1,3(2H)-dione (204B)

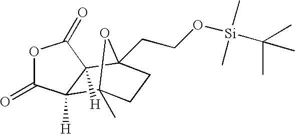

Compound 204A (4.0 g, 18.9 mmol) and maleic anhydride (1.42 g, 14.51 mmol) were dissolved in dichloroethane (10 mL) and stirred at 25° C. for 60 hours.

The volatiles were then removed in vacuo and the resulting orange oil was dissolved in absolute ethanol (50 mL) and Pd/C (10% Pd, 1.00 g) was added. Hydrogen was then introduced via a balloon. After 3 h, the reaction was filtered through celite rinsing with EtOAc and concentrated in vacuo. The crude anhydride was purified by rapid flash chromatography in SiO2 eluting with acetone/chloroform (0-2-4% acetone) to give 1.30 g of compound 204B as a clear oil, in addition to 3.00 g of the starting compound 204A. Characterization by proton NMR spectroscopy showed only the exo isomer. 1H NMR, 400 MHz, CDCl3, 3.83 (2H, t, J=6.0 Hz), 3.22 (1H, d, J=8.2 Hz), 3.06 (1H, d, J=8.2 Hz), 1.70-2.25 (6H, m), 1.55 (3H, s), 0.82 (9H, s), 0.00 (6H, s).

C. (3aα,4β,7β,7aα)-4-[4-[2-[[(1,1-Dimethylethyl)dimethylsilyl]-oxy]ethyl]octahydro-7-methyl-1,3-dioxo-4,7-epoxy-2H-isoindol-2-yl]-2-(trifluoromethyl)benzonitrile (204C)

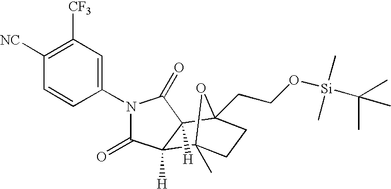

Compound 204B (0.250 g, 0.8 mmol) and 4-amino-2-trifluoromethyl-benzonitrile (0.124 g, 0.668 mmol) were suspended in dry toluene (2.0 mL) in a sealed tube. MgSO₄ (0.200 g) and triethylamine (0.5 mL) were then added and the tube was sealed and placed in a oil bath at 125° C. After 40 h, the reaction was cooled to 25° C., filtered and concentrated in vacuo. The crude material was purified by flash chromatography on SiO₂ eluting with CH₂Cl₂ to give 0.111 g of compound 204C as a yellow solid. HPLC: 92% at 4.203 min (retention time) (YMC S5 ODS column 4.6×50 mm eluting with 10-90% aqueous methanol over 4 minutes containing 0.1% TFA, 4 mL/min, monitoring at 220 nm). MS (ESI): m/z 531.1 [M+Na]⁺.

D. (3aα,4β,7β,7aα)-4-[Octahydro-4-(2-hydroxyethyl)-7-methyl-1,3-dioxo-4,7-epoxy-2H-isoindol-2-yl]-2-(trifluoromethyl)benzonitrile (204D)

Compound 204C (0.031 g, 0.061 mmol) was dissolved in THF (0.5 mL) and transferred to a polypropylene container followed by cooling to 0° C. HF-pyridine (˜47% HF, 0.1 mL) was then added. After 15 min, the reaction was complete as determined by LC and was poured into cold sat aqueous NaHCO₃. The mixture was extracted with CH₂Cl₂ (3×10 mL). The combined organic layers were washed with 1 N HCl (1×20 mL) and dried over anhydrous Na₂SO₄. Compound 204D was isolated as a yellow oil and compared to the material prepared in Example 25. No purification was necessary.

### EXAMPLE 205

### (3aα,4β,7β,7aα)- and (3aα,4α,7α,7aα)-4-[Octahydro-4-methyl-1,3-dioxo-7-(phenylmethyl)-4,7-epoxy-2H-isoindol-2-yl]-2-(trifluoromethyl)benzonitrile (205Ci and 205Cii, respectively)

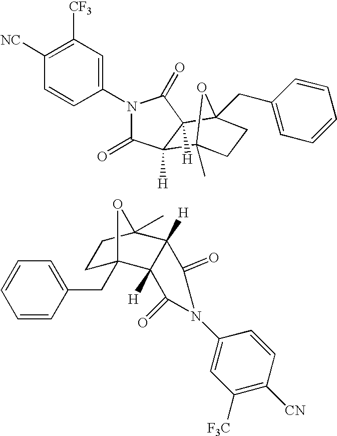

A. 2-Methyl-5-(phenylmethyl)-furan (205A)

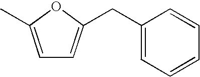

n-BuLi (1.8 ml, 4.51 mmol, 1.1 eq, 2.5 M in hexane) was added to a solution of 2-methyl-furan (0.37 ml, 4.10 mmol, 1 eq) in anhydrous THF (3 mL) at −25° C. The resulting solution was stirred at room temperature for 3 h and then cooled to −15° C. Benzyl bromide (0.59 ml, 4.92 mmol, 1.2 eq), which was passed through a plug of aluminum oxide, was added and the solution was warmed to rt and stirred overnight. Saturated NH₄Cl solution (5 mL) was added and the mixture was stirred for 1 h. The reaction mixture was then extracted by ether (2×) and the combined organic extracts were dried and concentrated under reduced pressure. Purification by flash chromatography on silica gel eluting with hexanes gave 323 mg (46%, 1.88 mmol) of compound 205A as colorless oil. HPLC: 95% at 3.72 min (retention time) (YMC S5 ODS column 4.6×50 mm, 10-90% aqueous methanol over 4 minutes containing 0.2% phosphoric acid, 4 mL/min, monitoring at 220 nm) and about 400 mg mixture of product and benzyl bromide (˜2:1 by HPLC).

B. (3aα,40,7β,7aα)- and (3aα,4α,7α,7aα)-4-[Octahydro-4-methyl-1,3-dioxo-7-(phenylmethyl)-4,7-epoxy-2H-isoindol-2-yl]-2-(trifluoromethyl) benzonitrile (205Bi and 205Bii, respectively)

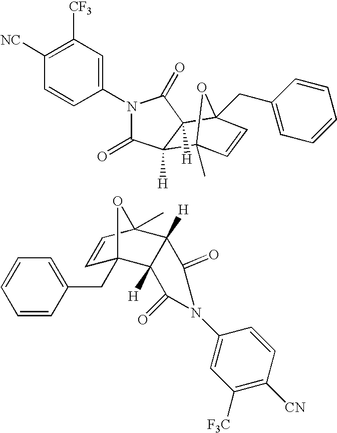

A solution of compound 205A (124 mg, 0.72 mmol, 1 eq) and 4-(2,5-dihydro-2,5-dioxo-1H-pyrrol-1-yl)-2-trifluoromethylbenzonitrile (290 mg, 1.09 mmol, 1.5 eq) in CH₂Cl₂ (2 mL) was stirred at room temperature. After 4 days, the reaction mixture was concentrated under reduced pressure. Purification by flash chromatography on silica gel eluting with CH₂Cl₂ gave 62 mg (0.14 mmol, 20%) of a mixture of compounds 205Bi and 205Bii as a white solid, which was used directly in the next step. HPLC: 93% at 3.69 min (retention time) (YMC S5 ODS column 4.6×50 mm, 10-90% aqueous methanol over 4 minutes containing 0.2% phosphoric acid, 4 mL/min, monitoring at 220 nm).

C. (3aα,4β,7β,7aα)- and (3aα,4α,7α,7aα)-4-[Octahydro-4-methyl-1,3-dioxo-7-(phenylmethyl)-4,7-epoxy-2H-isoindol-2-yl]-2-(trifluoromethyl)-benzonitrile (205Ci and 205Cii, respectively)

A solution of a mixture of compounds 205Bi and 205Bii (62 mg, 0.14 mmol, 1 eq) and 10% Pd/C (12 mg, cat.) in EtOH (3.5 mL) was stirred under a hydrogen atmosphere at room temperature for 2 h. The reaction mixture was filtered through celite and concentrated under reduced pressure. Purification by flash chromatography on silica gel eluting with 35% EtOAc/hexanes gave 22 mg (0.05 mmol, 35%) of compound 205Ci and 12 mg (0.027 mmols, 19%) of compound 205Cii. Compound 205Ci: HPLC: 98% at 3.75 min (retention time) (YMC S5 ODS column 4.6×50 mm, 10-90% aqueous methanol over 4 minutes containing 0.2% phosphoric acid, 4 mL/min, monitoring at 220 nm), MS (ESI): m/z 458.2 [M+NH₄]⁺. Compound 205Cii: HPLC: 97% at 3.78 min (YMC S5 ODS column 4.6×50 mm, 10-90% aqueous methanol over 4 minutes containing 0.2% phosphoric acid, 4 mL/min, monitoring at 220 nm), MS (ESI): m/z 473.45 [M+CH₃0H]⁺.

### EXAMPLE 206

### (3aα,4β,7β,7aα)-2-[4-Cyano-3-(trifluoromethyl)phenyl]octahydro-7-methyl-1,3-dioxo-4,7-epoxy-4H-isoindole-4-propanenitrile (206)

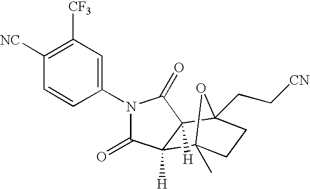

A solution of compound 36 (34 mg, 0.074 mmol) and NaCN (24 mg, 0.49 mmol) in DMSO (1 mL) was heated at 100° C. for 0.5 h. After cooling, the reaction mixture was poured into H₂O and the aqueous layer was extracted with EtOAc (2×). The combined organic layers were washed with H₂O (2×), dried over Na₂SO₄ and concentrated under reduced pressure. Purification by flash chromatography on SiO₂ eluting with 50% EtOAc/hexanes followed by preparative HPLC, 30.41 min (retention time) (YMC S5 ODS 30×250 mm, 10-90% aqueous methanol over 30 minutes containing 0.1% TFA, 25 mL/min, monitoring at 220 nm) gave 6.6 mg (22%) of compound 206 as a white solid. HPLC: 99% at 2.89 min (retention time) (YMC S5 ODS column 4.6×50 mm eluting with 10-90% aqueous methanol over 4 minutes containing 0.2% phosphoric acid, 4 mL/min, monitoring at 220 nm), MS (ES): m/z 402.1 [M−H]⁻.

### EXAMPLE 207

### (3aα,4β,7β,7aα)-4-[Octahydro4-methyl-7-[2-(4-morpholinyl)ethyl]-1,3-dioxo-4,7-epoxy-2H-isoindol-2-yl]-2-(trifluoromethyl)benzonitrile, trifluoroacetate (1:1) (207)

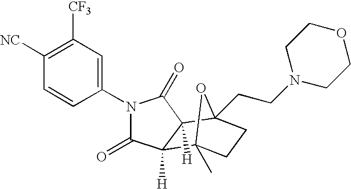

A solution of compound 36 (15.6 mg, 0.034 mmol) and morpholine (6 μL, 0.068 mmol) in toluene (1 mL) was heated at 100° C. overnight. After cooling, the reaction mixture was concentrated under reduced pressure. Purification by flash chromatography on SiO₂ eluting with 10% MeOH/CH₂Cl₂ followed by preparative HPLC, 23.96 min (retention time) (YMC S5 ODS 30×250 mm, 10-90% aqueous methanol over 30 minutes containing 0.1% TFA, mL/min, monitoring at 220 nm) gave 8.7 mg (55%) of compound 207 (TFA salt) as a white solid. HPLC: 99% at 2.02 min (retention time) (YMC S5 ODS column 4.6×50 mm eluting with 10-90% aqueous methanol over 4 minutes containing 0.2% phosphoric acid, 4 mL/min, monitoring at 220 nm), MS (ES): m/z 464.3 [M+H]⁺.

### EXAMPLE 208

### (3aα,4β,7β,7aα)-2-(5-Fluoro-1-naphthalenyl)hexahydro-4,7-dimethyl-4,7-epoxy-1H-isoindole-1,3(2H)-dione (208C)

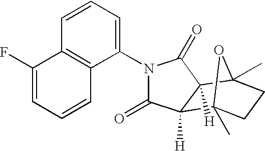

A. 1-Fluoro-5-nitronaphthalene (208A)

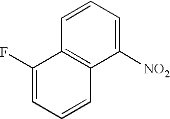

To a solution of 6N HCl (12 mL) was added 1.47 g (7.83 mmol) of finely powdered 5-nitro-1-naphthylamine, as described in J. Chem. Soc. 1187 (1949). The mixture was cooled to 0° C. and a cold solution of NaNO₂ (547 mg, 7.93 mmol) in 2 mL H₂O was added slowly so that the temperature was kept near 0° C. After the addition was complete, the reaction mixture was stirred for 30 min and filtered. The filtrate was cooled to 0° C. and treated with cold 4.5 M NaBF₄ solution (5 ml) to give complete precipitation of the diazonium borofluoride. The mixture was kept at 0° C. for 30 min before it was filtered and the precipitates were washed with cold 4.5 M NaBF₄ solution (5 mL), ice-cold ethanol (10 mL) and Et₂O (20 mL). The obtained solids were air dried to yield 1.74 g (77%) of the corresponding diazonium salt.

To 1.70 g (5.92 mmol) of the above diazonium borofluoride was added 5 g of sand and the components were thoroughly mixed. The reaction mixture was heated cautiously under reduced pressure until decomposition set in. Toward the end of the reaction the flask was further heated for 30 min to 130° C. to assure complete conversion. After cooling the reaction mixture was dissolved in acetone and the contents were preabsorbed on silica gel. Purification was achieved by flash chromatography (silica gel, EtOAc in hexanes 0 to 10%) to give 449 mg (50%) of compound 208A as a white solid.

B. 1-Amino-5-fluoronaphthalene (208B)

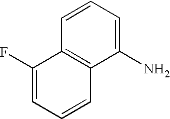

A solution of compound 208A (62 mg, 0.32 mmol) in 1 mL EtOH containing 0.1 mL 12N HCl was heated to reflux. Iron powder (62 mg, 1.11 mmol) was added in small portions and heating was continued for 2 h. The mixture was cooled, neutralized with 1N NaOH solution and the aqueous layer was extracted with CH₂Cl₂. The combined organic phases were dried over MgSO₄ and concentrated to leave a residue which was purified by flash chromatography (silica gel, EtOAc in hexanes 40 to 80%) yielding 42 mg (80%) of compound 208B as a yellow solid.

C. (3aα,4β,7β,7aα)-2-(5-Fluoro-1-naphthalenyl)hexahydro-4,7-dimethyl4,7epoxy-1H-isoindole-1,3(2H)-dione (208C)

Compound 208B (42 mg, 0.26 mmol), compound 20A (54 mg, 0.27 mmol), MgSO₄ (69 mg, 0.58 mmol) and triethylamine (191 μL, 1.37 mmol) were taken up in 2 mL of toluene and placed in a sealed tube. The sealed tube was heated to 135° C. for 14 h. The cooled reaction mixture was filtered through a short pad of Celite eluting with CH₂Cl₂ and the solvent was removed under reduced pressure. The residue was purified by preparative reverse phase HPLC (YMC S5 ODS 20×100 mm eluting with 30-100% aqueous methanol over 10 min containing 0.1% TFA, 20 mL/min) to give 15 mg (17%) of compound 208C as a light yellow solid. HPLC: 16% at 2.96 min & 77% at 3.06 min (retention time, atropisomers) (YMC S5 ODS column 4.6×50 mm eluting with 10-90% aqueous methanol over 4 minutes containing 0.2% phosphoric acid, 4 mL/min, monitoring at 220 nm), MS (ES): m/z 340.2 [M+H]⁺.

### EXAMPLE 209

### (3aα,4β,7β,7aα)-2-(5-Fluoro4-nitro-1-naphthalenyl)hexahydro-4,7-dimethyl-4,7-epoxy-1H-isoindole-1,3(2H)-dione (209C)

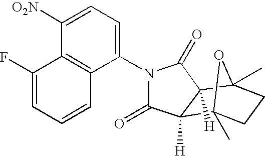

A. N-(5-Fluoro-1-naphthalenyl)acetamide (209A)

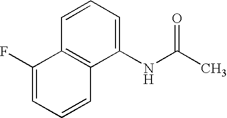

A solution of 141 mg (0.74 mmol) of compound 208A in 2 mL of AcOH was heated to reflux and treated with small portions of iron powder (118 mg, 2.11 mmol). The mixture was kept at reflux for 15 min before 73 μL (0.78 mmol) of Ac₂O was added. After an additional 15 min at reflux, the mixture was cooled and filtered eluting with CH₂Cl₂. The filtrate was then concentrated and the residue was purified by flash chromatography (silica gel, EtOAc in hexane 20 to 50%) to give compound 209A (145 mg, 97%) as a white solid.

B. 1-Amino-5-fluoro-4-nitronaphthalene (209B)

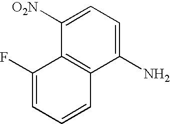

Compound 209A (133 mg, 0.66 mmol) was dissolved in 1 mL AcOH and the resulting solution was cooled to 10° C. At this temperature, 80 μL (2.00 mmol) of red fuming HNO₃ was added and stirring was continued for 15 min before the reaction was quenched by the addition of crushed ice. The aqueous layer was extracted with CH₂Cl₂ and the combined organic phases were dried over MgSO₄ and concentrated. The resulting residue was dissolved in 3 mL EtOH, heated to reflux and treated with 0.5 mL of 40% aqueous NaOH solution. Stirring was continued for 15 min before the reaction was cooled and diluted with H₂O. The aqueous layer was extracted with CH₂Cl₂ and the combined organic phases were dried over MgSO₄ and concentrated. The resulting residue was purified by flash chromatography (silica gel, EtOAc in hexane 40 to 70%) to afford 36 mg (27%) of compound 209B as a yellow solid.

C. 3aα,4β,7β,7aα)-2-(5-Fluoro-4-nitro-1-naphthalenyl)hexahydro-4,7-dimethyl-4,7-epoxy-1H-isoindole-1,3(2H)-dione (209C)

Compound 209B (36 mg, 0.18 mmol) was reacted in a sealed tube with compound 20A (38 mg, 0.19 mmol), MgSO₄ (46 mg, 0.39 mmol) and Et₃N (128 μL, 0.92 mmol) in 250 μL toluene according to the above procedure described in example 208C to give, after purification by preparative reverse phase HPLC (YMC S5 ODS 20×100 mm eluting with 30-100% aqueous methanol over 10 min containing 0.1% TFA, 20 mL/min), 27 mg (40%) of compound 209C as a yellow solid. HPLC: 8% at 2.88 min & 84% at 3.06 min (atropisomers, retention time) (YMC S5 ODS column 4.6×50 mm eluting with 10-90% aqueous methanol over 4 minutes containing 0.2% phosphoric acid, 4 mL/min, monitoring at 220 nm), MS (ES): m/z 402.0 [M+H]⁺.

### EXAMPLE 210

### (3aα,4β,7β,7aα)-2-(1,1-Dioxidobenzo[b]thiophen-3-yl)hexahydro-4,7-dimethyl-4,7-epoxy-1H-isoindole-1,3(2H)-dione (210)

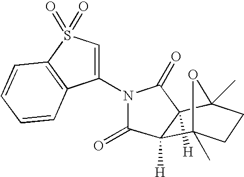

mCPBA (160 mg, 0.641 mmol, 70% pure) was added to a solution of compound 134 (70 mg, 0.214 mmol) in CH₂Cl₂ (2 mL) at rt. After the starting material was consumed, the reaction was quenched with sat. NaHCO₃, and extracted with CH₂Cl₂. The organic layer was washed with 1N NaOH, dried over Na₂SO₄ and concentrated under reduced pressure to give 63.9 mg (83%) of compound 210 as a white solid. HPLC: 99% at 3.81 min (retention time) (YMC S5 ODS column 4.6×50 mm eluting with 10-90% aqueous methanol over 4 minutes containing 0.2% phosphoric acid, 4 mL/min, monitoring at 220 nm), MS (ES): m/z 360.0 [M+H]⁺.

### EXAMPLE 211

### 4-(1,3,3a,4,7,7a-Hexahydro-4,6,7-trimethyl-1,3-dioxo-4,7-epoxy-2H-pyrrolo[3,4-c]pyridin-2-yl)-2-(trifluoromethyl)benzonitrile (211)

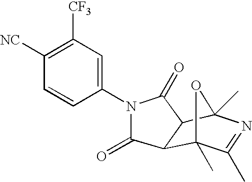

2,4,5-Trimethyl oxazole (0.48 mL, 4.14 mmol) was dissolved in toluene (2.0 mL) and 4-(2,5-dihydro-2,5-dioxo-1H-pyrrol-1-yl)-2-trifluoromethyl-benzonitrile (1.0 g 3.76 mmol) was added. The reaction mixture was stirred at 75° C. under nitrogen for 2.5 hrs. The solution was cooled to room temperature and the resulting precipitate was filtered and rinsed with toluene to give 0.51 g (35% yield) of compound 211 as a light grey solid. NMR analysis revelaed that compound 211 was one isomer (exo/endo) however the identity of the isomer could not be determined by NMR analysis. HPLC: 100% at 2.85 min (retention time) (YMC S5 ODS column 4.6×50 mm eluting with 10-90% aqueous methanol over 4 minutes containing 0.1% TFA, 4 mL/min, monitoring at 220 nm), MS (ES): m/z 378.42 [M+H]⁺.

### EXAMPLE 212

### (3aα,4β,7β,7aα)-Tetrahydro-4,7-dimethyl-2-[3-(trifluoromethyl)phenyl]-4,7-epoxy-1H-isoindole-1,3,5(2H,4H)-trione & (3aα,4α,7aα)-Tetrahydro-4,7-dimethyl-2-[3-(trifluoromethyl)phenyl]-4,7-epoxy-1H-isoindole-1,3,5(2H,4H)-trione (212i & 212ii, respectively)

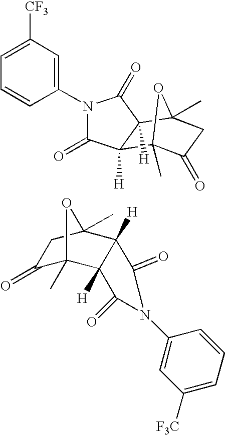

2,2-Dimethyl-3(H)-furanone (0.500 g, 4.46 mmol) and 1-[3-(trifluoromethyl)phenyl]-1H-pyrrole-2,5-dione (1.07 g, 4.46 mmol) were suspended in toluene (20 mL) in a sealed tube. The mixture was heated at 110° C. for 4 h and then cooled to 25° C. followed by concentration in vacuo. The resulting residue was purified by flash chromatography on SiO₂ eluting with methylene chloride to yield 0.411 g of compound 212i as a white solid and 0.193 g of compound 212ii as a white solid. The structural assignments were confirmed by 1-D NOE proton NMR experiments. Compound 212i: HPLC: 100% at 2.817 min (retention time) (YMC S5 ODS column 4.6×50 mm eluting with 10-90% aqueous methanol over 4 minutes containing 0.1% TFA, 4 mL/min, monitoring at 220 nm), MS (ES): m/z 376.0 [M+Na]⁺. Compound 212ii: HPLC: 100% at 3.013 min (retention time) (YMC S5 ODS column 4.6×50 mm eluting with 10-90% aqueous methanol over 4 minutes containing 0.2% phosphoric acid, 4 mL/min, monitoring at 220 nm), MS (ES): m/z 354.02 [M+H]⁺.

### EXAMPLE 213

### (3aα,4β,7β,7aα)-2-(5-Chloro-1-naphthalenyl)hexahydro-4,7-dimethyl-4,7-epoxy-1H-isoindole-1,3(2H)-dione (213B)

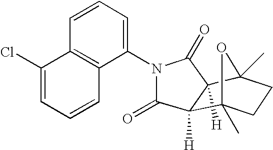

A. 1-Amino-5-chloronaphthalene (213A)

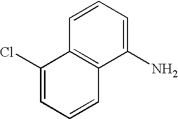

To a solution of 1.74 g (6.06 mmol) of the diazonium borofluoride (described in Example 208A) in acetone (7 mL) was added 693 mg (7.00 mmol) of CuCl in small portions. After the evolution of nitrogen had ceased the acetone was removed under reduced pressure and the residue was taken up in CH₂Cl₂ (30 mL). The organic phase was washed with H₂O (30 mL), dried over MgSO₄, concentrated and finally purified by flash chromatography (silica gel, EtOAc in hexane 0 to 15%) to give 754 mg (70%) of 1-chloro-5-nitronaphthalene.

The above synthesized 1-chloro-5-nitronaphthalene (540 mg, 2.6 mmol) was dissolved in 10 mL ACOH, followed by treatment with 415 mg (7.43 mmol) iron powder and subsequently acylated with Ac₂O (0.26 mL, 2.73 mmol) according to the procedure described in Example 209A to give 543 mg (95%) of 1-acetamino-5-chloronaphthalene.

A solution of the above synthesized 1-acetamino-5-chloronaphthalene (52 mg, 0.24 mmol) in 3 mL EtOH was heated to reflux and treated with 0.5 mL 40% aqueous NaOH solution. The mixture was refluxed until no more starting material could be detected, cooled and concentrated under reduced pressure. The residue was taken up in CH₂Cl₂ (50 mL) and was washed with H₂O (25 mL). The organic layer was dried over MgSO₄ and concentrated to leave 41 mg (98%) of compound 213A as a white solid.

B. (3aα,4β,7β,7aα)-2-(5-Chloro-1-naphthalenyl)hexahydro-4,7-dimethyl-4,7-epoxy-1H-isoindole-1,3(2H)-dione (213B)

Compound 213A (24 mg, 0.14 mmol) was reacted in a sealed tube with compound 20A (29 mg, 0.15 mmol), MgSO₄ (36 mg, 0.30 mmol) and Et₃N (100 μL, 0.71 mmol) in 250 μL toluene according to the procedure described in Example 208C to give, after purification by preparative reverse phase HPLC (YMC S5 ODS 20×100 mm eluting with 30-100% aqueous methanol over 10 min containing 0.1% TFA, 20 mL/min), 27 mg (40%) of compound 213B as a white solid. HPLC: 98% at 1.82 min (retention time) (YMC S5 TurboPack Pro column 4.6×33 mm eluting with 10-90% aqueous methanol over 2 minutes containing 0.2% phosphoric acid, 4 mL/min, monitoring at 220 nm), MS (ES): m/z 356.4 [M+H]⁺.

### EXAMPLE 214

### (3aα,4β,7β,7aα)-2-(5-Chloro-4-nitro-1-naphthalenyl)hexahydro-4,7-dimethyl-4,7-epoxy-1H-isoindole-1,3(2H)-dione (214B)

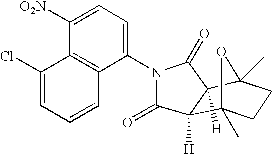

A. 1-Amino-5-chloro-4-nitronaphthalene (214A)

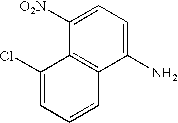

1-Acetamino-5-chloronaphthalene (150 mg, 0.68 mmol, prepared as described in Example 213A) was dissolved in 1 mL AcOH and treated with 82 μL of red fuming HNO₃ and subsequently deacylated with 1 mL 40% aqueous NaOH solution in 3 mL EtOH according to the procedure described in Example 209A to yield 49 mg (32%) of compound 214A as a yellow solid.

B. (3aα,4β,7β,7aα)-2-(5-Chloro-4-nitro-1-naphthalenyl)hexahydro-4,7-dimethyl-4,7-epoxy-1H-isoindole-1,3(2H)-dione (214B)

Compound 214A (27 mg, 0.12 mmol) was reacted in a sealed tube with compound 20A (26 mg, 0.13 mmol), MgSO₄ (32 mg, 0.27 mmol) and Et₃N (88 μL, 0.63 mmol) in 250 μL toluene according to the procedure described in Example 208C to give, after purification by preparative reverse phase HPLC (YMC S5 ODS 20×100 mm eluting with 30-100% aqueous methanol over 10 min containing 0.1% TFA, 20 mL/min) 22 mg (45%) of compound 214B as a yellow solid. HPLC: 24% at 3.06 min & 76% at 3.25 min (atropisomers, retention time) (YMC S5 ODS column 4.6×50 mm eluting with 10-90% aqueous methanol over 4 minutes containing 0.2% phosphoric acid, 4 mL/min, monitoring at 220 nm), MS (ES): m/z 418.0 [M+NH₄]⁺.

### EXAMPLE 215

### (3aα,4β,7β,7aα)-4-Ethylhexahydro-7-methyl-2-(4-nitro-1-naphthalenyl)-4,7epoxy-1H-isoindole-1,3(2H)-dione (215B)

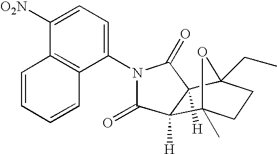

A. (3aα,4β,7β,7aα)-4-Ethylhexahydro-7-methyl-4,7-epoxyisobenzofuran-1,3-dione (215A)

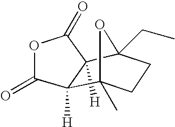

2-Ethyl-5-methylfuran (1.89 mL, 15.3 mmol) was dissolved in methylene chloride (10 mL) and maleic anhydride (1.00 g, 10.2 mmol) was added. The reaction was stirred at 25° C. for 18 h and then concentrated in vacuo. The resulting crude bicycle was dissolved in EtOAc (50 mL) and 10% Pd/C (0.40 g) was added. Hydrogen was then introduced via a balloon. After 4 h, the reaction was filtered through celite, rinsing with EtOAc. Concentration in vacuo gave the crude compound 215A (1.93 g) as a white solid. This material was taken on directly to the next reaction without purification.

B. (3aα,4β,7β,7aα)-4-Ethylhexahydro-7-methyl-2-(4-nitro-1-naphthalenyl)-4,7-epoxy-1H-isoindole-1,3(2H)-dione (215B)

Compound 215A (0.168 g, 0.798 mmol) and 4-nitro-1-naphthalamine (0.10 g, 0.53 mmol) were suspended in toluene (0.8 mL) and TEA (0.2 mL) and magnesim sulfate (0.1 g) were added. The mixture was heated at 135° C. in a sealed tube for 18 h. The reaction was then cooled to rt and filtered, rinsing with chloroform. Concentration gave the crude product which was purified by preparative TLC on SiO₂ eluting with methylene chloride. This gave compound 215B (0.077 g) as a yellow solid. HPLC: 100% at 3.260 min (retention time) (YMC S5 ODS column 4.6×50 mm eluting with 10-90% aqueous methanol over 4 minutes containing 0.2% phosphoric acid, 4 mL/min, monitoring at 220 nm), MS (ES): m/z 381.05 [M+H]⁺.

### EXAMPLE 216

### (3aα,4β,7β,7aα)-2-(4-Cyano-1-naphthalenyl)-N-(4-fluorophenyl)octahydro-7-methyl-1,3-dioxo-4,7-epoxy-4H-isoindole-4-acetamide (216B)

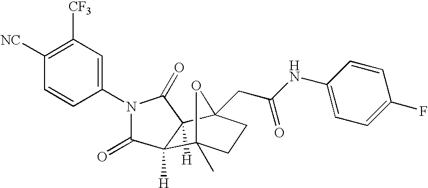

A. N-(4-Fluorophenyl)-5-methyl-2-furanacetamide (216A)

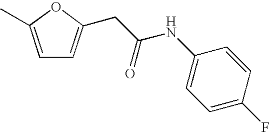

5-Methyl-2-furanacetic acid (1.00 g, 7.14 mmol, synthesized as described WO 9507893, Example 19) was dissolved in CH₃CN/DMF (4:1, 25 mL), 1-[3-(dimethylamino)propyl]-3-ethylcarbodiimide (1.37 g, 7.14 mmol) and 1-hydroxy-7-azabenzotriazole (0.972 g, 7.14 mmol) were then added followed by 4-fluoroaniline (0.676 mL, 7.14 mmol). After 3 h, the reaction was diluted with EtOAc (150 mL) and washed with 1 N HCl (1×30 mL), sat. aq NaHCO₃ (1×30 mL), brine (1×40 mL) and dried over sodium sulfate. Compound 216A (1.581 g) was isolated as a yellow foam after concentration in vacuo. No further purification was necessary. HPLC: 78% at 2.647 min (retention time) (YMC S5 ODS column 4.6×50 mm eluting with 10-90% aqueous methanol over 4 minutes containing 0.2% phosphoric acid, 4 mL/min, monitoring at 220 nm).

B. 3aα,4β,7β,7aα)-2-(4-Cyano-1-naphthalenyl)-N-(4-fluorophenyl)octahydro-7-methyl-1,3-dioxo-4,7-epoxy-4H-isoindole-4-acetamide (216B)

Compound 216A (0.200 g, 0.858 mmol) and 4-(2,5-dihydro-2,5-dioxo-1H-pyrrol-1-yl)-2-trifluoromethylbenzonitrile (0.164 g, 0.66 mmol) were dissolved in benzene and heated at 60° C. for 14 h. The reaction was then cooled and concentrated in vacuo. The resulting orange oil was dissolved in EtOAc (15 mL) and 10% Pd/C (0.050 g) was added. Hydrogen was then introduced via a balloon. After 3 h, the reaction was filtered through celite rinsing with EtOAc and concentrated in vacuo. The resulting crude material was purified by preparative TLC on silica eluting with 5% acetone in methylene chloride to give 0.166 g of compound 216B as a white solid. NMR spectroscopy showed only a single isomer which was determined to be exo by NOE experiments. HPLC: 95% at 3.200 min (retention time) (YMC S5 ODS column 4.6×50 mm eluting with 10-90% aqueous methanol over 4 minutes containing 0.2% phosphoric acid, 4 mL/min, monitoring at 220 nm), MS (ES): m/z 484.0 [M+H]⁺.

### EXAMPLE 217

### (3aα,4β,7β,7aα)-Hexahydro-4-methyl-2-(2-naphthalenyl)-4,7-epoxy-1H-isoindole-1,3(2H)-dione, faster eluting enantiomer & (3aα,4β,7β,7aα)-Hexahydro-4-methyl-2-(2-naphthalenyl)-4,7-epoxy-1H-isoindole-1,3(2H)-dione, slower eluting enantiomer (217i & 217ii, respectively)

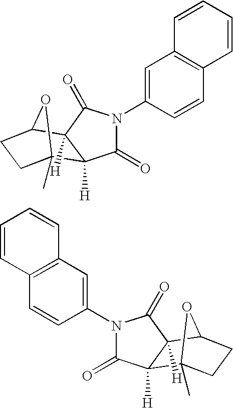

The racemic compound 137 was separated into the individual antipodes by chiral reverse phase liquid chromatography. A Chiralpak AD-R column (4.6×250 mm) was used eluting with 70% acetonitrile/30% water at 1 mL/min. UV detection at 220 nm was used. The faster eluting isomer, compound 217i (retention time=15.66 min), was found to be 99.9% ee and the slower eluting isomer, compound 217ii (retention time=15.66 min) was 99.6% ee by analytical chiral reverse phase chromatography.

### EXAMPLE 218

### (3aα4β,7β,7aα)-4-[4-[2-[[(4-Fluorophenyl)methyl]methylamino]ethyl]octahydro-7-methyl-1,3-dioxo-4,7-epoxy-2H-isoindol-2-yl]-2-(trifluoromethyl)benzonitrile (218B)

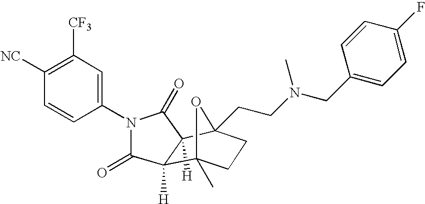

A. (4-Fluorobenzyl)methylamine & Bis(4-fluorobenzyl)methylamine (218A & 218A′)

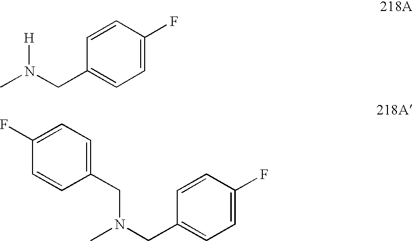

Compounds 218A & 218A′ were made in accordance with the procedure described in Singer, et al., J. Med. Chem. 29; 40-44 (1986). 4-Fluorobenzyl bromide (189 mg, 1.00 mmol) was refluxed in a solution of ethanol (1.5 mL) and methylamine (5 mL, 2 M solution in MeOH) for 3 h. An additional portion of methylamine (2 mL) was added and the mixture was refluxed for an additional hour. The solution was cooled and concentrated in vacuo, and the residue was dissolved in a mixture of 2N HCl (3 mL) and ether (1.5 mL). The layers were separated and the aqueous layer was extracted with an additional portion of ether. The aqueous solution was chilled to 0° C., titrated to pH 11 with NaOH and extracted with CH₂Cl₂. The extracts were dried over MgSO₄ and concentrated to give 120 mg of a 2.5:1 mixture of compounds 218A and compound 218A′ respectively. The crude mixture was taken on without further purification.

B. (3aα,4β,7β,7aα)-4-[4-[2-[[(4-Fluorophenyl)methyl]-methylamino]ethyl]octahydro-7-methyl-1,3-dioxo-4,7-epoxy-2H-isoindol-2-yl]-2-(trifluoromethyl)benzonitrile (218B)

A solution of compound 36 (34.3 mg, 0.075 mmol) and compounds 218A & 218A′ (21 mg, ˜0.088 mmol (of 218A)) in toluene (0.4 mL) was heated at 100° C. overnight. The reaction mixture was cooled to room temperature and then concentrated under reduced pressure. Purification by flash chromatography on silica gel eluting with 25% acetone/75% CH₂Cl₂ gave 30 mg (0.058 mmol, 77.7%) of 218B as a yellow solid. HPLC: 99% at 2.46 min (retention time) (YMC S5 ODS 4.6×50 mm, 10-90% aqueous methanol over 4 minutes containing 0.2% phosphoric acid, monitoring at 220 nm), MS (ES): m/z 516.26 [M+H]⁺.

### EXAMPLE 219

### (3aα,4β,5β,6β,7β,7aα)-4-(Octahydro-4,5,6,7-tetramethyl-1,3-dioxo-4,7-epoxy-2H-isoindol-2-yl)-2-(trifluoromethyl)benzonitrile (219D)

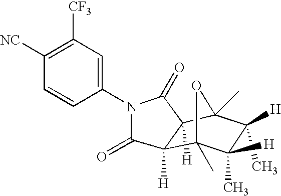

A. 2,3,4,5-Tetramethylfuran (219A)

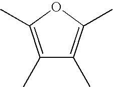

Compound 219A was made in accordance with the procedures described in Hancock et. al., J. Org. Chem. 42, 1850-1856 (1977) & Amarnath, et. al., J. Org. Chem., 60, 301-307 (1995). 2-Propanone (100 mL, 1.1 mol) was refluxed over PbO₂ (26.7 g, 0.11 mol) for 28 h. After cooling to rt, the reaction mixture was filtered and the residue was washed with acetone. The filtrate was concentrated under reduced pressure to remove the acetone and then distilled at 20 Torr. The fraction that came over between 100-120° C. was collected to give 6.75 g (42.5%) of 3,4-dimethylhexane-2,5-dione as a light yellow oil.

A solution of 3,4-dimethylhexane-2,5-dione (3.00 g, 21.1 mmol) and p-toluenesulfonic acid (401 mg, 2.11 mmol) in benzene (30 mL) was heated to reflux in a Dean-Stark trap overnight. The reaction mixture was distilled at atmospheric pressure to remove the excess benzene. The remaining mixture was transferred to a smaller flask and distilled at atmospheric pressure. The fraction that came over between 80-100° C. was collected to give 509 mg (19%) of compound 219A as a light yellow oil.

B. (3aα,4β,7β,7aα)-4-Ethyl-3a,4,7,7a-tetrahydro-4,5,6,7-tetramethyl-4,7-epoxyisobenzofuran-1,3-dione (219B)

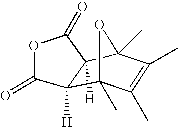

A solution of compound 219A (400 mg, 3.22 mmol) and maleic anhydride (442 mg, 4.51 mmol) in Et₂O (1.5 mL) was stirred at rt overnight. The reaction mixture was then placed in freezer for 5 days, after which time the resulting crystals were collected and dried to give 0.26 g (37%) of compound 219B as tan crystals. The crude compound 219B was taken on to the next step with out further purification.

C. (3aα,4β,5α,6α,7β,7aα)-4-Ethylhexahydro-4,5,6,7-tetramethyl-4,7-epoxyisobenzofuran-1,3-dione (219C)

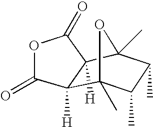

A solution of compound 219B (120 mg, 0.545 mmol) and 10% Pd/C (24 mg, cat.) in EtOAc (2 mL) was stirred under a balloon of hydrogen at room temperature overnight. The reaction mixture was filtered through celite and concentrated under reduced pressure to give 100 mg (0.446 mmol, 81.9%) of compound 219C as a white solid, which was carried on with no further purification.

D. (3aα,4β,5β,6β,7β,7aα)4-(Octahydro-4,5,6,7-tetramethyl-1,3-dioxo-4,7-epoxy-2H-isoindol-2-yl)-2-(trifluoromethyl)benzonitrile (219D)

A solution of compound 219C (44.4 mg, 0.2 mmol), 5-amino-2-cyanobenzotrifluoride (45 mg, 0.24 mmol), TEA (0.04 mL) and MgSO₄ (20 mg) in toluene (0.2 mL) was heated at 135° C. overnight. The reaction mixture was cooled to room temperature, filtered and then concentrated under reduced pressure. Purification by flash chromatography on silica gel eluting with 40% EtOAc/hexanes followed by washing the resulting solid with MeOH gave 17 mg (0.043 mmol, 21.7%) of compound 219D as a white solid. HPLC: 90% at 3.11 min (retention time) (YMC S5 ODS 4.6×50 mm, 10-90% aqueous methanol over 4 minutes containing 0.2% phosphoric acid, monitoring at 220 nm), MS (ES): m/z 391.2 [M−H]⁻.

### EXAMPLE 220

### (3aα,4β,7β,7aα)-4-[Octahydro-4-methyl-1,3-dioxo-7-[2-[4-(trifluoromethyl)phenoxy]ethyl]-4,7-epoxy-2H-isoindol-2-yl]-2-(trifluoromethyl)benzonitrile, faster eluting antipode & (3aα,4β,7β,7aα)-4-[Octahydro-4-methyl-1,3-dioxo-7-[2-[4-(trifluoromethyl)phenoxy]ethyl]-4,7-epoxy-2H-isoindol-2-yl]-2-(trifluoromethyl)benzonitrile, slower eluting enantiomer (220i & 220ii, respectively)

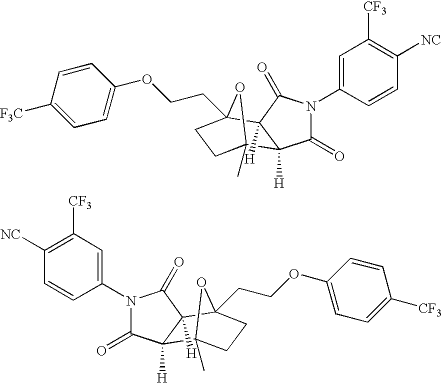

The racemic compound 35 was separated into the individual antipodes by chiral normal phase liquid chromatography. A Chiralpak AD column (50×500 mm) was used eluting with 85% hexanes/7.5% methanol/7.5% ethanol, @50 mL/min. UV detection at 220 nm was used. The faster eluting isomer compound 220i (retention time=55.86 min) was found to have 95.8% ee ([α]_{D}²⁵=−53.02°, C=3.134 mg/cc in CH₂Cl₂) and the slower eluting isomer compound 220ii (retention time=62.86 min) was 86% ee ([α]_{D}²⁵=+48.740, C=2.242 mg/cc in CH₂Cl₂) by analytical chiral normal phase chromatography.

### EXAMPLE 221

### (3aα,4β,5β,7β,7aα)-4-(Octahydro-5-hydroxy-4,7-dimethyl-1,3-dioxo-4,7-epoxy-2H-isoindol-2-yl)-2-(trifluoromethyl)benzonitrile (221B)

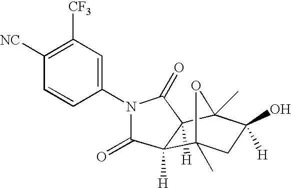

A. (3aα,4β,7β,7aα)-4-(hexahydro-4,7-dimethyl-1,3-dioxo-4,7-epoxy-2H-isoindol-2-yl)-2-(trifluoromethyl)benzonitrile (221Ai) & (3aα,4α,7α,7aα)-4-(hexahydro-4,7-dimethyl-1,3-dioxo-4,7-epoxy-2H-isoindol-2-yl)-2-(trifluoromethyl)benzonitrile (221Aii)

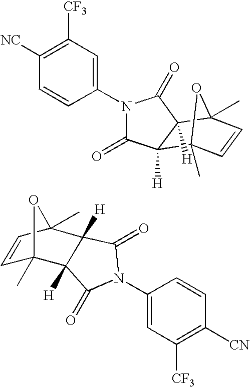

A solution of 2,5-dimethylfuran (0.8 mL, 7.51 mmol) and 4-(2,5-dihydro-2,5-dioxo-1H-pyrrol-1-yl)-2-trifluoromethylbenzonitrile (synthesized as described in Example 1B) (1.00 g, 3.75 mmol) in benzene (4 mL) was heated at 60° C. overnight. The reaction mixture was concentrated under reduced pressure and placed on a high vacuum pump until the oil solidified to give a 3:1 mixture (determined by LC and NMR) of compounds 221Ai & 221Aii, respectively, as a brown solid, which was used directly in the next step with out further purification.

B. (3aα,4β,5β,7β,7aα)-4-(Octahydro-5-hydroxy-4,7-dimethyl-1,3-dioxo-4,7-epoxy-2H-isoindol-2-yl)-2-(trifluoromethyl)benzonitrile (221B)

BH₃.THF (3.75 mL, 3.75 mmol, 1M in THF) was added to a solution of crude compounds 221Ai & 221Aii (3.75 mmol) in THF (12.5 mL) at 0° C. After the starting material was consumed the reaction mixture was concentrated under reduced pressure. The resulting residue was then dissolved in toluene (12.5 mL), Me₃NO (845 mg, 11.25 mmol) was added and the mixture was heated to reflux overnight. The reaction mixture was then cooled to rt, added to H₂O and extracted with EtOAc (3×). The combined organic layers were dried over MgSO₄ and concentrated under reduced pressure. Purification by flash chromatography on SiO₂ eluting with 75% EtOAc/hexanes gave 0.354 g (25%) of compound 221B as a tan powder. HPLC: 90% at 2.45 min (retention time) (YMC S5 ODS column 4.6×50 mm eluting with 10-90% aqueous methanol over 4 minutes containing 0.2% phosphoric acid, 4 mL/min, monitoring at 220 nm), MS (ES): m/z 381.11 [M+H]⁺.

### EXAMPLE 222

### (3aα,4β,5α,7β,7aα)-4-(Octahydro-5-hydroxy-4,7-dimethyl-1,3-dioxo-4,7-epoxy-2H-isoindol-2-yl)-2-(trifluoromethyl)benzonitrile (222D)

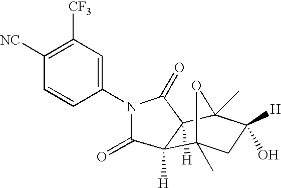

A. 3-[[(1,1-Dimethylethyl)dimethylsilyl]oxy]-2,5-dimethylfuran (222A)

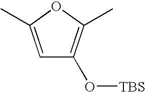

2,5-Dimethyl-3(3H)-furanone (2.00 g, 17.8 mmol) was dissolved in methylene chloride (180 mL). TEA (7.43 mL, 53.5 mmol) was added followed by TBSOTf (4.92 mL, 21.4 mmol) at 25° C. After 1 h, the reaction was concentrated in vacuo and the resulting slurry was run through a silica gel column conditioned with 3% TEA in hexanes. The product was eluted with 3% TEA/hexanes to give 3.6 g of compound 222A as an orange oil which was used directly in subsequent reactions.

B. (3aα,4β,7β,7aα)-4-[5-[[(1,1-Dimethylethyl)dimethylsilyl]oxy]-1,3,3a,4,7,7a-hexahydro-4,7-dimethyl-1,3-dioxo-4,7-epoxy-2H-isoindol-2-yl]-2-(trifluoromethyl)benzonitrile (222B)

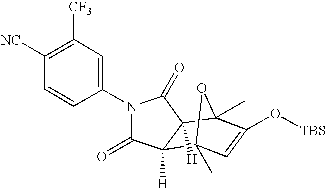

4-(2,5-Dihydro-2,5-dioxo-1H-pyrrol-1-yl)-2-trifluoromethylbenzonitrile (1.00 g, 3.85 mmol) was dissolved in benzene (5.0 mL) and the compound 222A (1.30 g, 5.77 mmol) was added. The reaction mixture was warmed to 60° C. for 2 h and then cooled to 25° C. The solution was then concentrated in vacuo to give compound 222B as a yellow oil which was carried on to the next reaction without purification. HPLC: 60% at 4.013 min (retention time) (YMC S5 ODS column 4.6×50 mm eluting with 10-90% aqueous methanol over 4 minutes containing 0.2% phosphoric acid, 4 mL/min, monitoring at 220 nm).

C. (3aα,4β,5α,7β,7aα)-4-[5-[[(1,1-Dimethylethyl)dimethylsilyl]oxy]octahydro-4,7-dimethyl-1,3-dioxo-4,7-epoxy-2H-isoindol-2-yl]-2-(trifluoromethyl)benzonitrile (222C)

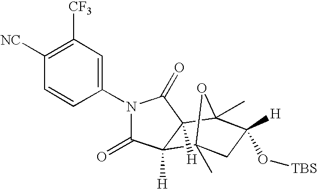

Crude compound 222B (3.85 mmol) was dissolved in ethyl acetate (75 mL) and 10% Pd/C (1.20 g) was added. Hydrogen was then introduced via a balloon. After 24 h, the reaction was filtered through celite rinsing with ethyl acetate and concentrated in vacuo to give a yellow oil. The crude product was purified by flash chromatography on silica gel eluting with methylene chloride/acetone (0%-1%-2% acetone) to give compound 222C as a yellow solid (0.710 g). HPLC: 100% at 4.160 min (retention time) (YMC S5 ODS column 4.6×50 mm eluting with 10-90% aqueous methanol over 4 minutes containing 0.2% phosphoric acid, 4 mL/min, monitoring at 220 nm), MS (ES): m/z 517.6 [M+Na]⁺.

D. (3aα,4β,5α,7β,7aα)-4-(Octahydro-5-hydroxy-4,7-dimethyl-1,3-dioxo-4,7-epoxy-2H-isoindol-2-yl)-2-(trifluoromethyl)benzonitrile (222D)

Compound 222C (0.040 g, 0.081 mmol) was dissolved in THF (1.0 mL) and HF•Pyridine (0.5 mL) was added. After 2 h, the reaction was carefully poured into cold saturated aq NaHCO₃. The mixture was then extracted with methylene chloride (3×10 mL). The combined organics were washed with 1 N HCl (1×10 mL) and dried over anhydrous sodium sulfate. Concentration in vacuo gave compound 222D as a yellow solid (0.031 g). NOE experiments confirmed the assigned isomer. HPLC: 98% at 2.777 min (retention time) (YMC S5 ODS column 4.6×50 mm eluting with 10-90% aqueous methanol over 4 minutes containing 0.2% phosphoric acid, 4 mL/min, monitoring at 220 nm), MS (ES): m/z 403.06 [M+Na]⁺.

### EXAMPLE 223

### (αR)-α-Methoxybenzeneacetic acid, 2-[(3aα,4β,7β,7aα)-2-(4-cyano-1-naphthalenyl)octahydro-7-methyl-1,3-dioxo-4,7-epoxy-4H-isoindol-4-y]ethyl ester (223C)

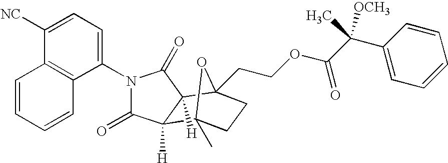

A. (3aα,4β,7β,7aα)-4-[4-[2-[[(1,1-Dimethylethyl)dimethylsilyl]oxy]ethyl]octahydro-7-methyl-1,3-dioxo-4,7-epoxy-2H-isoindol-2-yl]-1-naphthalenecarbonitrile (223A)

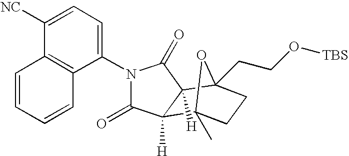

A solution of 4-amino-1-naphthalenecarbonitrile (19.2 g, 114 mmol) and maleic anhydride (14.0 g, 113 mmol) in AcOH (230 mL) was heated at 115° C. for 12 h. After cooling to rt, the reaction mixture was concentrated under reduced pressure then diluted with CH₂Cl₂ (2.5 L). The organic layer was washed 3× with H₂O (3 L), 1× with sat. aq Na₂CO₃ (1 L) and 1× with brine (1 L), dried over MgSO₄ and concentrated to ˜200 mL under reduced pressure. Purification by flash chromatography on cation exchange resin (60 g, CUBX13M6 from United Chemical Technologies) eluting with CH₂Cl₂ gave 25.0 g (88%) of 4-(2,5-Dihydro-2,5-dioxo-1H-1-yl)-1-naphthalenecarbonitrile as a yellow solid. HPLC 96% at 2.48 min (Phenomenex-prime S5-C18 column 4.6×50 mm, 10-90% aqueous methanol over 4 minutes containing 0.2% phosphoric acid, 4 mL/min, monitoring at 220 nm), MS (ES): m/z 249.25 [M+H]⁺.

4-(2,5-Dihydro-2,5-dioxo-1H-1-yl)-1-naphthalenecarbonitrile (1.00 g, 4.03 mmol) was suspended in benzene (6.0 mL) in a sealed tube and compound 204A (1.11 g, 5.24 mmol) was added. The reaction was heated at 60° C. for 16 h and then cooled to 25° C. The benzene was removed in vacuo to give a yellow solid. The solid was dissolved in ethyl acetate (40 mL) and Pd/C (10% Pd, 0.300 g) was added. Hydrogen was then introduced via a balloon. After 4 h, the reaction was filtered through celite rinsing with ethyl acetate. Concentration in vacuo gave a pale yellow solid which was purified by flash chromatography on silica gel eluting with acetone/chloroform (0%-1.5%-3% acetone) to give compound 223A (1.53 g) as a yellow foam. HPLC: 86% at 4.173 min (retention time) (YMC S5 ODS column 4.6×50 mm eluting with 10-90% aqueous methanol over 4 minutes containing 0.2% phosphoric acid, 4 mL/min, monitoring at 220 nm).

B. (3aα,4β,7β,7aα)-4-[Octahydro-4-(2-hydroxyethyl)-7-methyl-1,3-dioxo-4,7-epoxy-2H-isoindol-2-yl]-1-naphthalenecarbonitrile (223B)

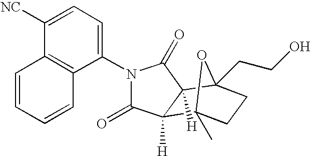

Compound 223A (1.37 g, 2.97 mmol) was dissolved in THF (8.0 mL) and transferred to a polypropylene bottle and cooled to 0° C. HF•Pyridine (2.0 mL) was then added. After 20 min, the reaction was carefully poured into cold sat. aq sodium bicarbonate and extracted with methylene chloride (3×30 mL). The organics were then washed with 1 N HCl and dried over anhydrous sodium sulfate. Concentration in vacuo gave the compound 223B (0.99 g) as a yellow foam which was not purified further. HPLC: 96% at 2.443 and 2.597 (atropisomers) min (retention time) (YMC S5 ODS column 4.6×50 mm eluting with 10-90% aqueous methanol over 4 minutes containing 0.2% phosphoric acid, 4 mL/min, monitoring at 220 nm), MS (ES): m/z 399.02 [M+Na]⁺.

C. (αR)-α-Methoxybenzeneacetic acid, 2-[(3aα,4β,7β,7aα)-2-(4-cyano-1-naphthalenyl)octahydro-7-methyl-1,3-dioxo-4,7-epoxy-4H-isoindol-4-y]ethyl ester (223C)

Compound 223B (0.200 g, 0.575 mmol) was added to a solution of WSDCC (0.138 g, 0.719 mmol) and (R)-mandelic acid (0.096 g, 0.575 mmol) in dichloromethane (6.0 mL). 4-DMAP (0.005 g) was then added and the reaction stirred at 25° C. for 4 h. The mixture was then diluted with dichloromethane and washed with 1 N HCl (2×10 mL), once with sodium bicarbonate (10 mL) and dried over anhydrous sodium sulfate. Concentration in vacuo gave compound 223C (0.220 g) as a yellow solid which was not purified further. HPLC: 100% at 3.283 min (retention time) (YMC S5 ODS column 4.6×50 mm eluting with 10-90% aqueous methanol over 4 minutes containing 0.2% phosphoric acid, 4 mL/min, monitoring at 220 nm), MS (ES): m/z 547.26 [M+Na]⁺.

### EXAMPLE 224

### (3aα,4β,7β,7aα)-2-(Methylthio)-4-(octahydro-4,7-dimethyl-1,3-dioxo-4,7-epoxy-2H-isoindol-2-yl)benzonitrile (224)

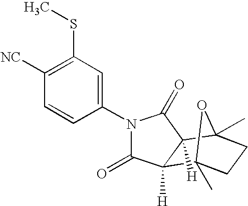

4-Amino-2-(methylthio)benzonitrile (100 mg, 0.61 mmol, synthesized as described in EP 40931 A1) was reacted in a sealed tube with compound 20A (131 mg, 0.67 mmol), MgSO₄ (161 mg, 1.34 mmol) and Et₃N (0.44 mL, 3.17 mmol) in 0.50 mL toluene according to the procedure described in Example 208C to give, after purification by preparative reverse phase HPLC (YMC S5 ODS 20×100 mm eluting with 30-100% aqueous methanol over 10 min containing 0.1% TFA, 20 mL/min), 137 mg (0.40 mmol, 66%) of compound 224 as a white solid. HPLC: 100% at 2.73 min (retention time) (YMC S5 ODS column 4.6×50 mm eluting with 10-90% aqueous methanol over 4 minutes containing 0.2% phosphoric acid, 4 mL/min, monitoring at 220 nm), MS (ES): m/z 401.0 [M−H+OAc]⁻.

### EXAMPLE 225

### (3aα,4β,7β,7aα)-2-(Methylsulfinyl)-4-(octahydro-4,7-dimethyl-1,3-dioxo-4,7-epoxy-2H-isoindol-2-yl)benzonitrile (225)

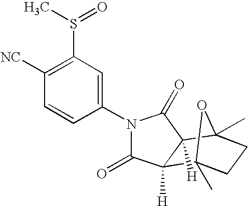

To an ice-cold suspension of compound 224 (30 mg, 0.09 mmol) in 2 mL of H₂O/MeOH (1:1) was added oxone (80 mg, 0.26 mmol) in one solid portion. The resulting mixture was stirred for 4 h at 0° C. before it was diluted with H₂O (10 mL) and extracted with CH₂Cl₂ (2×20 mL). The combined organic layers were dried and concentrated to leave a residue which was purified by filtering the material through a short pad of silica gel eluting with CH₂Cl₂ to yield 32 mg (0.09 mmol, 100%) of compound 225 as a colorless oil. HPLC: 99% at 2.01 min (retention time) (YMC S5 ODS column 4.6×50 mm eluting with 10-90% aqueous methanol over 4 minutes containing 0.2% phosphoric acid, 4 mL/min, monitoring at 220 nm), MS (ES): m/z 376.0 [M+NH₄]⁺.

### EXAMPLE 226

### (3aα,4β,7β,7aα)-2-(Methylsulfonyl)-4-(octahydro-4,7-dimethyl-1,3-dioxo-4,7-epoxy-2H-isoindol-2-yl)benzonitrile (226)

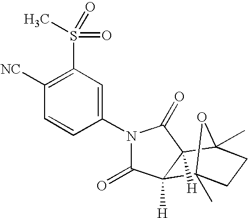

To a solution of compound 225 (48 mg, 0.14 mmol) in CH₂Cl₂ (2 mL) was added mCPBA (145 mg, 50% mixture, 0.42 mmol) in one solid portion. The resulting mixture was allowed to warm to room temperature and was stirred for 60 h at which time no more starting material could be detected by HPLC. The reaction was quenched by the addition of sat. NaHCO₃ solution (5 mL), the layers were separated and the aqueous layer was extracted with CH₂Cl₂ (20 mL). The combined organic phases were dried over MgSO₄ and concentrated. The remaining residue was purified by preparative reverse phase HPLC (YMC S5 ODS 20×100 mm eluting with 30-100% aqueous methanol over 10 min containing 0.1% TFA, 20 mL/min) to afford 48 mg (0.13 mmol, 92%) of compound 226 as a white solid. HPLC: 100% at 2.07 min (retention time) (YMC S5 ODS column 4.6×50 mm eluting with 10-90% aqueous methanol over 4 minutes containing 0.2% phosphoric acid, 4 mL/min, monitoring at 220 nm), MS (ES): m/z 392.0 [M+NH₄]⁺.

### EXAMPLE 227

### (3aα,4β,5β,7β,7aα)-7-[2-[[(1,1-Dimethylethyl)dimethylsilyl]oxy]ethyl]hexahydro-5-hydroxy-4-methyl-2-(4-nitro-1-naphthalenyl)-4,7-epoxy-1H-isoindole-1,3(2H)-dione (227B)

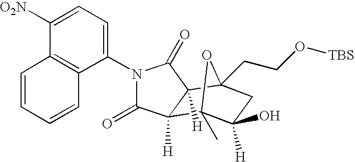

A. (3aα,4β,7β,7aα)-4-[2-[[(1,1-Dimethylethyl)dimethylsilyl]oxy]ethyl]-3a,4,7,7a-tetrahydro-7-methyl-2-(4-nitro-1-naphthalenyl)-4,7-epoxy-1H-isoindole-1,3(2H)-dione (227A)

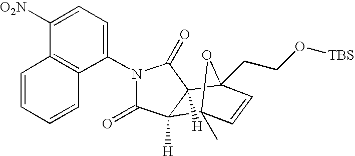

A solution of compound 204A (455 mg, 1.894 mmol, 2 eq) and 1-[4-nitronaphthalene]-1H-pyrrole-2,5-dione (254 mg, 0.947 mmol, 1 eq) (prepared as described for 4-(2,5-dihydro-2,5-dioxo-1H-1-yl)-1-naphthalenecarbonitrile, Example 223A) in benzene (2 mL) was heated at 60° C. overnight. The reaction mixture was concentrated under reduced pressure to give crude compound 227A as a brown solid, which was used directly in the next step with out further purification.

B. (3aα,4β,5β,7β,7aα)-7-[2-[[(1,1-Dimethylethyl)dimethylsilyl]oxy]ethyl]hexahydro-5-hydroxy-4-methyl-2-(4-nitro-1-naphthalenyl)-4,7-epoxy-1H-isoindole-1,3(2H)-dione (227B)

BH₃.THF (0.95 mL, 0.95 mmol, 1M in THF, 1 eq) was added to a solution of crude compound 227A (0.95 mmol, 1 eq) in THF (2 mL) at 0° C. After compound 227A was consumed, as was evident by HPLC, the reaction mixture was concentrated under reduced pressure. The resulting residue was then dissolved in toluene (2 mL), Me₃NO (71 mg, 2.84 mmol, 3 eq) was added and the mixture was heated to reflux over night. The reaction mixture was then cooled to rt, added to H₂O and extracted with EtOAc (3×). The combined organic layers were dried over MgSO₄ and concentrated under reduced pressure. Purification by flash chromatography on SiO₂ eluting with a mixture of 75% EtOAc/Hexanes, gave 130.2 mg (26%) of compound 227B as a brown solid. HPLC: 94% at 3.92 min (retention time) (YMC S5 ODS column 4.6×50 mm eluting with 10-90% aqueous methanol over 4 minutes containing 0.2% phosphoric acid, 4 mL/min, monitoring at 220 nm), MS (ES): m/z 527.5 [M+H]⁺.

### EXAMPLE 228

### (3aα,4β,5β,7β,7aα)-Hexahydro-5-hydroxy-7-(2-hydroxyethyl)-4-methyl-2-(4-nitro-1-naphthalenyl)-4,7-epoxy-1H-isoindole-1,3(2H)-dione (228)

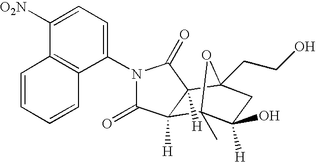

A mixture of TBAF (0.3 mL, 0.296 mmol, 1 M solution in THF) and HF (0.3 mL, 50% in H₂O) in CH₃CN (6 mL) was added to a solution of 227B (104 mg, 0.197 mmol) in THF (2 mL) at 0° C. The reaction mixture was stirred overnight at rt. After the starting material was consumed, as was evident by TLC, H₂O and EtOAc were added and the layers were separated. The aqueous layer was extracted with EtOAc (1×) and the combined organic layers were washed with H₂O (1×) and brine (1×), dried over Na₂SO₄ and concentrated under reduced pressure. Purification by flash chromatography on SiO₂ eluting with 5% MeOH/CH₂Cl₂ gave 61.2 mg (75%) of compound 228 as a yellow solid. HPLC: 99% at 2.47 min (retention time) (YMC S5 ODS column 4.6×50 mm eluting with 10-90% aqueous methanol over 4 minutes containing 0.2% phosphoric acid, 4 mL/min, monitoring at 220 nm), MS (ES): m/z 411.2 [M−H]⁻.

### EXAMPLE 229

### (3aα,4β,5β,7β,7aα)-7-[2-(4-Fluorophenoxy)ethyl]hexahydro-5-hydroxy-4-methyl-2-(4-nitro-1-naphthalenyl)-4,7-epoxy-1H-isoindole-1,3(2H)-dione (229)

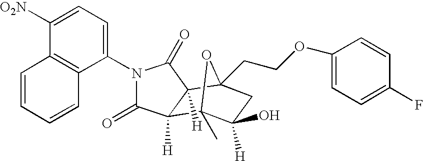

DBAD (37.7 mg, 0.164 mmol) was added to a solution of PPh₃ (43 mg, 0.164 mmol) in THF (1 mL). After stirring for 10 mins, 4-fluorophenol (18.3 mg, 0.164 mmol) was added and the reaction mixture was stirred for a further min. A solution of compound 228 (45 mg, 0.109 mmol) in THF (1 mL) was added and the mixture was stirred at rt overnight. HPLC showed the crude reaction mixture to contain mostly starting diol (compound 228), so this mixture was added to a preformed mixture as before of PPh₃ (86 mg), DBAD (75.4 mg) and phenol (36.6 mg) in THF (4 mL) at rt. Stirring was continued until all of compound 228 was consumed. The reaction was then concentrated under reduced pressure. Purification by preparative chromatography [HPLC at 15.2 min (retention time) (YMC S5 ODS A column 20×100 mm, 10-90% aqueous methanol over 15 minutes containing 0.1% TFA, 20 mL/min, monitoring at 220 nm)] gave 25.0 mg (45%) of compound 229 as a light yellow solid. HPLC: 99% at 3.53 min (retention time) (YMC S5 ODS column 4.6×50 mm eluting with 10-90% aqueous methanol over 4 minutes containing 0.2% phosphoric acid, 4 mL/min, monitoring at 220 nm), MS (ES): m/z 505.2 [M−H]⁻.

### EXAMPLE 230

### (3aα,4β,5β,6β,7β,7aα)-4-(Octahydro-5,6-dihydroxy-4,7-dimethyl-1,3-dioxo-4,7-epoxy-2H-isoindol-2-yl)-2-(trifluoromethyl)benzonitrile & (3aα,4β,5α,6α,7β,7aα)-4-(Octahydro-5,6-dihydroxy-4,7-dimethyl-1,3-dioxo-4,7-epoxy-2H-isoindol-2-yl)-2-(trifluoromethyl)benzonitrile (230Bi & 230Bii, Respectively)

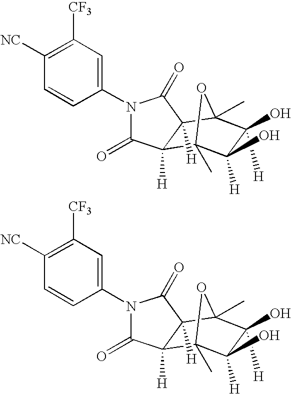

A. (3aα,4β,7β,7aα)-4-(1,3,3a,4,7,7a-Hexahydro-4,7-dimethyl-1,3-dioxo-4,7-epoxy-2H-isoindol-2-yl)-2-(trifluoromethyl)benzonitrile (230A)

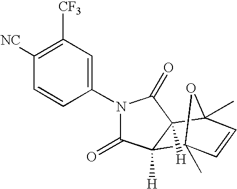

3,5-dimethyl furan (1.23 mL, 11.54 mmol) and 4-(2,5-Dihydro-2,5-dioxo-1H-pyrrol-1-yl)-2-trifluoromethylbenzonitrile (2.00 g, 7.69 mmol) were dissolved in benzene (10 mL) and heated at 60° C. for 18 h. The volatile organics were then removed in vacuo. The resulting crude compound 230A was carried on without purification. HPLC: 71% at 3.007 min (retention time) (YMC S5 ODS column 4.6×50 mm eluting with 10-90% aqueous methanol over 4 minutes containing 0.2% phosphoric acid, 4 mL/min, monitoring at 220 nm).

B. (3aα,4β,5β,6β,7β,7aα)-4-(Octahydro-5,6-dihydroxy-4,7-dimethyl-1,3-dioxo-4,7-epoxy-2H-isoindol-2-yl)-2-(trifluoromethyl)benzonitrile & (3aα,4β,5α,6α,7β,7aα)-4-(Octahydro-5,6-dihydroxy-4,7-dimethyl-1,3-dioxo-4,7-epoxy-2H-isoindol-2-yl)-2-(trifluoromethyl)benzonitrile (230Bi & 230Bii)

Compound 230A (0.100 g, 0.281 mmol) was dissolved in acetone and N-methylmorpholine-N-oxide (50% aq solution, 0.100 mL, 0.42 mmol) was added. OsO₄ (4% aq solution, 0.014 mmol) was then added. After 3 h at 25° C., the reaction was complete and sodium sulfite (0.250 g) was added with vigorous stirring. After 15 min, brine (10 mL) was added and the solution was extracted with EtOAc (3×15 mL). The organics were dried over anhydrous sodium sulfate and then concentrated in vacuo. The crude diol mixture was purified by preparative TLC eluting with 18% acetone in chloroform to give 0.038 g of compound 230Bi (beta face) and 0.012 g of compound 230Bii (alpha face) as pale yellow solids. Compound 230Bi: HPLC: 100% at 2.567 min (retention time) (YMC S5 ODS column 4.6×50 mm eluting with 10-90% aqueous methanol over 4 minutes containing 0.2% phosphoric acid, 4 mL/min, monitoring at 220 nm), MS (ES): m/z 397.08 [M+H]+. Compound 230Bii: HPLC: 100% at 2.417 min (retention time) (YMC S5 ODS column 4.6×50 mm eluting with 10-90% aqueous methanol over 4 minutes containing 0.2% phosphoric acid, 4 mL/min, monitoring at 220 nm), MS (ES): m/z 397.08 [M+H]⁺.

### EXAMPLE 231

### (3aα,4β,5β,6β,7β,7aα)-4-[Octahydro-5,6-dihydroxy-4-(hydroxyethyl)-7-methyl-1,3-dioxo-4,7-epoxy-2H-isoindol-2-yl]-1-naphthalenecarbonitrile, (231C)

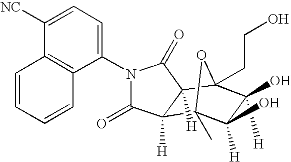

A. (3aα,4β,7β,7aα)-4-[4-[2-[[(1,1-Dimethylethyl)dimethylsilyl]oxy]ethyl]-1,3,3a,4,7,7a-hexahydro-7-methyl-1,3-dioxo-4,7-epoxy-2H-isoindol-2-yl]-1-naphthalenecarbonitrile (231A)

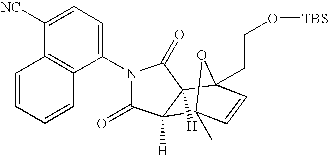

Compound 204A (29.03 g, 120 mmol) and 4-(2,5-dihydro-2,5-dioxo-1H-1-yl)-1-naphthalenecarbonitrile (20.0 g, 80.6 mmol) were suspended in benzene (80 mL) and heated to 60° C. for 14 h. The mixture was then concentrated in vacuo at 40° C. for 40 min. The resulting slurry was cooled to 25° C. and then suspended in MeOH (200 mL) and stirred at rt for 30 min. The solution was then cooled to 0° C. for 30 min and then filtered rinsing with cold It MeOH. The resulting solid was dried in vacuo to give 26.1 g of crude compound 231A as a white solid. The methanol solution was concentrated in vacuo and resuspended in MeOH (50 mL) and cooled to −20° C. for 4 h. The solution was then filtered rinsing with cold MeOH. The resulting solid was dried in vacuo to give 3.8 g of compound 231A as a white solid. HPLC: 95% at 4.227 min (retention time) (YMC S5 ODS column 4.6×50 mm eluting with 10-90% aqueous methanol over 4 minutes containing 0.2% phosphoric acid, 4 mL/min, monitoring at 220 nm)

B. (3aα,4β,5β,6β,7β,7aα)-4-[4-[2-[[(1,1-Dimethylethyl)dimethylsilyl]oxy]ethyl]octahydro-5,6-dihydroxy-7-methyl-1,3-dioxo-4,7-epoxy-2H-isoindol-2-yl]-1-naphthalenecarbonitrile (231B)

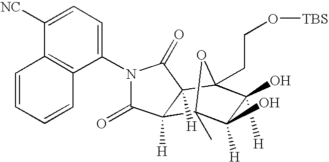

Compound 231A (0.400 g, 0.851 mmol) was dissolved in acetone (9.0 mL) and N-methylmorpholine-N-oxide (50% aq solution, 0.0.150 mL, 1.28 mmol) was added. OsO₄ (4% aq solution, 0.043 mmol) was then added. After 3 h at 25° C., the reaction was complete and sodium sulfite (1.0 g) was added with vigorous stirring. After 15 minutes, brine (30 mL) was added and the solution extracted with EtOAc (3×50 mL). The organics were dried over anhydrous sodium sulfate and then concentrated in vacuo. The crude diol was purified by flash chromatography on silica eluting with 5-25% acetone in chloroform to give 0.355 g of compound 231B as a yellow solid. HPLC: 93% at 3.903 min (retention time) (YMC S5 ODS column 4.6×50 mm eluting with 10-90% aqueous methanol over 4 minutes containing 0.2% phosphoric acid, 4 mL/min, monitoring at 220 nm), MS (ES): m/z 522.00 [M+H]⁺.

C. (3aα,4β,5β,6β,7β,7α)-4-[Octahydro-5,6-dihydroxy-4-(hydroxyethyl)-7-methyl-1,3-dioxo-4,7-epoxy-2H-isoindol-2-yl]-1-naphthalenecarbonitrile (231C)

Compound 231B (0.400 g, 0.766 mmol) was dissolved in THF (5.0 mL) and transferred to a polypropylene bottle and cooled to 0° C. HF·Pyridine (1.0 mL) was then added. After 20 min, the reaction was carefully poured into cold sat. aq sodium bicarbonate and extracted with methylene chloride (3×30 mL). The organics were then washed once with 1 N HCl and dried over anhydrous sodium sulfate. Concentration in vacuo gave the compound 231C (0.290 g) as a yellow foam which was not purified further. HPLC: 92% at 2.273 and 2.423 (atropisomers) min (retention time) (YMC S5 ODS column 4.6×50 mm eluting with 10-90% aqueous methanol over 4 minutes containing 0.2% phosphoric acid, 4 mL/min, monitoring at 220 nm), MS (ES): m/z 409.10 [M+H]⁺.

### EXAMPLE 232

### (3aα,4β,5β,6β7β,7aα)-4-[Octahydro-5,6-dihydroxy-4-methyl-1,3-dioxo-7-[2-[4-(trifluoromethyl)phenoxy]ethyl]-4,7-epoxy-2H-isoindol-2-yl]-2-(trifluoromethyl)benzonitrile, (232C)

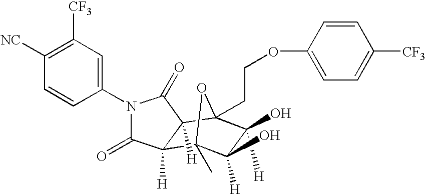

A. 2-Methyl-5-[2-[4-(trifluoromethyl)phenoxy]ethyl]furan (232A)

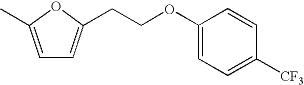

To a solution of triphenylphosphine (1.56 g, 5.95 mmol) in THF (40 mL) was added DBAD (1.37 g, 5.95 mmol). After 10 min, 4-trifluoromethylphenol (0.964 g, 5.95 mmol) was added. After 10 additional minutes, compound 21A (0.500 g, 3.97 mmol) was added. After 14 h at 25° C., the reaction was concentrated in vacuo and purified by flash chromatography on silica eluting with chloroform to give 0.713 g of compound 232A as a clear oil.

B. (3aα,4β,7β,7aα)-4-[1,3,3a,4,7,7a-hexahydro-4-methyl-1,3-dioxo-7-[2-[4-(trifluoromethyl)phenoxy]ethyl]-4,7-epoxy-2H-isoindol-2-yl]-2-(trifluoromethyl)benzonitrile (232B)

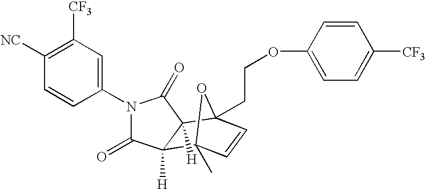

Compound 232A (0.301 g, 1.15 mmol) and 4-(2,5-dihydro-2,5-dioxo-1H-pyrrol-1-yl)-2-trifluoromethylbenzonitrile (0.220 g, 0.846 mmol) were suspended in benzene (1.5 mL) and heated to 60° C. for 14 h. The mixture was then concentrated in vacuo at 40° C. for 40 minutes. The crude product was purified by flash chromatography on silica eluting with 10-0% hexanes in methylene chloride to give 0.199 g of compound 232B as a yellow solid. Compound 232B was characterized as the exo diastereomer by NOE experiments. HPLC: 94% at 3.993 min (retention time) (YMC S5 ODS column 4.6×50 mm eluting with 10-90% aqueous methanol over 4 minutes containing 0.2% phosphoric acid, 4 mL/min, monitoring at 220 nm).

C. (3aα,4β,5β,6β,7β,7aα)-4-[Octahydro-5,6-dihydroxy-4-methyl-1,3-dioxo-7-[2-[4-(trifluoromethyl)phenoxy]ethyl]-4,7-epoxy-2H-isoindol-2-yl]-2-(trifluoromethyl)benzonitrile, (232C)

Compound 232B (0.075 g, 0.140 mmol) was dissolved in acetone (2.0 mL) and N-methylmorpholine-N-oxide (50% aq solution, 0.025 mL, 0.21 mmol) was added. OsO₄ (4% aq solution, 0.007 mmol) was then added. After 3 h at 25° C., the reaction was complete and sodium sulfite (0.25 g) was added with vigorous stirring. After 15 minutes, brine (5 mL) was added and the solution extracted with EtOAc (3×10 mL). The organics were dried over anhydrous sodium sulfate and then concentrated in vacuo. The crude diol was purified by preparative TLC on silica gel, eluting with 10% acetone in chloroform to give 0.038 g of compound 232C as a yellow solid. HPLC: 98% at 3.747 min (retention time) (YMC S5 ODS column 4.6×50 mm eluting with 10-90% aqueous methanol over 4 minutes containing 0.2% phosphoric acid, 4 mL/min, monitoring at 220 nm), MS (ES): m/z 593.08 [M+Na]⁺.

### EXAMPLE 233

### (3aα,4β,5β,5aβ,8aβ,8bα)-4-(Decahydro-5-hydroxy-4-methyl-1,3-dioxo-4,8a-epoxy-2H-furo[3,2-e]isoindol-2-yl)-1-naphthalenecarbonitrile, (233)

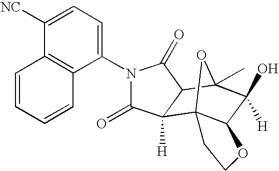

To a solution of triphenylphosphine (0.072 g, 0.276 mmol) in THF (3.0 mL) was added DBAD (0.063 g, 0.276 mmol). After 10 min, 4-cyanophenol (0.033 g , 0.276 mmol) was added. After 10 additional minutes, compound 231C (0.075 g, 0.184 mmol) was added. After 3 h at 25° C., the reaction was concentrated in vacuo and purified by preparative TLC on silica gel, eluting with 15% acetone in chloroform to give 0.068 g of compound 233 as a white solid. HPLC: 95% at 2.430 and 2.560 min (atropisomers) (retention time) (YMC S5 ODS column 4.6×50 mm, 10-90% aqueous methanol over 4 minutes containing 0.2% phosphoric acid, 4 mL/min, monitoring at 220 nm), MS (ES): m/z 391.09 [M+H]⁺.

### EXAMPLE 234

### (3aα,4β,7β,7aα)-2-(4-Cyano-1-naphthalenyl)octahydro-7-methyl-1,3-dioxo-4,7-epoxy-4H-isoindole-4-acetic acid, (234B)

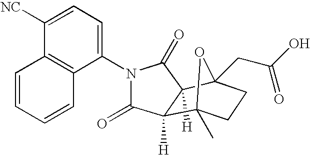

A. (3aα,4β,7β,7aα)-2(4-Cyano-1-naphthalenyl)-1,2,3,3a,7,7a-hexahydro-7-methyl-1,3-dioxo-4,7-epoxy-4H-isoindole-4-acetic acid (234A)

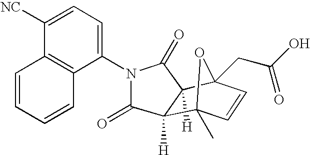

5-Methyl-2-furanacetic acid (0.500 g, 3.57 mmol) and 4-(2,5-dihydro-2,5-dioxo-1H-1-yl)-1-naphthalenecarbonitrile (0.899 g, 3.57 mmol) were dissolved in benzene (3.0 mL) and heated at 60° C. for 2 h and then cooled to 25° C. After 12 h, a white solid precipitated out of solution and was filtered and rinsed with diethyl ether to yield 1.20 g of compound 234A as a light yellow solid. NMR showed only one diastereomer. HPLC: 86% at 2.767 min (retention time) (YMC S5 ODS column 4.6×50 mm, 10-90% aqueous methanol over 4 minutes containing 0.2% phosphoric acid, 4 mL/min, monitoring at 220 nm), MS (ES): m/z 389.45 [M+H]⁺.

B (3aα,4β,7β,7aα)-2-(4-Cyano-1-naphthalenyl)octahydro-7-methyl-1,3-dioxo-4,7-epoxy-4H-isoindole-4-acetic acid, (234B)

Compound 234A (1.1 g, 2.82 mmol) was dissolved in EtOH/EtOAc (1:1, 50 mL) and Pd/C (10% Pd, 0.4 g) was added followed by H₂ via a balloon. After 5 h at 25° C., the reaction was filtered through celite rinsing with EtOAc and concentrated in vacuo to yield 1.00 g of compound 234B as a yellow solid. HPLC: 80% at 2.84 min (retention time) (YMC S5 ODS column 4.6×50 mm, 10-90% aqueous methanol over 4 minutes containing 0.2% phosphoric acid, 4 mL/min, monitoring at 220 nm), MS (ES): m/z 391.1[M+H]⁺.

### EXAMPLE 235

### (3aα,4β,7β,7aα)-2-(4-Cyano-1-naphthalenyl)octahydro-7-methyl-1,3-dioxo-4,7-epoxy-4H-isoindole-4-acetic acid, methyl ester, (235)

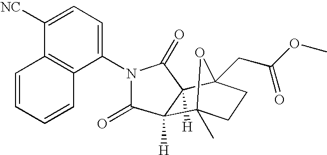

Compound 234B (0.050 g, 0.125 mmol) was dissolved in acetonitrile (2.0 mL), then DCC (0.025 g, 0.125 mmol) was added followed by HOAc (0.018 g, 0.125 mmol). 4-Fluorobenzyl alcohol (0.014 mL, 0.125 mmol) was added and the reaction was stirred for 3 h. The reaction was then concentrated in vacuo and purified by preparative reverse phase HPLC (YMC S5 ODS 20×100 mm, 10-90% aqueous methanol over 15 min containing 0.1% TFA, 20 mL/min, monitoring at 220 nm). Purification yielded 0.040 g of compound 235 as a white solid, rather than the expected benzyl ester. None of the anticipated benzyl ester was observed by NMR or LC-MS. HPLC: 100% at 3.033 min (retention time) (YMC S5 ODS column 4.6×50 mm, 10-90% aqueous methanol over 4 minutes containing 0.2% phosphoric acid, 4 mL/min, monitoring at 220 nm), MS (ES): m/z 405.51 [M+H]⁺.

### EXAMPLE 236

### (3aα,4β,7β,7aα)-2-(4-Cyano-1-naphthalenyl)-N-[(4-fluorophenyl)methyl]octahydro-7-methyl-1,3-dioxo-4,7-epoxy-4H-isoindole-4-acetamide, (236)

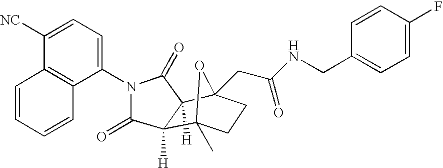

Compound 234B (0.100 g, 0.256 mmol) was dissolved in acetonitrile (4.0 mL). HOAc (0.035 g, 0.256 mmol) and DCC (0.049 g, 0.256 mmol) were then added followed by 4-fluorobenzylamine (0.030 mL, 0.256 mmol). After 4 h at 25° C., the reaction was concentrated in vacuo and purified by preparative HPLC (YMC S5 ODS 20×100 mm, 10-90% aqueous methanol over 15 minutes containing 0.1% TFA, 20 mL/min, monitoring at 220 nm) to yield 0.085 g of compound 236 as a white solid. HPLC: 100% at 3.277 min (retention time) (YMC S5 ODS column 4.6×50 mm, 10-90% aqueous methanol over 4 minutes containing 0.2% phosphoric acid, 4 mL/min, monitoring at 220 nm), MS (ES): m/z 498.43 [M+H]⁺.

### EXAMPLE 237

### (3aα,4β,7β,7aα)-N-[2-[2-(4-Cyano-1-naphthalenyl)octahydro-7-methyl-1,3-dioxo-4,7-epoxy-4H-isoindol-4-yl]ethyl]-4-fluorobenzamide, (237B)

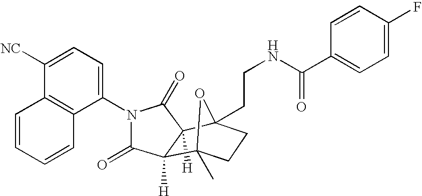

A. 4-Fluoro-N-[2-(5-methyl-2-furanyl)ethyl]benzamide (237A)

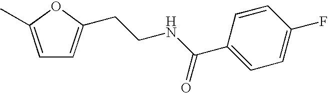

4-Fluorophenylacetyl chloride (0.29 mL, 2.44 mmol) was added dropwise to a solution of β-(5-methyl-2-furanyl)ethanamine (300 mg, 2.44 mmol, made according to the procedure of Yur'ev, Yu. K. et. al. J. Gen. Chem. USSR (Engl. Transl.) 33, 3444-8 (1963)) in THF (2.5 mL) at rt, then Et₃N (0.34 mL, 2.44 mmol) was added dropwise. Once the starting material was consumed, as was evident by HPLC, the reaction was quenched with H₂O and extracted with CH₂Cl₂. The combined organic layers were dried over MgSO₄ and concentrated under reduced pressure. Purification by flash chromatography eluting with 0%-50% EtOAc/hexane gradient gave 523 mg (95%) of compound 237A as a white solid. HPLC: 99% at 2.84 min (retention time) (Phenomenex-prime S5-C18 column 4.6×50 mm eluting with 10-90% aqueous methanol over 4 minutes containing 0.2% phosphoric acid, 4 mL/min, monitoring at 220 nm), MS (ES): m/z 248.15 [M+H]⁺.

B. (3aα,4β,7β,7aα)-N-[2-[2-(4-Cyano-1-naphthalenyl)octahydro-7-methyl-1,3-dioxo-4,7-epoxy-4H-isoindol-4-yl]ethyl]-4-fluorobenzamide, (237B)

A solution of compound 237A (221.5 mg, 0.896 mmol) and 4-(2,5-dihydro-2,5-dioxo-1H-1-yl)-1-naphthalenecarbonitrile (222.4 mg, 0.896 mmol) in benzene (4 mL) was heated at 60° C. overnight. The reaction mixture was concentrated under reduced pressure and dissolved in EtOAc (30 mL). 10% Pd/C (50 mg) was added and the mixture was stirred under a hydrogen balloon overnight. The reaction mixture was filtered through a pad of celite and concentrated under reduced pressure. Purification by flash chromatography eluting with 25%-75% EtOAc/hexane gradient gave 160.3 mg (36%) of compound 237B as an off-white solid. HPLC: 97% at 3.13 & 3.23 min (retention time) (Phenomenex-prime S5-C18 column 4.6×50 mm eluting with 10-90% aqueous methanol over 4 minutes containing 0.2% phosphoric acid, 4 mL/min, monitoring at 220 nm), MS (ES): m/z 498.11 [M+H]⁺.

### EXAMPLE 238

### [3aR-(3aα,4β,7β,7aα)]-4-[Octahydro-4-(2-hydroxyethyl)-7-methyl-1,3-dioxo-4,7-epoxy-2H-isoindol-2-yl]-1-naphthalenecarbonitrile & [3aS-(3aα,4β,7β,7aα)]-4-[Octahydro-4-(2-hydroxyethyl)-7-methyl-1,3-dioxo-4,7-epoxy-2H-isoindol-2-yl]-1-naphthalenecarbonitrile. (238i & 238ii)

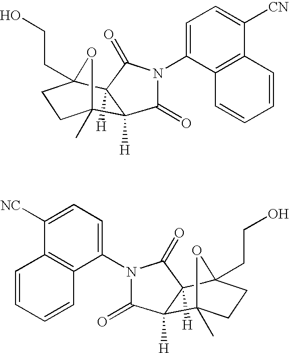

Racemic compound 223B was separated into its enantiomers by preparative chiral HPLC (CHIRALPAK AD 5×50 cm column; eluting with 20% MeOH/EtOH (1:1) in heptane (isocratic) at 50 mL/min, @ 220 nm) to give the faster eluting compound 238i (Chiral HPLC: 13.54 min; CHIRALPAK AD 4.6×250 mm column; eluting with 20% MeOH/EtOH (1:1) in heptane at 1 mL/min) and the slower eluting compound 238ii (Chiral HPLC: 14.99 min; CHIRALPAK AD 4.6×250 mm column; eluting with 20% MeOH/EtOH (1:1) in heptane at 1 mL/min). The absolute conformation for compounds 238i & 238ii was not established. For simplicity in nomenclature, compound 238i is designated herein as having an “R” configuration and compound 238ii as having an “S” configuration. Enantiomerically pure products derived from compound 238i are designated herein as having a “R” configuration and enantiomerically pure products derived from compound 238ii are designated herein as having an “S” configuration.

### EXAMPLE 239

### [3aR-(3aα,4β,7β,7aα)]-4-[4-[2-(3-Fluorophenoxy)ethyl]octahydro-7-methyl-1,3-dioxo-4,7-epoxy-2H-isoindol-2-yl]-1-naphthalenecarbonitrile & [3aS-(-(3aα,4β,7β,7aα)]-4-[4-[2-(3-Fluorophenoxy)ethyl]octahydro-7-methyl-1,3-dioxo-4,7-epoxy-2H-isoindol-2-yl]-1-naphthalenecarbonitrile, (239i & 239ii)

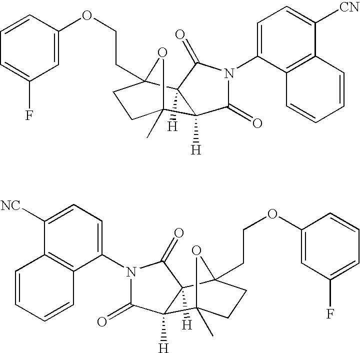

To a solution of triphenylphosphine (0.0524 g, 0.20 mmol) in THF (2.0 mL) was added DBAD (0.046 g, 0.2 mmol). After 10 min, 3-fluorophenol (0.018 mL, 0.2 mmol) was added. After 10 additional minutes, enantiomerically pure compound 238 i (0.050 g, 0.133 mmol) was added. After 3 h at 25° C., the reaction was concentrated in vacuo and purified by preparative HPLC (YMC S5 ODS 20×100 mm, 10-90% aqueous methanol over 15 minutes containing 0.2% TFA, 20 mL/min, monitoring at 220 nm) to give 0.031 g of compound 239i as a white solid. This process was repeated with enantiomerically pure compound 238ii to yield compound 239ii. Compound 239i: HPLC: 100% at 3.80 min (retention time) (YMC S5 ODS column 4.6×50 mm, 10-90% aqueous methanol over 4 minutes containing 0.2% phosphoric acid, 4 mL/min, monitoring at 220 nm), MS (ES): m/z 471.65 [M+H]⁺, [α]_{D}²⁵=−47.371 (c=4.412 mg/cc, CH₂Cl₂). Compound 239ii: HPLC: 100% at 3.80 min (retention time) (YMC S5 ODS column 4.6×50 mm, 10-90% aqueous methanol over 4 minutes containing 0.2% phosphoric acid, 4 mL/min, monitoring at 220 nm), MS (ES): m/z 471.65 [M+H]⁺, [α]_{D}²⁵=+24.3 (c=4.165 mg/cc, CH₂Cl₂).

### EXAMPLE 240

### (4-Fluorophenyl)carbamic acids, 2-[(3aα,4β,7β,7aα)-2-(4-cyano-1-naphthalenyl)octahydro-7-methyl-1,3-dioxo-4,7-epoxy-4H-isoindol-4-y]ethyl ester, (240)

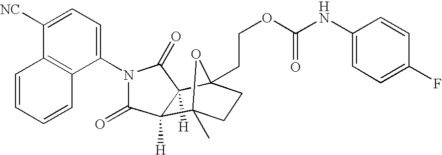

Compound 223B (0.100 g, 0.279 mmol) was dissolved in dichloroethane (3.0 mL) and 4-fluorophenylisocyanate (0.048 mL, 0.419 mmol) was added followed by heating to 60° C. After 2 h, the reaction was cooled to 25° C. and diluted with methylene chloride. The mixture was washed once with sat. aq sodium bicarbonate (20 mL) and then the organics were dried over anhydrous sodium sulfate. The crude material was purified by flash chromatography on silica eluting with 15% acetone in chloroform to give 0.098 g of compound 240 as a yellow foam. HPLC: 98% at 3.320 and 3.457 min (atropisomers) (retention time) (YMC S5 ODS column 4.6×50 mm, 10-90% aqueous methanol over 4 minutes containing 0.2% phosphoric acid, 4 mL/min, monitoring at 220 nm), MS (ES): m/z 514.13 [M+H]⁺.

### EXAMPLE 241

### (3aα,4β,7β,7aα)-4-[Octahydro-4-(2-hydroxyethyl)-1,3-dioxo-4,7-epoxy-2H-isoindol-2-yl]-1-naphthalenecarbonitrile, (241D)

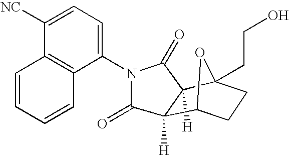

A. 2-[2-[[(1,1-Dimethlethyl)dimethylsilyl]oxy]ethyl]furan (241A)

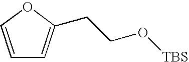

2-(2-Hydroxyethyl)furan (1.00 g, 8.93 mmol, Example 255A) was dissolved in DMF at 25° C. and imidazole (0.790 g, 11.61 mmol) was added. TBSCI (1.35 g, 8.93 mmol) was then added by portions over 5 minutes. After 2 h, the reaction was poured into diethyl ether (300 mL) and washed sequentially with water (1×100 mL), 1 N HCl (1×100 mL), and brine (1×100 mL). The combined organics were then dried over magnesium sulfate and concentrated in vacuo. Compound 241A was isolated as a clear oil (1.77 g) and was taken on without purification. HPLC: 100% at 4.233 min (retention time) (YMC S5 ODS column 4.6×50 mm, 10-90% aqueous methanol over 4 minutes containing 0.2% phosphoric acid, 4 mL/min, monitoring at 220 nm).

B. (3aα,4β,7β,7aα)-4-[4-[2-[[(1,1-Dimethylethyl)dimethylsilyl]oxy]ethyl]-1,3,3a,4,7,7a-hexahydro-1,3-dioxo-4,7-epoxy-2H-isoindol-2-yl]-1-naphthalenecarbonitrile (241B)

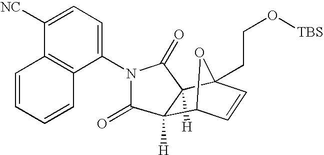

4-(2,5-Dihydro-2,5-dioxo-1H-1-yl)-1-naphthalenecarbonitrile (0.721 g, 3.40 mmol) was suspended in benzene (5.0 mL) in a sealed tube and compound 241A (1.00 g, 4.42 mmol) was added. The reaction was heated at 60° C. for 16 h and then cooled to 25° C. The benzene was removed in vacuo to give a yellow solid. The crude material was purified by flash chromatography on silica eluting with 1-5% acetone in chloroform to give 1.37 g of compound 241B as a yellow solid. NMR experiments confirmed exo isomer assignment. HPLC: 100% at 4.030 and 4.110 (atropsiomers) min (retention time) (YMC S5 ODS column 4.6×50 mm, 10-90% aqueous methanol over 4 minutes containing 0.2% phosphoric acid, 4 mL/min, monitoring at 220 nm).

C. (3aα,4β,7β,7aα)-4-[4-[2-[[(1,1-Dimethylethyl)dimethylsilyl]oxy]ethyl]octahydro-1,3-dioxo-4,7-epoxy-2H-isoindol-2-yl]-1-naphthalenecarbonitrile (241C)

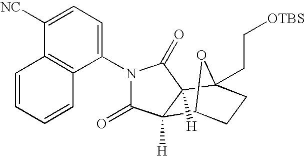

The compound 241B (0.500 g, 1.14 mmol) was dissolved in ethyl acetate (40 mL) and Pd/C (10% Pd, 0.200 g) was added. Hydrogen was then introduced via a balloon. After 4 h, the reaction was filtered through celite, rinsed with ethyl acetate and concentration in vacuo to yield a pale yellow solid, which was purified by flash chromatography on silica gel eluting with acetone/chloroform (0%-1.5%-3% acetone) to give compound 241C (0.450 g) as a yellow foam.

D. (3aα,4β,7β,7aα)-4-[Octahydro-4-(2-hydroxyethyl)-1,3-dioxo-4,7-epoxy-2H-isoindol-2-yl]-1-naphthalenecarbonitrile, (241D)

Compound 241C (0.283 g, 0.50 mmol) was dissolved in a solution of 2% 12N HCl in absolute ethanol (10 mL). After 1 h, the reaction was quenched with sat. aq sodium bicarbonate and extracted with methylene chloride (4×20 mL). The combined organics were dried over sodium sulfate and concentrated in vacuo to give 0.211 g of compound 241D as a white solid. HPLC: 100% at 2.14 min (retention time) (YMC S5 ODS column 4.6×50 mm, 10-90% aqueous methanol over 4 minutes containing 0.2% phosphoric acid, 4 mL/min, monitoring at 220 nm), MS (ES): m/z 363.45 [M+H]⁺.

### EXAMPLE 242

### (3aα,4β,6β,7β,7aα)-4-[4-[2-(4-Cyanophenoxy)ethyl]octahydro-6-hydroxy-1,3-dioxo-4,7-epoxy-2H-isoindol-2-yl]-1-naphthalenecarbonitrile, (242C)

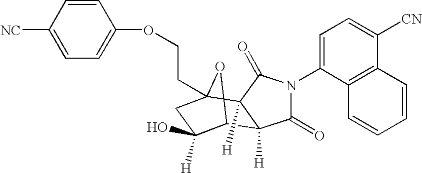

A. (3aα,4β,6β,7β,7aα)-4-[4-[2-[[(1,1-Dimethylethyl)dimethylsilyl]oxy]ethyl]octahydro-6-hydroxy-1,3-dioxo-4,7-epoxy-2H-isoindol-2-yl]-1-naphthalenecarbonitrile (242A)

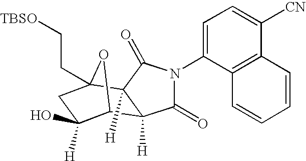

Compound 241B (1.00 g, 2.28 mmol) and Wilkinson's catalyst (0.105 g, 0.114 mmol) were stirred rapidly under vacuum at 25° C. for 1 h and then purged with N₂. THF (30 mL) was then added followed by catechol borane (0.487 mL, 4.57 mmol) after the olefin was completely dissolved. After 1 h, the reaction was cooled to 0° C. and a pH 7.2 phosphate buffer (33 mL) was added followed by EtOH (13 mL) and H₂O₂ (30% aq soln, 3.0 g). After 3 h at 0° C. the reaction was complete by LC and the mixture was extracted with methylene chloride (3×50 mL). The combined organics were washed with a 1:1 mixture of 10% sodium sulfite/1N NaOH (50 mL) and once with brine (50 mL). All aqueous phases were combined and extracted with methylene chloride (50 mL) and the organic phase combined with the previous extractions. All the organics were then dried over anhydrous sodium sulfate and then concentrated in vacuo. The crude material was purified by flash chromatography on silica eluting with 10-20% acetone in chloroform to give 0.634 g of compound 242A as a white foam. HPLC: 96% at 3.797 min (retention time) (YMC S5 ODS column 4.6×50 mm, 10-90% aqueous methanol over 4 minutes containing 0.2% phosphoric acid, 4 mL/min, monitoring at 220 nm), MS (ES): m/z 493.13 [M+H]⁺.

B. (3aα,4β,6β,7β,7aα)-4-[Octahydro-6-hydroxy-4-(2-hydroxyethyl)-1,3-dioxo-4,7-epoxy-2H-isoindol-2-yl]-1-naphthalenecarbonitrile (242B)

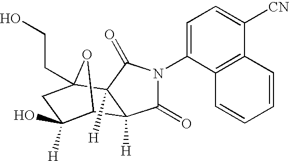

Compound 242A (0.400 g, 0.813 mmol) was dissolved in a solution of 2% 12N HCl in absolute ethanol (10 mL). After 1 h, the reaction was quenched with sat. aq sodium bicarbonate and extracted with EtOAc (4×20 mL). The combined organics were dried over sodium sulfate and concentrated in vacuo to give 0.305 g of compound 242B as a white solid. HPLC: 90% at 2.043 min (retention time) (YMC S5 ODS column 4.6×50 mm, 10-90% aqueous methanol over 4 minutes containing 0.2% phosphoric acid, 4 mL/min, monitoring at 220 nm), MS (ES): m/z 379.09 [M+H]⁺.

C. (3aα,4β,5β,7β,7aα)-4-[4-[2-(4-Cyanophenoxy)ethyl]octahydro-6-hydroxy-1,3-dioxo-4,7-epoxy-2H-isoindol-2-yl]-1-naphthalenecarbonitrile, (242C)

To a solution of triphenylphosphine (0.054 g, 0.207 mmol) in THF (2.0 mL) was added DBAD (0.048 g, 0.207 mmol). After 10 min, 4-cyanophenol (0.025 g, 0.207 mmol) was added. After 10 additional minutes, compound 242B (0.050 g, 0.138 mmol) was added. After 3 h at 25° C., the reaction was concentrated in vacuo and purified by preparative TLC on silica eluting with 25% acetone/chloroform to give 0.056 g of compound 242C as a white solid. HPLC: 90% at 2.987 min (retention time) (YMC S5 ODS column 4.6×50 mm, 10-90% aqueous methanol over 4 minutes containing 0.2% phosphoric acid, 4 mL/min, monitoring at 220 nm), MS (ES): m/z 480.10 [M+H]⁺.

### EXAMPLE 243

### [3aS-(3aα,4β,5β,7β,7aα)]-4-[Octahydro-5-hydroxy-7-(2-hydroxyethyl)-4-methyl-1,3-dioxo-4,7-epoxy-2H-isoindol-2-yl]-1-naphthalenecarbonitrile & [3aR-(3aα,4β,5β,7β,7aα)]-4-[Octahydro-5-hydroxy-7-(2-hydroxyethyl)-4-methyl-1,3-dioxo-4,7-epoxy-2H-isoindol-2-yl]-1-naphthalenecarbonitrile, (243Di & 243Dii)

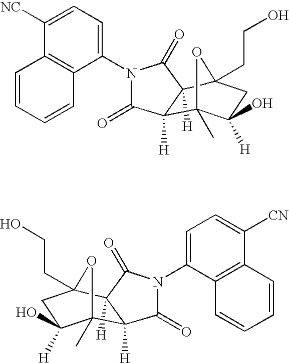

A. (3aα,4β,7β,7aα)-4-[4-[2-[[(1,1-Dimethylethyl)dimethylsilyl]oxy]ethyl]-1,3,3a,4,7,7a-hexahydro-1,3-dioxo-4,7-epoxy-2H-isoindol-2-yl]-1-naphthalenecarbonitrile (243A)

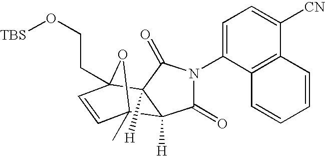

4-(2,5-Dihydro-2,5-dioxo-1H-1-yl)-1-naphthalenecarbonitrile (18.3 g, 68.7 mmol) was added to a solution of compound 204A (26.6 g, 110.6 mmol) in benzene (75 mL) and heated to 60° C. overnight. After cooling to rt, the reaction mixture was concentrated under reduced pressure. The residue was treated with MeOH (250 mL) with stirring at 0° C. for 10 min. The resulting solid was filtered, washed with cold MeOH (2×10 mL) and dried to give 26.7 g (79.5%) of compound 243A as a yellow solid. HPLC analysis of the above solid revealed it to be 95% pure (HPLC conditions: 95% at 2.48 min (Phenomenex-prime S5-C18 column, 4.6×50 mm, 10%-90% aqueous methanol over 4 minute gradient with 0.2% H₃PO₄, detecting at 220 nm)). The filtrate was then concentrated under reduced pressure and the resulting solid was chromatographed, eluting with 3% acetone/CHCl₃, to give an additional 4.36 g of compound 243A (13%), giving a total final yield of 92.5%.

B. (3aα,4β,5β,7β,7aα)-4-[7-[2-[[(1,1-Dimethylethyl)dimethylsilyl]oxy]ethyl]octahydro-5-hydroxy-4-methyl-1,3-dioxo-4,7-epoxy-2H-isoindol-2-yl]-1-naphthalenecarbonitrile (243B)

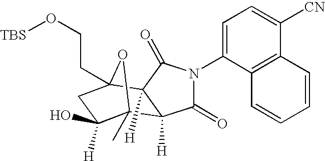

A mixture of 243A (10 g, 20.46 mmol) and RhCl(PPh₃)₃ (0.947 mg, 1.02 mmol) was evacuated and filled with argon (3×). THF (200 mL) was added and once all particulates had dissolved, catecholborane (4.4 mL, 40.93 mmol) was slowly added dropwise. When the formation of product ceased, as was determined by HPLC, the reaction mixture was cooled to 0° C. and quenched with phosphate buffer (330 mL, pH 7.2) then EtOH (130 mL) and H₂O₂ (300 mL, 30% aq sol) were added. Once boronate was consumed, the mixture was extracted with CH₂Cl₂ (3×) and the combined organic layers were washed with 1N NaOH, 10% aq NaHSO₃ (1:1, 1×) and brine (1×). The combined washes was extracted with CH₂Cl₂ (1×) and the combined organic layers were dried over Na₂SO₄. Purification by flash chromatography on silica gel eluting with 10% to 30% acetone/CHCl₃ gradient over 25 min gave 7.1 g (68%) of 243B as a light yellow solid. HPLC conditions: 98% at 3.82 min (Phenomenex-prime S5-C18 column 4.6×50 mm, 10%-90% aqueous methanol over 4 minute gradient with 0.2% H₃PO₄, detecting at 220 nm).

C. [3aR-(3aα,4β,5β,7β,7aα)]-4-[7-[2-[[(1,1-Dimethylethyl)dimethylsilyl]oxy]ethyl]octahydro-5-hydroxy-4-methyl-1,3-dioxo-4,7-epoxy-2H-isoindol-2-yl]-1-naphthalenecarbonitrile & [3aS-(3aα,4β,5β,7β,7aα)]-4-[7-[2-[[(1,1-Dimethylethyl)dimethylsilyl]oxy]ethyl]octahydro-5-hydroxy-4-methyl-1,3-dioxo-4,7-epoxy-2H-isoindol-2-yl]-1-naphthalenecarbonitrile (243Ci & 243Cii)

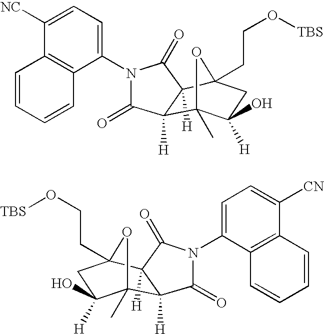

The racemic compound 243B was separated into the individual enantiomers by chiral normal phase liquid chromatography. A Chiralpak OD column (50×500 mm) was used, eluting with 13% EtOH/hexanes over 99 min at 50 mL/min detecting at 220 nm. The faster eluting isomer compound 243Ci had a retention time =45 min and the slower eluting isomer compound 243Cii had a retention time =66 min.

D. [3aS-(3aα,4β,5β,7β,7aα)]-4-[Octahydro-5-hydroxy-7-(2-hydroxyethyl)-4-methyl-1,3-dioxo-4,7-epoxy-2H-isoindol-2-yl]-1-naphthalenecarbonitrile & [3aR-(3aα,4β,5β,7β,7aα)]-4-[Octahydro-5-hydroxy-7-(2-hydroxyethyl)-4-methyl-1,3-dioxo-4,7-epoxy-2H-isoindol-2-yl]-1-naphthalenecarbonitrile, (243Di & 243Dii)

Compound 243Ci (0.84 g, 2.14 mmol) was dissolved in 2% 12 N HCl/EtOH (20 mL), stirred for 5 minutes and concentrated under reduced pressure. Purification by flash chromatography on silica gel eluting with 5-10% MeOH/CH₂Cl₂ gave 0.57 g (88%) of 243Di. Compound 243Di which came from the faster eluting isomer (243Ci) was found to be 99.7% ee by analytical chiral normal phase chromatography. HPLC conditions: 99.7% at 2.17 min (Chiralcel OJ 44.6×250 mm, 10 micron, 40° C., isocratic 80% Heptane/20% EtOH/MeOH (1:1), 1.0 mL/min., detection at 288 nm).

Compound 243Cii (0.86 g, 2.19 mmol) was dissolved in 2% 12N HCl/EtOH (20 mL), stirred for 5 minutes and concentrated under reduced pressure. Purification by flash chromatography on silica gel eluting with 5-10% MeOH/CH₂Cl₂ gave 0.60 g (90%) of 243Dii. Compound 243Dii which came from the slower eluting isomer (243Cii) was found to have 87.1% ee by analytical chiral normal phase chromatography. HPLC conditions: 87.1% at 18.4 min (Chiralcel OJ 44.6×250 mm, 10 micron, 40° C., isocratic 80% heptane/20% EtOH/MeOH (1:1), 1.0 mL/min., detection at 288 nm).

The absolute conformation for compounds 243Di & 243Dii was not determined. For simplicity in nomenclature, compound 243Di is designated herein as having an “S” configuration and compound 243Dii as having an “R” configuration. Enantiomerically pure products derived from compound 243Di are designated herein as having an “S” configuration and enantiomerically pure products derived from compound 243Dii are designated herein as having an “R” configuration

### EXAMPLE 244

### [3aS-(3aα,4β,5β,7β,7aα)]-4-[7-[2-(4-Cyanophenoxy)ethyl]octahydro-5-hydroxy-4-methyl-1,3-dioxo-4,7-epoxy-2H-isoindol-2-yl]-1-naphthalenecarbonitrile & [3aR-(3aα,4β,5β,7β,7aα)]-4-[7-[2-(4-Cyanophenoxy)ethyl]octahydro-5-hydroxy-4-methyl-3-dioxo-4,7-epoxy-2H-isoindol-2-yl]-1-naphthalenecarbonitrile, (244i & 244ii)

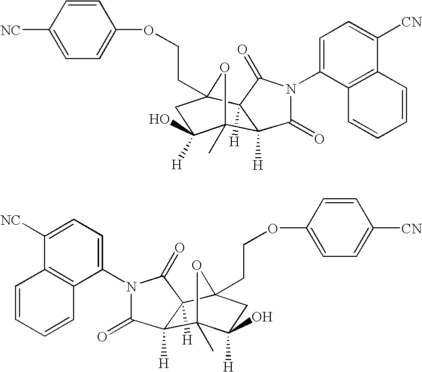

DBAD (26 mg, 0.115 mmol) was added to a solution of PPh₃ (30 mg, 0.115 mmol) in THF (0.65 mL). After stirring for 10 min, 4-cyanophenol (13.6 mg, 0.115 mmol) was added and the reaction mixture was stirred for a further 5 min. Compound 243Di (30 mg, 0.076 mmol) was added and the mixture was stirred at rt for 1 h. The reaction was concentrated under reduced pressure. Purification by flash chromatography on silica gel eluting with 30% acetone/70% CHCl₃ gave 23.1 mg (0.047 mmol, 61.7%) of compound 244i. HPLC conditions: 95% at 3.06 min (YMC S5 ODS 4.6×50 mm, 10%-90% aqueous methanol over 4 minute gradient with 0.2% H₃PO₄, detecting at 220 nm). MS (ES): m/z 494.09 [M+H]⁺. [α]_{D}=53.30°, C=4.5 mg/cc in THF, @ 589 nm)

DBAD (26 mg, 0.115 mmol) was added to a solution of PPh₃ (30 mg, 0.115 mmol) in THF (0.65 mL). After stirring for 10 min, 4-cyanophenol (13.6 mg, 0.115 mmol) was added and the reaction mixture was stirred for a further 5 min. Compound 243Dii (30 mg, 0.076 mmol) was added and the mixture was stirred at rt for 1 h. The reaction was concentrated under reduced pressure. Purification by flash chromatography on silica gel eluting with 30% acetone/70% CHCl₃ gave 20.3 mg (0.041 mmol, 54.2%) of compound 244ii. HPLC conditions: 90% at 3.07 min (YMC S5 ODS 4.6×50 mm, 10%-90% aqueous methanol over 4 minute gradient with 0.2% H₃PO₄, detecting at 220 nm). MS (ES): m/z 494.09 [M+H]⁺. [α]_{D}=−42.87°, C=6.6 mg/cc in THF, @ 589 nm)

### EXAMPLE 245

### (3aα,4β,7β,7aα)-4-[4-[2-(4-Cyanophenoxy)ethyl]-7-ethyloctahydro-1,3-dioxo-4,7-epoxy-2H-isoindol-2-yl]-1-naphthalenecarbonitrile. (245D)

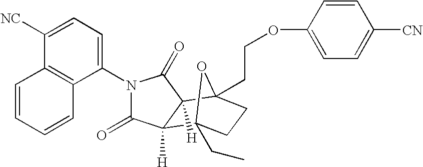

A. 2-Ethyl-5-(2-hydroxyethyl)furan (245A)

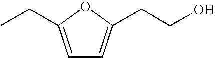

n-BuLi (2.5 M in hexane, 4.4 mL, 11 mmol) was added to a solution of 2-ethylfuran (1.05 mL, 10 mmol) in THF (10 mL) at −25° C. The solution was warmed to rt and stirred for 3 h. Ethylene oxide (0.75 mL) was added at −78° C. The reaction was stirred for 0.5 h at −15° C. and overnight at rt. Aqueous sat. NH₄Cl was added and the mixture was extracted with ether (3×). The combined extracts were washed with water (1×) and brine (1×) and dried over Na₂SO₄. Purification by flash chromatography on silica gel eluting with 30% EtOAc/70% hexane gave 1.12 g (8.02 mmol, 80.2%) of compound 245A as a yellow oil.

B. (3aα,4β,7β,7aα)-4-[4-Ethyl-1,3,3a,4,7,7a-hexahydro-7-(2-hydroxyethyl)-1,3-dioxo-4,7-epoxy-2H-isoindol-2-yl]-1-naphthalenecarbonitrile (245B)

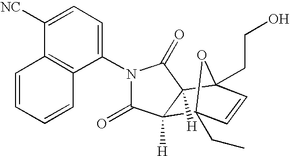

A solution of compound 245A (280 mg, 2.00 mmol) and the 4-(2,5-dihydro-2,5-dioxo-1H-1-yl)-1-naphthalenecarbonitrile (496 mg, 2.00 mmol) in benzene (2 mL) was stirred at 60° C. for 2 h. The reaction mixture was concentrated under reduced pressure. The yellow solid, compound 245B, was used directly in the next step.

C. (3aα,4β,7β,7aα)-4-[4-Ethyloctahydro-7-(2-hydroxyethyl)-1,3-dioxo-4,7-epoxy-2H-isoindol-2-yl]-1-naphthalenecarbonitrile (245C)

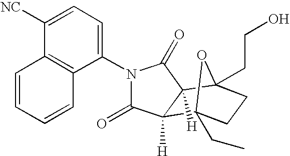

A mixture of compound 245B (764 mg, 1.97 mmol) and 10% Pd/C (115 mg, cat.) in EtOAc (36 mL) was stirred under a hydrogen atmosphere at rt for 2 h. The reaction mixture was filtered through celite and concentrated under reduced pressure to give 779 mg of crude compound 245C. Purification of this crude product by flash chromatography on silica gel eluting with 70% EtOAc/30% hexane gave 235 mg (0.6 mmol, 30.1%) of compound 245C. HPLC conditions: 99% at 2.84 min (YMC S5 ODS 4.6×50 mm, 10%-90% aqueous methanol over 4 minute gradient with 0.2% H₃PO₄, detecting at 220 nm). MS (ES): m/z 391.12 [M+H]⁺.

D. (3aα,4β,7β,7aα)-4-[4-[2-(4-Cyanophenoxy)ethyl]-7-ethyloctahydro-1,3-dioxo-4,7-epoxy-2H-isoindol-2-yl]-1-naphthalenecarbonitrile (245D)

DBAD (44.2 mg, 0.192 mmol) was added to a solution of PPh₃ (50.4 mg, 0.192 mmol) in THF (1 mL). After stirring for 10 mins, 4-cyanophenol (23 mg, 0.192 mmol) was added and the reaction mixture was stirred for an additional 5 mins. Compound 245C (50 mg, 0.128 mmol) was added and the mixture was stirred at rt for 2 h. The reaction was concentrated under reduced pressure. Purification by flash chromatography on silica gel eluting with 40% EtOAc/60% hexane gave 43 mg (0.087 mmol, 68.4%) of compound 245D as a white solid. HPLC conditions: 99% at 3.65 min (YMC S5 ODS 4.6×50 mm, 10%-90% aqueous methanol over 4 minute gradient with 0.2% H₃PO₄, detecting at 220 nm). MS (ES): m/z 492.16 [M+H]⁺.

### EXAMPLE 246

### (3aα,4β,7β,7aα)-4-[2-(Acetyloxy)ethyl]-2-(4-cyano-1-naphthalenyl)hexahydro-7-methyl-4,7-epoxy-1H-isoindole-1,3(2H)-dione, (246)

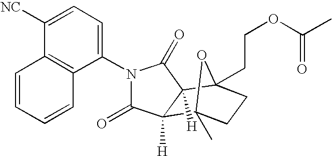

Compound 223B (0.100 g, 0.279 mmol) was dissolved in methylene chloride (3.0 mL) at 25° C. and pyridine (0.071 mL, 0.837 mmol) and 4-DMAP (1.0 mg) were added. Acetic anhydride (0.053 mL, 0.559 mmol) was then added and the reaction was stirred for 20 h at 25° C. After 20 h, sat. aq sodium bicarbonate was added and the reaction was stirred for 30 min. The mixture was then extracted with methylene chloride (2×20 mL). The organics were then washed once with 1N HCl (10 mL) and then dried over anhydrous sodium sulfate. After concentration in vacuo, the crude material was purified by preparative TLC on silica eluting with 12% acetone in chloroform to give 0.073 g of compound 246 as a yellow foam. HPLC: 95% at 2.837 and 3.027 min (atropisomers) (retention time) (YMC S5 ODS column 4.6×50 mm, 10-90% aqueous methanol over 4 minutes containing 0.2% phosphoric acid, 4 mL/min, monitoring at 220 nm), MS (ES): m/z 441.10 [M+Na]⁺.

### EXAMPLE 247

### (3aα,4β,7β,7aα)-4-[Octahydro-4-methyl-1,3-dioxo-7-(2-oxoethyl)-4,7-epoxy-2H-isoindol-2-yl]-1-naphthalenecarbonitrile, (247)

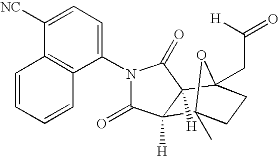

Oxalyl chloride (2.0 M soln, 1.73 mL, 3.5 mmol) was added to dry methylene chloride (10 mL) and cooled to −78° C. DMSO (0.283 mL, 3.99 mmol) was then added dropwise with the evolution of gas. After 15 min, compound 223B (1.00 g, 2.66 mmol) was then added in methylene chloride (10 mL). After 15 min, TEA (1.10 mL, 7.98 mmol) was added and the reaction was slowly warmed to 25° C. Water (30 mL) was then added and the mixture was diluted with methylene chloride (100 mL). The organics were then washed once with 1 N HCl (30 mL), once with water (30 mL) and once with brine (30 mL) and then dried over anhydrous sodium sulfate. The crude product was isolated by concentration in vacuo to yield compound 247 as an orange foam. Crude compound 247 was taken on directly to the next reaction. HPLC: 100% at 2.70 min (retention time) (YMC S5 ODS column 4.6×50 mm, 10-90% aqueous methanol over 4 minutes containing 0.2% phosphoric acid, 4 mL/min, monitoring at 220 nm), MS (ES): m/z 483.65 [M+H]⁺.

### EXAMPLE 248

### [3aα,4β(E),7β,7aα]-4-[4-[3-(4-Cyanophenyl)-2-propenyl]octahydro-7-methyl-1,3-dioxo-4,7-epoxy-2H-isoindol-2-yl]-1-naphthalenecarbonitrile & [3aα,4β(Z),7β,7aα]-4-[4-[3-(4-Cyanophenyl)-2-propenyl]octahydro-7-methyl-1,3-dioxo-4,7-epoxy-2H-isoindol-2-yl]-1-naphthalenecarbonitrile (248i & 248ii)

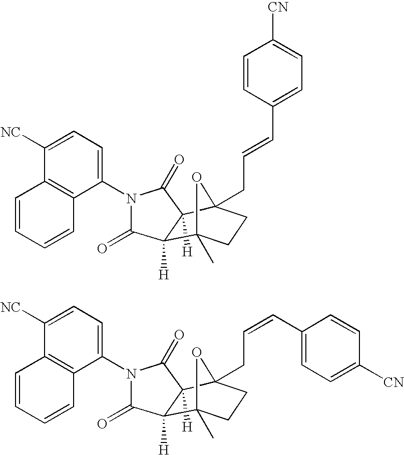

(4-cyanobenzyl)-triphenylphosphonium chloride (0.072 g, 0.174 mmol) was suspended in THF (2.0 mL) and cooled to 0° C. n-BuLi (1.6 M soln, 0.092 mL, 0.147 mmol) was then added dropwise resulting in a homogenous solution. The solution warmed to 25° C. for 15 min and then cooled to 0° C. Compound 247 (0.050 g, 0.134 mmol) was then added in THF. After 1 h, the reaction was quenched with sat. aq ammonium chloride and then extracted with methylene chloride (3×20 mL). The combined organics were dried over anhydrous sodium sulfate and then concentrated in vacuo. The crude material was purified by preparative TLC eluting with 5% acetone in chloroform to give 0.010 g of a mixture of compounds 248i & 248ii as a white solid. A 1:1 mixture of E and Z olefin isomers characterized by NMR spectroscopy. HPLC: 100% at 3.517 min (retention time) (YMC S5 ODS column 4.6×50 mm, 10-90% aqueous methanol over 4 minutes containing 0.2% phosphoric acid, 4 mL/min, monitoring at 220 nm), MS (ES): m/z 474.2 [M+H]⁺.

### EXAMPLE 249

### (3aα,4β,7β,7aα)-4-[4-[3-(4-Cyanophenyl)propyl]octahydro-7-methyl-1,3-dioxo-4,7-epoxy-2H-isoindol-2-yl]-1-naphthalenecarbonitrile, (249)

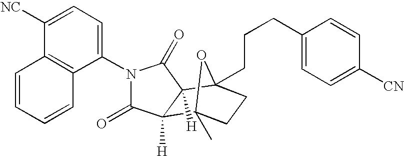

The mixture of compounds 248i & 248ii (0.008 g, 0.017 mmol) was dissolved in EtOH (3.0 mL) and Pd/C (10% Pd, 0.008 g) was added. H₂ was then introduced via a balloon. After 18 h, the reaction was filtered through celite, eluting with EtOAc, followed by concentration in vacuo. Compound 249 was isolated as a white solid (0.007 g). HPLC: 90% at 3.520 min (retention time) (YMC S5 ODS column 4.6×50 mm, 10-90% aqueous methanol over 4 minutes containing 0.2% phosphoric acid, 4 mL/min, monitoring at 220 nm), MS (ES): m/z 476.13 [M+H]⁺.

### EXAMPLE 250

### (3aα,4β,7β,7aα)-4-[4-[2-[(6-Chloro-1,2-benzisoxazol-3-yl)oxy]ethyl]octahydro-7-methyl-1,3-dioxo-4,7-epoxy-2H-isoindol-2-yl]-1-naphthalenecarbonitrile, (250)

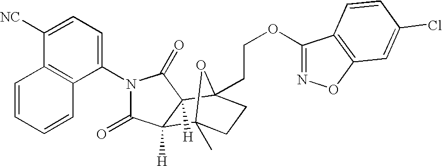

To a solution of PPh₃ (52 mg, 0.20 mmol) in 0.5 mL THF was added DBAD (46 mg, 0.20 mmol) as one solid portion. The resulting mixture was stirred for 10 min before 6-chloro-3-hydroxy-1,2-benzisoxazole (34 mg, 0.20 mmol) was added. Stirring was continued for 10 min before a solution of compound 223B (50 mg, 0.13 mmol) in 0.5 mL THF was introduced via canula. The resulting mixture was stirred at ambient temperature for 24 h, concentrated and purified by preparative reverse phase HPLC (YMC S5 ODS 20×100 mm column; eluting with 30-100% aqueous MeOH containing 0.1% TFA over 10 min at 20 mL/min) to yield a white solid. The obtained solids were dissolved in CH₂Cl₂, washed with sat. NaHCO₃ solution, dried over Na₂SO₄ and concentrated to yield 50 mg (71%) of compound 250 as a colorless oil. HPLC: 26% at 3.89 min and 74% at 4.02 min (mixture of atropisomers, retention time) (YMC S5 ODS column 4.6×50 mm Ballistic, 10-90% aqueous methanol over 4 minutes containing 0.2% H₃PO₄, 4 mL/min, monitoring at 220 nm), MS (ES): m/z 528.4 [M+H]⁺.

### EXAMPLE 251

### (3aα,4β,7β,7aα)-4-[Octahydro-4-methyl-7-[2-[(6-nitro-1H-indazol-3-yl)oxy]ethyl]-1,3-dioxo-4,7-epoxy-2H-isoindol-2-yl]-1-naphthalenecarbonitrile, (251)

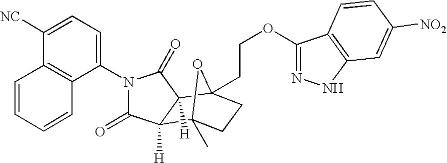

To a solution of compound 223B (50 mg, 0.13 mmol) in toluene (1 mL) was added ADDP (50 mg, 0.20 mmol), 6-nitro-3-indazolinone (36 mg, 0.20 mmol) and n-Bu₃P (50 μL, 0.2 mmol). The resulting mixture was heated to 80° C. for 24 h, concentrated and purified by a combination of preparative reverse phase HPLC (YMC S5 ODS 20×100 mm column; eluting with 30-100% aqueous MeOH containing 0.1% TFA over 10 min at 20 mL/min) and flash chromatography (silica gel, 25% acetone in CHCl₃) to give 17 mg (25%) of compound 251 as a yellow solid. HPLC: 24% at 3.60 min and 76% at 3.74 min (mixture of atropisomers, retention time) (YMC S5 ODS column 4.6×50 mm Ballistic, 10-90% aqueous methanol over 4 minutes containing 0.2% H₃PO₄, 4 mL/min, monitoring at 220 nm), MS (ES): m/z 537.6 [M+H]⁺.

### EXAMPLE 252

### [3aS-(3aα,4β,5β,7β,7aα)]-4-[7-[2-(1,2-Benzisoxazol-3-yloxy)ethyl]octahydro-5-hydroxy-4-methyl-1,3-dioxo-4,7-epoxy-2H-isoindol-2-yl]-1-naphthalenecarbonitrile, (252)

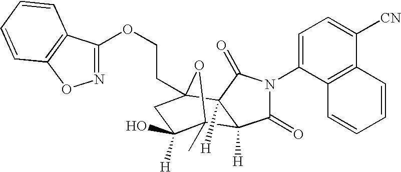

PPh₃ (47 mg, 0.18 mmol), DBAD (41 mg, 0.18 mmol), 3-hydroxy-1,2-benzisoxazole (24 mg, 0.18 mmol) and compound 243Di (35 mg, 0.09 mmol) were reacted according to the procedure given for compound 250. Purification was achieved by reverse phase HPLC (YMC S5 ODS 20×100 mm column; eluting with 30-100% aqueous MeOH containing 0.1% TFA over 10 min at 20 mL/min) to yield a white solid. The obtained solids were dissolved in CH₂Cl₂, washed with sat. NaHCO₃ solution, dried over Na₂SO₄ and concentrated finishing 29 mg (64%) of compound 252 as a colorless oil. HPLC: 96% at 3.29 min (mixture of atropisomers, retention time) (YMC S5 ODS column 4.6×50 mm Ballistic, 0-100% aqueous methanol over 4 minutes containing 0.2% H₃PO₄, 4 mL/min, monitoring at 220 nm), MS (ES): m/z 510.2 [M+H]⁺.

### EXAMPLE 253

### [3aR-(3aα,4β,5β,7β,7aα)]-4-[7-[2-(1.2-Benzisoxazol-3-yloxy)ethyl]octahydro-5-hydroxy-4-methyl-1,3-dioxo-4,7-epoxy-2H-isoindol-2-yl]-1-naphthalenecarbonitrile, (253)

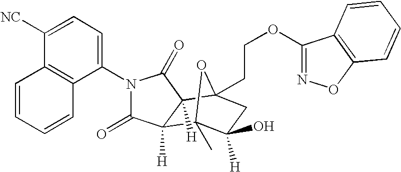

PPh₃ (47 mg, 0.18 mmol), DBAD (41 mg, 0.18 mmol), 3-hydroxy-1,2-benzisoxazole (24 mg, 0.18 mmol) and compound 243Dii (35 mg, 0.09 mmol) were reacted according to the procedure given for compound 250. Purification was achieved by reverse phase HPLC (YMC S5 ODS 20×100 mm column; eluting with 30-100% aqueous MeOH containing 0.1% TFA over 10 min at 20 mL/min) to yield a white solid. The obtained solids were dissolved in CH₂Cl₂, washed with sat. NaHCO₃ solution, dried over Na₂SO₄ and concentrated furnishing 23 mg (51%) of compound 253 as a colorless oil. HPLC: 95% at 3.29 min (mixture of atropisomers, retention time) (YMC S5 ODS column 4.6×50 mm Ballistic, 0-100% aqueous methanol over 4 minutes containing 0.2% H₃PO₄, 4 mL/min, monitoring at 220 nm), MS (ES): m/z 510.4 [M+H]⁺.

### EXAMPLE 254

### (3aα,4β,5β,7β,7aα)]-4-(Octahydro-5-hydroxy-4,7-dimethyl-1,3-dioxo-4,7-epoxy-2H-isoindol-2-yl)-2-(trifluoromethyl)benzonitrile & (3aα,4β,5β,7β,7aα)]-4-(Octahydro-5-hydroxy-4,7-dimethyl-1.3-dioxo-4,7-epoxy-2H-isoindol-2-yl)-2-(trifluoromethyl)benzonitrile, (254i & 254ii)

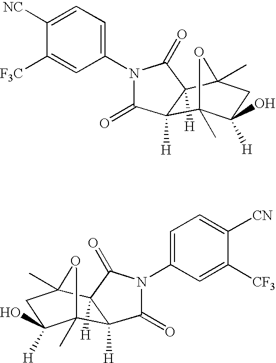

Racemic compound 221B was separated into its enantiomers by preparative chiral HPLC (CHIRALPAK AD 5×50 cm column; eluting with 20% MeOH/EtOH (1:1) in heptane (isocratic) at 50 mL/min) to give the faster eluting compound 254i (Chiral HPLC: 10.02 min; CHIRALPAK AD 4.6×250 mm column; eluting with 20% MeOH/EtOH (1:1) in heptane at 1 mL/min) and the slower eluting 254ii (Chiral HPLC: 14.74 min; CHIRALPAK AD 4.6×250 mm column; eluting with 20% MeOH/EtOH (1:1) in heptane at 1 mL/min).

### EXAMPLE 255

### (3aα,4β,7β,7aα)-2-(4-Cyano-1-naphthalenyl)octahydro-1,3-dioxo-7-[2-(phenylmethoxy)ethyl]-4,7-epoxy-4H-isoindole-4-propanenitrile & (3aα,4α,7α,7aα)-2-(4-Cyano-1-naphthalenyl)octahydro-1,3-dioxo-7-[2-(phenylmethoxy)ethyl]-4,7-epoxy-4H-isoindole-4-propanenitrile, (255Hi & 255Hii)

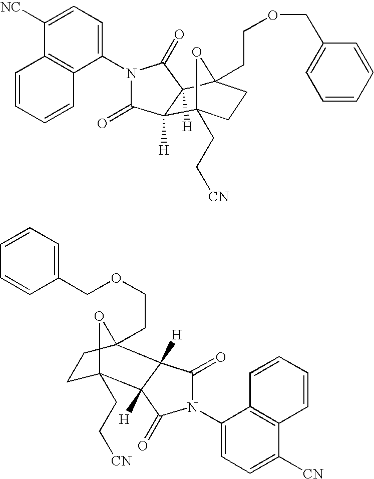

A. 2-(2-Hydroxyethyl)furan (255A)

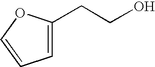

2-(2-Hydroxyethyl)furan was made in accordance with the following reference: Harmata, M, et al. J. Org. Chem. 60, 5077-5092 (1995). n-BuLi (2.5 M in hexane, 44 mL, 110 mmol) was added to a solution of furan (8 mL, 110 mmol) in 100 mL of THF at −78° C. The solution was stirred at 0° C. for 4 h and then ethylene oxide (7.5 mL) was added at −78° C. The reaction mixture was stirred at −15° C. for 1 h and then overnight at rt. The reaction was quenched with sat. NH₄Cl and extracted with ether (3×). The combined extracts were washed with water (1X) and brine (1×). The ether solution was dried over Na₂SO₄ and concentrated under reduced pressure. Purification by flash chromatography on silica gel eluting with 40% EtOAc/60% hexane gave 5.4 g (48.2 mmol, 43.8%) of compound 255A as a light brown oil.

B. 2-[2-[[(1,1-Dimethlethyl)dimethylsilyl]oxy]ethyl]furan (255B)

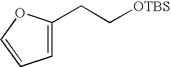

Imidazole (3.65 g, 53.6 mmol) and TBSCl (6.47 g, 42.9 mmol) were added to the solution of compound 255A (4.00 g, 35.7 mmol) in 50 mL of DMF. The mixture was stirred at rt for 2 h and then the reaction mixture was poured into ether. The ether solution was washed with water (1×), 1 N HC1 (1×), water (1×) and brine (1×). The organic layer was dried over Na₂SO₄ and concentrated under reduced pressure. Purification by flash chromatography on silica gel eluting with 30% CH₂Cl₂/70% hexane gave 7.4 g (32.7 mmol, 91.7%) of 255B as a colorless oil.

C. 2-[2-[[(1,1-Dimethlethyl)dimethylsilyl]oxy]ethyl]-5-(2-hydroxyethyl)furan (255C)

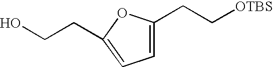

t-BuLi (1.2 M in pentane, 10 mL, 16.99 mmol) was added to a stirred solution of 255B (3.49 g, 15.44 mmol) in 13 mL of THF at −78° C. dropwise. The mixture was stirred for an additional 4 h at 0° C. Ethylene oxide (1.05 mL) was added at −78° C. to the reaction solution. The mixture was warmed to rt and stirred overnight. Aqueous sat. NH₄Cl was added and most of the THF was removed under reduced pressure. The mixture was extracted with ether (3×) and the combined organic layers were washed with water (1×) and brine (1×) and dried over Na₂SO₄. Purification by flash chromatography on silica gel eluting with 5% EtOAc/95% CH₂Cl₂ gave 2.8 g (10.4 mmol, 67%) of compound 255C as a yellow oil.

D. 2-[2-[[(1,1-Dimethlethyl)dimethylsilyl]oxy]ethyl]-5-[2-(phenylmethoxy)ethyl]furan (255D)

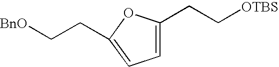

The alcohol 255C (1.00 g, 3.7 mmol) in 12 mL of THF was treated with 60% NaH (177.8 mg, 4.44 mmol), benzyl bromide (0.53 mL, 4.44 mmol) and tetrabutylammonium iodide (50 mg, 5%) for 3 h at rt. Water was added and the mixture was extracted with EtOAc (3×). The combined extracts were washed with water (1×) and brine (1×) and dried over Na₂SO₄. Purification by flash chromatography on silica gel eluting with 20% hexane/80% CH₂Cl₂ gave 1.10 g (3.05 mmol, 82.6%) of compound 255D as a yellow oil.

E. 2-(2-Hydroxyethyl)-5-[2-(phenylmethoxy)ethyl]furan (255E)

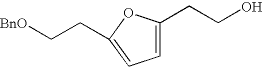

Tetrabutylammonium fluoride (1.0M in THF, 3.06 mL, 3.06 mmol) was added to the solution of compound 255D (1.1 g, 3.06 mmol) in 10 mL of THF at 0° C. The reaction mixture was stirred at rt for 10 minutes, quenched by sat. NH₄Cl and extracted with ether (3×). The combined extracts were dried over Na₂SO₄. Purification by flash chromatography on silica gel eluting with 10% EtOAc/90% CH₂Cl₂ gave 750 mg (3.05 mmol, 99.6%) of compound 255E as a light yellow oil.

F. 5-[2-(Phenylmethoxy)ethyl]furan-2-propanenitrile (255F)

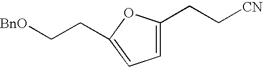

DEAD (1.285 mL, 8.17 mmol) was added to a stirred solution of Ph₃P (2.14 g, 8.17 mmol) in 12 mL of dry THF at 0° C. The solution was stirred for 30 min at rt and compound 255E (670 mg, 2.72 mmol) was added. The reaction was stirred for 15 min and acetone cyanohydrin (0.745 mL, 8.17 mmol) was added at −15° C. The reaction was stirred for 30 min at −15° C., then at rt overnight. The mixture was then concentrated under reduced pressure. Purification by flash chromatography on silica gel eluting with 100% CH₂Cl₂ gave 180 mg (0.705 mmol, 26%) of compound 255F as a colorless oil.

G. (3aα,4β,7β,7aα)-2-(4-Cyano-1-naphthalenyl)-1,2,3,3a,7,7a-hexahydro-1,3-dioxo-7-[2-(phenylmethoxy)ethyl]-4,7-epoxy-4H-isoindole-4-propanenitrile (255G)

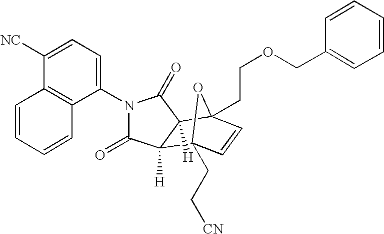

A solution of compound 255F (180 mg, 0.706 mmol) and 4-(2,5-dihydro-2,5-dioxo-1H-1-yl)-1-naphthalenecarbonitrile (263 mg, 1.06 mmol) in CH₂Cl₂ (3 mL) was stirred at rt for 3 days. The reaction mixture was concentrated under reduced pressure. Purification by flash chromatography on silica gel eluting with 5% EtOAc/CH₂Cl₂ gave 318 mg (0.63 mmol, 89.6%) of compound 255G as a light gray solid which was used directly in the next step.

H. (3aα,4β,7β,7aα)-2-(4-Cyano-1-naphthalenyl)octahydro-1,3-dioxo-7-[2-(phenylmethoxy)ethyl]-4,7-epoxy-4H-isoindole4-propanenitrile & (3aα,4α,7α,7aα)-2-(4-Cyano-1-naphthalenyl)octahydro-1,3-dioxo-7-[2-(phenylmethoxy)ethyl]4,7-epoxy-4H-isoindole-4-propanenitrile (255Hi & 255Hii)

A mixture of compound 255G (318 mg, 0.63 mmol) and 10% Pd/C (64 mg) in EtOH (10 mL) and EtOAc (5 mL) was stirred under a hydrogen atmosphere at rt overnight. The reaction mixture was filtered through celite and concentrated under reduced pressure to give 320 mg of crude compounds 255Hi & 255Hii. Purification of 25 mg of this crude product by flash chromatography on silica gel eluting with 55% EtOAc/hexane gave 6.5 mg (0.013 mmol, 26% (based on 25 mg)) of compound 255Hi & 8.1 mg (0.016 mmol, 32.4% (based on 25 mg)) of compound 255Hii. Compound 255Hi: HPLC conditions: 98% at 3.57 min (YMC S5 ODS 4.6×50 mm, 10%-90% aqueous methanol over 4 minute gradient with 0.2% H₃PO₄, detecting at 220 nm, MS (ES): m/z 506.15 [M+H]⁺. Compound 255Hii: HPLC conditions: 98% at 3.51 min (YMC S5 ODS 4.6×50 mm, 10%-90% aqueous methanol over 4 minute gradient with 0.2% H₃PO₄, detecting at 220 nm), MS (ES): m/z 506.15 [M+H]⁺.

### EXAMPLE 256

### (3aα,4β,7β,7aα)-2-(4-Cyano-1-naphthalenyl)octahydro-7-(2-hydroxyethyl)-1,3-dioxo4,7-epoxy-4H-isoindole-4-propanenitrile & (3aα,4α,7α,7aα)-2-(4-Cyano-1-naphthalenyl)octahydro-7-(2-hydroxyethyl)-1,3-dioxo-4,7-epoxy-4H-isoindole-4-propanenitrile, (256i & 256ii)

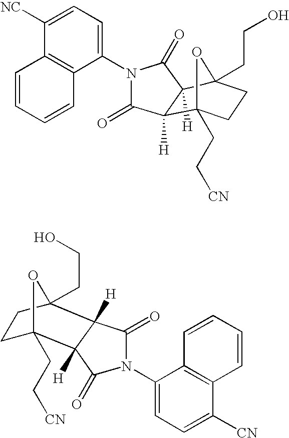

A mixture of compounds 255Hi & 255Hii (200 mg, 0.396 mmol) and PdCl₂ (8.4 mg, cat.) in EtOH (1 mL) and EtOAc (3 mL) was stirred under a hydrogen atmosphere (30 psi) at rt overnight. The reaction mixture was filtered through celite and concentrated under reduced pressure. Purification by flash chromatography on silica gel eluting with 5% MeOH/CH₂Cl₂ followed by a second column eluting with 100% EtOAc gave 28.9 mg (0.0696 mmol, 17.6%) of compound 256ii and 26.5 mg (0.0639 mmol, 16.1%) of compound 256i. Compound 256ii: HPLC conditions: 90% at 2.44 min (YMC S5 ODS 4.6×50 mm, 10%-90% aqueous methanol over 4 minute gradient with 0.2% H₃PO₄, detecting at 220 nm.), MS (ES): m/z 416.11 [M+H]⁺. Compound 256i: HPLC conditions: 99% at 2.47 min (YMC S5 ODS 4.6×50 mm, 10%-90% aqueous methanol over 4 minute gradient with 0.2% H₃PO₄, detecting at 220 nm), MS (ES): m/z 416.11 [M+H]⁺.

### EXAMPLE 257

### (3aα,4β,7β,7aα)-2-(4-Cyano-1-naphthalenyl)-7-[2-(4-fluorophenoxy)ethyl]octahydro-1,3-dioxo-4,7-epoxy4H-isoindole-4-propanenitrile. (257)

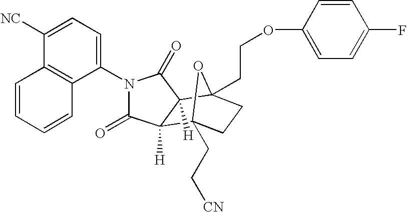

DBAD (15 mg, 0.065 mmol) was added to a solution of PPh₃ (17 mg, 0.065 mmol) in THF (0.3 mL). After stirring for 10 mins, 4-fluorophenol (7.33 mg, 0.065 mmol) was added and the reaction mixture was stirred for a further 5 mins. Compound 256i (18.1 mg, 0.044 mmol) was added and the mixture was stirred at rt for 3 h. The reaction was concentrated under reduced pressure. Purification by flash chromatography on silica gel eluting with 60% EtOAc/30% hexane gave 5.9 mg (0.0116 mmol, 26.34%) of compound 257. HPLC conditions: 98% at 3.59 min (YMC S5 ODS 4.6×50 mm, 10%-90% aqueous methanol over 4 minute gradient with 0.2% H₃PO₄, detecting at 220 nm). MS (ES): m/z 510.14 [M+H]⁺.

### EXAMPLE 258

### (3aα,4β,7β,7aα)-2-(7-Chloro-2,1,3-benzoxadiazol-4-yl)hexahydro-4,7-dimethyl-4,7-epoxy-1H-isoindole-1,3(2H)-dione, (258)

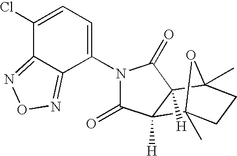

A. 4-Amino-7-chloro-2,1,3-benzoxadiazole (258A)

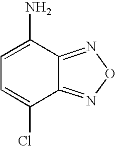

A solution of 1.0 g (5.02 mmol) of 4-chloro-7-nitrobenzofurazan in 20 mL AcOH, 10 mL EtOAc and 2 mL H₂O was heated to 50° C. and treated with iron powder (1.4 g, 251 mmol). The mixture was heated at 80° C. for 30 min and then allowed to cool to rt. The mixture was filtered through celite eluting with EtOAc. The filtrate was washed with sat. aq NaHCO₃, dried over MgSO₄, and concentrated to give compound 258A (0.80 g, 94%) as a red solid.

B. (3aα,4β,7β,7aα)-2-(7-Chloro-2,1,3-benzoxadiazol-4-yl)hexahydro-4,7-dimethyl-4,7-epoxy-1H-isoindole-1,3(2H)-dione, (258B)

Compound 258A (42 mg, 0.25 mmol) was reacted in a sealed tube with compound 20A (73.5 mg, 0.375 mmol), MgSO₄ (75 mg, 0.625 mmol) and Et₃N (170 μL, 1.25 mmol) in 250 μL toluene according to the above procedure described in example 208C to give after purification by preparative reverse phase HPLC (YMC S5 ODS 20×100 mm eluting with 30-100% aqueous methanol containing 0.1% TFA over 12 min, 20 mL/min) 23 mg (26%) of compound 258B as a yellow solid. HPLC: 97.6% at 2.87 min (retention time) (YMC S5 ODS column 4.6×50 mm eluting with 10-90% aqueous methanol containing 0.2% phosphoric acid over 4 minutes, 4 mL/min, monitoring at 220 nm), MS (DCl): m/z 347.9 [M]⁺.

### EXAMPLE 259

### (3aα,4β,7β,7aα)-2-(7-Chloro-2-methyl-4-benzofuranyl)hexahydro-4,7-dimethyl-4,7-epoxy-1H-isoindole-1,3(2H)-dione, (259)

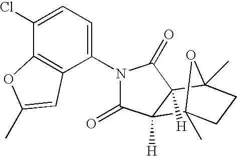

7-Chloro-2-methyl-4-benzofuranamine (38 mg, 0.25 mmol, prepared in accordance with the procedure described by Enomoto and Takemura in EP 0476697 A1) was reacted in a sealed tube with compound 20A (73.5 mg, 0.375 mmol), MgSO₄ (75 mg, 0.625 mmol) and Et₃N (170 μL, 1.25 mmol) in 250 μL toluene according to the procedure described in example 208C to give, after purification by preparative reverse phase HPLC (YMC S5 ODS 20×100 mm eluting with 30-100 aqueous methanol containing 0.1% TFA over 12 min, 20 mL/min), 42 mg (47%) of compound 259 as a white solid. HPLC: 98% at 3.45 min (retention time) (YMC S5 ODS column 4.6×50 mm eluting with 10-90% aqueous methanol over 4 minutes containing 0.2% phosphoric acid, 4 mL/min, monitoring at 220 nm), MS (DCl): m/z 359.9 [M]⁺.

### EXAMPLE 260

### (3aα,4β,7β,7aα-2-(7-Chloro-2-methylbenzo[b]thiophen-4-yl)hexahydro-4,7-dimethyl-4,7-epoxy-1H-isoindole-1,3(2H)-dione, (260)

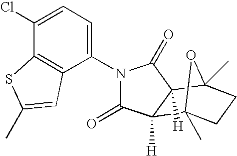

A. 1-Chloro-2-(2-chloro-allylsulfanyl)-4-nitro-benzene (260A)

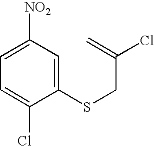

A solution of 2-chloro-5-nitro-benzenethiol (1.0 g, 5.27 mmol, prepared in accordance with the procedure described by Still et. al., Synth. Comm. 13, 1181 (1983)) in 15 mL DMF was treated with 2,3-dichloropropene (693 μL, 7.52 mmol) and K₂CO₃ (433 mg, 3.13 mmol). The mixture was heated at 80° C. for 2 h and then allowed to cool to rt. EtOAc (200 mL) and H₂O (100 mL) were added. The organic phase was washed with H₂O (2×250 mL), saturated aqueous NaCl (100 mL), dried over MgSO₄, and concentrated. The crude material was purified by flash column chromatography on silica gel eluting with 20% EtOAc in hexanes to give compound 260A (1.09 g, 89%) as an orange oil.

B. 4-Amino-7-chloro-2-methylbenzo[b]thiophene (260B)

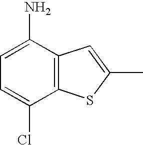

A solution of 1.09 g (4.67 mmol) of compound 260A in 20 mL AcOH with 10 mL EtOAc and 2 mL H₂O was heated to 80° C. and treated with iron powder (1.3 g, 23.4 mmol). The mixture was heated at 80° C. for 40 min and then allowed to cool to rt. The mixture was filtered through celite eluting with EtOAc. The filtrate was washed with sat. aq NaHCO₃, dried over MgSO₄, and concentrated in vacuo. N,N-diethylaniline (10 mL) was added, and the reaction was heated at 215° C. for 6 h. After cooling to rt, 1N aqueous HCl (20 mL) was added, and the reaction was stirred at room temperature for 2 h. The mixture was extracted with EtOAc (3×30 mL). The organic phase was washed with saturated aqueous NaHCO₃, dried over MgSO₄, and concentrated. The crude material was purified by flash column chromatography on silica gel eluting with 25% EtOAc in hexanes to give compound 260B (320 mg, 35%) as a beige solid.

C. (3aα,4β,7β,7aα)-2-(7-Chloro-2-methylbenzo[b]thiophen-4-yl)hexahydro-4,7-dimethyl-4,7-epoxy-1H-isoindole-1,3(2H)-dione, (260C)

Compound 260B (49 mg, 0.25 mmol) was reacted in a sealed tube with compound 20A (73.5 mg, 0.38 mmol), MgSO₄ (75 mg, 0.63 mmol) and Et₃N (170 μL, 1.25 mmol) in 250 μL toluene according to the procedure described in example 208C to give, after purification by preparative reverse phase HPLC (YMC S5 ODS 20×100 mm eluting with 30-100% aqueous methanol over 12 min containing 0.1% TFA, 20 mL/min), 28 mg (30%) of compound 260C as a pale yellow solid. HPLC: 96% at 3.18 min (retention time) (YMC S5 ODS column 4.6×50 mm eluting with 10-90% aqueous methanol over 4 minutes containing 0.2% phosphoric acid, 4 mL/min, monitoring at 220 nm), MS (DCl): m/z 376.0 [M]⁺.

### EXAMPLE 261

### [3aα,4β,7β,7aα]-4-[2-(4-Cyano-1-naphthalenyl)octahydro-7-methyl-1,3-dioxo-4,7-epoxy-4H-isoindol-4-yl]-2-butenoic acid, phenylmethyl ester, (261)

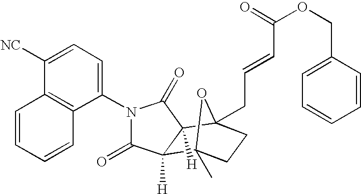

Compound 247 (0.500 g, 0.134 mmol) was dissolved in THF (20 mL) and benzyl(triphenylphosphoranylidene) (0.55 g, 0.134 mmol) was added. The reaction mixture was stirred at 67° C. for 2 h and then concentrated under reduced pressure. Purification by flash chromatography on SiO₂ eluting with 5% acetone/95% CHCl₃ gave 0.65 g of compound 261 as a yellow solid. HPLC: 99% at 3.717 min (retention time) (YMC S5 ODS column 4.6×50 mm eluting with 10-90% aqueous methanol over 4 minutes containing 0.2% phosphoric acid, 4 mL/min, monitoring at 220 nm), MS (ES): m/z 507.1 [M+H]⁺.

### EXAMPLE 262

### (3aα,4β,7β,7aα)-2-(4-Cyano-1-naphthalenyl)octahydro-7-methyl-1,3-dioxo-4,7-epoxy-4H-isoindole-4-butanoic acid, (262)

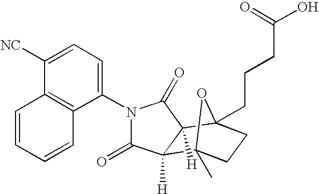

Compound 261 (0.60 g, 1.19 mmol) was dissolved in EtOH/EtOAc (5 mL/5 mL) and 10% Pd/C (0.30 g) was added. Hydrogen was then introduced via a balloon. After 8 h the reaction was filtered through celite and then concentrated under reduced pressure to give compound 262 (0.47 g) as a white solid. HPLC: 98% at 2.81 min (retention time) (YMC S5 ODS column 4.6×50 mm eluting with 10-90% aqueous methanol over 4 minutes containing 0.2% phosphoric acid, 4 mL/min, monitoring at 220 nm), MS (ES): m/z 419.1 [M+H]⁺.

### EXAMPLE 263

### (3aα,4β,7β,7aα)-2-(4-Cyano-1-naphthalenyl)-N-(4-fluorophenyl)octahydro-7-methyl-1,3-dioxo-4,7-epoxy-4H-isoindole-4-butanamide, (263)

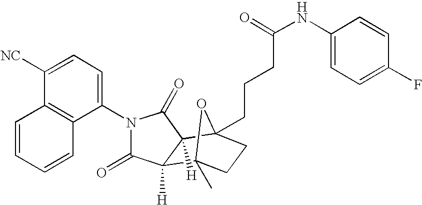

Compound 262 (0.030 g, 0.072 mmol) was dissolved in CH₃CN (1 mL). DCC (0.014 g, 0.072 mmol) and HOAc (0.0098 g, 0.072 mmol) were then added, followed by 4-flouroaniline (0.007 mL, 0.072 mmol). The reaction mixture was stirred under argon for 14 h and the crude material was dissolved in MeOH, purified by preparative HPLC (YMC VP-ODS column, 20×100 mm, eluting with 20% B to 100% B in 15 minutes and hold @ 100% B for 10 minutes). Compound 263 (0.020 g) was isolated as white solid. HPLC: 100% at 3.217 min (retention time) (YMC S5 ODS column 4.6×50 mm eluting with 10-90% aqueous methanol over 4 minutes containing 0.2% phosphoric acid, 4 mL/min, monitoring at 220 nm), MS (ES): m/z 512.1 [M+H]⁺.

### EXAMPLE 264

### [3aS-(3aα,4β,5β,7β,7aα)]-4-[7-[2-(Acetyloxy)ethyl]octahydro-5-hydroxy-4-methyl-1,3-dioxo-4,7-epoxy-2H-isoindol-2-yl]-1-naphthalenecarbonitrile & [3aR-(3aα,4β,5β,7β,7aα)]-4-[Octahydro-5-hydroxy-7-(2-hydroxyethyl)-4-methyl-1,3-dioxo-4,7-epoxy-2H-isoindol-2-yl]-1-naphthalenecarbonitrile, (264 & 243Dii)

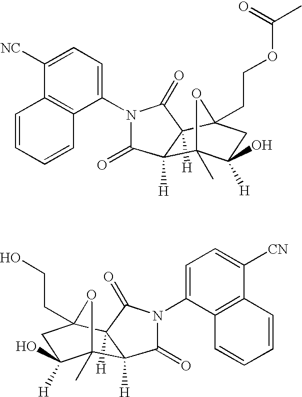

A racemic mixture of compounds 243Di & 243Dii (1.90 gram) were dissolved in 100 mL of anhydrous THF in a 2 L flask. Anhydrous tert-butyl-methyl ether (900 mL) and vinyl acetate (40 mL) were transferred into the flask with stirring and lipase (20 g, typeII, crude, from porcine pancreas; Sigma, Cat# L3126) was added. The reaction mixture was stirred for 21 hr at rt at which point an additional 5 grams of the lipase and 20 mL of vinyl acetate were added. The reaction was stirred at rt for an additional 19 h, stored at 4° C. without stirring for 36 h and then stirred at rt for another 22 h (until the desired % ee was apparent by chiral HPLC). To monitor the reaction, 200 uL of the mixture was withdrawn and centrifuged. The supernatant (100 uL) was dried under nitrogen and the resulting residue was dissolved in 100 uL of EtOH and subjected to HPLC analysis:

1) Reverse phase HPLC: Column, YMC-ODS AQ 150×4.6; flow rate, 1.2 mL/min; sample size, 10 uL solvent A,: 1 mM HCl in water; solvent B, MeCN; monitored at 300 nm Gradient: Time(min) 0 8 8.5 9.5 10 12 B% 30 60 85 85 30 30

2) Chiral-HPLC: Column, CHIRALCEL OJ 4.6×250 mm mobile phase, Hexane/MeOH/EtOH (8:1:1) flow rate, 1 mL/min; sample size, 20 uL monitored at both 220 and 300 nm performed at 25° C. & 40° C. (for ee% determination of reaction mixture)

The enzyme was removed by filtration and filtrate was concentrated under vacuum. The resulting mixture was dissolved in CHCl₃ and adsorbed onto silica gel (63-200 microns). These solids were applied to a VLC funnel (3 cm I.D., VLC is vacuum liquid chromatography using glass funnels having 24/40 joints at the bottom) containing a 5 cm bed height of silica gel (25-40 microns) and a step gradient was carried out. The gradient was 100% CHCl₃ in the first 3 fractions, followed by CHCl₃-1% MeOH (3 fractions), CHCl₃-2% MeOH (3 fractions), CHCl₃-3% MeOH (3 fractions), CHCl₃-4% MeOH (3 fractions), and finally with CHCl₃-5% MeOH (3 fractions). The volume of the fractions was 100 mL until reaching CHCl₃-3% MeOH and from that point on it was 200 mL. Compound 264 elutes in the last two fractions of 100% CHCl₃ and until the first fraction of CHCl₃-2% MeOH. Compound 243Dii elutes starting with the second fraction of CHCl₃-2% MeOH, and continues to the first fraction of CHCl₃-5% MeOH. The crude compound 243Dii contained a small amount of a colored impurity which was removed by a Sephadex collumn [LH-20 swollen in CHCl₃-MeOH (2:1), column (2.5 cm I.D. & 90 cm long) to yield 632 mg of compound 243Dii. Compound 264: HPLC conditions: 98% at 7.2 min (method 1), chiral HPLC conditions: 29.0 min @ 25° C. (method 2). Compound 243Dii: HPLC conditions: 98% at 4.6 min (method 1), chiral HPLC conditions: 96% ee at 25.7 min (@ 25° C.) & 19.8 min (@ 40° C.) (method 2).

### EXAMPLE 265

### (3aα,4β,7β,7aα(E)1-4-[Octahydro-4-methyl-1,3-dioxo-7-(4-oxo-4-phenyl-2-butenyl)-4,7-epoxy-2H-isoindol-2-yl]-1-naphthalenecarbonitrile, (265)

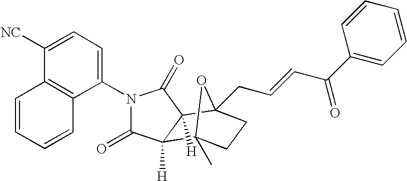

The compound 247 (0.050 g, 0.134 mmol) was dissolved in THF (1.5 mL) and (phenacylidene)triphenylphosphorane (0.051 g, 0.134 mmol) was added. The reaction mixture was stirred at 67° C. for 24 h and then cooled to 23° C. and concentrated in vacuo. The crude material was then purified by preparative HPLC. (YMC VP-ODS column, 20×100 mm, eluting with 20% B to 100% B in 15 minutes and hold @ 100% B for 10 minutes.) to give compound 265 (0.040 g) as white solid. HPLC: 100% at 3.503 min (retention time) (YMC S5 ODS column 4.6×50 mm eluting with 10-90% aqueous methanol over 4 minutes containing 0.2% phosphoric acid, 4 mL/min, monitoring at 220 nm), MS (ES): m/z 477.1 [M+H]⁺.

### EXAMPLE 266

### (3aα,4β,7β,7aα(E)]-4-[Octahydro-4-methyl-1,3-dioxo-7-(4-oxo-4-phenyl-2-butenyl)-4,7-epoxy-2H-isoindol-2-yl]-1-naphthalenecarbonitrile, (266)

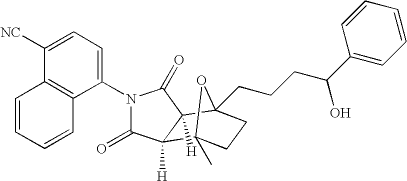

Compound 265 (0.010 g, 0.021 mmol) was dissolved in EtOH (2.0 mL) and Pd/C (10% Pd, 0.005 g) was added. Hydrogen was then introduced via a balloon and the reaction was stirred at 25° C. for 3 h. The reaction was then filtered through celite rinsing with EtOAc and concentrated in vacuo to give compound 266 as a tan solid (0.009 g). No purification was necessary. HPLC: 100% at 3.38 min (retention time) (YMC S5 ODS column 4.6×50 mm eluting with 10-90% aqueous methanol over 4 minutes containing 0.2% phosphoric acid, 4 mL/min, monitoring at 220 nm), MS (ES): m/z 503.2 [M+Na]⁺.

### EXAMPLES 267 to 378

Additional compounds of the present invention were prepared by procedures analogous to those described above. The compounds of Examples 267 to 378 have the following structure (L is a bond):

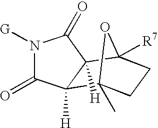

where G, R⁷, the compound name, retention time, molecular mass, and the procedure employed, are set forth in Table 5. The absolute configuration for the following compounds was not determined. For simplicity in nomenclature, compound 238i is designated herein as having an “R” configuration and compound 238ii as having an “S” configuration. Enantiomerically pure products derived from compound 238i are designated herein as having an “R” configuration and enantiomerically pure products derived from compound 238ii are designated herein as having an “S” configuration.

The chromatography techniques used to determine the compound retention times of Table 5 are as follows: LCMS=YMC S5 ODS column, 4.6×50 mm eluting with 10-90% MeOH/H₂O over 4 minutes containing 0.1% TFA; 4 mL/min, monitoring at 220 nm. LCMS*=YMC S5 ODS column, 4.6×50 mm eluting with 10-90% MeOH/H₂O over 2 minutes containing 0.1% TFA; 4 mL/min, monitoring at 220 nm. LC=YMC S5 ODS column 4.6×50 mm eluting with 10-90% MeOH/H₂O over 4 minutes containing 0.2% phosphoric acid, 4 mL/min, monitoring at 220 nm. The molecular mass of the compounds listed in Table 5 were determined by MS (ES) by the formula m/z.

TABLE 5

| | | | | Retention | |
|---|---|---|---|---|---|
| | | | | Time | |
| | | | | Min./ | Procedure |
| Ex. | | | Compound | Molecular | of |
| No | G | R⁷ | Name | Mass | Ex. |
| 267 | 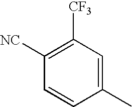 | 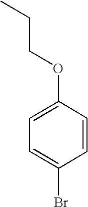 | (3aα,4β,7β,7a α)-(4-[7[2-(4- Bromophenoxy) ethyl]octahydro- 4-methyl- 1,3-dioxo-4,7- epoxy-2H- isoindol-2-yl]-2- (trifluoromethyl) benzonitrile.. | 397 LCMS 549.0 [M + H]⁺ | 204, 35 |
| 268 | 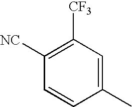 | 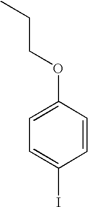 | (3aα,4β,7β,7a α)-4- [Octahydro-7- [2-(4-iodophenoxy)ethyl]-4-methyl- 1,3-dioxo-4,7- epoxy-2H- isoindol-2-yl]- 2-(trifluoromethyl) benzonitrile. | 4.09 LCMS 597.0 [M + H]⁺ | 204, 35 |
| 269 | 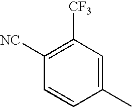 | 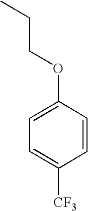 | (3aα,4β,7β,7a β)-4- [Octahydro-4- methyl-1,3- dioxo-7-[2-[4- (trifluoromethyl phenoxy]ethyl]-4,7-epoxy- 2H-isoindol-2- yl]-2-(trifluoromethyl) benzonitrile. | 3.95 LC | 204, 35 |
| 270 | 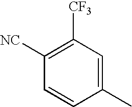 | 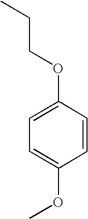 | (3aα,4β,7β,7a β)-4- [Octahydro-7- [2-(4- methoxypheno xy)ethyl]-4- methyl-1,3- dioxo-4,7- epoxy-2H- isoindol-2-yl]- 2-(trifluoromethyl) benzonitrile. | 3.66 LC | 204, 35 |
| 271 | 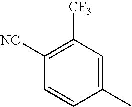 | 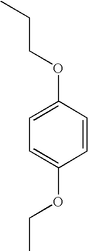 | (3aα,4β,7β,7a α)-(4-[7[2-(4- Ethoxyphenoxy) ethyl]octahydro-4-methyl- 1,3-dioxo-4,7- epoxy-2H- isoindol-2-yl]- 2-(trifluoromethyl) benzonitrile. | 3.81 LC | 204, 35 |
| 272 | 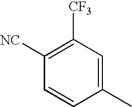 | 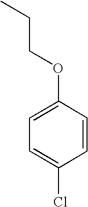 | (3aα,4β,7β,7a α)-4-[7-[2(-4- Clorophenoxy) ethyl]octahydro- 4-methyl- 1,3-dioxo-4,7- epoxy-2H- isoindol-2-yl]-2-(trifluoromethyl) benzonitrile. | 3.97 LCMS 522.2 [M + H]⁺ | 204, 35 |
| 273 | 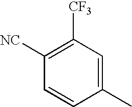 | 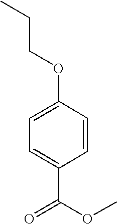 | (3aα,4β,7β,7a α)-4-[2-[2-[4- Cyano-3- (trifluoromethyl) phenyl]octahydro- 7-methyl- 1,3-dioxo-4,7-epoxy-4H- isoindol-4- yl]ethoxy]benz oic acid, methyl ester. | 3.77 LCMS 529.2 [M + H]⁺ | 204, 35 |
| 274 | 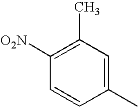 | 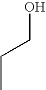 | (3aα,4β,7β,7a α)-Hexahydro- 4-(2- hydroxyethyl)- 7-methyl-2-(3- methyl-4- nitrophenyl)- 4,7-epoxy-1H- isoindole- 1,3(2H)-dione. | 2.44 LC | 204, 35 |
| 275 | 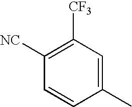 | 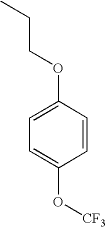 | (3aα,4β,7β,7a α)-4- [Octahydro-4- methyl-1,3- dioxo-7-[2-[4- (trifluoromethoxy) phenoxy]ethyl]-4,7- epoxy-2H- isoindol-2-yl]- 2- (trifluoromethyl) benzonitrile. | 3.97 LC | 204, 35 |
| 276 | 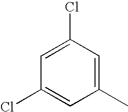 | 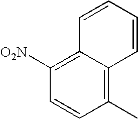 | (3aα,4β,7β,7a α)-2-(3,5- Dichlorophenyl) hexahydro- 4,7-dimethyl-4,7-epoxy-1H- isoindole- 1,3(2H)-dione. | 3.31 LCMS 341.2 [M + H]⁺ | 20 |
| 277 | 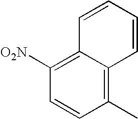 | CH₃ | (3aα,4β,7β,7a α)-Hexahydro- 4,7-dimethyl- 2-(4-nitro-1- naphthalenyl)- 4,7-epoxy-1H- isoindole- 1,3(2H)-dione. | 3.04 LCMS | 20 |
| 278 | 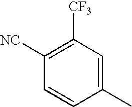 | 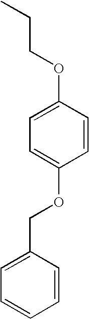 | (3aα,4β,7β,7a α)-4- [Octahydro-4- methyl-1,3- dioxo-7-[2-[14- (phenylmethoxy) phenoxy]ethyl]4,7-expoxy- 2H-isoindol-2- yl]-2- (trifluoromethyl) benzonitrile. | 4.06 LC | 204, 35 |
| 279 | 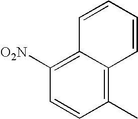 | 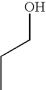 | (3aα,4β,7β,7a α)-Hexahydro- 4-(2- hydroxyethyl)- 7-methyl-2-(4- nitro-1- naphthalenyl)- 4,7-expoxy-1H- indole- 1,3(2H)-dione. | 2.607 & 2.743 rotational isomers LC | 204, 35 |
| 280 | 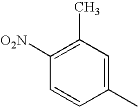 | 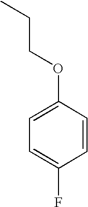 | (3aα,4β,7β,7a β)-4-[2-(4- Fluorophenoxy) ethyl]hexahydro -7-methyl- 2-(3-methyl-4- nitrophenyl)- 4,7-epoxy-1H- isoindole- 1,3(2H)-dione. | 3.68 LC | 204, 35 |
| 281 | 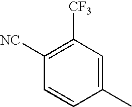 | 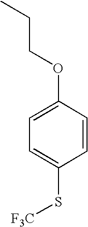 | (3aα,4β,7β,7a β)-4- [Octahydro-4- dioxo-7-[2-[4- methyl-1,3- (trifluoromethyl) thio]phenoxy]ethyl]-4,7- epoxy-2H- isoindol-2-yl]- 2- (trifluoromethyl) benzonitrile. | 4.11 LC | 204, 35 |
| 282 | 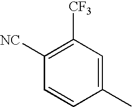 | 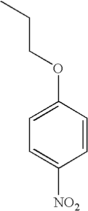 | (3aα,4β,7β,7a β)-4- [Octahydro-4- methyl-7-[2-(4- nitrophenoxy) ethyl]-1,3- dioxo-4,7- epoxy-2H- isoindol-2-yl]- 2- (trifluoromethyl) benzonitrile. | 3.68 LC | 204, 35 |
| 283 | 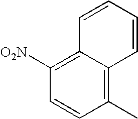 | 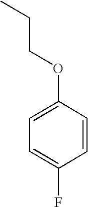 | (3aα,4β,7β,7a β)-4-[2-(4- Fluorophenoxy ethyl]hexahydro- 7-methyl- 2-(4-nitro-1- naphthalenyl)- 4,7-epoxy-1H- isoindole- 1,3(2H)-dione. | 3.68 & 3.80 rotational isomes LC | 204, 35 |
| 284 | 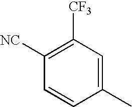 | 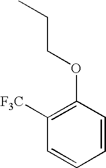 | (3aα,4β,7β,7a β)-4- [Octahydro-7- methyl-1,3- dioxo-7-[2-[2- (trifluoromethyl) phenoxy]ethyl]-4,7-epoxy- 2H-isoindol-2- yl]-2- (trifluoromethyl) benzonitrile. | 3.89 LC | 204, 35 |
| 285 | 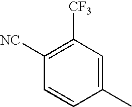 | 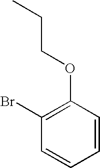 | (3aα,4β,7β,7a β)-4-[4-[2-(2- Bromophenoxy) ethyl]octahydro- 7-methyl- 1,3-dioxo-4,7- epoxy-2H- isoindol-2-yl]- 2- (trifluoromethyl) benzonitrile. | 3.91 LC | 204, 35 |
| 286 | 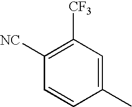 | 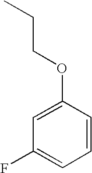 | (3aα,4β,7β,7a α)-4-[4-[2-(3- Fluorophenoxy) ethyl]octahydro- 7-methyl- 1,3-dioxo-4,7- epoxy-2H- isoindol-2-yl]- 2- (trifluoromethyl) benzonitrile. | 3.78 LC | 204, 35 |
| 287 | 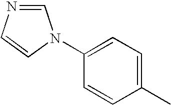 | H | (3aα,4β,7β,7a α)-Hexahydro- 2-[4-(1H- imidazol-1- yl)phenyl]-4- methyl-4,7- epoxy-1H- isoindole- 1,3(2H)-dione. | 1.16 LC | 3 |
| 288 | 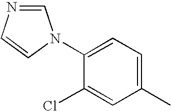 | H | (3aα,4β,7β,7a α)-2-[3- Chloro-4-(2- thiazolyl)phen yl]hexahydro- 4-methyl-4,7- epoxy-1H- isoindole- 1,3(2H)-dione. | 2.81 LC | 3 |
| 289 | 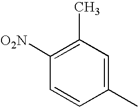 | CH₃ | (3aα,4β,7β,7a α)-Hexahydro- 4,7-dimethyl- 2-(3-methyl-4- nitrophenyl)- 4,7-epoxy-1H- isoindole- 1,3(2H)-dione. | 2.74 LC | 20 |
| 290 | 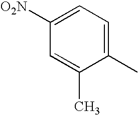 | CH₃ | (3aα,4β,7β,7a α)-Hexahydro- 4,7-dimethyl- 2-(2-methyl-4- nitrophenyl)- 4,7-epoxy-1H- isoindole- 1,3(2H)-dione. | 2.71 LC | 20 |
| 291 | 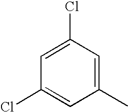 | 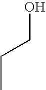 | (3aα,4β,7β,7a α)-2-(3,5- Dichloropheny l)hexahydro-4- (2- hydroxyethyl)- 7-methyl-4,7- epoxy-1H- isoindole- 1,3(2H)-dione. | 2.98 LC | 204 |
| 292 | 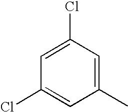 | 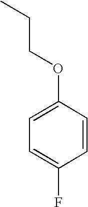 | (3aα,4β,7β,7a α)-2-(3,5- Dichloropheny l)-4-[2-(4- fluorophenoxy )ethyl]hexahyd ro-7-methyl- 4,7-epoxy-1H- isoindole- 1,3(2H)-dione. | 4.03 LC | 204, 35 |
| 293 | 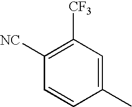 | 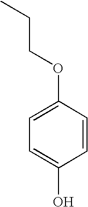 | (3aα,4β,7β,7a α)-4- [Octahydro-4- [2-(4- hydroxypheno xy)ethyl]-7- methyl-1,3- dioxo-4,7- epoxy-2H- isoindol-2-yl]- 2- (trifluoromethy l)benzonitrile. | 3.25 LC | 204, 35 |
| 294 | 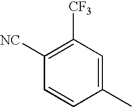 | 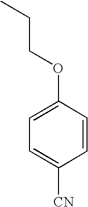 | (3aα,4β,7β,7a α)-4-[4-[2-)4- Cyanophenox y)ethyl]octahy dro-7-methy- 1,3-dioxo-4,7- epoxy-2H- isoindol-2-yl]- 2- (trifluoromethy l)benzonitrile. | 3.51 LC | 204, 35 |
| 295 | 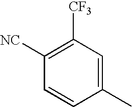 | 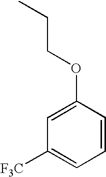 | (3aα,4β,7β,7a α)-4- [Octahydro-4- methyl-1,3- dioxo-7-[2-[3- (trifluoromethy l)phenoxy]ethy l]4,7-epoxy- 2H-isoindol-2- yl]-2- (trifluoromethy l)benzonitrile. | 3.85 LC | 204, 35 |
| 296 | 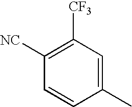 | 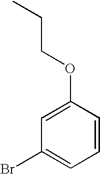 | (3aα,4β,7β,7a α)-4-[4-[2-(3- Bromophenox y)ethyl]octahy dro-7-methyl- 1,3-dioxo-4,7- epoxy-2H- isoindol-2-yl]- 2- (trifluoromethy l)benzonitrile. | 3.84 LC | 204, 35 |
| 297 | 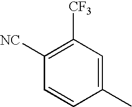 | 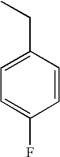 | (3aα,4β,7β,7a α)-4-[4-[(4- Fluorophenyl) methyl]octahy dro-7-methyl- 1,3-dioxo-4,7- epoxy-2H- isoindol-2-yl]- 2- (trifluoromethy l)benzonitrile. | 3.73 LC | 205 |
| 298 | 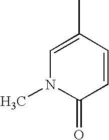 | CH₃ | (3aα,4β,7β,7a α)-2-(1,6- Dihydro-1- methyl-6-oxo- 3- pyridinyl)hexa hydro-4,7- dimethyl-4,7- epoxy-1H- isoindole- 1,3(2H)-dione. | 1.61 LC | 20 |
| 299 | 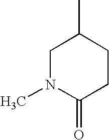 | CH₃ | (3aα,4β,7β,7a α)-Hexahydro- 4,7-dimethyl- 2-(1-methyl-6- oxo-3- piperidinyl)- 4,7-epoxy-1H- isoindole- 1,3(2H)-dione. | 1.73 LC | 20 |
| 300 | 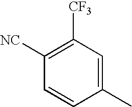 | 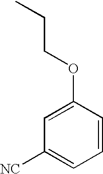 | (3aα,4β,7β,7a α)-4-[4-[2-(3- Cyanophenox y)ethyl]octahy dro-7-methyl- 1,3-dioxo-4,7- epoxy-2H- isoindol-2-yl]- 2- (trifluoromethy l)benzonitrile. | 3.46 LC | 20 |
| 301 | 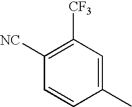 | 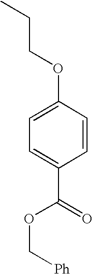 | (3aα,4β,7β,7a α)-4-[2-[4- Cyano-3- (trifluoromethy l)phenyl]octah ydro-7-methyl- 1,3-dioxo-4,7- epoxy-4H- isoindol-4- yl]ethoxy]benz oic acid, phenylmethyl ester. | 4.01 LC | 204, 35 |
| 302 | 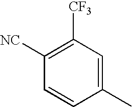 | 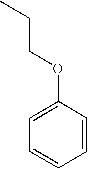 | (3aα,4β,7β,7a α)-4- [Octahydro-4- methyl-1,3- dioxo-7-(2- phenoxyethyl)- 4,7-epoxy-2H- isoindol-2-yl]- 2- (trifluoromethy l)benzonitrile. | 3.57 LC | 204, 35 |
| 303 | 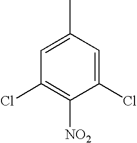 | CH₃ | (3aα,4β,7β,7a α)-2-(3,5- Dichloro-4- nitrophenyl)he xahydro-4,7- dimethyl-4,7- epoxy-1H- isoindole- 1,3(2H)-dione. | 3.40 LC | 20 |
| 304 | 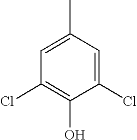 | CH₃ | (3aα,4β,7β,7a α)-2-(3,5- Dichloro-4- hydroxyphenyl )hexahydro- 4,7-dimethyl- 4,7-epoxy-1H- isoindole- 1,3(2H)-dione. | 2.58 LC | 20 |
| 305 | 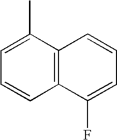 | CH₃ | (3aα,4β,7β,7a α)-2-(5-Fluoro- 1- naphthalenyl)h exahydro-4,7- dimethyl-4,7- epoxy-1H- isoindole- 1,3(2H)-dione. | 2.96 & 3.06 rotational isomers LC | 20 |
| 306 | 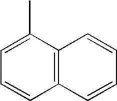 | CH₃ | (3aα,4β,7β,7a α)-Hexahydro- 4,7-dimethyl- 2-(1- naphthalenyl)- 4,7-epoxy-1H- isoindole- 1,3(2H)-dione. | 2.60 & 2.73 rotational isomers LC | 20 |
| 307 | 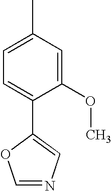 | CH₃ | (3aα,4β,7β,7a α)-Hexahydro- 2-[3-methoxy- 4-(5- oxazolyl)phen yl]-4,7- dimethyl-4,7- epoxy-1H- isoindole- 1,3(2H)-dione. | 2.62 rotational isomers LC | 20 |
| 308 | 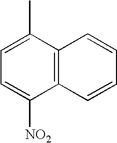 | 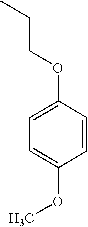 | (3aα,4β,7β,7a α)-Hexahydro- 4-[2-(4- methoxypheno xy)ethyl]-7- methyl-2-(4- nitro-1- naphthalenyl)- 4,7-epoxy-1H- isoindole- 1,3(2H)-dione. | 3.42 & 3.55 rotational isomers LC | 204, 35 |
| 309 | 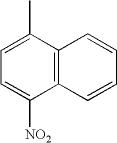 | 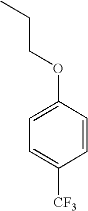 | (3aα,4β,7β,7a α)-Hexahydro- 4-methyl-2-(4- nitro-1- naphthalenyl)- 7-[2-[4- (trifluoromethy l)phenoxy]ethy l]-4,7-epoxy- 1H-isoindole- 1,3(2H)-dione. | 3.81 & 3.93 rotational isomers LC | 204, 35 |
| 310 | 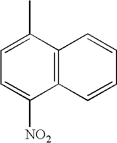 | 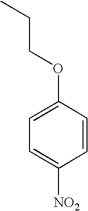 | (3aα,4β,7β,7a α)-Hexahydro- 4-methyl-2-(4- nitro-1- naphthalenyl)- 7-[2-(4- nitrophenoxy) ethyl]-4,7- epoxy-1H- isoindole- 1,3(2H)-dione. | 3.48 & 3.61 rotational isomers LC | 204, 35 |
| 311 | 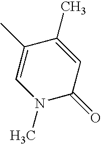 | CH₃ | (3aα,4β,7β,7a α)-2-(1,6- Dihydro-1,4- dimethyl-6- oxo-3- pyridinyl)hexa hydro-4,7- dimethyl-4,7- epoxy-1H- isoindole- 1,3(2H)-dione. | 1.89 LC | 20 |
| 312 | 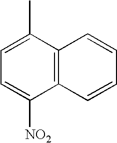 | 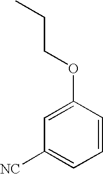 | (3aα,4β,7β,7a α)-4- [Octahydro-7- methyl-2-(4- nitro-1- naphthalenyl)- 1,3-dioxo-4,7- epoxy-4H- isoindol-4- yl]ethoxy]benz onitrile. | 3.63 LC | 204, 35 |
| 313 | 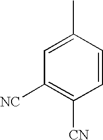 | CH₃ | (3aα,4β,7β,7a α)-4- (Octahydro- 4,7-dimethyl- 1,3-dioxo-4,7- epoxy-2H- isoindol-2-yl)- 1,2- benzenedicarb onitrile. | 2.38 LC | 20 |
| 314 | 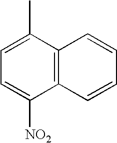 | 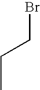 | (3aα,4β,7β,7a α)-4-(2- Bromoethyl)he xahydro-7- methyl-2-(4- nitro-1- naphthalenyl)- 4,7-epoxy-1H- isoindole- 1,3(2H)-dione. | 3.52 LC | 36 |
| 315 | 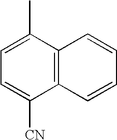 | 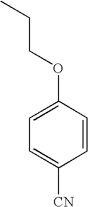 | (3aα,4β,7β,7a α)-4-[4-[2-(4- Cyanophenox y)ethyl]octahy dro-7-methyl- 1,3-dioxo-4,7- epoxy-2H- isoindol-2-yl]- 1- naphthaleneca rbonitrile. | 3.19 & 3.35 rotational isomers LC | 223, 35 |
| 316 | 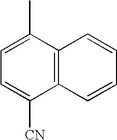 | 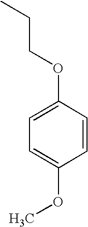 | (3aα,4β,7β,7a α)-4- [Octahydro-4- [2-(4- methoxypheno xy)ethyl]-7- methyl-1,3- dioxo-4,7- epoxy-2H- isoindol-2-yl]- 1- naphthaleneca rbonitrile. | 3.34 & 3.50 rotational isomers LC | 223, 35 |
| 317 | 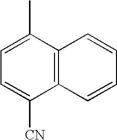 | 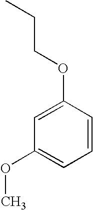 | (3aα,4β,7β,7a α)-4- [Octahydro-4- [2-(3- methoxypheno xy)ethyl]-7- methyl-1,3- dioxo-4,7- epoxy-2H- isoindol-2-yl]- 1- naphthaleneca rbonitrile. | 3.34 & 3.50 rotational isomers LC | 223, 35 |
| 318 | 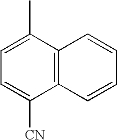 | 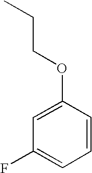 | (3aα,4β,7β,7a α)-4-[4-[2-(3- Fluorophenox y)ethyl]octahy dro-7-methyl- 1,3-dioxo-4,7- epoxy-2H- isoindol-2-yl]- 1- naphthaleneca rbonitrile. | 3.46 & 3.61 rotational isomers LC | 223, 35 |
| 319 | 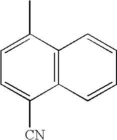 | 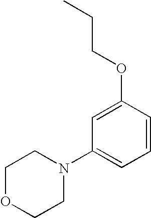 | (3aα,4β,7β,7a α)-4- [Octahydro-4- methyl-7-[2-[3- (4- morpholinyl)ph enoxy]ethyl]- 1,3-dioxo-4,7- epoxy-2H- isoindol-2-yl]- 1- naphthaleneca rbonitrile. | 3.01 & 3.18 rotational isomers LC | 223, 35 |
| 320 | 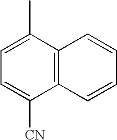 | 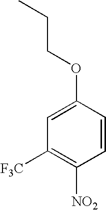 | (3aα,4β,7β,7a α)-4- [Octahydro-4- methyl-7-[2-[4- nitro-3- (triflouorometh yl)phenoxy]eth yl]-1,3-dioxo- 4,7-epoxy-2H- isoindol-2-yl]- 1- naphthaleneca rbonitrile. | 3.70 & 3.83 rotational isomers LC | 223, 35 |
| 321 | 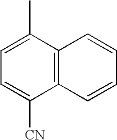 | 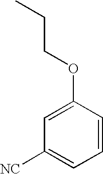 | (3aα,4β,7β,7a α)-4-[4-[2-(3- Cyanophenox y)ethyl]octahy dro-7-methyl- 1,3-dioxo-4,7- epoxy-2H- isoindol-2-yl]- 1- naphthaleneca rbonitrile. | 3.39 & 3.55 rotational isomers LC | 223, 35 |
| 322 | 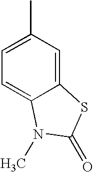 | CH₃ | (3aα,4β,7β,7a α)-2-(2,3- Dihydro-3- methyl-2-oxo- 6- benzothiazolyl )hexahydro- 4,7-dimethyl- 4,7-epoxy-1H- isoindole- 1,3(2H)-dione. | 2.34 rotational isomers LC | 20 |
| 323 | 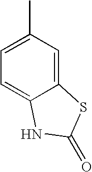 | CH₃ | (3aα,4β,7β,7a α)-2-(2,3- Dihydro-3- methyl-2-oxo- 6- benzothiazolyl )hexahydro- 4,7-dimethyl- 4,7-epoxy-1H- isoindole- 1,3(2H)-dione. | 2.16 LC | 20 |
| 324 | 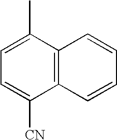 | 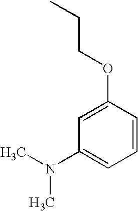 | (3aα,4β,7β,7a α)-4-[4-[2-[3- (Dimethylamin o)phenoxy]eth yl]octahydro- 7-methyl-1,3- dioxo-4,7- epoxy-2H- isoindol-2-yl]- 1- naphthaleneca rbonitrile. | 2.63 & 2.79 rotational isomers LC | 223, 35 |
| 325 | 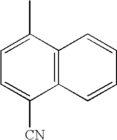 | 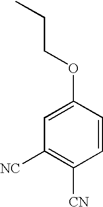 (3aα,4β,7β,7a α)-4-[2-[4 Cyano-3- (trifluoromethy l)phenyl]octah ydro-7-methyl- 1,3-dioxo-4,7- epoxy-4H- isoindol-4- yl]ethoxy]-1,2- benzenedicarb onitrile. | 3.42 rotational isomers LC | 223, 35 | |
| 326 | 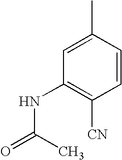 | CH₃ | (3aα,4β,7β,7a α)-N-[2- Cyano-5- (octahydro- 4,7-dimethyl- 1,3-dioxo-4,7- epoxy-2H- isoindol-2- yl)phenyl]acet amide. | 1.94 LC | 20 |
| 327 | 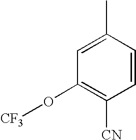 | CH₃ | (3aα,4β,7β,7a α)-4- (Octahydro- 4,7-dimethyl- 1,3-dioxo-4,7- epoxy-2H- isoindol-2-yl)- 2- (trifluorometho xy)benzonitrile | 3.52 LC | 20 |
| 328 | 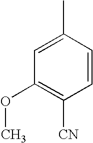 | CH₃ | (3aα,4β,7β,7a α)-2- Methoxy- 4-(octahydro- 4,7-dimethyl- 1,3-dioxo-4,7- epoxy-2H- isoindol-2- yl)benzonitrile. | 2.47 LC | 20 |
| 329 | 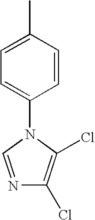 | CH₃ | (3aα,4β,7β,7a α)-2-[4-(4,5- Dichloro-1H- imidazol-1- yl)phenyl]hexa hydro-4,7- dimethyl-4,7- epoxy-1H- isoindole- 1,3(2H)-dione. | 3.09 LC | 20 |
| 330 | 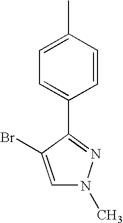 | CH₃ | (3aα,4β,7β,7a α)-2-[4-(4- Bromo-1- methyl-1H- pyrazol-3- yl)phenyl]hexa hydro-4,7- dimethyl-4,7- epoxy-1H- isoindole- 1,3(2H)-dione. | 3.04 LC | 20 |
| 331 | 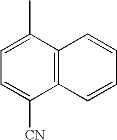 | 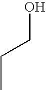 | (3aα,4β,7β,7a α)-4- [Octahydro-4- (2- hydroxyethyl)- 7-methyl-1,3- dioxo-4,7- epoxy-2H- isoindol-2-yl]- 1- naphthaleneca rbonitrile. | 2.44 & 2.60 rotational isomers LC | 223, 35 |
| 332 | 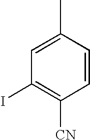 | CH₃ | (3aα,4β,7β,7a α)-2-Iodo-4- (Octahydro- 4,7-dimethyl- 1,3-dioxo-4,7- epoxy-2H- isoindol-2- yl)benzonitrile. | 2.78 rotational isomers LC | 20 |
| 333 | 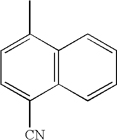 | 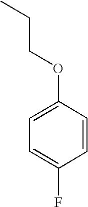 | (3aα,4β,7β,7a α)-4-[4-[2-(4- Fluorophenox ly)ethyl]octahy dro-7-methyl- 1,3-dioxo-4,7- epoxy-2H- isoindol-2-yl]- 1- naphthaleneca rbonitrile. | 3.39 & 3.53 rotational isomers LC | 223, 35 |
| 334 | 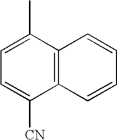 | 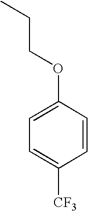 | (3aα,4β,7β,7a α)-4- [Octahydro-4- methyl-1,3- dioxo-7-[2-[4- (trifluoromethy l)phenoxy]ethy l]-4,7-epoxy- 2H-isoindol-2- yl]-1- naphthaleneca rbonitrile. | 3.66 & 3.78 rotational isomers LC | 223, 35 |
| 335 | 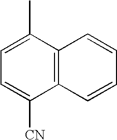 | 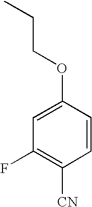 | (3aα,4β,7β,7a α)-4-[4-[2-(4- Cyano-3- fluorophenoxy )ethyl]octahydr o-7-methyl- 1,3-dioxo-4,7- epoxy-2H- isoindol-2-yl]- 1- naphthaleneca rbonitrile. | 3.26 & 3.41 rotational isomers LC | 223, 35 |
| 336 | 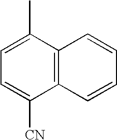 | 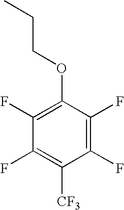 | (3aα,4β,7β,7a α)-4- [Octahydro-4- methyl-1,3- dioxo-7-[2- [2,3,5,6- tetrafluoro-4- (trifluoromethy l)phenoxy]ethy l]4,7-epoxy- 2H-isoindol-2- yl]-1- naphthaleneca rbonitrile. | 3.94 & 4.01 rotational isomers LC | 223, 35 |
| 337 | 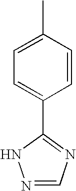 | CH₃ | (3aα,4β,7β,7a α)-Hexahydro- 4,7-dimethyl- 2-[4-(1H-1,2,4- triazol-3- yl)phenyl]-4,7- epoxy-1H- isoindole- 1,3(2H)-dione. | 2.06 LC | 20 |
| 338 | 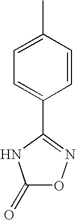 | CH₃ | (3aα,4β,7β,7a α)-2-[4-(4,5- Dihydro-5- oxo-1,2,4- oxadiazol-3- yl)phenyl]hexa hydro-4,7- dimethyl-4,7- epoxy-1H- isoindole- 1,3(2H)-dione. | 2.42 LC | 20 |
| 339 | 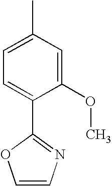 | CH₃ | (3aα,4β,7β,7a α)-Hexahydro- 2-[3-methoxy- 4-(2- oxazolyl)phen yl]-4,7- dimethyl-4,7- epoxy-1H- isoindole- 1,3(2H)-dione. | 2.51 LC | 20 |
| 340 | 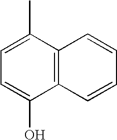 | CH₃ | (3aα,4β,7β,7a α)-Hexahydro- 2-(4-hydroxy- 1- naphthalenyl)- 4,7-dimethyl- 4,7-epoxy-1H- isoindole- 1,3(2H)-dione. | 2.30 LC | 20 |
| 341 | 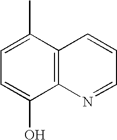 | CH₃ | (3aα,4β,7β,7a α)-Hexahydro- 2-(8-hydroxy- 5-quinolinyl)- 4,7-dimethyl- 4,7-epoxy-1H- isoindole- 1,3(2H)-dione, trifluoroacetat e(1:1). | 1.49 LC | 20 |
| 342 | 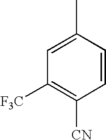 | 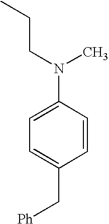 | (3aα,4β,7β,7a α)-4- [Octahydro-4- methyl-1,3- dioxo-7-[2- [methyl(phenyl methyl)amino]ethyl]-4,7- epoxy-2H- isoindol-2-yl]- 2- (trifluoromethy l)benzonitrile. | 2.42 LC | 223, 35 |
| 343 | 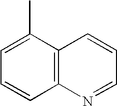 | CH₃ | (3aα,4β,7β,7a α)-Hexahydro- 4,7-dimethyl- quinolinyl)-4,7- epoxy-1H- isoindole- 1,3(2H)-dione. | 1.69 LC | 20 |
| 344 | 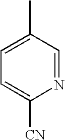 | CH₃ | (3aα,4β,7β,7a α)-5- (Octahydro- 4,7-dimethyl- 1,3-dioxo-4,7- epoxy-2H- isoindol-2-yl)- 2- pyridinecarbon itrile. | 2.18 LC | 20 |
| 345 | 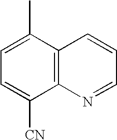 | CH₃ | (3aα,4β,7β,7a α)-5- (Octahydro- 4,7-dimethyl- 1,3-dioxo-4,7- epoxy-2H- isoindol-2-yl)- 8- quinolinecarbo nitrile. | 2.31 LC | 20 |
| 346 | 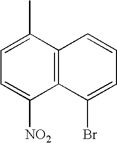 | CH₃ | (3aα,4β,7β,7a α)-2-(5- Bromo-4-nitro- 1- naphthalenyl)h exahydro-4,7- dimethyl-4,7- epoxy-1H- isoindole- 1,3(2H)-dione. | 3.10 & 3.29 rotational isomers LC | 20 |
| 347 | 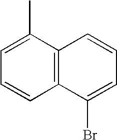 | CH₃ | (3aα,4β,7β,7a α)-2-(5- Bromo-1- naphthalenyl)h exahydro-4,7- dimethyl-4,7- epoxy-1H- isoindole- 1,3(2H)-dione. | 3.28 & 3.40 rotational isomers LC | 20 |
| 348 | 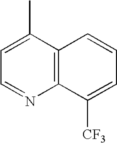 | CH₃ | (3aα,4β,7β,7a α)-Hexahydro- 4,7-dimethyl- (trifluoromethy l)-4-quinolinyl]- 4,7-epoxy-1H- isoindole- 1,3(2H)-dione. | 3.08 LC | 20 |
| 349 | 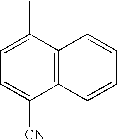 | 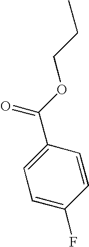 | 4- Fluorobenzoic acid,2- [(3aα,4β,7β,7a α)-2-(4-cyano- 1- naphthalenyl)o ctahydro-7- methyl-1,3- dioxo-4,7- epoxy-4H- isoindol-4- y]ethyl ester. | 3.64 & 3.77 rotational isomers LC | 223 |
| 350 | 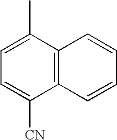 | 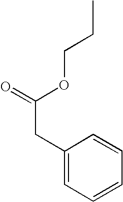 | Benzeneacetic acid, 2-[(3aα,4β,7β,7a α)-2-(4-cyano- 1- naphthalenyl)o ctahydro-7- methyl-1,3- dioxo-4,7- epoxy-4H- isoindol-4- y]ethyl ester. | 3.53 & 3.67 rotational isomers LC | 223 |
| 351 | 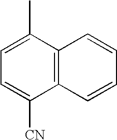 | 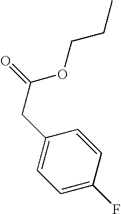 | 4- Fluorobenzen eacetic acid, 2-[(3aα,4β,7β,7a α)-2-(4-cyano- 1- naphthalenyl)o ctahydro-7- methyl-1,3- dioxo-4,7- epoxy-4H- isoindol-4- y]ethyl ester. | 3.53 & 3.66 rotational isomers LC | 223 |
| 352 | 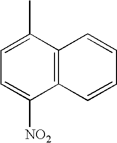 | 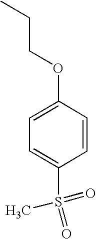 | (3aα,4β,7β,7a α)-Hexahydro- [4- (methylsulfony l)phenoxy]ethy l]-2-(4-nitro-1- naphthalenyl)- 4,7-epoxy-1H- isoindole- 1,3(2H)-dione. | 3.31 LC | 223, 35 |
| 353 | 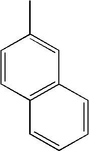 | CH₃ | (3aα,4β,7β,7a α)-Hexahydro- naphthalenyl)- 4,7-dimethyl- 4,7-epoxy-1H- isoindole- 1,3(2H)-dione. | 2.94 LC | 20 |
| 354 | 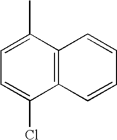 | CH₃ | (3aα,4β,7β,7a α)-2-(4- Chloro-1- naphthalenyl)h exahydro-4,7- dimethyl-4,7- epoxy-1H- isoindole- 1,3(2H)-dione. | 3.22 & 3.34 rotational isomers LC | 20 |
| 355 | 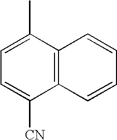 | 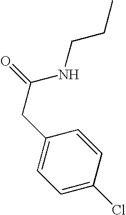 | (3aα,4β,7β,7a α)-N-[(4- Chlorophenyl) methyl]-2-(4- cyano-1- naphthalenyl)o ctahydro-7- methyl-1,3- dioxo-4,7- epoxy-4H- isoindole-4- acetamide. | 3.52 LC | 237 |
| 356 | 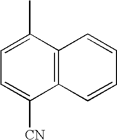 | 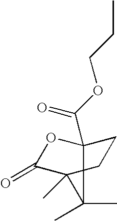 | 4,7,7- Trimethyl-3- oxo-2- oxabicyclo[2.2 .1]heptane-1- carboxylic acid, 2- [(3aα,4β,7β,7a α)-2-(4-cyano- 1- naphthalenyl)o ctahydro-7- methyl-1,3- dioxo-4,7- epoxy-4H- isoindol-4- y]ethyl ester. | 3.45 LC | 223 |
| 357 | 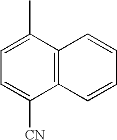 | 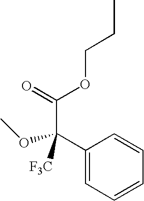 | (αS)-α- Methoxy-α- (trifluoromethy l)benzeneaceti c acid, 2- [(3aα,4β,7β,7a α)-2-(4-cyano- 1- naphthalenyl)o ctahydro-7- methyl-1,3- dioxo-4,7- epoxy-4H- isoindol-4- y]ethyl ester. | 3.91 LC | 223 |
| 358 | 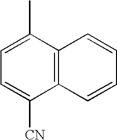 | 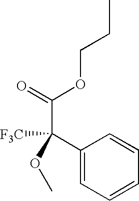 | (αR)-α- Methoxy-α- (trifluoromethy l)benzeneaceti c acid, 2- [(3aα,4β,7β,7a α)-2-(4-cyano- 1- naphthalenyl)o ctahydro-7- methyl-1,3- dioxo-4,7- epoxy-4H- isoindol-4- y]ethyl ester. | 2.00 LC | 223 |
| 359 | 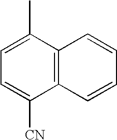 | 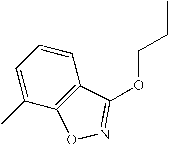 | (3aα,4β,7β,7a α)-4- [Octahydro-4- methyl-7-[2- [(7-methyl-1,2- benzisoxazol- 3-yl)oxy]ethyl]- 1,3-dioxo-4,7- epoxy-2H- isoindol-2-yl]- 1- naphthaleneca rbonitrile. | 3.79 & 3.92 LC Rotationale isomers | 250 |
| 360 | 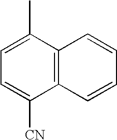 | 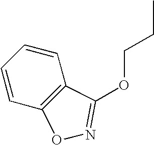 | (3aα,4β,7β,7a α)-4-[4-[2-(1,2- Benzisoxazol- 3- yloxy)ethyl]oct ahydro-7- methyl-1,3- dioxo-4,7- epoxy-2H- isoindol-2-yl]- 1- naphthaleneca rbonitrile. | 3.55 & 3.70 LC Rotationale Isomers | 250 |
| 361 | 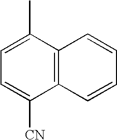 | 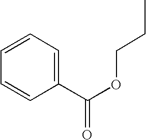 | (3aα,4β,7β,7a α)-4-[2- (Benzoyloxy)e thyl]-2-(4- cyano-1- naphthalenyl)h exahydro-7- methyl-4,7- epoxy-1H- isoindole- 1,3(2H)-dione. | 3.51 & 3.66 LC Rotationale isomers | 223 |
| 362 | 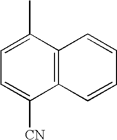 | 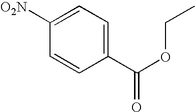 | (3aα,4β,7β,7a α)-2-(4- Cyano-1- naphthalenyl)- nitrobenzoyl)o xy]ethyl]hexah ydro-7-methyl- 4,7-epoxy-1H- isoindole- 1,3(2H)-dione. | 3.52 & 3.67 LC Rotationale Isomers | 223 |
| 363 | 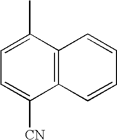 | 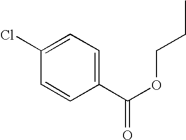 | 4- Chlorobenzoic acid, 2- [(3aα,4β,7β,7a α)-2-(4-cyano- 1- naphthalenyl)o ctahydro-7- methyl-1,3- dioxo-4,7- epoxy-4H- isoindol-4- y]ethyl ester. | 3.79 LC | 223 |
| 364 | 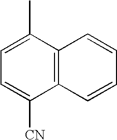 | 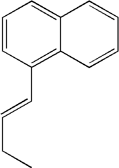 | (3aα,4β,7β,7a α(E)]-4- [Octahydro-4- methyl-7-[3-(1- naphthalenyl)- 2-propenyl]- 1,3-dioxo-4,7- epoxy-2H- isoindol-2-yl]- 1- naphthaleneca rbonitrile. | 4.14 LC 49.13 [M + H]⁺ | 248 |
| 365 | 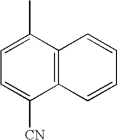 | 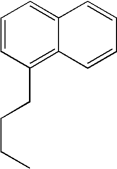 | (3aα,4β,7β,7a α)-4- [Octahydro-4- methyl-7-[3-(-1- naphthalenyl)p ropyl]-1,3- dioxo-4,7- epoxy-2H- isoindol-2-yl]- 1- naphthaleneca rbonitrile. | 4.14 LC 501.44 [M + H]⁺ | 248, 249 |
| 366 | 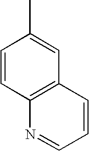 | CH₃ | (3aα,4β,7β,7a α)-Hexahydro- 4,7-dimethyl- 2-(2-methyl-6- quinolinyl)-4,7- epoxy-1H- isoindole- 1,3(2H)-dione. | 1.25 LC 337.0 [M + H]⁺ | 20 |
| 367 | 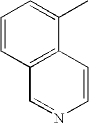 | CH₃ | (3aα,4β,7β,7a α)-Hexahydro- 2-(5- isoquinolinyl)- 4,7-dimethyl- 4,7-epoxy-1H- isoindole- 1,3(2H)-dione. | 1.06 & 1.29 LC Rotationale Isomers 323.0 [M + H]⁺ | 20 |
| 368 | 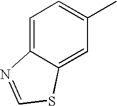 | CH₃ | (3aα,4β,7β,7a α)-2-(6- Benzothiazolyl )hexahydro- 4,7-dimethyl- 4,7-epoxy-1H- isoindole- 1,3(2H)-dione. | 2.15 LC 329.0 [M + H]⁺ | 20 |
| 369 | 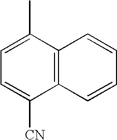 | 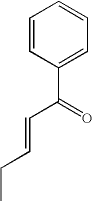 | (3aα,4β,7β,7a α(E)]-4- [Octahydro-4- methyl-1,3- dioxo-7-(4- oxo-4-phenyl- 2-butenyl)-4,7- epoxy-2H- isoindol-2-yl]- 1- naphthaleneca rbonitrile. | 3.50 LC 482.14 [M + H]⁺ | 248 |
| 370 | 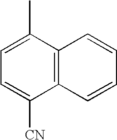 | 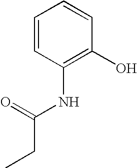 | (3aα,4β,7β,7a α)-2-(4- Cyano-1- naphthalenyl)o ctahydro-N-(2- hydroxyphenyl )-7-methyl-1,3- dioxo-4,7- epoxy-4H- isoindole-4- acetamide. | 3.07 LC 482.14 [M + H]⁺ | 236 |
| 371 | 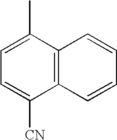 | 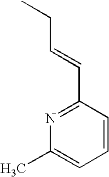 | (3aα,4β,7β,7a α]-4- [Octahydro-4- methyl-7-[3-(6- methyl-2- pyridinyl)-2- propenyl]-1,3- dioxo-4,7- epoxy-2H- isoindol-2-yl]- 1- naphthaleneca rbonitrile. | 2.28 LC 464.19 [M + H]⁺ | 248 |
| 372 | 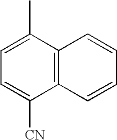 | 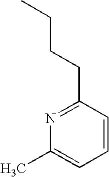 | (3aα,4β,7β,7a α)-4- [Octahydro-4- methyl-7-[3-(6- methyl-2- pyridinyl)propy l]-1,3-dioxo- 4,7-epoxy-2H- isoindol-2-yl]- 1- naphthaleneca rbonitrile. | 2.19 LC 466.32 [M + H]⁺ | 248, 249 |
| 373 | 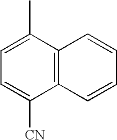 | 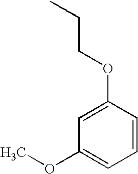 | [3aR- (3aα,4β,7β,7a α)-]-4- [Octahydro-4- [2-(3- methoxypheno xy)ethyl]-7- methyl-1,3- dioxo-4,7- epoxy-2H- isoindol-2-yl]- 1- naphthaleneca rbonitrile. | 3.73 LC 483.65 [M + H]⁺ | 238i, 239i |
| 374 | 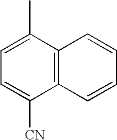 | 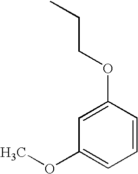 | [3aS- (3aα,4β,7β,7a α)]-4- [Octahydro-4- methoxypheno xy)ethyl]-7- methyl-1,3- epoxy-2H- isoindol-2-yl]- 1- naphthaleneca rbonitrile. | 3.73 LC | 238ii, 239ii |
| 375 | 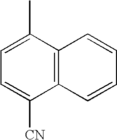 | 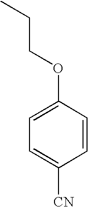 | [3aR- (3aα,4β,7β,7a α)]-4-[4-[2-(4- Cyanophenox y)ethyl]octahy dro-7-methyl- 1,3-dioxo-4,7- epoxy-2H- isoindol-2-yl]- 1- naphthaleneca rbonitrile. | 3.33 & 3.49 LC Rotationale Isomers | 238i, 239i |
| 376 | 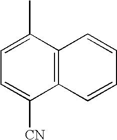 | 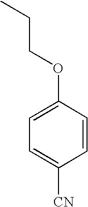 | [3aS- (3aα,4β,7β,7a α)]-4-[4-[2-(4- Cyanophenox y)ethyl]octahy dro-7-methyl- 1,3-dioxo-4,7- epoxy-2H- isoindol-2-yl]- 1- naphthaleneca rbonitrile. | 3.73 LC 483.65 [M + H]⁺ | 238ii, 239ii |
| 377 | 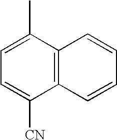 | 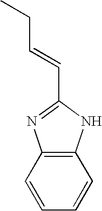 | [3aα,4β(E),7β, 7aα]-4-[4-[3- (1H- Benzimidazol- 2-yl)-2- propenyl]octah ydro-7-methyl- 1,3-dioxo-4,7- epoxy-2H- isoindol-2-yl]- 1- naphthaleneca rbonitrile | 2.48 LC 489.26 [M + H]⁺ | 248 |
| 378 | 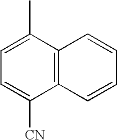 | 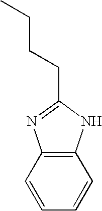 | (3aα,4β,7β,7a α)-4-[4-[3-(1H- Benzimidazol- 2- yl)propyl]octah ydro-7-methyl- 1,3-dioxo-4,7- epoxy-2H- isoindol-2-yl]- 1- naphthaleneca rbonitrile | 2.37 LC 491.26 [M + H]⁺ | 249 |

### EXAMPLES 379 to 381

Additional compounds of the present invention were prepared by procedures analogous to those described above. The compounds of Examples 379 to 381 have the following structure (L is a bond):

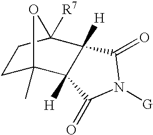

where G, R⁷, the compound name, retention time, molecular mass, and the procedure employed, are set forth in Table 6. The chromatography techniques used to determine the compound retention times of Table 6 are as follows: LCMS=YMC S5 ODS column, 4.6×50 mm eluting with 10-90% MeOH/H₂O over 4 minutes containing 0.1% TFA; 4 mL/min, monitoring at 220 nm. LCMS*=YMC S5 ODS column, 4.6×50 mm eluting with 10-90% MeOH/H₂O over 2 minutes containing 0.1% TFA; 4 mL/min, monitoring at 220 nm. LC=YMC S5 ODS column 4.6×50 mm eluting with 10-90% MeOH/H₂O over 4 minutes containing 0.2% phosphoric acid, 4 mL/min, monitoring at 220 nm.

The molecular mass of the compounds listed in Table 6 were determined by MS (ES) by the formula m/z.

TABLE 6

| | | | | Retention | |
|---|---|---|---|---|---|
| | | | | Time | |
| | | | | Min./ | |
| Ex. | | | Compound | Molecular | Procedure |
| No | G | R⁷ | Name | Mass | of Ex. |
| 379 | 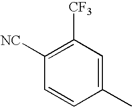 | 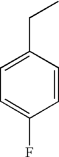 | (3aα,4α,7α,7aα)-4-[4-[(4- Fluorophenyl)methyl]octahydro-7- methyl-1,3-dioxo-4,7-epoxy-2H-isoindol- 2-yl]-2-(trifluoromethyl)benzonitrile. | 3.75 LC | 205 |
| 380 | 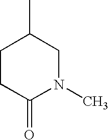 | CH₃ | (3aα,4α,7α,7aα)-Hexahydro-4,7- dimethyl-2-(1-methyl-6-oxo-3- piperidinyl)-4,7-epoxy-1H-isoindole- 1,3(2H)-dione. | 1.88 LC | 27 |
| 381 | 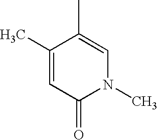 | CH₃ | (3aα,4α,7α,7aα)-2-(1,6-Dihydro- 1,4-dimethyl-6-oxo-3- pyridinyl)hexahydro-4,7-dimethyl-4,7- epoxy-1H-isoindole-1,3(2H)-dione. | 1.91 LC | 27 |

### EXAMPLES 382 to 383

Additional compounds of the present invention were prepared by procedures analogous to those described above. The compounds of Examples 382 to 383 have the structure, compound name, retention time, molecular mass, and were prepared by the procedure employed, set forth in the following Table 7. The chromatography techniques used to determine the compound retention times of Table 7 are as follows: LCMS=YMC S5 ODS column, 4.6×50 mm eluting with 10-90% MeOH/H₂O over 4 minutes containing 0.1% TFA; 4 mL/min, monitoring at 220 nm. LCMS*=YMC S5 ODS column, 4.6×50 mm eluting with 10-90% MeOH/H₂O over 2 minutes containing 0.1% TFA; 4 mL/min, monitoring at 220 nm. LC=YMC S5 ODS column 4.6×50 mm eluting with 10-90% MeOH/H₂O over 4 minutes containing 0.2% phosphoric acid, 4 mL/min, monitoring at 220 nm. The molecular mass of the compounds listed in Table 7 were determined by MS (ES) by the formula m/z.

TABLE 7

| | | | Retention | Procedure |
|---|---|---|---|---|
| Ex. | | Compound | Time | of |
| No. | Structure | Name | Min. | Example |
| 382 | 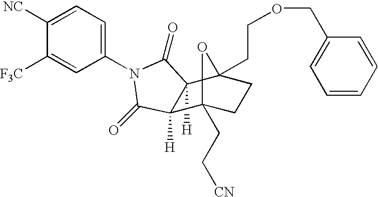 | (3aα,4β,7β,7aα)-2-[4-Cyano-3- (trifluoromethyl)phenyl]octahydro- 1,3-dioxo-7-[2-(phenylmethoxy) ethyl]-4,7-epoxy-4H-isoindole-4- propanenitrile. | 3.63 LC | 255 |
| 383 | 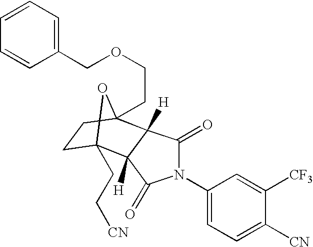 | (3aα,4β,7β,7aα)-2-[4-Cyano-3- (trifluoromethyl)phenyl]octahydro- 1,3-dioxo-7-[2-(phenylmethoxy) ethyl]-4,7-epoxy-4H-isoindole-4- propanenitrile. | 3.64 LC | 255 |

### EXAMPLES 384 to 418

Additional compounds of the present invention were prepared by procedures analogous to those described above. The compounds of Examples 384 to 418 have the following structure (L is a bond):

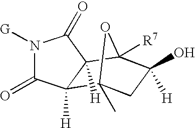

where G, R⁷, the compound name, retention time, molecular mass, and the procedure employed, are set forth in Table 8. The absolute configuration for the following compounds was not determined. For simplicity in nomenclature, compound 243Di is designated herein as having an “S” configuration and compound 243Dii as having an “R” configuration.

Enantiomerically pure products derived from compound 243Di are designated herein as having an “S” configuration and enantiomerically pure products derived from compound 243Dii are designated herein as having an “R” configuration.

The chromatography techniques used to determine the compound retention times of Table 8 are as follows: LCMS=YMC S5 ODS column, 4.6×50 mm eluting with 10-90% MeOH/H₂O over 4 minutes containing 0.1% TFA; 4 mL/min, monitoring at 220 nm. LCMS*=YMC S5 ODS column, 4.6×50 mm eluting with 10-90% MeOH/H₂O over 2 minutes containing 0.1% TFA; 4 mL/min, monitoring at 220 nm. LC=YMC S5 ODS column 4.6×50 mm eluting with 10-90% MeOH/H₂O over 4 minutes containing 0.2% phosphoric acid, 4 mL/min, monitoring at 220 nm.

The molecular mass of the compounds listed in Table 8 were determined by MS (ES) by the formula m/z.

TABLE 8

| | | | | Retention | |
|---|---|---|---|---|---|
| | | | | Time | |
| | | | | Min./ | |
| Ex. | | | Compound | Molecular | Procedure |
| No | G | R⁷ | Name | Mass | of Ex. |
| 384 | 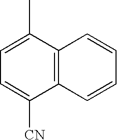 | 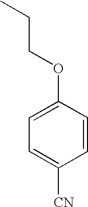 | (3aα,4β,7β,7aα)-4-[7-[2-(4- Cyanophenoxy)ethyl]octahydro- 5-hydroxy-4-methyl-1,3- dioxo-4,7-epoxy-2H-isoindol-2- yl]-1-naphthalenecarbonitrile. | 3.18 LC 494.40 [M + H]⁺ | 227, 228 229 |
| 385 | 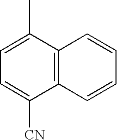 | 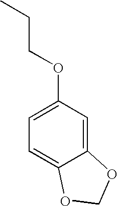 | [3aS-(3aα,4β,7β,7aα)]-4-[7-[2-(1,3- Benzodioxol-5-yloxy)ethyl]octahydro- 5-hydroxy-4-methyl-1,3- dioxo-4,7-epoxy-2H-isoindol-2- yl]-1-naphthalenecarbonitrile. | 3.19 LC 571.3 [M − H + OAc]⁻ | 234Di, 243I |
| 386 | 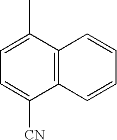 | 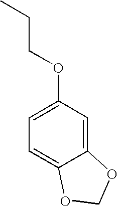 | [3aR-(3aα,4β,7β,7aα)]-4-[7-[2-(1,3- Benzodioxol-5-yloxy)ethyl]octahydro- 5-hydroxy-4-methyl-1,3- dioxo-4,7-epoxy-2H-isoindol-2- yl]-1-naphthalenecarbonitrile. | 3.22 LC 571.2 [M − H + OAc]⁻ | 234Dii, 243ii |
| 387 | 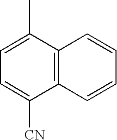 | 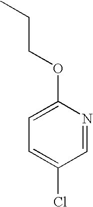 | [3aS-(3aα,4β,7β,7aα)]-4-[7-[2-[(5- Chloro-2-pyridinyl)oxy]ethyl]octahydro- 5-hydroxy-4-methyl-1,3- dioxo-4,7-epoxy-2H-isoindol-2- yl]-1-naphthalenecarbonitrile. | 3.37 LC 562.2 [M − H + OAc]⁻ | 234Di, 243I |
| 388 | 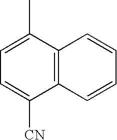 | 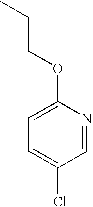 | [3aR-(3aα,4β,7β,7aα)]-4-[7-[2-[(5- Chloro-2-pyridinyl)oxy]ethyl]octahydro- 5-hydroxy-4-methyl-1,3- dioxo-4,7-epoxy-2H-isoindol-2- yl]-1-naphthalenecarbonitrile. | 3.37 LC 504.0 [M + H]⁺ | 234Dii, 243ii |
| 389 | 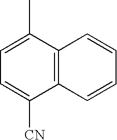 | 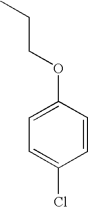 | [3aS-(3aα,4β,7β,7aα)]-4-[7-[2-(4- Chlorophenoxy)ethyl]octahydro- 5-hydroxy-4-methyl-1,3- dioxo-4,7-epoxy-2H-isoindol-2- yl]-1-naphthalenecarbonitrile. | 3.51 LC 503.08 [M + H]⁺ | 234Di, 243I |
| 390 | 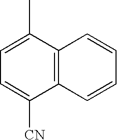 | 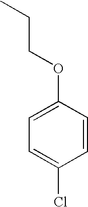 | [3aR-(3aα,4β,7β,7aα)]-4-[7-[2-(4- Chlorophenoxy)ethyl]octahydro- 5-hydroxy-4-methyl-1,3- dioxo-4,7-epoxy-2H-isoindol-2- yl]-1-naphthalenecarbonitrile. | 3.51 LC 503.08 [M + H]⁺ | 234Dii, 243ii |
| 391 | 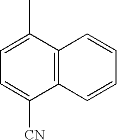 | 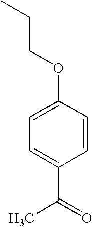 | [3aS-(3aα,4β,7β,7aα)]-4-[7-[2-(4- Acetylphenoxy)ethyl]octahydro- 5-hydroxy-4-methyl-1,3- dioxo-4,7-epoxy-2H-isoindol-2- yl]-1-naphthalenecarbonitrile. | 3.05 LC 511.13 [M + H]⁺ | 234Di, 243I |
| 392 | 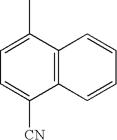 | 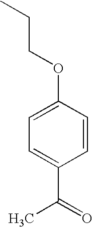 | [3aR-(3aα,4β,7β,7aα)]-4-[7-[2-(4- Acetylphenoxy)ethyl]octahydro- 5-hydroxy-4-methyl-1,3- dioxo-4,7-epoxy-2H-isoindol-2- yl]-1-naphthalenecarbonitrile. | 3.05 LC 503.13 [M + H]⁺ | 234Dii, 243ii |
| 393 | 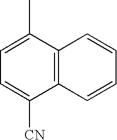 | 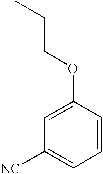 | [3aS-(3aα,4β,7β,7aα)]-4-[7-[2-(3- Cyanophenoxy)ethyl]octahydro- 5-hydroxy-4-methyl-1,3- dioxo-4,7-epoxy-2H-isoindol-2- yl]-1-naphthalenecarbonitrile. | 3.09 LC 494.13 [M + H]⁺ | 234Di, 243I |
| 394 | 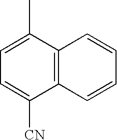 | 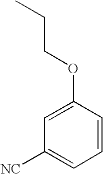 | [3aR-(3aα,4β,7β,7aα)]-4-[7-[2-(3- Cyanophenoxy)ethyl]octahydro- 5-hydroxy-4-methyl-1,3- dioxo-4,7-epoxy-2H-isoindol-2- yl]-1-naphthalenecarbonitrile. | 3.09 LC 494.13 [M + H]⁺ | 234Dii, 243ii |
| 395 | 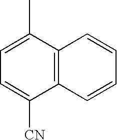 | 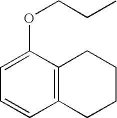 | [3aS-(3aα,4β,7β,7aα)]-4-[Octahydro-5- hydroxy-4-methyl-1,3-dioxo-7-[2- [(5,6,7,8-tetrahydro-1-naphtha- lenyl)oxy]ethyl]-4,7-epoxy-2H-isoindol- 2-yl]-1-naphthalenecarbonitrile. | 3.85 LC 523.17 [M + H]⁺ | 234Di, 243I |
| 396 | 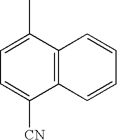 | 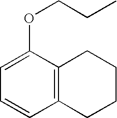 | [3aR-(3aα,4β,7β,7aα)]-4-[Octahydro-5- hydroxy-4-methyl-1,3-dioxo-7-[2- [(5,6,7,8-tetrahydro-1-naphtha- lenyl)oxy]ethyl]-4,7-epoxy-2H-isoindol- 2-yl]-1-naphthalenecarbonitrile. | 3.85 LC 523.17 [M + H]⁺ | 234Dii, 243ii |
| 397 | 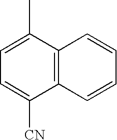 | 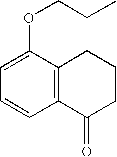 | [3aS-(3aα,4β,7β,7aα)]-4-[Octahydro-5- hydroxy-4-methyl-1,3-dioxo-7-[2- [(5,6,7,8-tetrahydro-5-oxo-1- naphthalenyl)oxy]ethyl]-4,7-epoxy-2H- isoindol-2-yl]-1-naphthalenecarbonitrile. | 3.29 LC 537.13 [M + H]⁺ | 234Di, 243I |
| 398 | 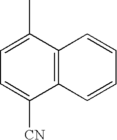 | 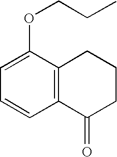 | [3aR-(3aα,4β,7β,7aα)]-4-[Octahydro-5- hydroxy-4-methyl-1,3-dioxo-7-[2- [(5,6,7,8-tetrahydro-5-oxo-1- naphthalenyl)oxy]ethyl]-4,7-epoxy-2H- isoindol-2-yl]-1-naphthalenecarbonitrile. | 3.29 LC 537.13 [M + H]⁺ | 234Dii, 243ii |
| 399 | 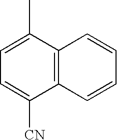 | 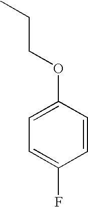 | [3aS-(3aα,4β,7β,7aα)]-4-[7-[2-(4- Fluorophenoxy)ethyl]octahydro- 5-hydroxy-4-methyl-1,3- dioxo-4,7-epoxy-2H-isoindol-2- yl]-1-naphthalenecarbonitrile. | 3.28 LC 487.11 [M + H]⁺ | 234Di, 243I |
| 400 | 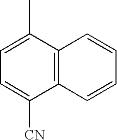 | 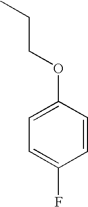 | [3aR-(3aα,4β,7β,7aα)]-4-[7-[2-(4- Fluorophenoxy)ethyl]octahydro- 5-hydroxy-4-methyl-1,3- dioxo-4,7-epoxy-2H-isoindol-2- yl]-1-naphthalenecarbonitrile. | 3.27 LC 487.11 [M + H]⁺ | 234Dii, 243ii |
| 401 | 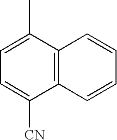 | 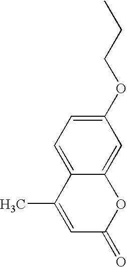 | [3aS-(3aα,4β,7β,7aα)]-4-[Octahydro-5- hydroxy-4-methyl-7-[2-[(4-methyl-2-oxo- 2H-1-benzopyran-7-yl)oxy]ethyl]-1,3- dioxo-4,7-epoxy-2H-isoindol-2-yl]-1- naphthalenecarbonitrile. | 3.15 LC 551.15 [M + H]⁺ | 234Di, 243I |
| 402 | 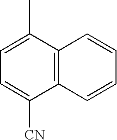 | 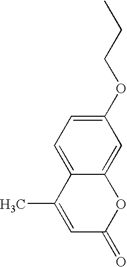 | [3aR-(3aα,4β,7β,7aα)]-4-[Octahydro-5- hydroxy-4-methyl-7-[2-[(4-methyl-2-oxo- 2H-1-benzopyran-7-yl)oxy]ethyl]-1,3- dioxo-4,7-epoxy-2H-isoindol-2-yl]-1- naphthalenecarbonitrile. | 3.16 LC 551.10 [M + H]⁺ | 234Dii, 243ii |
| 403 | 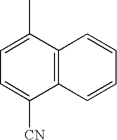 | 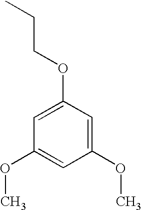 | [3aS-(3aα,4β,7β,7aα)]-4-[7-[2-(3,5- Dimethoxyphenoxy)ethyl]octahydro- 5-hydroxy-4-methyl-1,3- dioxo-4,7-epoxy-2H-isoindol-2- yl]-1-naphthalenecarbonitrile. | 3.28 LC 529.19 [M + H]⁺ | 234Di, 243I |
| 404 | 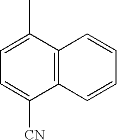 | 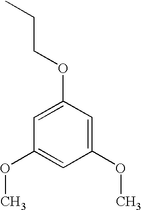 | [3aR-(3aα,4β,7β,7aα)]-4-[7-[2-(3,5- Dimethoxyphenoxy)ethyl]octahydro- 5-hydroxy-4-methyl-1,3- dioxo-4,7-epoxy-2H-isoindol-2- yl]-1-naphthalenecarbonitrile. | 3.26 LC 529.12 [M + H]⁺ | 234Dii, 243ii |
| 405 | 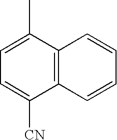 | 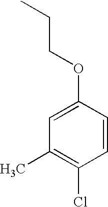 | [3aR-(3aα,4β,7β,7aα)]-]-4-[7-[2-(4- Chloro-3-methylphenoxy)ethyl]octahydro- 5-hydroxy-4-methyl-1,3- dioxo-4,7-epoxy-2H-isoindol-2- yl]-1-naphthalenecarbonitrile. | 3.68 LC 517.33 [M + H]⁺ | 234Dii, 243ii |
| 406 | 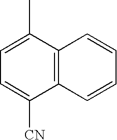 | 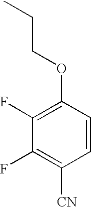 | [3aR-(3aα,4β,7β,7aα)]-4-[7-[2-(4- Cyano-2,3-difluorophenoxy)ethyl]octa- hydro-5-hydroxy-4-methyl-1,3- dioxo-4,7-epoxy-2H-isoindol-2- yl]-1-naphthalenecarbonitrile. | 3.23 LC 530.13 [M + H]⁺ | 234Dii, 243ii |
| 407 | 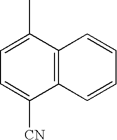 | 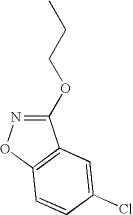 | [3aS-(3aα,4β,7β,7aα)]-4-[7-[2-[(5- Chloro-1,2-benzisoxazol-3- yl)oxy]ethyl]octahydro-5-hydroxy-4- methyl-1,3-dioxo-4,7-epoxy-2H-isoindol- 2-yl]-1-naphthalenecarbonitrile. | 3.59 LC 602.1 [M − H + OAc]⁻ | 234Di, 252 |
| 408 | 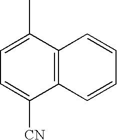 | 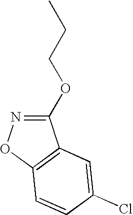 | [3aR-(3aα,4β,7β,7aα)]-4-[7-[2-[(5- Chloro-1,2-benzisoxazol-3- yl)oxy]ethyl]octahydro-5-hydroxy-4- methyl-1,3-dioxo-4,7-epoxy-2H-isoindol- 2-yl]-1-naphthalenecarbonitrile. | 3.57 LC 602.0 [M − H + OAc]⁻ | 243Dii, 253 |
| 409 | 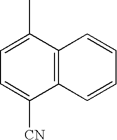 | 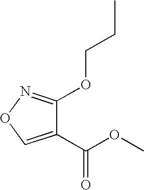 | [3aR-(3aα,4β,7β,7aα)]-3-[2-[2-(4- Cyano-1-naphthalenyl)octahydro-6- hydroxy-7-methyl-1,3-dioxo-4,7-epoxy- 4H-isoindol-4-yl]ethoxy]-5- isoxazolecarboxylic acid, methyl ester. | 2.90 LC 518.27 [M + H]⁺ | 243Dii, 253 |
| 410 | 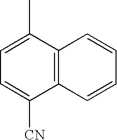 | 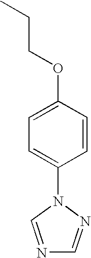 | [3aR-(3aα,4β,7β,7aα)]-4-[Octahydro-5- hydroxy-4-methyl-1,3-dioxo-7-[2-[4- (1H-1,2,4-triazol-1-yl)phenoxy]ethyl]- 4,7-epoxy-2H-isoindol-2-yl]-1- naphthalenecarbonitrile. | 2.93 LC 536.30 [M + H]⁺ | 243Dii, 244ii |
| 411 | 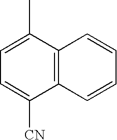 | 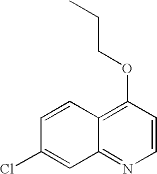 | [3aS-(3aα,4β,7β,7aα)]-4-[7-[2-[(7- Chloro-4-quinolinyl)oxy]ethyl]octahydro- 5-hydroxy-4-methyl-1,3-dioxo-4,7-epoxy- 2H-isoindol-2-yl]-1-naphthalene- carbonitrile, trifluoroacetate (1:1). | 2.52 LC 554.13 [M + H]⁺ | 243Di, 243i |
| 412 | 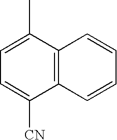 | 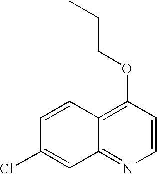 | [3aR-(3aα,4β,7β,7aα)]-4-[7-[2-[(7- Chloro-4-quinolinyl)oxy]ethyl]octahydro- 5-hydroxy-4-methyl-1,3-dioxo-4,7-epoxy- 2H-isoindol-2-yl]-1-naphthalene- carbonitrile, trifluoroacetate (1:1). | 2.53 LC 554.27 [M + H]⁺ | 243Dii, 244ii |
| 413 | 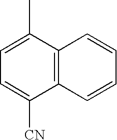 | 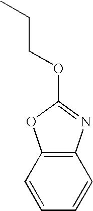 | [3aR-(3aα,4β,5β,7β,7aα)]-4-[7-[2-(2- Benzoxazolyloxy)ethyl]octahydro- 5-hydroxy-4-methyl-1,3-dioxo-4,7- epoxy-2H-isoindol-2-yl]-1- naphthalenecarbonitrile. | 3.13 LC 568.1 [M − H + OAc]⁻ | 243Dii, 244ii |
| 414 | 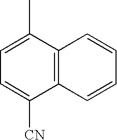 | 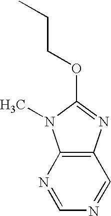 | [3aR-(3aα,4β,5β,7β,7aα)]-4- [Octahydro-5-hydroxy-4-methyl-7-[2-[(9- methyl-9H-purin-8-yl)oxy]ethyl]- 1,3-dioxo-4,7-epoxy-2H-isoindol-2-yl]-1- naphthalenecarbonitrile | 2.34 LC 525.2 [M + H]⁺ | 243Dii, 244ii |
| 415 | 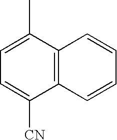 | 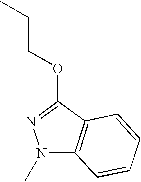 | [3aR-(3aα,4β,5β,7β,7aα)]-4-[Octahydro- 5-hydroxy-4-methyl-7-[2-[(1- methyl-1H-indazol-3-yl)oxy]ethyl]-1,3- dioxo-4,7-epoxy-2H-isoindol-2-yl]-1- naphthalenecarbonitrile | 3.33 LC | 251, 253 |
| 416 | 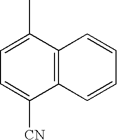 | 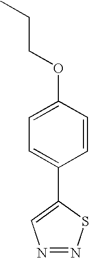 | [3aS-(3aα,4β,5β,7β,7aα)]-4-[Octahydro- 5-hydroxy-4-methyl-7-[2-[4-(1,2,3- thiadiazol-5-yl)phenoxy]ethyl]-1,3- dioxo-4,7-epoxy-2H-isoindol-2-yl]-1- naphthalenecarbonitrile | 3.17 LC 553.10 [M + H]⁺ | 243Dii, 244ii |
| 417 | 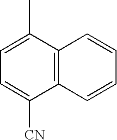 | 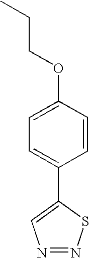 | [3aR-(3aα,4β,5β,7β,7aα)]-4-[Octahydro- 5-hydroxy-4-methyl-7-[2-[4-(1,2,3- thiadiazol-5-yl)phenoxy]ethyl]-1,3- dioxo-4,7-epoxy-2H-isoindol-2-yl]-1- naphthalenecarbonitrile | 3.20 LC 553.25 [M + H]⁺ | 243Dii, 244ii |
| 418 | 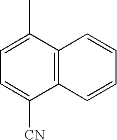 | 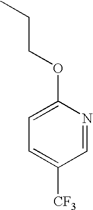 | [3aS-(3aα,4β,5β,7β,7aα)]-4-[Octahydro- 5-hydroxy-4-methyl-1,3-dioxo-7-[2-[[5- (trifluoromethyl)-2-pyridinyl]oxy]ethyl]- 4,7-epoxy-2H-isoindol-2-yl]-1- naphthalenecarbonitrile | 3.45 LC 538.23 [M + H]⁺ | 243Dii, 244ii |
| 419 | 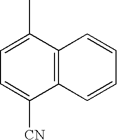 | 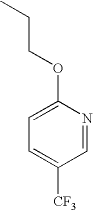 | [3aR-(3aα,4β,5β,7β,7aα)]-4-[Octahydro- 5-hydroxy-4-methyl-1,3-dioxo-7-[2-[[5- (trifluoromethyl)-2-pyridinyl]oxy]ethyl]- 4,7-epoxy-2H-isoindol-2-yl]-1- naphthalenecarbonitrile | 3.45 LC 538.23 [M + H]⁺ | 243Dii, 244ii |
| 420 | 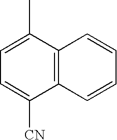 | 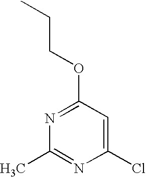 | [3aS-(3aα,4β,5β,7β,7aα)]-4-[7-[2-[(6- Chloro-2-methyl-4-pyrimidi- nyl)oxy]ethyl]octahydro-5-hydroxy-4- methyl-1,3-dioxo-4,7-epoxy-2H-isoindol- 2-yl]-1-naphthalenecarbonitrile | 3.02 LC | 243Dii, 244ii |
| 421 | 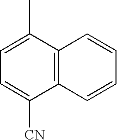 | 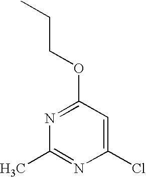 | [3aR-(3aα,4β,5β,7β,7aα)]-4-[7-[2-[(6- Chloro-2-methyl-4-pyrimidi- nyl)oxy]ethyl]octahydro-5-hydroxy-4- methyl-1,3-dioxo-4,7-epoxy-2H-isoindol- 2-yl]-1-naphthalenecarbonitrile | 3.02 LC | 243Dii, 244ii |

### EXAMPLE 422

### (3aα,4β,7β,7aα)-2-(7-Bromo-2,1,3-benzoxadiazol4-yl)hexahydro-4,7-dimethyl-4,7-epoxy-1H-isoindole-1,3(2H)-dione (422C)

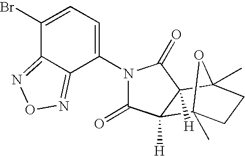

A. 4-Bromo-7-nitrobenzofurazan (422A)

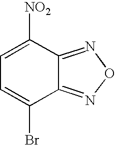

To a solution of 2,6-dibromoaniline (1.0 g, 4.0 mmol) in CHCl₃ (8 mL) was added a suspension of mCPBA (70% by HPLC, 1.4 g, 8.0 mmol) in CHCl₃ (8 mL) and the resulting mixture was stirred for 24 h at rt. The reaction mixture was diluted with CHCl₃ and washed successively with 2% Na₂S₂O₃ solution, 5% Na₂CO₃ solution and brine. The organic layer was dried over Na₂SO₄ and concentrated under reduced pressure to leave a solid, which was suspended, into DMSO (15 mL). To this suspension was added a solution of NaN. (272 mg, 4.19 mmol) in DMSO (15 mL) at rt. The resulting mixture was stirred at rt until most of the nitrogen had evolved and was then quickly heated to 120° C. for 3 min. The reaction mixture was cooled and poured onto crushed ice (100 g). After standing for 1 h the precipitates were filtered off, dried in vacuo and redissolved in concentrated H₂SO₄ (5 mL). To this solution was added a solution of NaNO₃ (400 mg, 4.7 mmol) in 50% H₂SO₄ (1.6 mL) and the temperature was maintained at 60° C. After the addition was complete, the mixture was heated to 85° C. for 30 min, cooled to rt and poured onto crushed ice (40 g). EtOAc was added, the layers were separated and the aqueous layer was extracted with EtOAc. The combined organic layers were dried over Na₂SO₄ and concentrated to leave a solid which was purified by flash chromatography (silica gel, EtOAc (20%) in hexanes) affording compound 422A (785 mg, 81%) as a tan solid.

B. 4-Bromo-7-aminobenzofurazan (422B)

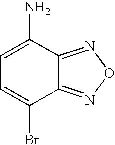

A solution of compound 422A (563 mg, 2.31 mmol) in AcOH (5 mL) was heated to 70° C. and Fe⁰ powder (258 mg, 4.62 mmol) was added in one portion. The resulting dark reaction mixture was stirred for 15 min, cooled to rt and concentrated under reduced pressure. The residue was taken up in EtOAc and the resulting solution was washed with sat. Na₂CO₃ solution. The organic layer was dried over Na₂SO₄, concentrated and purified by flash chromatography (silica gel, EtOAc in hexanes 10 to 60%) yielding compound 422B (470 mg, 95%) as a red solid.

C. (3aα,4β,7β,7aα)-2-(7-Bromo-2,1,3-benzoxadiazol-4-yl)hexahydro-4,7-dimethyl-4,7-epoxy-1H-isoindole-1,3(2H)-dione (422C)

A mixture of compound 422B (43 mg, 0.20 mmol), compound 20A (45 mg, 0.23 mmol), MgSO₄ (60 mg, 0.50 mmol), Et₃N (139 μL, 1.0 mmol) and 1,2-dimethoxyethane (300 μL) were placed in a sealed tube and heated to 135° C. for 14 h. After cooling to rt the mixture was filtered through celite eluting with MeOH to yield a dark solid which was purified by flash chromatography (silica gel, EtOAc in hexanes 5 to 40%) furnishing compound 422C (42 mg, 54%) as a yellow solid. HPLC: 99% at 2.96 min (retention time) (YMC S5 ODS column 4.6×50 mm Ballistic, 10-90% aqueous methanol over 4 minutes containing 0.2% H₃PO₄, 4 mL/min, monitoring at 220 nm). ¹H NMR (acetone-d₆, 400 MHz): δ=8.00 (d, J=7.5 Hz, 1H), 7.45 (d, J=7.5 Hz, 1H), 3.31 (s, 2H), 1.98-1.93 (m, 2H), 1.74-1.69 (m, 2H), 1.57 (s, 6H).

### EXAMPLE 423

### (3aα,4β,7β,7aα)-7-[Octahydro-4,7-dimethyl-1,3-dioxo-4,7-epoxy-2H-isoindol-2-yl]-2,1,3-benzoxadiazole-4-carbonitrile (423)

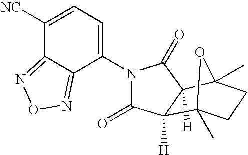

To a solution of compound 422C (42 mg, 0.11 mmol) in DMA (1 mL) was added CuCN (20 mg, 0.22 mmol) and the resulting mixture was heated to 150° C. for 5 h. The mixture was allowed to cool to rt and partitioned between EtOAc and aqueous NaCN solution (5 g/50 mL). The layers were separated and the aqueous layer was extracted once with EtOAc. The combined organic phases were dried over Na₂SO₄, concentrated and purified by flash chromatography (silica gel, EtOAc in hexanes 20 to 70%) to give compound 423 (13 mg, 35%) as a yellow oil. HPLC: 99% at 2.66 min (retention time) (YMC S5 ODS column 4.6×50 mm Ballistic, 10-90% aqueous methanol over 4 minutes containing 0.2% H₃PO₄, 4 mL/min, monitoring at 220 nm), MS (ES): m/z 396.9 [M−H+OAc]⁻.

### EXAMPLE 424

### (3aα,4β,7β,7aα)-7-[Octahydro-4,7-dimethyl-1,3-dioxo-4,7-epoxy-2H-isoindol-2-yl]-2,1,3-benzothiadiazole-4-carbonitrile (424B)

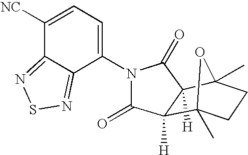

A. 4-Cyano-7-amino-benzothiadiazole (424A)

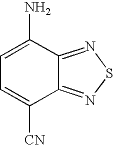

A solution of 2-cyano-5-nitrophenylenediamine (78 mg, 0.44 mmol, prepared as described in WO 0076501) in SOCl₂ (2 mL) was heated to reflux for 3 h. The resulting mixture was allowed to cool to rt and was then poured into ice/water. CH₂Cl₂ was added, the layers were separated and the aqueous layer was extracted twice with CH₂Cl₂. The combined organic phases were dried over MgSO₄, concentrated and purified by flash chromatography (silica gel, EtOAc in hexanes 50%) to give 4-cyano-7-nitrobenzothiadiazole. This material was dissolved in AcOH (2 mL) containing EtOAc (1 mL) and H₂O (0.2 mL) and heated to 70° C. At this temperature Fe⁰ powder (78 mg, 1.41 mmol) was added in one solid portion and the dark mixture was stirred for 20 min and then cooled to rt. The reaction mixture was filtered through Celite eluting with EtOAc, washed with sat. Na₂CO₃ solution, dried over MgSO₄ and concentrated. Purification was achieved by flash chromatography (silica gel, EtOAc in hexanes 20 to 70%) to yield compound 424A (47 mg, 61%) as a brown solid.

B. (3aα4β,7β,7aα)-7-[Octahydro-4,7-dimethyl-1,3-dioxo-4,7-epoxy-2H-isoindol-2-yl]-2,1,3-benzothiadiazole-4-carbonitrile (424B)

A mixture of compound 424A (35 mg, 0.20 mmol), compound 20A (45 mg, 0.23 mmol), MgSO₄ (60 mg, 0.50 mmol), Et₃N (139 μL, 1.0 mmol) and DME (200 μL) was placed in a sealed tube and heated to 135° C. for 14 h. After cooling to rt the mixture was filtered through Celite eluting with MeOH to yield a dark solid which was purified by a combination of flash chromatography (silica gel, EtOAc in hexanes 10 to 50%) and reverse phase preparative HPLC (YMC S5 ODS 20×100 mm eluting with 27-100% aqueous methanol over 10 min containing 0.1% TFA, 20 mL/min) furnishing compound 424B (36 mg, 51%) as a yellow solid. HPLC: 98% at 2.45 min (retention time) (YMC S5 ODS column 4.6×50 mm Ballistic, 10-90% aqueous methanol over 4 minutes containing 0.2% H₃PO₄, 4 mL/min, monitoring at 220 nm), MS (DCI): m/z 355.0 [M+H]⁺.

### EXAMPLE 425

### (3aα,4β,7β,7aα)-N-[2-[2-(4-Cyano-1-naphthalenyl)octahydro-7-methyl-1,3-dioxo-4,7-epoxy-4H-isoindol-4-yl]ethyl]-4-fluoro-N-methylbenzamide (425B)

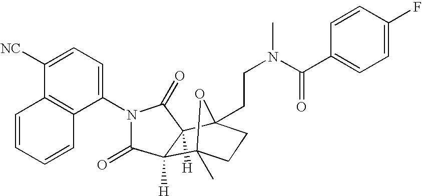

A. 4-Fluoro-N-methyl-N-[2-(5-methyl-furan-2-yl)-ethyl]-benzamide (425A)

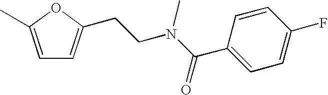

NaH (60% dispersion in oil, 65 mg, 1.63 mmol) was added portion-wise to a solution of 4-fluoro-N-[2-(5-methyl-2-furanyl)ethyl]benzamide (269 mg, 1.09 mmol, 237A) in THF (5 mL). After gas evolution ceased, iodomethane (0.14 mL, 2.18 mmol) was added drop-wise. Once HPLC analysis showed the reaction to be 50% complete, the mixture was concentrated under reduced pressure and resubjected to the above conditions. After all the starting material was consumed, H₂O was added and the resulting mixture was extracted with EtOAc (2×5 mL). The combined organic layers were dried over Na₂SO₄ and concentrated under reduced pressure. Purification by flash chromatography eluting with 20% acetone/CHCl₃ gave 238 mg (84%) of compound 425A. HPLC: 98% at 2.94 min (retention time) (Phenomenex-prime S5-C18 column 4.6×50 mm eluting with 10-90% aqueous methanol over 4 minutes containing 0.2% phosphoric acid, 4 mL/min, monitoring at 220 nm), MS (ES): m/z [M+H]=262.38.

B. (3aα,4β,7β,7aα)-N-[2-[2-(4-Cyano-1-naphthalenyl)octahydro-7-methyl-1,3-dioxo-4,7-epoxy-4H-isoindol-4-yl]ethyl]-4-fluoro-N-methylbenzamide (425B)

A solution of compound 425A (183 mg, 0.75 mmol) and 4-(2,5-dihydro-2,5-dioxo-1H-1-yl)-1-naphthalenecarbonitrile (174 mg, 0.75 mmol) in benzene (1 mL) was heated at 60° C. for 15 hr. The reaction mixture was concentrated under reduced pressure to give 357 mg crude intermediate. The crude intermediate (156 mg) was dissolved in EtOAc (6 mL) and 10% Pd/C (16 mg) was added and the mixture was stirred under a hydrogen balloon overnight. The reaction mixture was filtered through a pad of celite and concentrated under reduced pressure. Purification by reverse phase preparative chromatography (YMC S5 ODS 20×100 mm, 20-100% aqueous methanol over 15 minutes containing 0.1% TFA, 20 mL/min, monitoring at 220 mn) gave 160.3 mg (72%) of compound 425B as an off-white solid. HPLC: 99% at 3.23 min (retention time) (Phenomenex-prime S5-C18 column 4.6×50 mm eluting with 10-90% aqueous methanol over 4 minutes containing 0.2% phosphoric acid, 4 mL/min, monitoring at 220 nm), MS (ES): m/z [M+H]=512.19.

### EXAMPLE 426

### (3aα,4β,7β,7aα)-Hexahydro-4,7-dimethyl-2-[4-(2,2,2-trifluoro-1-hydroxyethyl)phenyl]-4,7-epoxy-1H-isoindole-1,3(2H)-dione (426B)

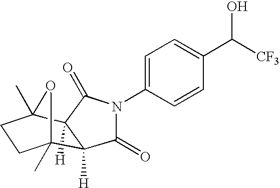

A. 1-(4-Amino-phenyl)-2,2,2-trifluoroethanol (426A)

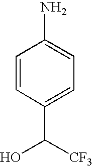

Compound 426A was made according to the procedure described in Stewart, R. et. al., Can. J Chem. 58, 2491-2496 (1980). NaBH₄ (47 mg, 1.235 mmol) was added to a solution of p-aminotrifluoroacetophenone (155.7 mg, 0.823 mmol, synthesized as described by Klabunde, K. J. et. al., J Org. Chem. 35, 1711-1712 (1970)) in isopropanol (3 mL) at rt. After 30 min the reaction was quenched with phosphate buffer (pH 7.2), diluted with H₂O and extracted with EtOAc (2×10 mL). The combined organic layers were dried over Na₂SO₄ and concentrated under reduced pressure to give 154 mg (98%) of compound 426A as a tan solid. The material was used directly in the next step without purification. HPLC: 99% at 0.42 min (retention time) (YMC S5 ODS column 4.6×50 mm eluting with 10-90% aqueous methanol over 4 minutes containing 0.2% phosphoric acid, 4 mL/min, monitoring at 220 nm), MS (ES): m/z [M+H]=192.13.

B. (3aα,4β,7β,7aα)-Hexahydro-4,7-dimethyl-2-[4-(2,2,2-trifluoro-1-hydroxyethyl)phenyl]4,7-epoxy-1H-isoindole-1,3(2H)-dione (426B)

A mixture of compound 426A (75.3 mg, 0.394), compound 20A (51.5 mg, 0.262 mmol), triethylamine (0.15 mL) and MgSO₄ (50 mg) in toluene (1 mL) was heated in a sealed tube to 135° C. for 15 hr. The mixture was filtered and concentrated under reduced pressure. Purification by reverse phase preparative chromatography (YMC S5 ODS 20×100 mm, 20-100% aqueous methanol over 15 minutes containing 0.1% TFA, 20 mL/min, monitoring at 220 nm) gave 63.1 mg (65%) of compound 426B as a white solid. HPLC: 98% at 2.49 min (retention time) (Phenomenex-prime S5-C18 column 4.6×50 mm eluting with 10-90% aqueous methanol over 4 minutes containing 0.2% phosphoric acid, 4 mL/min, monitoring at 220 nm), MS (ES): m/z [M+H]=370.16.

### EXAMPLE 427

### (3aα,4β,7β,7aα)-4-[4-[2-[[(1,1-Dimethylethyl)dimethylsilyl]oxy]ethyl]-1,3,3a,4,7,7a-hexahydro-7-methyl-1,3-dioxo-4,7-epoxy-2H-isoindol-2-yl]-2-(trifluoromethyl)benzonitrile & (3aα,4α,7α,7aα)-4-[4-[2-[[(1-Dimethylethyl)dimethylsilyl]oxy]ethyl]-1,3,3a,4,7,7a-hexahydro-7-methyl-1,3-dioxo-4,7-epoxy-2H-isoindol-2-yl]-2-(trifluoromethyl)benzonitrile (427i & 427ii)

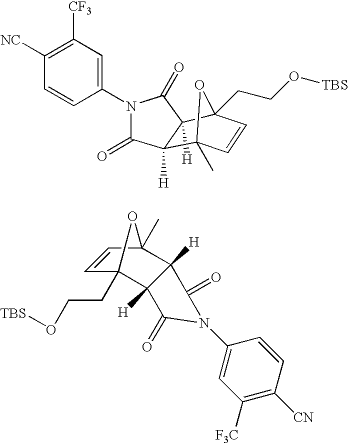

Compound 204A (2.00 g, 8.50 mmol) and 4-(2,5-Dihydro-2,5-dioxo-1H-pyrrol-1-yl)-2-trifluoromethylbenzonitrile (1.50 g, 5.60 mmol) were mixed in benzene (5.0 mL) and heated at 60° C. for 14 h, then cooled to 25° C. The solvent was removed at 40° C. under vacuum for 1 h to give the crude material which was purified by flash chromatography on SiO₂ eluting with 0.5% EtOAc/CH₂Cl₂ to give 2.0 g of compound 427i and 1.3 g of compound 427ii, both as light brown solids. Compound 427i: HPLC: 95% at 4.200 min (retention time) (YMC S5 ODS column 4.6×50 mm eluting with 10-90% aqueous methanol over 4 minutes containing 0.2% phosphoric acid, 4 mL/min, monitoring at 220 nm), MS (ES): m/z 507.1 [M+H]⁺. Compound 427ii: HPLC: 95% at 4.20 min (retention time) (YMC S5 ODS column 4.6×50 mm eluting with 10-90% aqueous methanol over 4 minutes containing 0.2% phosphoric acid, 4 mL/min, monitoring at 220 nm), MS (ES): m/z 507.1 [M+H]⁺.

### EXAMPLE 428

### [3aR-(3aα,4β,5β,7β,7aα)]-4-[7-[2-[[(1-Dimethylethyl)dimethylsilyl]oxy]ethyl]octahydro-5-hydroxy-4-methyl-1,3-dioxo-4,7-epoxy-2H-isoindol-2-yl]-2-(trifluoromethyl)benzonitrile & [3aS-(3aα,4β,5β,7β,7aα)]-4-[7-[2-[[(1,1-Dimethylethyl)dimethylsilyl]oxy]ethyl]octahydro-5-hydroxy-4-methyl-1,3-dioxo-4,7-epoxy-2H-isoindol-2-yl]-2-trifluoromethyl)benzonitrile (428i & 428ii)

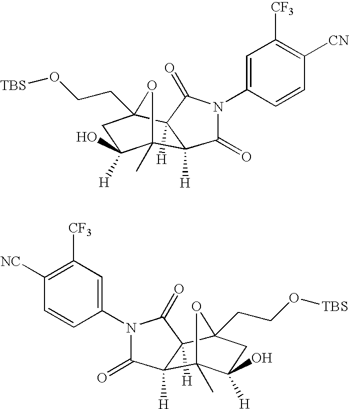

Compound 427i (1.40 g, 2.77 mmol) and RhCl(PPh₃)₃ (0.128 g, 0.14 mmol) were mixed in a flask. The flask was then evacuated and filled with argon three times, followed by the syringe addition of THF (3.0 mL). Once all particulates were dissolved, catecholborane (0.59 mL, 5.54 mmol) was added dropwise. The reaction mixture was stirred at 25° C. under argon for 30 min, then cooled to 0° C. Phosphate buffer (pH=7, 20 mL) was added, followed by EtOH (10 mL), 30% H₂O₂/H₂O (2 mL). The reaction mixture was stirred at 0° C. for 3 h, then extracted with dichloromethane (3×25 mL). The combined organic layers were washed with 1 N NaOH (25 mL), 10% Na₂SO₃ (25 mL) and brine (25 mL). The crude material was then concentrated and purified by flash chromatography on SiO₂ eluting with 2% EtOAc/CH₂Cl₂ to 10% EtOAc/CH₂Cl₂ to give 0.63 g of a racemic mixture of compounds 428i & 428ii as a light yellow solid. HPLC: 99% at 3.867 min (retention time) (YMC S5 ODS column 4.6×50 mm eluting with 10-90% aqueous methanol over 4 minutes containing 0.2% phosphoric acid, 4 mL/min, monitoring at 220 nm), MS (ES): m/z 525.1 [M+H]

The racemic mixture of compounds 428i & 428ii was separated by normal phase preparative chiral HPLC using a Chiracel OD column (5 cm×50 cm), eluting with 13% solvent B (EtOH) in solvent A (Hexane), flow rate: 50 mL/min. Compound 428i eluted from 34 min to 38 min and compound 428ii eluted from 44 min to 49 min. Enantiomeric excess was determined by chiral HPLC. Compound 428i: >99% ee (12.576 min (retention time) (Chiralcel OJ column 4.6×250 mm eluting with isocratic 85% heptane/15% MeOH/ethanol (1:1), 1 mL/min, monitoring at 220 nm, 40° C.). Compound 428ii: 99% ee (18.133 min (retention time) (Chiralcel OJ column 4.6×250 mm eluting with isocratic 85% heptane/15% MeOH/ethanol (1:1), 1 mL/min, monitoring at 220 nm, 40° C.).

The absolute configurations for compounds 428i & 428ii were not established. For simplicity in nomenclature, compound 428i is designated herein as having an “R” configuration and compound 428ii as having an “S” configuration. Enantiomerically pure products derived from compound 428i are designated herein as having a “R” configuration and enantiomerically pure products derived from compound 428ii are designated herein as having an “S” configuration.

### EXAMPLE 429

### [3aR-(3aα,4β,5β,7β,7aα)]-4-[Octahydro-5-hydroxy-7-(2-hydroxyethyl)-4-methyl-1,3-dioxo-4,7-epoxy-2H-isoindol-2-yl]-2-(trifluoromethyl)benzonitrile & [3aS-(3aα,4β,5β,7β,7aα)]-4-[Octahydro-5-hydroxy-7-(2-hydroxyethyl)-4-methyl-1,3-dioxo-4,7-epoxy-2H-isoindol-2-yl]-2-(trifluoromethyl)benzonitrile (4291 & 429ii)

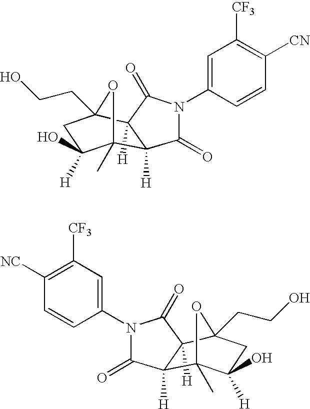

Compound 428i (180 mg, 0.34 mmol) was dissolved in 2% HCl/EtOH (5.0 mL). After 30 min, saturated NaHCO₃ was added and the aqueous layer was extracted with dichloromethane (20 mL×3), washed with brine and dried over Na₂SO₄ to give 135 mg of compound 4291 as a white solid. HPLC: 99% at 2.257 min (retention time) (YMC S5 ODS column 4.6×50 mm eluting with 10-90% aqueous methanol over 4 minutes containing 0.2% phosphoric acid, 4 mL/min, monitoring at 220 nm), MS (ES): m/z 411.1 [M+H]⁺.

The above procedure was repeated with compound 428ii to yield the desired diol compound 429ii in similar yield.

### EXAMPLE 430

### [3aR-(3aα,4β,5β,7β,7aα)]-4-[7-[2-[(5-Chloro-2-pyridinyl)oxy]ethyl]octahydro-5-hydroxy-4-methyl-1,3-dioxo-4,7-epoxy-2H-isoindol-2-yl]-2-(trifluoromethyl)benzonitrile (430)

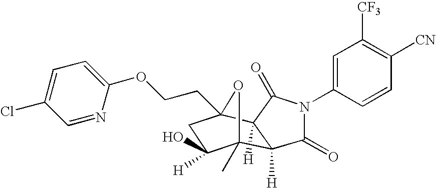

Triphenylphosphine (0.026 g, 0.098 mmol) and DBAD (0.023 g, 0.098 mmol) were mixed in THF (0.5 mL). After allowing the previous mixture to react for 15 min, 2-hydroxy-6-chloropyridine (0.016 g, 0.100 mmol) was added, the mixture was allowed to stir for 10 min and compound 429i (0.020 g, 0.049 mmol) was added. The reaction mixture was stirred at 25° C. for 2 h and then the crude material was purified by preparative TLC, eluting with 10% acetone/CHCl₃, to give 0.014 g of compound 430 as a light brown solid. HPLC: 100% at 3.370 min (retention time) (YMC S5 ODS column 4.6×50 mm eluting with 10-90% aqueous methanol over 4 minutes containing 0.1% TFA, 4 mL/min, monitoring at 220 nm), MS (ES): m/z 522.08 [M+H]⁺.

### EXAMPLE 431

### [3aS-(3aα,4β,5β,7β,7aα)]-4-[7-[2-[(5-Chloro-2-pyridinyl)oxy]ethyl]octahydro-5-hydroxy-4-methyl-1,3-dioxo-4,7-epoxy-2H-isoindol-2-yl]-2-(trifluoromethyl)benzonitrile (431)

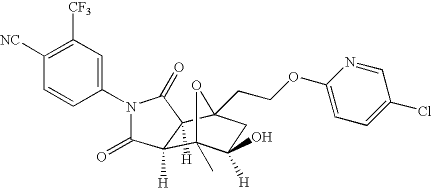

Triphenylphosphine (0.026 g, 0.098 mmol) and DBAD (0.023 g, 0.098 mmol) were mixed in THF (0.5 mL). After allowing the previous mixture to react for 15 min, 2-hydroxy-6-chloropyridine (0.016 g, 0.100 mmol) was added, the mixture was allowed to stir for 10 min and compound 429ii (0.020 g, 0.049 mmol) was added. The reaction mixture was stirred at 25° C. for 2 h and then the crude material was purified by preparative TLC, eluting with 10% acetone/CHCl₃, to give 0.015 g of compound 431 as a light brown solid. HPLC: 100% at 3.370 min (retention time) (YMC S5 ODS column 4.6×50 mm eluting with 10-90% aqueous methanol over 4 minutes containing 0.1% TFA, 4 mL/min, monitoring at 220 nm), MS (ES): m/z 522.08 [M+H]⁺.

### EXAMPLE 432

### (3aα,4β,7β,7aα)-2-(4-Cyano-1-naphthalenyl)octahydro-N-(2-hydroxyphenyl)-7-methyl-1,3-dioxo-4,7-epoxy-4H-isoindole-4-butanamide (432)

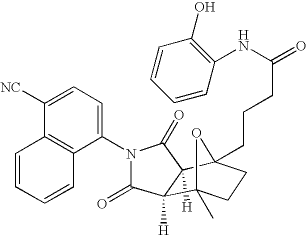

Compound 262 (0.100 g, 0.239 mmol) was dissolved in DMF (anhydrous, 1.5 mL), BOP (0.211 g, 0.478 mmol) was added followed by 2-aminophenol (0.052 g, 0.478 mmol) and N-methyl morpholine (0.052 mL, 0.478 mmol). The reaction mixture was stirred at 25° C. under argon for 3 h, then the crude material was purified by reverse phase preparative-HPLC (YMC S5 ODS 20×100 mm, 20-100% aqueous methanol over 15 minutes containing 0.1% TFA, 20 mL/min, monitoring at 220 nm) to give 0.060 g of compound 432 as a light brown solid. HPLC: 100% at 3.037 min (retention time) (YMC S5 ODS column 4.6×50 mm eluting with 10-90% aqueous methanol over 4 minutes containing 0.1% TFA, 4 mL/min, monitoring at 220 nm), MS (ES): m/z 510.34 [M+H]⁺.

### EXAMPLE 433

### (3aα,4β,7β,7aα)-4-[4-[3-(2-Benzoxazolyl)propyl]octahydro-7-methyl-1,3-dioxo-4,7-epoxy-2H-isoindol-2-yl]-1-naphthalenecarbonitrile (433)

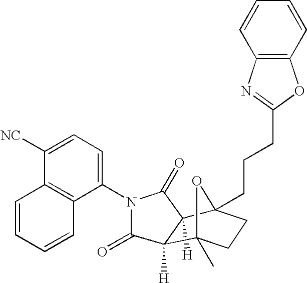

Triphenylphosphine (0.031 g, 0.118 mmol) and DBAD (0.027 g, 0.118 mmol) were mixed in THF (0.5 mL). After allowing the previous mixture to react for 15 min, compound 432 (0.030 g, 0.059 mmol) was added. The reaction mixture was stirred at 25° C. for 2 h and then the crude material was purified by reverse phase preparative-HPLC (YMC S5 ODS 20×100 mm, 20-100% aqueous methanol over 15 minutes containing 0.1% TFA, 20 mL/min, monitoring at 220 nm) to give 0.018 g of compound 433 as a light brown solid. HPLC: 100% at 3.357 min (retention time) (YMC S5 ODS column 4.6×50 mm eluting with 10-90% aqueous methanol over 4 minutes containing 0.1% TFA, 4 mL/min, monitoring at 220 nm), MS (ES): m/z 492.37 [M+H]⁺.

### EXAMPLE 434

### (3aα,4β,5β,7β,7aα)-4-[4-Ethyloctahydro-5-hydroxy-7-(2-hydroxyethyl)-1,3-dioxo-4,7-epoxy-2H-isoindol-2-yl]-1-naphthalenecarbonitrile (434C)

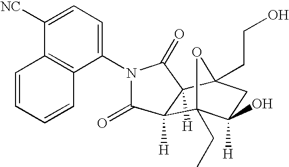

A. tert-Butyl-[2-(5-ethyl-furan-2-yl)-ethoxy]-dimethyl-silane (434A)

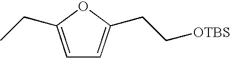

Imidazole (255 mg, 3.75 mmol) and TBSCl (414 mg, 2.75 mmol) were added to the solution of 245A (350 mg, 2.5 mmol) in DMF (4 mL). The mixture was stirred at rt for 15 hr and then 100 mg (0.66 mmol) of additional TBSCl was added to drive the reaction to completion. After stirring for an additional hour, the reaction mixture was diluted with diethylether (100 mL) and washed with water (20 mL), 1 N HCl (20 mL), water (20 mL) and brine (20 mL). The organic layer was dried over Na₂SO₄ and concentrated under reduced pressure to give 509 mg of compound 434A (80.3%) as a yellow oil.

B. (3aα,4β,7β,7aα)-4-[4-[2-[[(1,1-Dimethylethyl)-dimethylsilyl]oxy]ethyl]-4-ethyl-1,3,3a,4,7,7a-hexahydro-1,3-dioxo-4,7-epoxy-2H-isoindol-2-yl]-1-naphthalenecarbonitrile (434B)

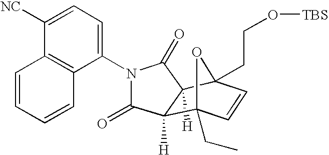

A solution of compound 434A (509 mg, 2.00 mmol) and 4-(2,5-dihydro-2,5-dioxo-1H-1-yl)-1-naphthalenecarbonitrile (498 mg, 2.00 mmol) in benzene (2 mL) was heated at 60° C. for 18 h. The reaction mixture was concentrated under reduced pressure to give 992 mg (99%) of crude compound 434B, which was used directly in the next step without further purification.

C. (3aα,4β,5β,7β,7aα)-4-[4-Ethyloctahydro-5-hydroxy-7-(2-hydroxyethyl)-1,3-dioxo-4,7-epoxy-2H-isoindol-2-yl]-1-naphthalenecarbonitrile (434C)

A mixture of compound 434B (992 mg, 1.98 mmol) and RhCl₂(PPh₃)₃ (183 mg, 0.198 mmol) was evacuated and filled with argon (3×). THF (20 mL) was added and once all particulates had dissolved, catecholborane (0.42 mL, 3.96 mmol) was slowly added dropwise. When the formation of product ceased, as was determined by HPLC, the reaction mixture was cooled to 0° C. and quenched with phosphate buffer (34 mL, pH 7.2) followed by the addition of EtOH (19 mL) and H₂O₂ (2.9 mL, 30% aq sol). After 2 h, additional phosphate buffer (6.8 mL, pH 7.2), EtOH (3.8 mL) and H₂O₂(0.6 mL) were added. The reaction mixture was stirred at rt for 3 h. Once the boronate intermediate was consumed, the mixture was extracted with CH₂Cl₂ (300 mL) and the combined organic layers were washed with 1 N NaOH, 10% aq NaHSO₃ and brine. The combined organic layers were dried over Na₂SO₄. Purification by flash chromatography on silica gel eluting with 10% MeOH/CH₂Cl₂ gave 75 mg (9.3%) of compound 434C as a gray solid. HPLC conditions: 97% at 2.43 min (Phenomenex-prime S5-C18 column 4.6×50 mm, 10%-90% aqueous methanol over4 minute gradient with 0.2% H₃PO₄, detecting at 220 nm). MS (ES): m/z 407.18 [M+H]⁺.

### EXAMPLE 435

### (3aα,4≢2,5β,7β,7aα)-4-[7-[2-(4-Cyanophenoxy)ethyl]-4-ethyloctahydro-5-hydroxy-1,3-dioxo-4,7-epoxy-2H-isoindol-2-yl]-1-naphthalenecarbonitrile (435)

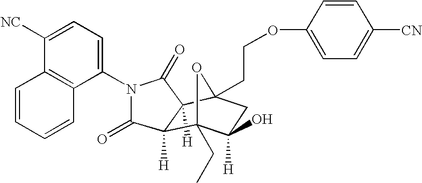

DBAD (39.6 mg, 0.172 mmol) was added to a solution of PPh₃ (45.1 mg, 0.172 mmol) in THF (0.8 mL). After stirring for 10 min, 4-cyanophenol (20.5 mg, 0.172 mmol) was added and the reaction mixture was stirred for an additional 5 min. Compound 434C (25.0 mg, 0.062 mmol) was added and the mixture was stirred at rt for 2 h. The reaction was concentrated under reduced pressure. Purification by Prep TLC eluting with 10% acetone/CHCl3 gave 18.1 mg (0.036 mmol, 57.6%) of compound 435. HPLC conditions: 96% at 3.15 min (YMC S5 ODS 4.6×50 mm, 10%-90% aqueous methanol over 4 minute gradient with 0.2% H₃PO₄, detecting at 220 nm). MS (ES): m/z 508.14 [M+H]⁺.

### EXAMPLE 436

### (3aα,4β,7β,7aα)-2-(4-Cyano-1-naphthyalenyl)octahydro-N-(2-hydroxyphenyl)-7-methyl-1,3-dioxo-4,7-expoxy-4H-isoindole-4-ethanamide (436)

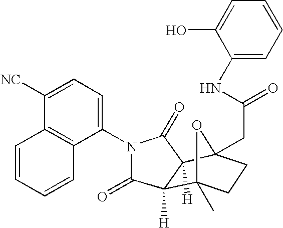

Compound 234B (0.100 g, 0.256 mmol) was dissolved in DMF (anhydrous, 1.5 mL), BOP (0.225 g, 0.51 mmol) was added followed by 2-aminophenol (0.056 g, 0.51 mmol) and N-methyl morpholine (0.056 mL, 0.51 mmol). The reaction mixture was stirred at 25° C. under argon for 3 h, then the crude material was purified by reverse phase preparative-HPLC (YMC S5 ODS 20×100 mm, 20-100% aqueous methanol over 15 minutes containing 0.1% TFA, 20 mL/min, monitoring at 220 nm) to give 0.078 g of compound 436 as a light brown solid. HPLC: 100% at 3.037 min (retention time) (YMC S5 ODS column 4.6×50 mm eluting with 10-90% aqueous methanol over 4 minutes containing 0.1% TFA, 4 mL/min, monitoring at 220 nm), MS (ES): m/z 482.34 [M+H]⁺.

### EXAMPLE 437

### (3aα,4β,7β,7aα)-4-[4-(2-Benzoxazolylmethyl)octahydro-7-methyl-1,3-dioxo-4,7-epoxy-2H-isoindol-2-yl]-1-naphthalenecarbonitrile (437)

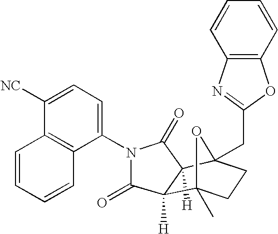

Triphenylphosphine (0.082 g, 0.312 mmol) and DBAD (0.072 g, 0.312 mmol) were mixed in THF (0.5 mL). After allowing the previous mixture to react for 15 mins, compound 436 (0.075 g, 0.156 mmol) was added. The reaction mixture was stirred at 25° C. for 2 h and then the crude material was purified by reverse phase preparative-HPLC (YMC S5 ODS 20×100 mm, 20-100% aqueous methanol over 15 minutes containing 0.1% TFA, 20 mL/min, monitoring at 220 nm) to give 0.052 g of compound 437 as a light brown solid. HPLC: 100% at 3.443 min (retention time) (YMC S5 ODS column 4.6×50 mm eluting with 10-90% aqueous methanol over 4 minutes containing 0.1% TFA, 4 mL/min, monitoring at 220 nm), MS (ES): m/z 464.18 [M+H]⁺.

### EXAMPLE 438

### (3aα,4β,7β,7aα)-Hexahydro-4,7-dimethyl-2-[4-[2,2,2-trifluoro-1-hydroxy-1-(trifluoromethyl)ethyl]phenyl]-4,7-epoxy-1H-isoindole-1,3(2H)-dione (438)

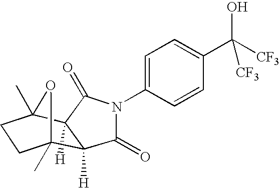

A mixture of 2-(4′-aminophenyl)-1,1,1,3,3,3-hexafluoro-2-propanol (95.7 mg, 0.369), compound 20A (48.3 mg, 0.246 mmol), triethylamine (0.15 mL) and MgSO₄ (50 mg) in toluene (1 mL) was heated in a sealed tube to 135° C. overnight. The mixture was filtered and concentrated under reduced pressure. Purification by reverse phase preparative chromatography (YMC S5 ODS 20×100 mm, 20-100% aqueous methanol over 15 minutes containing 0.1% TFA, 20 mL/min, monitoring at 220 nm) gave 44.0 mg (41%) of compound 438 as a white solid. HPLC: 99% at 3.10 min (retention time) (Phenomenex-prime S5-C18 column 4.6×50 mm eluting with 10-90% aqueous methanol over 4 minutes containing 0.2% phosphoric acid, 4 mL/min, monitoring at 220 nm), MS (ES): m/z [M+H]=438.14.

### EXAMPLES 439 TO 454

Additional compounds of the present invention were prepared by procedures analogous to those described above. The compounds of Examples 439 to 454 have the following structure (L is a bond):

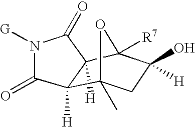

where G, R⁷, the compound name, retention time, molecular mass, and the procedure employed, are set forth in Table 9. The absolute configuration for the following compounds was not determined. For simplicity in nomenclature, compound 243Di is designated herein as having an “S” configuration and compound 243Dii as having an “R” configuration. Enantiomerically pure products derived from compound 243Di are designated herein as having an “S” configuration and enantiomerically pure products derived from compound 243Dii are designated herein as having an “K” configuration. Similarly, compound 428i is designated herein as having an “S” configuration and compound 428ii as having an “R” configuration. Enantiomerically pure products derived from compound 428i are designated herein as having an “S” configuration and enantiomerically pure products derived from compound 428ii are designated herein as having an “R” configuration.

The chromatography techniques used to determine the compound retention times of Table 9 are as follows: LCMS=YMC S5 ODS column, 4.6×50 mm eluting with 10-90% MeOH/H₂O over 4 minutes containing 0.1% TFA; 4 mL/min, monitoring at 220 nm. LCMS*=YMC S5 ODS column, 4.6×50 mm eluting with 10-90% MeOH/H₂O over 2 minutes containing 0.1% TFA; 4 mL/min, monitoring at 220 nm. LC=YMC S5 ODS column 4.6×50 mm eluting with 10-90% MeOH/H₂O over 4 minutes containing 0.2% phosphoric acid, 4 mL/min, monitoring at 220 nm. The molecular mass of the compounds listed in Table 9 were determined by MS (ES) by the formula m/z.

TABLE 9

| | | | | Retention | |
|---|---|---|---|---|---|
| | | | | Time | |
| | | | | Min./ | |
| Ex. | | | Compound | Molecular | Procedure |
| No | G | R⁷ | Name | Mass | of Ex. |
| 439 | 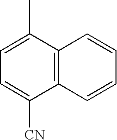 | 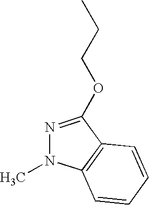 | [3aR-(3aα,4β,5β,7β,7aα)]-4-[Octahydro- 5-hydroxy-4-methyl-7-[2-[(1- methyl-1H-indazol-3-yl)oxy]ethyl]-1,3- dioxo-4,7-epoxy-2H-isoindol-2-yl]-1- naphthalenecarbonitrile | 3.33 LC 523.3 [M + H]⁺ | 251, 253 |
| 440 | 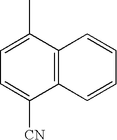 | 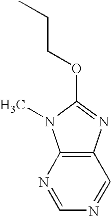 | [3aR-(3aα,4β,5β,7β,7aα)]-4-[Octahydro- 5-hydroxy-4-methyl-7-[2-[(9- methyl-9H-purin-8-yl)oxy]ethyl]-1,3- dioxo-4,7-epoxy-2H-isoindol-2-yl]-1- naphthalenecarbonitrile | 2.34 LC 525.2 [M + H]⁺ | 251, 253 |
| 441 | 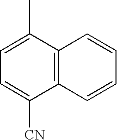 | 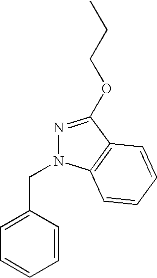 | [3aR-(3aα,4β,5β,7β,7aα)]-4-[Octahydro- 5-hydroxy-4-methyl-1,3-dioxo-7-[2-[[1- (phenylmethyl)-1H-indazol-3- yl)oxy]ethyl]-4,7-epoxy-2H-isoindol- 2-yl]-1-naphthalenecarbonitrile | 3.73 LC | 243Dii, 244Dii |
| 442 | 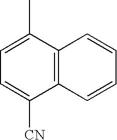 | 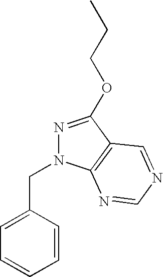 | [3aR-(3aα,4β,5β,7β,7aα)]-4-[Octahydro- 5-hydroxy-4-methyl-1,3-dioxo-7-[2-[[1- (phenylmethyl)-1H-pyrazolo[3,4- d]pyrimidin-3-yl]oxy]ethyl]-4,7-epoxy- 2H-isoindol-2-yl]-1-naphthalene- carbonitrile | 3.37 LC | 251, 253 |
| 443 | 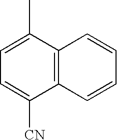 | 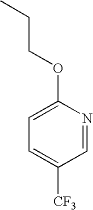 | [3aS-(3aα,4β,5β,7β,7aα)]-4-[Octahydro- 5-hydroxy-4-methyl-1,3-dioxo-7-[2-[[5- (trifluoromethyl)-2-pyridinyl]oxy]ethyl]- 4,7-epoxy-2H-isoindol-2-yl]-1- naphthalenecarbonitrile | 3.45 LC 538.23 [M + H]⁺ | 243Di, 244Di |
| 444 | 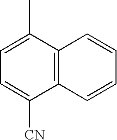 | 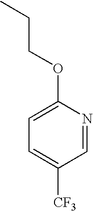 | [3aR-(3aα,4β,5β,7β,7aα)]-4-[Octahydro- 5-hydroxy-4-methyl-1,3-dioxo-7-[2-[[5- (trifluoromethyl)-2-pyridinyl]oxy]ethyl]- 4,7-epoxy-2H-isoindol-2-yl]-1- naphthalenecarbonitrile | 3.46 LC 538.24 [M + H]⁺ | 243Dii, 244Dii |
| 445 | 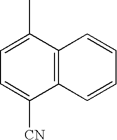 | 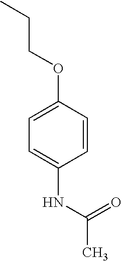 | [3aR-(3aα,4β,5β,7β,7aα)]-N-[4-[2- [2-(4-Cyano-1-naphthalenyl)octahydro-5- hydroxy-4-methyl-1,3-dioxo-4,7-epoxy- 7H-isoindol-7-yl]ethoxylphenyl]acetamide | 2.747 LC 526.28 [M + H]⁺ | 243Dii, 244Dii |
| 446 | 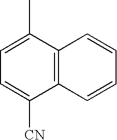 | 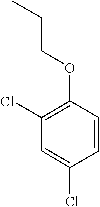 | [3aR-(3aα,4β,5β,7β,7aα)]-4-[7-[2- (2,4-Dichlorophenoxy)ethyl]octahydro- 5-hydroxy-4-methyl-1,3-dioxo-4,7- epoxy-2H-isoindol-2-yl]-1- naphthalenecarbonitrile | 3.71 LC 537.17 [M + H]⁺ | 243Dii, 244Dii |
| 447 | 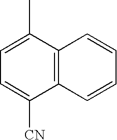 | 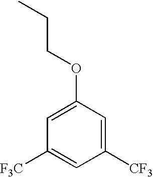 | [3aR-(3aα,4β,5β,7β,7aα)]-4-[7-[2- [3,5-Bis(trifluoromethyl)phenoxy]eth- yl]octahydro-5-hydroxy-4-methyl-1,3- dioxo-4,7-epoxy-2H-isoindol-2-yl]-1- naphthalenecarbonitrile | 3.89 LC 605.25 [M + H]⁺ | 243Dii, 244Dii |
| 448 | 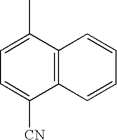 | 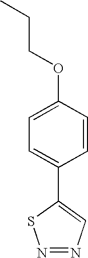 | [3aS-(3aα,4β,5β,7β,7aα)]-4-[Octahydro- 5-hydroxy-4-methyl-1,3-dioxo-7-[2-[4- (1,2,3-thiadiazol-5-yl)phenoxy]ethyl]-4,7- epoxy-2H-isoindol-2-yl]-1- naphthalenecarbonitrile | 3.14 LC 553.1 [M + H]⁺ | 243Di, 244Di |
| 449 | 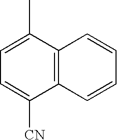 | 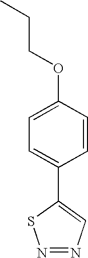 | [3aR-(3aα,4β,5β,7β,7aα)]-4-[Octahydro- 5-hydroxy-4-methyl-1,3-dioxo-7-[2-[4- (1,2,3-thiadiazol-5-yl)phenoxy]ethyl]-4,7- epoxy-2H-isoindol-2-yl]-1- naphthalenecarbonitrile | 3.15 LC 553.23 [M + H]⁺ | 243Dii, 244Dii |
| 450 | 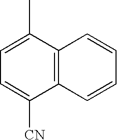 | 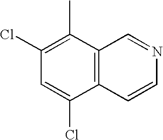 | [3aR-(3aα,4β,5β,7β,7aα)]-4-[7-[2- [(5,7-Dichloro-8-quinolinyl)oxy]eth- yl]octahydro-5-hydroxy-4-methyl-1,3- dioxo-4,7-epoxy-2H-isoindol-2-yl]-1- naphthalenecarbonitrile, trifluoroacetate (1:1) | 3.70 LC 588.26 [M + H]⁺ | 243Dii, 244Dii |
| 451 | 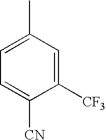 | 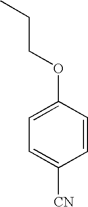 | [3aS-(3aα,4β,5β,7β,7aα)]-4-[7-[2- (4-Cyanophenoxy)ethyl]octahydro-5- hydroxy-4-methyl-1,3-dioxo-4,7-epoxy- 2H-isoindol-2-yl]-2-(trifluoro- methyl)benzonitrile | 3.087 LC 512.13 [M + H]⁺ | 431 |
| 452 | 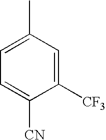 | 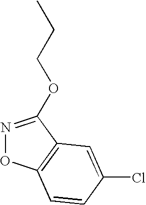 | [3aS-(3aα,4β,5β,7β,7aα)]-4-[7-[2- [(5-Chloro-1,2-benzisoxazol-3-yl)oxy]eth- yl]octahydro-5-hydroxy-4-methyl-1,3- dioxo-4,7-epoxy-2H-isoindol-2-yl]-2- (trifluoromethyl)benzonitrile | 3.563 LC 562.08 [M + H]⁺ | 431 |
| 453 | 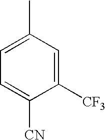 | 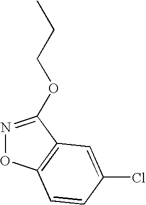 | [3aR-(3aα,4β,5β,7β,7aα)]-4-[7-[2- [(5-Chloro-1,2-benzisoxazol-3-yl)oxy]eth- yl]octahydro-5-hydroxy-4-methyl-1,3- dioxo-4,7-epoxy-2H-isoindol-2-yl]-2- (trifluoromethyl)benzonitrile | 3.57 LC 562.08 [M + H]⁺ | 430 |
| 454 | 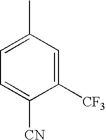 | 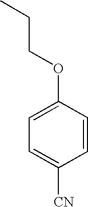 | [3aR-(3aα,4β,5β,7β,7aα)]-4-[7-[2- (4-Cyanophenoxy)ethyl]octahydro-5- hydroxy-4-methyl-1,3-dioxo-4,7-epoxy- 2H-isoindol-2-yl]-2-(trifluoro- methyl)benzonitrile | 3.087 LC 512.08 [M + H]⁺ | 430 |

### EXAMPLES 455 TO 457

Additional compounds of the present invention were prepared by procedures analogous to those described above. The compounds of Examples 455 to 457 have the following structure (L is a bond):

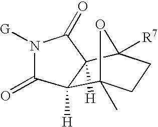

where G, R⁷, the compound name, retention time, molecular mass, and the procedure employed, are set forth in Table 10. The absolute configuration for the following compounds was not determined. For simplicity in nomenclature, compound 238i is designated herein as having an “R” configuration and compound 238ii as having an “S” configuration. Enantiomerically pure products derived from compound 238i are designated herein as having an “R” configuration and enantiomerically pure products derived from compound 238ii are designated herein as having an “S” configuration.

The chromatography techniques used to determine the compound retention times of Table 10 are as follows: LCMS=YMC S5 ODS column, 4.6×50 mm eluting with 10-90% MeOH/H₂O over 4 minutes containing 0. 1% TFA; 4 mL/min, monitoring at 220 nm. LCMS*=YMC S5 ODS column, 4.6×50 mm eluting with 10-90% MeOH/H₂O over 2 minutes containing 0.1% TFA; 4 mL/min, monitoring at 220 nm. LC=YMC S5 ODS column 4.6×50 mm eluting with 10-90% MeOH/H₂O over 4 minutes containing 0.2% phosphoric acid, 4 mL/min, monitoring at 220 nm. The molecular mass of the compounds listed in Table 10 were determined by MS (ES) by the formula nm/z.

TABLE 10

| | | | | Retention | |
|---|---|---|---|---|---|
| | | | | Time | |
| | | | | Min./ | Procedure |
| Ex. | | | Compound | Molecular | of |
| No | G | R⁷ | Name | Mass | Ex. |
| 455 | 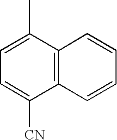 | 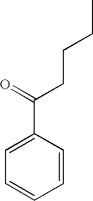 | (3aα,4β,5β,7β,7aα)-4-[Octa- hydro-4-meth- yl-1,3-di- oxo-7-(4-oxo-4-phenyl- butyl)-4,7-epoxy-2H-iso- indol-2-yl]-1-naph- thalenecarbonitrile | 3.53 LC 479.35 [M + H]⁺ | 265, 249 |
| 456 | 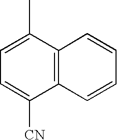 | 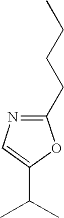 | (3aα,4β,5β,7β,7aα)-4-[Octa- hydro-4-meth- yl-7-[3-[5-(1-meth- ylethyl)-2-oxa- zolyl]propyl]-1,3-di- oxo-4,7-epoxy-2H-iso- indol-2-yl]-1-naph- thalenecarbonitrile | 3.547 LC 484.28 [M + H]⁺ | 248, 249 |
| 457 | 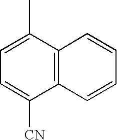 | 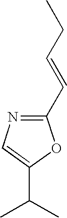 | [3aα,4β,5β,7β,7aα(E)]-4-[Octa- hydro-4-meth- yl-7-[3-[5-(1-meth- ylethyl)-2-oxa- zolyl]-2-pro- penyl]-1,3-di- oxo-4,7-epoxy-2H-iso- indol-2-yl]-1-naph- thalenecarbonitrile | 3.66 LC 482.28 [M + H]⁺ | 248, 249 |

### EXAMPLE 458

### (3aα,4β,5β,7β,7aα)-4-(Octahydro-5-hydroxy-4,7-dimethyl-1,3-dioxo-4,7-epoxy-2H-isoindol-2-yl)-2-(trifluoromethyl)benzonitrile & (3aα,4β,5α,7β,7aα)-4-(Octahydro-5-hydroxy-4,7-dimethyl-1,3-dioxo-4,7-epoxy-2H-isoindol-2-yl)-2-(trifluoromethyl)benzonitrile (221B & 222D)

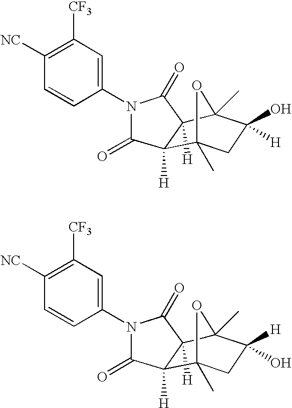

Compound 20B was converted to compounds 221B and 222D (also synthesized as compounds 221B and 222D) by biotransformation.

Compound 20B was hydroxylated by Amycolatopsis orientalis (ATCC 43491). A 1 ml culture from a frozen vial was used to inoculate 100 ml medium in a 500 mL portion Erlenmeyer flask and the flask was incubated at 28° C., at 200 rpm for 3 days. A 10 mL portion of this culture was used to inoculate 100 mL medium in a 500 ml Erlenmeyer flask and the flask was incubated at 28° C., at 200 rpm for 1 day. 10 mL portions of the 1-day culture were distributed to each of three 50 ml flasks. Compound 20B (3 mg in 0.1 mL methanol) was added to each culture and the incubations were continued for 3 days. The culture broth in each flask was extracted with 20 mL ethyl acetate, and the pooled ethyl acetate extracts were evaporated to dryness at 40° C. under a nitrogen stream. The residue was dissolved in 1.2 mL methanol and analyzed by HPLC, LC/MS and LC/NMR. The solution contained 2.5 mg of remaining Compound 20B, 1.6 mg of compound 221B, and 1.3 mg of compound 222D. MS and NMR analyses were in agreement with the structures shown above.

Medium: 0.5% toasted nutrisoy, 2% glucose, 0.5% yeast extract, 0.5% K₂HPO₄, 0.5% NaCl, adjusted to pH 7 with HCl (R. V. Smith and J. P. Rosazza, Arch. Biochem. Biophys., 161, 551-558 (1974)

HPLC Analysis

Column: Phenomenex Luna C18, 150×2 mm, 51μ

mobile phase: solvent A: 95% 20 mM ammonium acetate pH 5.1, 5% acetonitrile solvent B: 95% acetonitrile, 5% 20 mM ammonium acetate pH 5.1

linear gradient going from 100% solvent A to 5% solvent A in 25 minutes followed by equilibration at 100% solvent A for 8 minutes.

temperature: 40° C.

detection: 250 nm

injection volume: 1 μL

retention times: compound 20B, 20.8 min; compound 221B, 16.5 min; compound 222D, 17.8 min

HPLC Conditions

Chiral HPLC conditions were employed for the separation of enantiomers and achiral HPLC conditions were employed for the separation of diastereomers of the hydroxylated analogs of compound 20B (i.e., compounds 221B and 222D and compounds 254i and 254ii)

Two methods were used under chiral HPLC conditions, reverse phase (RP) for chiral analysis of biotransformation products in biological samples and normal phase (NP) for non-biological samples.

Chiral RP-HPLC Condition

| | |
|---|---|
| Column: | CHIRALPAK AD-R |
| | 4.6 × 250 mm, 10 μ |
| Temperature: | 40° C. |
| Injection Volume: | 5 or 20 μL |
| Mobile Phase: | A: MeCN |
| | B: H₂O |
| | Isocratic, 30% of A, 18 min. |
| Flow Rate: | 1 mL/min. |
| UV Detection: | 242 nm |

Chiral NP-HPLC Condition

| | |
|---|---|
| Column: | CHIRALPAK AD |
| | 4.6 × 250 mm, 10μ |
| Temperature: | 25° C. |
| Injection Volume: | 5 or 20 μL |
| Mobile Phase: | A: Heptane |
| | B: MeOH/Ethanol |
| | (1:1) |
| | Isocratic, 80% of A, 20 min. |
| Flow Rate: | 1 mL/min. |
| UV Detection: | 242 nm |

Under these conditions compounds 254i and 254ii had retention times of 8.5 minutes and 9.85 minutes, respectively.

Reverse phase HPLC was employed for the separation of the diastereomeric compounds 221B and 222D:

Mobile Phase:

| | |
|---|---|
| Solvent A: | 95% 20 mM ammonium acetate pH 5.1, 5% acetonitrile |
| Solvent B: | 95% acetonitrile, 5% 20 mM ammonium acetate pH 5.1 |

Gradient:

Linear gradient going from 100% solvent A to 5% solvent A in 25 minutes followed by equilibration at 100% solvent A for 8 minutes. Total run time of 36 minutes.

Flow Rate:

0.2 ml/min

Column:

Phenomenex Luna 5 micron C₁₈ 150×2.0 mm id

Detection:

UV detection at 242 nm

Under these conditions, compounds 221B and 222D had retention times of 18.983 min and 20.362 min, respectively.

### EXAMPLE 459

### (3aα,4β,5β,7β,7aα)-4-[Octahydro-5-hydroxy-7-(2-hydroxyethyl)-4-methyl-1,3-dioxo-4,7-epoxy-2H-isoindol-2-yl]-1-naphthalenecarbonitrile (459)

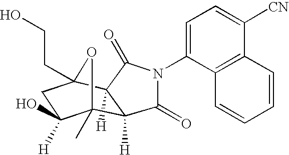

Compounds 223A and 331 were converted to compound 459 by biotransformation.

Microbial Hydroxylation of Compound 223A

1. Reaction

To a 500 mL flask containing 100 ml of the transformation medium was added one frozen vial (approximately 2 ml) of Streptomyces griseus ATCC 10137. The transformation medium was prepared as follows: to a 2 L plastic beaker was added 20 g of dextrose, 5.0 g of yeast extract, 5.0 g of soybean meal, 5.0 g of sodium chloride, 5.0 g of potassium phosphate (diabasic) and 1 L of deionized water, and the mixture was stirred at room temperature for 3 to 30 min. The pH of the mixture was then adjusted to 7.0 with 1 N HCl or 1 N NaOH. The resulting mixture was dispensed into 500 ml flasks (100 ml per flask). The flasks were covered with Bio/Wrap and autoclaved at 121° C. for 15 min. and cooled down to room temperature before use.

The culture was incubated at 28° C. and at 250 rpm for 24 hours. Ten mL of the resulting culture was transferred to a 50 mL flask, to which 1 mg of compound 223A in 0.2 ml ethanol was added. The flask was incubated at 28° C. and 250 rpm for 24 hours, and the reaction culture was extracted with EtOAc (10 ml). The EtOAc extract was dried under N₂ and the residue was dissolved in 1 ml of MeOH (reaction extract).

2. Product Analysis

HPLC:

10 μL of the reaction extract was injected into HPLC column (YMC ODS-AQ C-18 column, 150×6.0 mm i.d.). The column was eluted with 1 mM HCl in water/CH₃CN at 1.2 mL/min flow rate: 30 to 60% CH₃CN over 8 min, 60 to 85% CH₃CN over 0.5 min, 85% CH₃CN for 1 min, 85 to 30% CH₃CN over 0.5 min. The eluents were monitored at 300 nm. Two major peaks with about a 1 to 1 area ratio were observed, which had the same UV spectra as those of compounds 459 and 331, and had retention times of 4.55 min and 7.23 min, respectively, matching the retention times of authentic samples of compound 459 (4.53 min) and compound 331 (7.2 min).

LC/MS

The reaction extract: two major UV peaks.

Peak 1, Tr4.68 min: 391 [M+H]⁺, 343, 319, 303, 289

Peak 2, Tr 5.35 min: 375 [M+H]⁺, 345

### Authentic Samples

Compound 459, Tr4.82 min: 391 [M+H]⁺, 343, 319, 289

Compound 331, Tr5.48 min: 375 [M+H]⁺, 345

Microbial Hydroxylation of Compound 331

To a 500 mL flask containing 100 ml of the transformation medium was added one frozen vial (approximately 2 ml) of Streptomyces griseus ATCC 10137. The transformation medium was prepared as follows: to a 2 L plastic beaker was added 20 g of dextrose, 5.0 g of yeast extract, 5.0 g of soybean meal, 5.0 g of sodium chloride, 5.0 g of potassium phosphate (dibasic) and one L of deionized water, and the mixture was stirred at room temperature for 3 to 30 min. The pH of the mixture was then adjusted to 7.0 with 1 N HCl or 1 N NaOH. The resulting mixture was dispensed into 500 mL flasks (100 ml per flask). The flasks were covered with Bio/Wrap and autoclaved at 121° C. for 15 min. and cooled down to room temperature before use.

The culture was incubated at 28° C. and 250 rpm for 3 days. One mL of the resulting culture was added to a 500 mL flask containing 100 mL of the transformation medium and the flask was incubated at 28° C. and 250 rpm for 24 hours. Ten mL of the resulting culture was transferred to a 50 mL flask, to which 1 mg of compound 331 in 0.2 mL ethanol was added. The flask was incubated at 28° C. and 250 rpm for 23 hours. HPLC analysis showed that the peak area ratio of compound 459 to compound 331 in the reaction culture was about 1.1/1.

### EXAMPLE 460

### (1aα,2β,2aα,5aα,6βb,6aα)-4-[2-[2-[[(1,1-Dimethylethyl)dimethylsilyl]oxy]ethyl]octahydro-6-methyl-3,5-dioxo-2,6-epoxy-4H-oxireno[f]isoindol-4-yl]-1-naphthalenecarbonitrile (460)

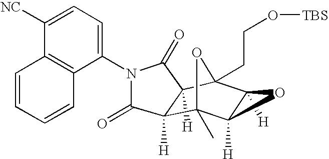

Compound 231A (2.00 g, 4. 10 mmol) was dissolved in dicholomethane (40 ml) and cooled to 0° C. mCPBA (2.36 g, 8.20 mmol) was added. The reaction mixture was then warmed up to room temperature and stirred under argon for 18 hours, followed by the addition of 10% Na₂SO₃ (25 ml) and saturated NaHCO₃ (25 ml). After stirring for 20 minutes, the organic layer was separated and the aqueous layer was extracted with dicholomethane (3×50 ml). The combined organic layers were washed with brine, dried over Na₂SO₄ and concentrated to give 2.0 g compound 460 as light yellow solid. HPLC: 99% at 4.00 min (retention time) (Phenomenex-prime S5-C 18 column 4.6×50 mm eluting with 10-90% aqueous methanol over 4 minutes containing 0.2% phosphoric acid, 4 mL/min, monitoring at 220 nm), MS (ES): m/z [M+H]=505.19

### EXAMPLE 461

### [3aR-(3aα,4β,7β,7aα)]-4-[4-Ethyloctahydro-7-(2-hydroxyethyl)-1,3-dioxo-4,7-epoxy-2H-isoindol-2-yl]-1-naphthalenecarbonitrile & [3aS-(3aα,4β,7β,7aα)]-4-[4-Ethyloctahydro-7-(2-hydroxyethyl)-1,3-dioxo-4,7-epoxy-2H-isoindol-2-yl]-1-naphthalenecarbonitril (461i & 461ii)

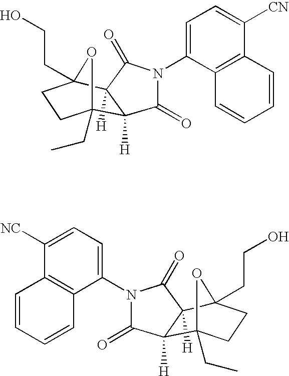

The racemic mixture of compounds 245C was separated by normal phase preparative chiral HPLC using a Chiracel AD column (5 cm×50 cm), eluting with 20% solvent B (50% MeOH/EtOH) in solvent A (Heptane), flow rate: 50 mL/min. Compound 461i eluted from 80 min to 100 min and compound 461ii eluted from 125 min to 150 min.

The absolute conformation for compounds 461i and 461ii was not determined. For simplicity in nomenclature, compound 461i is designated herein as having an “R” configuration and compound 461ii as having an “S” configuration. Enantiomerically pure products derived from compound 461i are designated herein as having an “R” configuration and enantiomerically pure products derived from compound 461ii are designated herein as having an “S” configuration.

### EXAMPLE 462

### [3aR-(3aα,4β,7β,7aα)]-4-[4-[2(4-Cyanophenoxy)ethyl]-7-ethyloctahydro-1,3-dioxo-4,7-epoxy-2H-isoindol-2-yl]-1-naphthalenecarbonitrile (462)

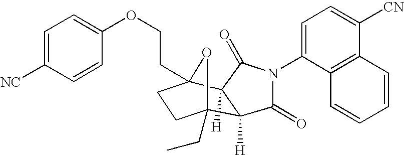

DBAD (29.5 mg, 0.128 mmol) was added to a solution of PPh₃ (33.6 mg, 0.128 mmol) in THF (0.5 mL). After stirring for 10 mins, 4-cyanophenol (15.2 mg, 0.128 mmol) was added and the reaction mixture was stirred for an additional 5 mins. Compound 461i (18.3 mg, 0.047 mmol) was added and the mixture was stirred at rt for 2 h. The reaction was concentrated under reduced pressure. Purification by flash chromatography on silica gel eluting with 40% EtOAc/hexane gave 16.9 mg (0.034 mmol, 73.2%) of compound 462. HPLC conditions: 98% at 3.64 min (YMC S5 ODS 4.6×50 mm, 10%-90% aqueous methanol over 4 minute gradient with 0.2% H₃PO₄, detecting at 220 nm). MS (ES): m/z 492.23 [M+H]⁺.

### EXAMPLE 463

### [3aS-(3aα,4β,7β,7aα)]-4-[4-[2-(4-Cyanophenoxy)ethyl]-7-ethyloctahydro-1,3-dioxo-4,7-epoxy-2H-isoindol-2-yl]-1-naphthalenecarbonitrile (463)

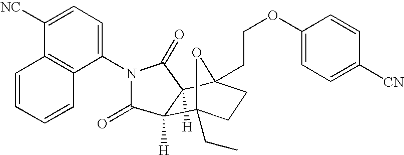

DBAD (29.5 mg, 0.128 mmol) was added to a solution of PPh₃ (33.6 mg, 0.128 mmol) in THF (0.5 mL). After stirring for 10 mins, 4-cyanophenol (15.2 mg, 0.128 mmol) was added and the reaction mixture was stirred for an additional 5 mins. Compound 461ii (18.3 mg, 0.047 mmol) was added and the mixture was stirred at rt for 2 h. The reaction was concentrated under reduced pressure. Purification by flash chromatography on silica gel eluting with 40% EtOAc/hexane gave 18.1 mg (0.037 mmol, 78.4%) of compound 463. HPLC conditions: 97% at 3.63 min (YMC S5 ODS 4.6×50 mm, 10%-90% aqueous methanol over 4 minute gradient with 0.2% H₃PO₄, detecting at 220 nm). MS (ES): m/z 492.17 [M+H]⁺.

We claim:

1. A compound of the following formula: 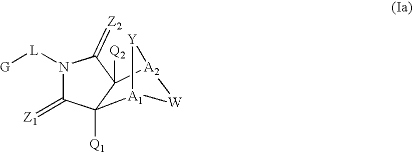
wherein the symbols have the following meanings and are, for each occurrence, independently selected:
G is an aryl or heterocyclo group, where said group is mono- or polycyclic, and which is optionally substituted at one or more positions;
Z₁ is O, S, NH, or NR⁶;
Z₂ is O, S, NH, or NR⁶;
A₁ is CR⁷ or N;
A₂ is CR⁷ or N;
Y′ is J—J′—J″ where J is (CR⁷R⁷′)n and n=0-3, J′ is a bond or O, S, S═O, SO₂, NH, NR⁷, CR⁷R⁷′, R²P═O, R²P═S, R²OP═O, R²NHP═O, OP═OOR², OP═ONHR², OSO₂, NHNH, NHNR⁶, NR⁶NH, N═N, cycloalkyl or substituted cycloalkyl, cycloalkenyl or substituted cycloalkenyl, or heterocyclo or substituted heterocyclo, and J″ is (CR⁷R⁷′)n and n=0-3, where Y is not a bond; and
W′ is CR⁷R⁷′—CR⁷R⁷′, CR⁷R⁷′—C═O, NR⁹—CR⁷R⁷′, N═CR⁸, N═N, NR⁹-NR⁹′, cycloalkyl or substituted cycloalkyl, cycloalkenyl or substituted cycloalkenyl, heterocyclo or substituted heterocyclo, or aryl or substituted aryl, wherein, when W′ is not NR⁹—CR⁷R⁷′, N═CR⁸, N═N, NR⁹—NR⁹′, or heterocyclo or substituted heterocyclo, then J′ must be O, S, S═O, SO₂, NH, NR⁷, OP═OOR², OP═ONHR², OSO₂, NHNH, NHNR⁶, NR⁶NH, or N═N; or alternatively,
Y′ is NR⁷−CR⁷R⁷′ and W′ is CR⁸═CR⁸′; or, alternatively,
Y′ is CR⁷R⁷′—C═O and W′ is NR⁹−CR⁷R⁷′;
Q₁ is H, alkyl or substituted alkyl, alkenyl or substituted alkenyl, cycloalkyl or substituted cycloalkyl, cycloalkenyl or substituted cycloalkenyl, heterocycloalkyl or substituted heterocycloalkyl, arylalkyl or substituted arylalkyl, alkynyl or substituted alkynyl, aryl or substituted aryl, heterocyclo or substituted heterocyclo, halo, CN, R¹OC═O, R⁴C═O, R⁵R⁶NC═O, HOCR⁷R⁷′, nitro, R¹OCH₂, R¹O, NH₂, C═OSR¹, SO₂R¹ or NR⁴R⁵;
Q₂ is H, alkyl or substituted alkyl, alkenyl or substituted alkenyl, cycloalkyl or substituted cycloalkyl, cycloalkenyl or substituted cycloalkenyl, heterocycloalkyl or substituted heterocycloalkyl, arylalkyl or substituted arylalkyl, alkynyl or substituted alkynyl, aryl or substituted aryl, heterocyclo or substituted heterocyclo, halo, CN, R¹OC═O, R⁴C═O, R⁵R⁶NC═O, HOCR⁷R⁷′, nitro, R¹OCH₂, R¹O, NH₂, C═OSR¹, SO₂R¹ or NR⁴R⁵;
L is a bond, (CR⁷R⁷′)n, NH, NR⁵ or NR⁵(CR⁷R⁷′)n, where n=0-3;
R¹ and R¹′ are each independently H, alkyl or substituted alkyl, cycloalkyl or substituted cycloalkyl, cycloalkenyl or substituted cycloalkenyl, heterocyclo or substituted heterocyclo, cycloalkylalkyl or substituted cycloalkyalkyl, cycloalkenylalkyl or substituted cycloalkenylalkyl, heterocycloalkyl or substituted heterocycloalkyl, aryl or substituted aryl, arylalkyl or substituted arylalkyl;
R² is alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl;
R³ and R³′ are each independently H, alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, halo, CN, hydroxylamine, hydroxamide, alkoxy or substituted alkoxy, amino, NR¹R², thiol, alkylthio or substituted alkylthio;
R⁴ is H, alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, R¹C═O, R¹NHC═O, SO₂OR¹, or SO₂NR¹R¹′;
R⁵ is alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, R¹C═O, R¹NHC═O, SO₂R¹, SO₂OR¹, or SO₂NR¹R¹′;
R⁶ is alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, CN, OH, OR¹, R¹C═O, R¹NHC═O, SO₂R¹, SO₂OR¹, or SO₂NR¹R¹′;
R⁷ and R⁷′ are each independently H, alkyl or substituted alkyl, alkenyl or substituted alkenyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, halo, CN, OR¹, nitro, hydroxylamine, hydroxylamide, amino, NHR⁴, NR²R⁵, NOR¹, thiol, alkylthio or substituted alkylthio, R¹C═O, R¹OC═O, R¹NHC═O, SO₂R¹, SOR¹, PO₃R¹R¹′, R¹R¹′NC═O, C═OSR¹, SO₂R¹, SO₂OR¹, or SO₂NR¹R¹′, or, wherein A₁ or A₂ contains a group R⁷ and W contains a group R⁷, said R⁷ groups of A₁ or A₂ and W together form a heterocyclic ring;
R⁸ and R⁸′ are each independently H, alkyl or substituted alkyl, alkenyl or substituted alkenyl, cycloalkyl or substituted cycloalkyl, cycloalkenyl or substituted cycloalkenyl, heterocyclo or substituted heterocyclo, cycloalkylalkyl or substituted cycloalkyalkyl, cycloalkenylalkyl or substituted cycloalkenylalkyl, heterocycloalkyl or substituted heterocycloalkyl, aryl or substituted aryl, arylalkyl or substituted arylalkyl, nitro, halo, CN, OR¹, amino, NHR⁴, NR²R⁵, NOR¹, alkylthio or substituted alkylthio, C═OSR¹, R¹OC═O, R¹C═O, R¹NHC═O, R¹R¹′NC═O, SO₂OR¹, S═OR¹, SO₂R¹, PO₃R¹R¹′, or SO₂NR¹R¹′; and
R⁹ and R⁹′ are each independently H, alkyl or substituted alkyl, alkenyl or substituted alkenyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, CN, OH, OR¹, R¹C═O, R¹OC═O, R¹NHC═O, SO₂R¹, SO₂OR¹, or SO₂NR¹R¹′;
with the provisos that:
(1) when Y′ is —O—, Q₁ and Q₂ are hydrogen, Z₁ and Z₂ are O, W′ is —CH₂—CH₂—, and A₁ and A₂ are CH, then G—L is not phenyl, monosubstituted phenyl or phenyl which is substituted with two or more of the following groups: methoxy, halo, NO₂, methyl, CH₃—S—, OH, CO₂H, trifluoromethyl, —C(O)—C₆H₅, NH₂, 4-7-epoxy, hexahydro-1H-isoindole-1,3(2H)dione, or —C(O)—CH₃;
(2) when Y′ is —O—, Q₁ and Q₂ are hydrogen, Z₁ and Z₂ are O, W′ is CH₂—CH₂, and one of A₁ and A₂ is CH and the other is CR⁷, then G—L is not unsubstituted phenyl;
(3) when Y′ is —O—, Q₁ and Q₂ are hydrogen, Z₁ and Z₂ are O, W′ is CH₂—CH₂, and one of A₁ and A₂ is CH and the other is C—CH₃, then G—L is not phenyl substituted with chloro and/or methyl;
(4) when Y′ is —O— or —S—, Q₁ and Q₂ are hydrogen, Z₁ and Z₂ are O, W′ is CH₂—CH₂, and one of A₁ and A₂ is CH and the other is CH or C-alkyl, then G—L is not N-substituted piperazine-alkyl- or N-substituted imidazolidine-alkyl-;
(5) when Y′ is —O—; Q₁ and Q₂ are hydrogen, Z₁ and Z₂ are O, W′ is CH₂—CH₂, and A₁ and A₂ are CH, then G—L is not oxazole or triazole;
(6) when Y′ is —O—; Q₁ and Q₂ are hydrogen or methyl, Z₁ and Z₂ are O, W′ is CH₂—CH₂, and A₁ and A₂ are CH or C—CH₃, then G—L is not thiazole or substituted thiazole;
(7) when Y′ contains a group J′ selected from S, S═O, SO₂, NH, NR⁷, R²P═O, R²P═S, R²OP═O, R²NHP═O, OP═OOR², OP═ONHR², OSO₂, NHNH, NHR⁶, NR⁶NH or N═N, W′ is CR⁷R⁷′—CR⁷R⁷′, and Z₁ and Z₂ are O, then G—L is not unsubstituted phenyl;
(8) when Y is NR⁷, W′ is unsubstituted or substituted phenyl, and Q₁ and Q₂ are hydrogen, then Z₁ and Z₂ are not O;
(9) when Y′ is —O—, Q₁ and Q₂ are hydrogen, Z₁ and Z₂ are O, W′ is dihydroisoxazole bearing an optionally substituted phenyl group, and A₁ and A₂ are CH, then G—L is not unsubstituted phenyl or dichlorophenyl;
(10) when Y′ is O, Q₁ and Q₂ are hydrogen, Z₁ and Z₂ are O, W′ is ethylene oxide, and A₁ and A₂ are CH, then G—L is not methylphenyl or chlorophenyl;
(11) when Y′ is NR⁷—CR⁷R⁷′, W′ is CR⁸═CR⁸′, Q₁ and Q₂ are hydrogen, A₁ and A₂ are CH, C—CH₃, C—CH₂—C₆H₅ or C—CH₂—CH₃, and Z₁ and Z₂ are O, then G—L is not unsubstituted phenyl, monosubstituted phenyl or methylpyridinyl;
(12) when Y′ is CR⁷R⁷′—C═O, W′ is NR⁹—CR⁷R⁷′, Q₁ and Q₂ are hydrogen, A₁ and A₂ are CH, and Z₁ and Z₂ are O, then G—L is not unsubstituted phenyl;
(13) when Y′ is CHR⁷′—NR⁷ where R⁷′ is unsubstituted phenyl, methoxy or ethoxy and R⁷ is unsubstituted phenyl, methyl or —C(O)—C₆H₅, W′ is dimethoxyphenylene or unsubstituted phenylene, Z₁ and Z₂ are O, Q₁ and Q₂ are hydrogen, A₁ and A₂ are CH, C—CN, C—C(O)—C₆H₅, or —C(O)— dimethoxyphenyl, then G—L is not unsubstituted phenyl;
(14) the compound of formula Ia is not 6,10-epithio-4H-thieno-[3′,4′:5,6]cyclooct[1,2-f]isoindole-7,9(5H,8H)dione, 8-(3,5-dichlorophenyl)-6,6a,9a, 10,11,12,-hexahydro-1,3,6,10-tetramethyl-2,2,13-trioxide, (6R,6aR,9aS,10S);
(15) when Y′ is O, W′ is —CH₂—CH₂—, Q₁ and Q₂ are methyl, Z₁ and Z₂ are O, and A₁ and A₂ are CH, then G—L is not unsubstituted phenyl, phenyl substituted with methoxy, phenyl-alkyl-, or morpholine-alkyl, nor is the compound bridged to itself through a group L which is alkylene to form a bis compound;
(16) when Y′ is —O—, Q₁ and Q₂ are hydrogen, Z_{and Z}₂ are O, W′ is CR⁷R⁷′—CR⁷R⁷′, and A₁ and A₂ are CH, then G—L is not an unsubstituted phenyl group; and
(17) when Y′ is —O—, Q₁ and Q₂ are hydrogen, Z₁ and Z₂ are O, W′ is cyclopentyl, cyclohexyl, 3-phenyl-2-isoxazoline or CR⁷R⁷′—CR⁷R⁷′ where R⁷ and R⁷′ are each independently defined as Cl, Br, H and 4-butyrolactone and R⁷ and R⁷′ are not all simultaneously H, and A₁ and A₂ are CH, then G—L is not an unsubstituted naphthyl ring or a monosubstituted phenyl ring, where said substituent is methoxy, Br, Cl, NO₂, methyl, ethyl, CH₂-phenyl, S-phenyl, or O-phenyl.

2. The compound of claim 1 wherein
G is an aryl or heterocyclo group, where said group is mono- or polycyclic, and which is optionally substituted at one or more positions;
Z₁ is O, S, NH, or NR⁶;
Z₂ is O, S, NH, or NR⁶;
A₁ is CR⁷ or N;
A₂ is CR⁷ or N;
Y′ is J—J′—J″ where J is (CR⁷R⁷′)n and n=0-3, J′ is a bond or O, S, S═O, SO₂, NH, NR⁷, CR⁷R⁷′, R²P═O, R²P═S, R²OP═O, R²NHP═O, OP═OOR², OP═ONHR², OSO₂, NHNH, NHNR⁶, NR⁶NH, N═N, cycloalkyl or substituted cycloalkyl, cycloalkenyl or substituted cycloalkenyl, or heterocyclo or substituted heterocyclo, and J″ is (CR⁷R⁷′)n and n=0-3, where Y′ is not a bond;
W′ is CR⁷R⁷′—CR⁷R⁷′, CR⁷R⁷′—C═O, NR⁹—CR⁷R⁷′, N═CR⁸, N═N, NR⁹—NR⁹′, cycloalkyl or substituted cycloalkyl, cycloalkenyl or substituted cycloalkenyl, heterocyclo or substituted heterocyclo, or aryl or substituted aryl, wherein, when W′ is not NR⁹—CR⁷R⁷′, N═CR⁸, N═N, NR⁹—NR⁹′, or heterocyclo or substituted heterocyclo, then J′ must be O, S, S═O, SO₂, NH, NR⁷, OP═OOR², OP═ONHR², OSO₂, NHNH, NHNR⁶, NR⁶NH, or N═N;
Q₁ is H, alkyl or substituted alkyl, alkenyl or substituted alkenyl, cycloalkyl or substituted cycloalkyl, cycloalkenyl or substituted cycloalkenyl, heterocycloalkyl or substituted heterocycloalkyl, arylalkyl or substituted arylalkyl, alkynyl or substituted alkynyl, aryl or substituted aryl, heterocyclo or substituted heterocyclo, halo, CN, R¹OC═O, R⁴C═O, R⁵R⁶NC═O, HOCR⁷R⁷′, nitro, R¹OCH₂, R¹O, NH₂, or NR⁴R⁵;
Q₂ is H, alkyl or substituted alkyl, alkenyl or substituted alkenyl, cycloalkyl or substituted cycloalkyl, cycloalkenyl or substituted cycloalkenyl, heterocycloalkyl or substituted heterocycloalkyl, arylalkyl or substituted arylalkyl, alkynyl or substituted alkynyl, aryl or substituted aryl, heterocyclo or substituted heterocyclo, halo, CN, R¹OC═O, R⁴C═O, R⁵R⁶NC═O, HOCR⁷R⁷′, nitro, R¹OCH₂, R¹O, NH₂, or NR⁴R⁵;
L is a bond, (CR⁷R⁷′)n, NH, NR⁵ or NR⁵(CR⁷R⁷′)n, where n=0-3;
R¹ and R¹′ are each independently H, alkyl or substituted alkyl, cycloalkyl or substituted cycloalkyl, cycloalkenyl or substituted cycloalkenyl, heterocyclo or substituted heterocyclo, cycloalkylalkyl or substituted cycloalkyalkyl, cycloalkenylalkyl or substituted cycloalkenylalkyl, heterocycloalkyl or substituted heterocycloalkyl, aryl or substituted aryl, arylalkyl or substituted arylalkyl;
R² is alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl;
R³ and R³′ are each independently H, alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, halo, CN, hydroxylamine, hydroxamide, alkoxy or substituted alkoxy, amino, NR¹R², thiol, alkylthio or substituted alkylthio;
R⁴ is H, alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, R¹C═O, R¹NHC═O, or SO₂NR¹R¹′;
R⁵ is alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, R¹C═O, R¹NHC═O, SO₂R¹, or SO₂NR¹R¹′;
R⁶ is alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, CN, OH, OR¹, R¹C═O, R¹NHC═O, SO₂R¹, or SO₂NR¹R¹′;
R⁷ and R⁷′ are each independently H, alkyl or substituted alkyl, alkenyl or substituted alkenyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, halo, CN, OR¹, nitro, hydroxylamine, hydroxylamide, amino, NHR⁴, NR²R⁵, NOR¹, thiol, alkylthio or substituted alkylthio, R¹C═O, R¹OC═O, R¹NHC═O, SOR¹, PO₃R¹R¹′, R¹R¹′NC═O, C═OSR¹, SO₂R¹, or SO₂NR¹R¹′;
R⁸ and R⁸′ are each independently H, alkyl or substituted alkyl, alkenyl or substituted alkenyl, cycloalkyl or substituted cycloalkyl, cycloalkenyl or substituted cycloalkenyl, heterocyclo or substituted heterocyclo, cycloalkylalkyl or substituted cycloalkyalkyl, cycloalkenylalkyl or substituted cycloalkenylalkyl, heterocycloalkyl or substituted heterocycloalkyl, aryl or substituted aryl, arylalkyl or substituted arylalkyl, nitro, halo, CN, OR¹, amino, NHR⁴, NR²R⁵, NOR¹, alkylthio or substituted alkylthio, C═OSR¹, R¹OC═O, R¹C═O, R¹NHC═O, R¹R¹′NC═O, S═OR¹, SO₂R¹, PO₃R¹R¹′, or SO₂NR¹R¹′;
R⁹ and R⁹′ are each independently H, alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, CN, OH, OR¹, R¹C═O, R¹OC═O, R¹NHC═O, or SO₂NR¹R¹′;
with the provisos (1) to (17) of said formula Ia, and further where (i) when Y′ is —O— and W′ is CR⁷R⁷′—R⁷R⁷′, A₁ and A₂ are not simultaneously CH; and (ii) when L is a bond, G is not an unsubstituted phenyl group.

3. The compound of claim 1, wherein
G is an aryl or heterocyclo group, where said group is mono- or polycyclic, and which is optionally substituted at one or more positions;
Z₁ is O;
Z₂ is O;
A₁ is CR⁷;
A₂ is CR⁷;
Y′ is J—J′—J″ where J is (CR⁷R⁷′)n and n=0-3, J′ is a bond or O, S, S═O, SO₂, NH, NR⁷, CR⁷R⁷′, R²P═O, R²P═S, R²OP═O, R²NHP═O, OP═OOR², OP═ONHR², OSO₂, NHNH, NHNR⁶, NR⁶NH, N═N, cycloalkyl or substituted cycloalkyl, cycloalkenyl or substituted cycloalkenyl, or heterocyclo or substituted heterocyclo, and J″ is (CR⁷R⁷′)n and n=0-3, where Y′ is not a bond;
W is CR⁷R⁷′—CR⁷R⁷′, CR⁷R⁷′—C═O, NR⁹—CR⁷R⁷′, N═CR⁸, N═N, NR⁹—N⁹′, cycloalkyl or substituted cycloalkyl, cycloalkenyl or substituted cycloalkenyl, heterocyclo or substituted heterocyclo, or aryl or substituted aryl, wherein, when W is not NR⁹—CR⁷R⁷′, N═CR⁸, N═N, NR⁹—NR⁹′, or heterocyclo or substituted heterocyclo, then J′ must be O, S, S═O, SO₂, NH, NR⁷, OP═OOR², OP═ONHR², OSO₂, NHNH, NHNR⁶, NR⁶NH, or N═N;
Q₁ is H, alkyl or substituted alkyl, alkenyl or substituted alkenyl, cycloalkyl or substituted cycloalkyl, cycloalkenyl or substituted cycloalkenyl, heterocycloalkyl or substituted heterocycloalkyl, arylalkyl or substituted arylalkyl, alkynyl or substituted alkynyl, aryl or substituted aryl, heterocyclo or substituted heterocyclo, halo, CN, R⁴C═O, R⁵R⁶NC═O, HOCR⁷R⁷′, nitro, R¹OCH₂, R¹O, NH₂, or NR⁴R⁵;
Q₂ is H, alkyl or substituted alkyl, alkenyl or substituted alkenyl, cycloalkyl or substituted cycloalkyl, cycloalkenyl or substituted cycloalkenyl, heterocycloalkyl or substituted heterocycloalkyl, arylalkyl or substituted arylalkyl, alkynyl or substituted alkynyl, aryl or substituted aryl, heterocyclo or substituted heterocyclo, halo, CN, R⁴C═O, R⁵R⁶NC═O, HOCR⁷R⁷′, nitro, R¹OCH₂, R¹O, NH₂, or NR⁴R⁵;
L is a bond;
R¹ and R¹′ are each independently H, alkyl or substituted alkyl, cycloalkyl or substituted cycloalkyl, cycloalkenyl or substituted cycloalkenyl, heterocyclo or substituted heterocyclo, cycloalkylalkyl or substituted cycloalkyalkyl, cycloalkenylalkyl or substituted cycloalkenylalkyl, heterocycloalkyl or substituted heterocycloalkyl, aryl or substituted aryl, arylalkyl or substituted arylalkyl;
R² is alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl;
R³ and R³′ are each independently H, alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, halo, CN, alkoxy or substituted alkoxy, amino, NR¹R², alkylthio or substituted alkylthio;
R⁴ is H, alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, R¹C═O, R¹NHC═O, or SO₂NR¹R¹′;
R⁵ is alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, R¹C═O, R¹NHC═O, SO₂R¹, or SO₂NR¹R¹′;
R⁶ is alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, CN, OH, OR¹, R¹C═O, R¹NHC═O, SO₂R¹, or SO₂NR¹R¹′;
R⁷ and R⁷′ are each independently H, alkyl or substituted alkyl, alkenyl or substituted alkenyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, halo, CN, OR¹, nitro, amino, NHR⁴, NR²R⁵, alkylthio or substituted alkylthio, R¹C═O, R¹NHC═O, SO₂R¹, R¹R¹′NC═O, or SO₂NR¹R¹′;
R⁸ and R⁸′ are each independently H, alkyl or substituted alkyl, alkenyl or substituted alkenyl, cycloalkyl or substituted cycloalkyl, cycloalkenyl or substituted cycloalkenyl, heterocyclo or substituted heterocyclo, cycloalkylalkyl or substituted cycloalkyalkyl, cycloalkenylalkyl or substituted cycloalkenylalkyl, heterocycloalkyl or substituted heterocycloalkyl, aryl or substituted aryl, arylalkyl or substituted arylalkyl, nitro, halo, CN, OR¹, amino, NHR⁴, NR²R⁵, alkylthio or substituted alkylthio, R¹C═O, R¹NHC═O, R¹R¹′NC═O, SO₂R¹, or SO₂NR¹R¹′; and
R⁹ and R⁹′ are each independently H, alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, CN, OH, OR¹, R¹C═O, R¹NHC═O, or SO₂NR¹R¹′;
with the provisos (1) to (17) of said formula Ia, and further where (i) when Y′ is —O— and W′ is CR⁷R⁷′—CR⁷R⁷′, A₁ and A₂ are not simultaneously CH; and (ii) when L is a bond, G is not an unsubstituted phenyl group.

4. A compound selected from the group consisting of:
(3aα,4α,7α,7aα)-2-(4-Bromo-3-methylphenyl)tetrahydro-4,7-ethanothiopyrano[3,4-c]pyrrole-1,3,8(2H ,4H)-trione (1C);
(3aα,4α,7α,7aα)-2-(4-Bromo-3-methylphenyl)tetrahydro-4,7-ethanothiopyrano[3,4-c]pyrrole-1,3,8(2H,4H)-trione 5,5-dioxide (2);
(3aα,4β,7β,7aα)-2-(3-Chlorophenyl)hexahydro-4-methyl-4,7-epoxy-1H-isoindole-1,3(2H)-dione (3);
(3aα,4α,7α,7aα)- and (3aα,4β,7β,7aα)-4-[(Acetyloxy)methyl]-3a,4,7,7a-tetrahydro-2-[3-(trifluoromethyl)phenyl]-4,7-epoxy-1H-isoindole-1,3(2H)-dione (4i & 4ii, respectively);
(3aα,4α,7α,7aα)- and (3aα,4β,7β,7aα)-4-[(Acetyloxy)methyl]-Hexahydro-2-[3-(trifluoromethyl)phenyl]-4,7-epoxy-1H-isoindole-1,3(2H)-dione (5i & 5ii, respectively);
(3aα,4α,7α,7aα)- and (3aα,4β,7β,7aα)-3a,4,7,7a-Tetrahydro-5-(hydroxymethyl)-2-[3-(trifluoromethyl)phenyl]-4,7-epoxy-1H-isoindole-1,3(2H)-dione (6i & 6ii, respectively);
(3aα,4α,7α,7aα)-3a,4,7,7a-Tetrahydro-5-(hydroxymethyl)-4-methyl-2-[3-(trifluoromethyl)phenyl]-4,7-epoxy-1H-isoindole-1,3(2H)-dione (7);
(3aα,4β,7β,7aα)-2-[3,5-Bis(trifluoromethyl)phenyl]hexahydro-4,7-epoxy-1H-isoindole-1,3(2H)-dione (8);
(3aα,4α,7α,7aα)-2-(4-Bromophenyl)octahydro-1,3-dioxo-4,7-etheno-5H-pyrrolo[3,4-c]pyridine-5-carboxylic acid phenyl ester (9);
(3aα,4α,7α,7aα)-2-(4-Bromophenyl)octahydro-1,3-dioxo-4,7-etheno-5H-pyrrolo[3,4-c]pyridine-5-carboxylic acid phenylmethyl ester (10);
(3aα,4α,7α,7aα)-Hexahydro-2-[3-(trifluoromethyl)phenyl]-4,7-ethano-1H-pyrrolo[3,4-c]pyridine-1,3(2H)-dione trifluoroacetate (11);
(3aα,4α,7α,7aα)-5-Acetylhexahydro-2-[3-(trifluoromethyl)phenyl]-4,7-ethano-1H-pyrrolo[3,4-c]pyridine-1,3(2H)-dione (12);
(3aα,4α,7α,7aα)-5-Benzoylhexahydro-2-[3-(trifluoromethyl)phenyl]-4,7-ethano-1H-pyrrolo[3,4-c]pyridine-1,3(2H)-dione (13);
(3aα,4α,7α,7aα)-Hexahydro-5-methyl-2-[3-(trifluoromethyl)phenyl]-4,7-ethano-1H-pyrrolo[3,4-c]pyridine-1,3(2H)-dione (14);
(3aα,4α,7α,7aα)-Hexahydro-5-(phenylmethyl)-2-[3-(trifluoromethyl)phenyl]-4,7-ethano-1H-pyrrolo[3,4-c]pyridine-1,3(2H)-dione trifluoroacetate (15);
(3aα,4α,7α,7aα)-Hexahydro-5-propyl-2-[3-(trifluoromethyl)phenyl]-4,7-ethano-1H-pyrrolo[3,4-c]pyridine-1,3(2H)-dione trifluoroacetate (16);
(3aα,4α,4aβ,5aβ,6α,6aα)-2-[4-Cyano-3-(trifluoromethyl)phenyl]decahydro-1,3-dioxo-4,6-(iminomethano)cycloprop[f]isoindole-7-carboxylic acid phenylmethyl ester (17);
(3aα4α,4aβ,5aβ,6α,6aα)-4-[Decahydro-1,3-dioxo-4,6-(iminomethano)cycloprop[f]isoindol-2-yl]-2-(trifluoromethyl)benzonitrile (18);
(3aα,4α,4aβ,5aβ,6α,6aα)-4-[Decahydro-7-methyl-1,3-dioxo-4,6-(iminomethano)cycloprop[f]isoindol-2-yl]-2-(trifluoromethyl)benzonitrile (19);
(3aβ,4α,7β,7aα)-4-(Octahydro-4,7-dimethyl-1,3-dioxo-4,7-epoxy-2H-isoindol-2-yl)-2-(trifluoromethyl)benzonitrile (20B);
(3aα,4β,7β,7aα)-N-[4-[[2-[2-[4-Cyano-3-(trifluoromethyl)phenyl]octahydro-7-methyl-1,3-dioxo-4,7-epoxy-4H-isoindol-4-yl]ethyl]thio]phenyl]acetamide (21 E);
(3aα,4β,7β,7aα)-N-[4-[[2-[2-[4-Cyano-3-(trifluoromethyl)phenyl]octahydro-7-methyl-1,3-dioxo-4,7-epoxy-4H-isoindol-4-yl]ethyl]sulfinyl]phenyl]acetamide (22);
(3aα,4β,7β,7aα)-N-[4-[[2-[2-[4-Cyano-3-(trifluoromethyl)phenyl]octahydro-7-methyl-1,3-dioxo-4,7-epoxy-4H-isoindol-4-yl]ethyl]sulfonyl]phenyl]acetamide (23);
(3aα,4β,7β,7aα)- and (3aα,4α,7α,7aα)-N-[2-[2-[4-Cyano-3-(trifluoromethyl)phenyl]octahydro-7-methyl-1,3-dioxo-4,7-epoxy-4H-isoindol-4-yl]ethyl]benzenesulfonamide (24Ci & 24Cii, respectively);
(3aα,4β,7β,7aα)-4-[Octahydro-4-(2-hydroxyethyl)-7-methyl-1,3-dioxo-4,7-epoxy-2H-isoindol-2-yl]-2-(trifluoromethyl)benzonitrile (25B);
(3aα,4α,7α,7aα)- and (3aα,4β,7β,7aα)-N-[4-[2-[2-[4-Cyano-3-(trifluoromethyl)phenyl]octahydro-7-methyl-1,3-dioxo-4,7-epoxy-4H-isoindol-4-yl]ethoxy]phenyl]acetamide (26Ci & 26Cii, respectively);
(3aα,4α,7α,7aα)-Hexahydro-2-(2-naphthalenyl)-4,7-epoxy-1H-isoindole-1,3(2H)-dione (27D);
(1aα,2β,2aα,5aα,6β,6aα)-Hexahydro-4-(2-naphthalenyl)-2,6-epoxy-3H-oxireno[f]isoindole-3,5(4H)-dione (28B);
(3aα,4α,7α,7aα)-2-[4-Bromo-3-(trifluoromethyl)phenyl]-3a,4,7,7a-tetrahydro-4,7-dimethyl-4,7-epithio-1H-isoindole-1,3(2H)-dione 8-oxide (29);
(3aα,4α,7α,7aα)-2-[4-Bromo-3-(trifluoromethyl)phenyl]-3a,4,7,7a-tetrahydro-4,7-epithio-1H-isoindole-1,3(2H)-dione 8-oxide (30);
(3aα,4α,7α,7aα)-Hexahydro-2-[3-(trifluoromethyl)phenyl]-4,7-imino-1H-isoindole-1,3(2H)-dione (31D);
(3aα,4β,7β,7aα)- and (3aα,4α,7α,7aα)-3a,4,7,7a-Tetrahydro-4,7-dimethyl-2-[3-(trifluoromethyl)phenyl]-4,7-epoxy-1H-isoindole-1,3(2H)-dione (32i & 32ii, respectively);
(3aα,4α,7α,7aα)-Hexahydro-4,7-dimethyl-2-[3-(trifluoromethyl)phenyl]-4,7-epoxy-1H-isoindole-1,3(2H)-dione (33);
(3aα,4α,7α,7aα)-Tetrahydro-5-methyl-2-(4-nitro-1-naphthalenyl)-4,7-etheno-1H-pyrrolo[3,4-c]pyridine-1,3,6(2H,5H)-trione (34B);
(3aα,4β,7β,7aα)-4-[4-[2-(4-Fluorophenoxy)ethyl]octahydro-7-methyl-1,3-dioxo-4,7-epoxy-2H-isoindol-2-yl]-2-(trifluoromethyl)benzonitrile (35);
(3aα,4β,7β,7aα)-4-[4-(2-Bromoethyl]octahydro-7-methyl-1,3-dioxo-4,7-epoxy-2H-isoindol-2-yl]-2-(trifluoromethyl)benzonitrile (36);
(3aα,4β,7β,7aα)-Hexahydro-4,7-dimethyl-2-(3-methyl-4-nitrophenyl)-4,7-epoxy-1H-isoindole-1,3(2H)-dione (37);
(3aα,4β,7β,7aα)-2-(2-Fluorenyl)hexahydro-4,7-epoxy-1H-isoindole-1,3(2H)-dione;
(3aα,4β,7β,7aα)-2-[3-Chloro-4-(4-morpholinyl)phenyl]hexahydro-4,7-epoxy-1H-isoindole-1,3(2H)-dione;
(3aα,4β,7β,7aα)-2-(2,3-Dihydro-1H-inden-5-yl)hexahydro-4,7-epoxy-1H-isoindole-1,3(2H)-dione;
(3aα,4β,7β,7aα)-2-(4-Bromo-1-naphthalenyl)hexahydro-4,7-epoxy-1H-isoindole-1,3(2H)-dione;
(3aα,4β,7β,7aα)-2-(4-Chloro-1-naphthalenyl)hexahydro-4,7-epoxy-1H-isoindole-1,3(2H)-dione;
(3aα,4β,7β,7aα)-2-(5-Amino-1-naphthalenyl)hexahydro-4,7-epoxy-1H-isoindole-1,3(2H)-dione;
(3aα,4β,7β,7aα)-Hexahydro-2-(7-hydroxy-1-naphthalenyl)-4,7-epoxy-1H-isoindole-1,3(2H)-dione;
(3aα,4β,7β,7aα)-Hexahydro-2-(4-nitro-1-naphthalenyl)-4,7-epoxy-1H-isoindole-1,3(2H)-dione;
(3aα,4β,7β,7aα)-Hexahydro-2-(1H-indol-5-yl)-4,7-epoxy-1H-isoindole-1,3(2H)-dione;
(3aα,4β,7β,7aα)-Hexahydro-2-(1H-indazol-6-yl)-4,7-epoxy-1H-isoindole-1,3(2H)-dione;
(3aα,4β,7β,7aα)-2-(1,3-Benzodioxol-5-yl)hexahydro-4,7-epoxy-1H-isoindole-1,3(2H)-dione;
(3aα,4β,7β,7aα)-2-[4-Amino-3-(trifluoromethyl)phenyl]hexahydro-4,7-epoxy-1H-isoindole-1,3(2H)-dione;
(3aα,4β,7β,7aα)-2-(3-Chloro-4-iodophenyl)hexahydro-4,7-epoxy-1H-isoindole-1,3(2H)-dione;
(3aα,4β,7β,7aα)-Hexahydro-2-(8-quinolinyl)-4,7-epoxy-1H-isoindole-1,3(2H)-dione;
(3aα,4β,7β,7aα)-2-(2,3-Dihydro-1,4-benzodioxin-6-yl)hexahydro-4,7-epoxy-1H-isoindole-1,3(2H)-dione;
(3aα,4β,7β,7aα)-Hexahydro-2-[2-oxo-4-(trifluoromethyl)-2H-1-benzopyran-7-yl]-4,7-epoxy-1H-isoindole-1,3(2H)-dione;
(3aα,4β,7β,7aα)-Hexahydro-2-(4-methyl-2-oxo-2H-1-benzopyran-7-yl)-4,7-epoxy-1H-isoindole-1,3(2H)dione;
(3aα,4β,7β,7aα)-2-(2,5-Dimethoxy-4-nitrophenyl)hexahydro-4,7-epoxy-1H-isoindole-1,3(2H)-dione;
(3aα,4β,7β,7aα)-2,3,5,6-Tetrafluoro-4-(octahydro-1,3-dioxo-4,7-epoxy-2H-isoindol-2-yl)benzonitrile;
(3aα,4β,7β,7aα)-Hexahydro-2-(2,4,5-trifluorophenyl)-4,7-epoxy-1H-isoindole-1,3(2H)-dione;
(3aα,4β,7β,7aα)-Hexahydro-2-(2,4,5-trichlorophenyl)-4,7-epoxy-1H-isoindole-1,3(2H)-dione;
(3aα,4β,7β,7aα)-2-(2-Amino-4,5-dichlorophenyl)hexahydro-4,7-epoxy-1H-isoindole-1,3(2H)-dione;
(3aα,4β,7β,7aα)-2-(3,4-Difluorophenyl)hexahydro-4,7-epoxy-1H-isoindole-1,3(2H)-dione;
(3aα,4β,7β,7aα)-1-Acetyl-2,3-dihydro-6-(octahydro-1,3-dioxo-4,7-epoxy-2H-isoindol-2-yl)-1H-indole;
(3aα,4β,7β,7aα)-2-(3-Chloro-4-fluorophenyl)hexahydro-4,7-epoxy-1H-isoindole-1,3(2H)-dione;
(3aα,4β,7β,7aα)-2-(3,4-Dichlorophenyl)hexahydro-4,7-epoxy-1H-isoindole-1,3(2H)-dione;
(3aα,4β,7β,7aα)-Hexahydro-2-(3,4,5-trichlorophenyl)-4,7-epoxy-1H-isoindole-1,3(2H)-dione;
(3aα,4β,7β,7aα)-2-(3-Chloro-4-methoxyphenyl)hexahydro-4,7-epoxy-1H-isoindole-1,3(2H)-dione;
(3aα,4β,7β,7aα)-2-(3-Chloro-4-methylphenyl)hexahydro-4,7-epoxy-1H-isoindole-1,3(2H)-dione;
(3aα,4β,7β,7aα)-Hexahydro-2-(2-methyl-1-naphthalenyl)4,7-epoxy-1H-isoindole-1,3(2H)-dione;
(3aα,4β,7β,7aα)-2-(4-Chloro-3-methylphenyl)hexahydro-4,7-epoxy-1H-isoindole-1,3(2H)-dione;
(3aα,4β,7β,7aα)-2-(3,4-Dimethylphenyl)hexahydro-4,7-epoxy-1H-isoindole-1,3(2H)-dione;
(3aα,4β,7β,7aα)-2-[4-Bromo-3-(trifluoromethyl)phenyl]hexahydro-4,7-epoxy-1H-isoindole-1,3(2H)-dione;
(3aα,4β,7β,7aα)-2-(4-Bromo-3-methylphenyl)hexahydro-4,7-epoxy-1H-isoindole-1,3(2H)-dione;
(3aα,4β,7β,7aα)-2-(4-Fluoro-3-nitrophenyl)hexahydro-4,7-epoxy-1H-isoindole-1,3(2H)-dione;
(3aα,4β,7β,7aα)-2-[4-Fluoro-3-(trifluoromethyl)phenyl]hexahydro-4,7-epoxy-1H-isoindole-1,3(2H)-dione;
(3aα,4β,7β,7aα)-2-(4-Chloro-3-nitrophenyl)hexahydro-4,7-epoxy-1H-isoindole-1,3(2H)-dione;
(3aα,4β,7β,7aα)-2-[4-Chloro-3-(trifluoromethyl)phenyl]hexahydro-4,7-epoxy-1H-isoindole-1,3(2H)-dione;
(3aα,4β,7β,7aα)-2-(4-Chloro-2-methoxy-5-methylphenyl)hexahydro-4,7-epoxy-1H-isoindole-1,3(2H)-dione;
(3aα,4β,7β,7aα)-2-(4-Amino-3-nitrophenyl)hexahydro-4,7-epoxy-1H-isoindole-1,3(2H)-dione;
(3aα,4β,7β,7aα)-Hexahydro-2-(4-methyl-3-nitrophenyl)-4,7-epoxy-1H-isoindole-1,3(2H)-dione;
(3aα,4β,7β,7aα)-2-(3,4-Dimethoxyphenyl)hexahydro-4,7-epoxy-1H-isoindole-1,3(2H)-dione;
(3aα,4β,7β,7aα)-Hexahydro-2-(3-hydroxy-4-methoxyphenyl)-4,7-epoxy-1H-isoindole-1,3(2H)-dione;
(3aα,4β,7β,7aα)-Hexahydro-2-(4-methyl-5-nitro-2-pyridinyl)-4,7-epoxy-1H-isoindole-1,3(2H)-dione;
(3aα,4β,7β,7aα)-2-Chloro-4-(octahydro-1,3-dioxo-4,7-epoxy-2H-isoindol-2-yl)-α-phenylbenzeneacetonitrile;
(3aα,4β,7β,7aα)-Hexahydro-2-(2-methoxy-3-dibenzofuranyl)-4,7-epoxy-1H-isoindole-1,3(2H)-dione;
(3aα,4β,7β,7aα)-Hexahydro-2-(2,3,4-trifluorophenyl)-4,7-epoxy-1H-isoindole-1,3(2H)-dione;
(3aα,4β,7β,7aα)-2-(2,3-Dihydro-2-methyl-1,3-dioxo-1H-isoindol-5-yl)hexahydro-4,7-epoxy-1H-isoindole-1,3(2H)-dione;
(3aα,4β,7β,7aα)-2-(4-Bromo-2,3,5,6-tetrafluorophenyl)hexahydro-4,7-epoxy-1H-isoindole-1,3(2H)-dione;
(3aα,4β,7β,7aα)-Hexahydro-2-(2-hydroxy-1-naphthalenyl)-4,7-epoxy-1H-isoindole-1,3(2H)-dione;
(3aα,4β,7β,7aα)-2-[2,5-Dichloro-4-(1H-pyrrol-1-yl)phenyl]hexahydro-4,7-epoxy-1H-isoindole-1,3(2H)-dione;
(3aα,4β,7β,7aα)-Hexahydro-2-[4-(methoxymethyl)-2-oxo-2H-1-benzopyran-7-yl]-4,7-epoxy-1H-isoindole-1,3(2H)-dione;
(3aα,4β,7β,7aα)-2-(6-Benzothiazolyl)hexahydro-4,7-epoxy-1H-isoindole-1,3(2H)-dione;
(3aα,4β,7β,7aα)-2-Methoxy-4-(octahydro-1,3-dioxo-4,7-epoxy-2H-isoindol-2-yl)benzoic acid methyl ester;
(3aα,4β,7β,7aα)-2-Methyl-5-(octahydro-1,3-dioxo-4,7-epoxy-2H-isoindol-2-yl)benzonitrile;
(3aα,4β,7β,7aα)-Hexahydro-2-(2-oxo-2H-1-benzopyran-6-yl)-4,7-epoxy-1H-isoindole-1,3(2H)-dione;
(3aα,4β,7β,7aα)-Hexahydro-2-(2,3,5,6-tetramethyl-4-nitrophenyl)-4,7-epoxy-1H-isoindole-1,3(2H)-dione;
(3aα,4β,7β,7aα)-Hexahydro-2-(2,4,5-trimethylphenyl)-4,7-epoxy-1H-isoindole-1,3(2H)-dione;
(3aα,4β,7β,7aα)-2-(4-Fluoro-3-methylphenyl)hexahydro-4,7-epoxy-1H-isoindole-1,3(2H)-dione;
(3aα,4β,7β,7aα)-Hexahydro-2-(3-methoxy-4-methylphenyl)-4,7-epoxy-1H-isoindole-1,3(2H)-dione;
(3aα,4β,7β,7aα)-N-Ethyl-2-methyl-5-(octahydro-1,3-dioxo-4,7-epoxy-2H-isoindol-2-yl)-N-phenylbenzenesulfonamide;
(3aα,4β,7β,7aα)-2,6-Dibromo-4-(octahydro-1,3-dioxo-4,7-epoxy-2H-isoindol-2-yl)benzenesulfonamide;
(3aα,4β,7β,7aα)-2,4-Dimethyl-6-(octahydro-1,3-dioxo-4,7-epoxy-2H-isoindol-2-yl)-3-pyridinecarbonitrile;
(3aα,4β,7β,7aα)-2-(2,3-Dimethyl-1H-indol-5-yl)hexahydro-4,7-epoxy-1H-isoindole-1,3(2H)-dione;
(3aα,4β,7β,7aα)-2-(3-Dibenzofuranyl)hexahydro-4,7-epoxy-1H-isoindole-1,3(2H)-dione;
(3aα,4β,7β,7aα)-Hexahydro-2-(2′-hydroxy[1,1′:3′,1″-terphenyl]-5′-yl)-4,7-epoxy-1H-isoindole-1,3(2H)-dione;
(3aα,4β,7β,7aα)-Hexahydro-2-(5,6,7,8-tetrahydro-3-hydroxy-2-naphthalenyl)-4,7-epoxy-1H-isoindole-1,3(2H)-dione;
(3aα,4β,7β,7aα)-2-(2,3-Dihydro-1H-indol-6-yl)hexahydro-4,7-epoxy-1H-isoindole-1,3(2H)-dione;
(3aα,4β,7β,7aα)-2-(1,3-Dihydro-2,2-dioxidobenzo[c]thiophen-5-yl)hexahydro-4,7-epoxy-1H-isoindole-1,3(2H)-dione;
(3aα,4β,7β,7aα)-Hexahydro-2-(2-hydroxy-4,5-dimethylphenyl)-4,7-epoxy-1H-isoindole-1,3(2H)-dione;
(3aα,4β,7β,7aα)-2-(2,3-Dihydro-2,2,3,3-tetrafluoro-1,4-benzodioxin-6-yl)hexahydro-4,7-epoxy-1H-isoindole-1,3(2H)-dione;
(3aα,4β,7β,7aα)-Hexahydro-2-(1H-indazol-5-yl)-4,7-epoxy-1H-isoindole-1,3(2H)-dione;
(3aα,4β,7β,7aα)-2-(4-Amino-2,3,5,6-tetrafluorophenyl)hexahydro-4,7-epoxy-1H-isoindole-1,3(2H)-dione;
(3aα,4β,7β,7aα)-2-(4-Bromo-3-chlorophenyl)hexahydro-4,7-epoxy-1H-isoindole-1,3(2H)-dione;
(3aα,4β,7β,7aα)-Hexahydro-2-(5-hydroxy-1-naphthalenyl)-4,7-epoxy-1H-isoindole-1,3(2H)-dione;
(3aα,4β,7β,7aα)-4-(Octahydro-1,3-dioxo-4,7-epoxy-2H-isoindol-2-yl)-2-(trifluoromethyl)benzonitrile;
(3aα,4β,7β,7aα)-2-(4-Morpholinyl)-5-(octahydro-1,3-dioxo-4,7-epoxy-2H-isoindol-2-yl)benzoic acid methyl ester;
(3aα,4β,7β,7aα)-2-Fluoro-5-(octahydro-1,3-dioxo-4,7-epoxy-2H-isoindol-2-yl)benzonitrile;
(3aα,4β,7β,7aα)-Hexahydro-2-(2-naphthalenyl)-4,7-epoxy-1H-isoindole-1,3(2H)-dione;
(3aα,4β,7β,7aα)-2-(9-Ethyl-9H-carbazol-3-yl)hexahydro-4,7-epoxy-1H-isoindole-1,3(2H)-dione;
(3aα,4β,7β,7aα)-2-[1,2-Dihydro-8-methyl-2-oxo-4-(trifluoromethyl)-7-quinolinyl]hexahydro-4,7-epoxy-1H-isoindole-1,3(2H)-dione;
(3aα,4α,7α,7aα)-Hexahydro-2-[3-(trifluoromethyl)phenyl]-4,7-epoxy-1H-isoindole-1,3(2H)-dione;
(3aα,4α,7α,7aα)-Hexahydro-2-(4-nitro-1-naphthalenyl)-4,7-epoxy-1H-isoindole-1,3(2H)-dione;
(3aα,4β,7β,7aα)-2-(4-Bromo-3-methylphenyl)-3a,4, 7,77a-tetrahydro-4,7-epoxy-1H-isoindole-1,3(2H)-dione;
(3aα,4β,7β,7aα)-3a,4,7,7a-Tetrahydro-2-(2-naphthalenyl)-4,7-epoxy-1H-isoindole-1,3(2H)-dione;
(3aα,4β,7β,7aα)-2-(9-Ethyl-9H-carbazol-3-yl)-3a,4,7,7a-tetrahydro-4,7-epoxy-1H-isoindole-1,3(2H)-dione;
(3aα,4β,7β,7aα)-2-[4-Fluoro-3-(trifluoromethyl)phenyl]-3a,4,7,7a-tetrahydro-4,7-epoxy-1H-isoindole-1,3(2H)-dione;
(3aα,4β,7β,7aα)-2-[1,2-Dihydro-8-methyl-2-oxo-4-(trifluoromethyl)-7-quinolinyl]-3a,4,7,7a-tetrahydro-4,7-epoxy-1H-isoindole-1,3(2H)-dione;
(3aα,4α,7α,7aα)-4-[(Acetyloxy)methyl]-2-(4-bromo-3-methylphenyl)-3a,4,7,7a-tetrahydro-4,7-epoxy-1H-isoindole-1,3(2H)-dione;
(3aα,4β,7β,7aα)-4-[(Acetyloxy)methyl]-2-(4-bromo-3-methylphenyl)-3a,4,7,7a-tetrahydro-4,7-epoxy-1H-isoindole-1,3(2H)-dione.;
(3aα,4β,7β,7aα)-Hexahydro-4,7-dimethyl-2-[3-(trifluoromethyl)phenyl]-4,7-epoxy-1H-isoindole-1,3(2H)-dione;
(3aα,4β,7β,7aα)-4-(Octahydro-4,7-dimethyl-1,3-dioxo-4,7-epoxy-2H-isoindol-2-yl)-1-naphthalenecarbonitrile;
(3aα,4β,7β,7aα)-(Benzo[b]thiophen-3-yl)hexahydro-4,7-dimethyl-4,7-epoxy-1H-isoindole-1,3(2H)-dione;
(3aα,4β,7β,7aα)-Hexahydro-4,7-dimethyl-2-[4-nitro-3-(trifluoromethyl)phenyl]-4,7-epoxy-1H-isoindole-1,3(2H)-dione;
(3aα,4β,7β,7aα)-4-(1,3,3a,4,7,7a-Hexahydro-4,7-dimethyl-1,3-dioxo-4,7-epoxy-2H-isoindol-2-yl)-1-naphthalenecarbonitrile;
(3aα,4α,7α,7aα)-Hexahydro-4-methyl-2-(2-naphthalenyl)-4,7-epoxy-1H-isoindole-1,3(2H)-dione;
(3aα,4β,7β,7aα)-2-(4-Bromo-3-methylphenyl)hexahydro-4-methyl-4,7-epoxy-1H-isoindole-1,3(2H)-dione;
(3aα,4β,7β,7aα)-Hexahydro-4-methyl-2-[3-(trifluoromethyl)phenyl]-4,7-epoxy-1H-isoindole-1,3(2H)-dione;
(3aα,4β,7β,7aα)-2-(3,5-Dichlorophenyl)hexahydro-4-methyl-4,7-epoxy-1H-isoindole-1,3(2H)-dione;
(3aα,4β,7β,7aα)-2-(3-Chloro-4-fluorophenyl)hexahydro-4-methyl-4,7-epoxy-1H-isoindole-1,3(2H)-dione;
(3aα,4β,7β,7aα)-2-Methoxy-4-(octahydro-1,3-dioxo-4-methyl-4,7-epoxy-2H-isoindol-2-yl)-1-naphthalenecarbonitrile;
(3aα,4β,7β,7aα)-Hexahydro-4-methyl-2-[4-nitro-3-(trifluoromethyl)phenyl]-4,7-epoxy-1H-isoindole-1,3(2H)-dione;
(3aα,4β,7β,7aα)-Hexahydro-2-[4-(1H-imidazol-1-yl)phenyl]-4-methyl-4,7-epoxy-1H-isoindole-1,3(2H)-dione;
(3aα,4β,7β,7aα)-2-[3-Chloro-4-(2-thiazolyl)phenyl]hexahydro-4-methyl-4,7-epoxy-1H-isoindole-1,3(2H)-dione;
(3aα,4α,7α,7aα)-2-(3,5-Dichlorophenyl)hexahydro-4,7-imino-1H-isoindole-1,3(2H)-dione;
(3aα,4α,7α,7aα)-2-(4-Bromo-1-naphthalenyl)hexahydro-4,7-imino-1H-isoindole-1,3(2H)-dione;
(3aα,4α,7α,7aα)-2-(4-Bromo-3-methylphenyl)hexahydro-4,7-imino-1H-isoindole-1,3(2H)-dione;
(3aα,4α,7α,7aα)-Hexahydro-2-(4-nitro-1-naphthalenyl)-4,7-imino-1H-isoindole-1,3(2H)-dione;
(3aα,4α,7α,7aα)-8-Acetyl-2-(3,5-dichlorophenyl)hexahydro-4,7-imino-1H-isoindole-1,3(2H)-dione;
(3aα,4α,7α,7aα)-Octahydro-1,3-dioxo-2-[3-(trifluoromethyl)phenyl]-4,7-ethano-5H-pyrrolo[3,4-c]pyridine-5-carboxylic acid phenyl ester;
(3aα,4α,7α,7aα)-4-(Octahydro-1,3-dioxo-4,7-ethano-2H-pyrrolo[3,4-c]pyridin-2-yl)-1-naphthalenecarbonitrile;
(3aα,4α,7α,7aα)-4-(Octahydro-5-methyl-1,3-dioxo-4,7-ethano-2H-pyrrolo[3,4-c]pyridin-2-yl)-1-naphthalenecarbonitrile;
(3aα,4α,7α,7aα)-2-(4-Cyano-1-naphthalenyl)octahydro-1,3-dioxo-4,7-etheno-5H-pyrrolo[3,4-c]pyridine-5-carboxylic acid phenylmethyl ester;
(3aα,4α,7α,7aα)-4-(Octahydro-1,3-dioxo-4,7-ethano-2H-pyrrolo[3,4-c]pyridin-2-yl)-2-(trifluoromethyl)benzonitrile;
(3aα,4α,7α,7aα)-4-(Octahydro-5-methyl-1,3-dioxo-4,7-ethano-2H-pyrrolo[3,4-c]pyridin-2-yl)-2-(trifluoromethyl)benzonitrile;
(3aα,4α,7α,7aα)-2-[4-Cyano-3-(trifluoromethyl)phenyl]octahydro-1,3-dioxo-4,7-etheno-5H-pyrrolo[3,4-c]pyridine-5-carboxylic acid phenylmethyl ester;
(3aα,4α,7α,7aα)-2-[4-Bromo-3-(trifluoromethyl)phenyl]tetrahydro-5-methyl-4,7-etheno-1H-pyrrolo[3,4-c]pyridine-1,3,6(2H,5H)-trione;
(3aα,4α,7α,7aα)-Tetrahydro-5-methyl-2-[3-(trifluoromethyl)phenyl]-4,7-etheno-1H-pyrrolo[3,4-c]pyridine-1,3,6(2H,5H)-trione;
(3aα,4α,7α,7aα)-Tetrahydro-5-methyl-2-(2-naphthalenyl)-4,7-etheno-1H-pyrrolo[3,4-c]pyridine-1,3,6(2H ,5H)-trione;
(1aα,2β,2aα,5aα,6β,6aα)-Hexahydro-4-[3-(trifluoromethyl)phenyl]-2,6-epoxy-3H-oxireno[f]isoindole-3,5(4H)-dione;
(1aα,2β,2aα,5aα,6β,6aα)-4-(3,5-Dichlorophenyl)hexahydro-2,6-epoxy-3H-oxireno[f]isoindole-3,5(4H)-dione;
(1aα,2β,2aα,5aα,6β,6aα)-Hexahydro-4-(4-nitro-1-naphthalenyl)-2,6-epoxy-3H-oxireno[f]isoindole-3,5(4H)-dione;
(1aα,2β,2aα,5aα,6β,6aα)-4-(3,4-Dichlorophenyl)hexahydro-2,6-epoxy-3H-oxireno[f]isoindole-3,5(4H)-dione;
2-[4-(4-Bromophenoxy)phenyl]-3a,4,7,7a-tetrahydro-4,7-dimethyl-4,7-epoxy-1H-isoindole-1,3(2H)-dione;
3a,4,7,7a-Tetrahydro-2-(2-methoxyphenyl)-4,7-dimethyl-4,7-epoxy-1H-isoindole-1,3(2H)-;
[(1,2,3,3a,7,7a-Hexahydro-2-phenyl-4,7-epoxy-4H-isoindol-4-yl)methyl]carbamic acid (3,5-dimethoxyphenyl)methyl ester;
2-(2,4-Dimethylphenyl)-3a,4,7,7a-tetrahydro-4-(hydroxymethyl)-4,7-epoxy-1H-isoindole-1,3(2H)-dione;
2-(1,3-Benzodioxol-5-yl)-3a,4,7,7a-tetrahydro-4-methyl-4,7-epoxy-1H-isoindole-1,3(2H)-dione;
4-[Bis(acetyloxy)methyl]-2-(3-bromophenyl)-3a,4,7,7a-tetrahydro-4,7-epoxy-1H-isoindole-1,3(2H)-dione;
N-[[1,2,3,3a,7,7a-Hexahydro-2-(2,4,6-trimethylphenyl)-4,7-epoxy-4H-isoindol-4-yl]methyl]-2,2-dimethylpropanamide;
3a,4,7,7a-Tetrahydro-4-(hydroxymethyl)-2-[2-(trifluoromethyl)phenyl]-4,7-epoxy-1H-isoindole-1,3(2H)-dione;
3a,4,7,7a-Tetrahydro-4-(hydroxymethyl)-2-(1-naphthalenyl)-4,7-epoxy-1H-isoindole-1,3(2H)-dione;
2-Chloro-5-(1,3,3a,4,7, 7a-hexahydro4,7-dimethyl-4,7-epoxy-2H-isoindol-2-yl)benzoic acid methyl ester;
4-[Bis(acetyloxy)methyl]-2-(4-bromo-2-nitrophenyl)-3a,4,7,7a-tetrahydro-4,7-epoxy-1H-isoindole-1,3(2H)-dione;
3a,4,7,7a-Tetrahydro-4-methyl-2-(4-methyl-3-nitrophenyl)-4,7-epoxy-1H-isoindole-1,3(2H)-dione;
2-[2-Chloro-5-(trifluoromethyl)phenyl]-3a,4,7,7a-tetrahydro-4-methyl-4,7-epoxy-1H-isoindole-1,3(2H)-dione;
2-[4-Chloro-3-(trifluoromethyl)phenyl]-3a,4,7,7a-tetrahydro-4,7-dimethyl-4,7-epoxy-1H-isoindole-1,3(2H)-dione;
2-(1,3,3a,4,7,7a-Hexahydro-4-methyl-4,7-epoxy-2H-isoindol-2-yl)benzonitrile;
2-(4-Fluorophenyl)-3a,4,7,7a-tetrahydro-4-methyl-4,7-epoxy-1H-isoindole-1,3(2H)-dione;
2,2,2-Trifluoro-N-[(1,2,3,3a,7,7a-hexahydro-2-phenyl-4,7-epoxy-4H-isoindol-4-yl)methyl]acetamide;
3a,4,7,7a-Tetrahydro-4,7-dimethyl-2-(4-methyl-3-nitrophenyl)-4,7-epoxy-1H-isoindole-1,3(2H)-dione;
2-Chloro-5-[1,3,3a,4,7,7a-hexahydro-4-(hydroxymethyl)-4,7-epoxy-2H-isoindol-2-yl]benzoic acid;
3a,4,7,7a-Tetrahydro-4,7-dimethyl-2-(4-nitrophenyl)-4,7-epoxy-1H-isoindole-1,3(2H)-dione;
3a,4,7,7a-Tetrahydro-2-(2-mercaptophenyl)-4,7-epoxy-1H-isoitidole-1,3(2H)-dione;
3a,4,7,7a-Tetrahydro-2-[2-[(phenylmethyl)thio]phenyl]-4,7-epoxy-1H-isoindole-1,3(2H)-dione;
[[2-(4-Chlorophenyl)-1,2,3,3a,7,7a-hexahydro-4,7-epoxy-4H-isoindol-4-yl]methyl]carbamic acid 2-methylpropyl ester;
4-(1,1-Dimethylethyl)-N-[[1,2,3,3a,7,7a-hexahydro-2-(4-methylphenyl)-4,7-epoxy-4H-isoindol-4 yl]methyl]benzamide;
2,4-Dichloro-N-[[1,2,3,3a,7,7a-hexahydro-2-(4-nitrophenyl)-4,7-epoxy-4H-isoindol-4-yl]methyl]benzamide;
N-[[2-(4-Chlorophenyl)-1,2,3,3a,7,7a-hexahydro-4,7-epoxy-4H-isoindol-4-yl]methyl]-2,4,6-trimethylbenzenesulfonamide;
[(1,2,3,3a,7,7a-Hexahydro-2-phenyl-4,7-epoxy-4H-isoindol-4-yl)methyl]carbamic acid 1,1-dimethylethyl ester;
N-[(1,2,3,3a,7,7a-Hexahydro-2-phenyl-4,7-epoxy-4H-isoindol-4-yl)methyl]-2-phenoxyacetamide;
N-[[1,2,3,3a,7,7a-Hexahydro-2-(4-nitrophenyl)-4,7-epoxy-4H-isoindol-4-yl]methyl]-2,2-dimethylpropanamide;
2-(2,4-Dichlorophenoxy)-N-[[1,2,3,3a,7,7a-hexahydro-2-(4-nitrophenyl)-4,7-epoxy-4H-isoindol-4-yl]methyl]acetamide;
N-[[1,2,3,3a,7,7a-Hexahydro-2-(4-methylphenyl)-4,7-epoxy-4H-isoindol-4-yl]methyl]-3 ,5-dimethoxybenzamide;
N-[[2-(4-Chlorophenyl)-1,2,3,3a,7,7a-hexahydro-4,7-epoxy-4H-isoindol-4-yl]methyl]-2-nitrobenzenesulfonamide;
(3aα,4β,7β,7aα)-Hexahydro-2-[(1S)-1-phenylethyl]-4,7-epoxy-1H-isoindole-1,3(2H)-dione;
(3aα,4β,7β,7aα)-Hexahydro-2-[(1S)-2-hydroxy-1-phenylethyl]-4,7-epoxy-1H-isoindole-1,3(2H)-dione;
(3aα,4β,7β,7aα)-2-[(1S)-2-(Acetyloxy)-1-phenylethyl]-3a,4,7,7a-tetrahydro-4,7-epoxy-1H-isoindole-1,3(2H)-dione;
(3aα,4α,7α,7aα)-3a,4,7,7a-Tetrahydro-2-[(1S)-1-phenylethyl]-4,7-epoxy-1H-isoindole-1,3(2H)-dione;
(3aα,4β,7β,7aα)-Hexahydro-2-[(1R)-1-phenylethyl]-4,7-epoxy-1H-isoindole-1,3(2H)-dione;
(3aα,4β,7β,7aα)-4-[[[(Octahydro-1,3-dioxo-4,7-epoxy-2H-isoindol-2-yl)methyl]amino]benzoic acid;
(3aα,4β,7β,7aα)-Hexahydro-2-(4-morpholinylmethyl)-4,7-epoxy-1H-isoindole-1,3(2H)-dione
(3aα,4β,7β,7aα)-4-[Octahydro-4-(2-hydroxyethyl)-7-methyl-1,3-dioxo-4,7-epoxy-2H-isoindol-2-yl]-2-(trifluoromethyl)benzonitrile;
(3aα,4β,7β,7aα)- and (3aα,4α,7α,7aα)-4-[Octahydro-4-methyl-1,3-dioxo-7-(phenylmethyl)-4,7-epoxy-2H-isoindol-2-yl]-2-(trifluoromethyl)benzonitrile,
(3aα,4β,7β,7aα)-(4-[7-[2-(4-Bromophenoxy)ethyl]octahydro-4-methyl-1,3-dioxo-4,7-epoxy-2H-isoindol-2-yl]-2-(trifluoromethyl)benzonitrile;
(3aα,4β,7β,7aα)-4-[Octahydro-7-[2-(4-iodophenoxy)ethyl]-4-methyl-1,3-dioxo-4,7-epoxy-2H-isoindol-2-yl]-2-(trifluoromethyl)benzonitrile;
(3aα,4β,7β,7aα)-4-[Octahydro-4-methyl-1,3-dioxo-7-[2-[4-(trifluoromethyl)phenoxy]ethyl]-4,7-epoxy-2H-isoindol-2-yl]-2-(trifluoromethyl)benzonitrile;
(3aα,4β,7β,7aα)-4-[Octahydro-7-[2-(4-methoxyphenoxy)ethyl]-4-methyl-1,3-dioxo-4,7-epoxy-2H-isoindol-2-yl]-2-(trifluoromethyl)benzonitrile;
(3aα,4β,7β,7aα)-4-[7-[2-(4-Ethoxyphenoxy)ethyl]octahydro-4-methyl-1,3-dioxo-4,7-epoxy-2H-isoindol-2-yl]-2-(trifluoromethyl)benzonitrile;
(3aα,4β,7β,7aα)-4-[7-[2-(4-Chlorophenoxy)ethyl]octahydro-4-methyl-1,3-dioxo-4,7-epoxy-2H-isoindol-2-yl]-2-(trifluoromethyl)benzonitrile;
(3aα,4β,7β,7aα)-4-[2-[2-[4-Cyano-3-(trifluoromethyl)phenyl]octahydro-7-methyl-1,3-dioxo-4,7-epoxy-4H-isoindol-4-yl]ethoxy]benzoic acid, methyl ester;
(3aα,4β,7β,7aα)-Hexahydro-4-(2-hydroxyethyl)-7-methyl-2-(3-methyl-4-nitrophenyl)-4,7-epoxy-1H-isoindole-1,3(2H)-dione;
(3aα,4β,7β,7aα)-4-[Octahydro-4-methyl-1,3-dioxo-7-[2-[4-(trifluoromethoxy)phenoxy]ethyl]-4,7-epoxy-2H-isoindol-2-yl]-2-(trifluoromethyl)benzonitrile;
(3aα,4β,7β,7aα)-2-(3,5-Dichlorophenyl)hexahydro-4,7-dimethyl-4,7-epoxy-1H-isoindole-1,3(2H)-dione;
(3aα,4β,7β,7aα)-Hexahydro-4,7-dimethyl-2-(4-nitro-1-naphthalenyl)-4,7-epoxy-1H-isoindole-1,3(2H)-dione;
(3aα,4β,7β,7aα)-2-[4-Cyano-3-(trifluoromethyl)phenyl]octahydro-7-methyl-1,3-dioxo-4,7-epoxy-4H-isoindole-4-propanenitrile;
(3aα,4β,7β,7aα)-4-[Octahydro-4-methyl-7-[2-(4-morpholinyl)ethyl]-1,3-dioxo-4,7-epoxy-2H-isoindol-2-yl]-2-(trifluoromethyl)benzonitrile, trifluoroacetate;
(3aα,4β,7β,7aα)-2-(5-Fluoro-1-naphthalenyl)hexahydro-4,7-dimethyl-4,7-epoxy-1H-isoindole-1,3(2H)-dione;
(3aα,4β,7β,7aα)-2-(5-Fluoro-4-nitro-1-naphthalenyl)hexahydro-4,7-dimethyl-4,7-epoxy-1H-isoindole-1,3(2H)-dione;
(3aα,4β,7β,7aα)-2-(1,1-Dioxidobenzo[b]thiophen-3-yl)hexahydro-4,7-dimethyl-4,7-epoxy-1H-isoindole-1,3(2H)-dione;
4-(1,3,3a,4,7,7a-Hexahydro-4,6,7-trimethyl-1,3-dioxo-4,7-epoxy-2H-pyrrolo[3,4-c]pyridin-2-yl)-2-(trifluoromethyl)benzonitrile;
(3aα,4β,7β,7aα)-Tetrahydro-4,7-dimethyl-2-[3-(trifluoromethyl)phenyl]-4,7-epoxy-1H-isoindole-1,3,5(2H,4H)-trione;
(3aα,4α,7α,7aα)-Tetrahydro-4,7-dimethyl-2-[3-(trifluoromethyl)phenyl]-4,7-epoxy-1H-isoindole-1,3,5(2H,4H)-trione;
(3aα,4β,7β,7aα)-2-(5-Chloro-1-naphthalenyl)hexahydro-4,7-dimethyl-4,7-epoxy-1H-isoindole-1,3(2H)-dione;
(3aα,4β,7β,7aα)-2-(5-Chloro-4-nitro-1-naphthalenyl)hexahydro-4,7-dimethyl-4,7-epoxy-1H-isoindole-1,3(2H)-dione;
(3aα,4β,7β,7aα)-4-Ethylhexahydro-7-methyl-2-(4-nitro-1-naphthalenyl)-4,7-epoxy-1H-isoindole-1,3(2H)-dione;
(3aα,4β,7β,7aα)-2-(4-Cyano-1-naphthalenyl)-N-(4-fluorophenyl)octahydro-7-methyl-1,3-dioxo-4,7-epoxy-4H-isoindole-4-acetamide;
(3aα,4β,7β,7aα)-Hexahydro-4-methyl-2-(2-naphthalenyl)-4,7-epoxy-1H-isoindole-1,3(2H)-dione, faster eluting enantiomer;
(3aα,4β,7β,7aα)-Hexahydro-4-methyl-2-(2-naphthalenyl)-4,7-epoxy-1H-isoindole-1,3(2H)-dione, slower eluting enantiomer;
(3aα,4β,7β,7aα)-4-[4-[2-[[(4-Fluorophenyl)methyl]methylamino]ethyl]octahydro-7-methyl-1,3-dioxo-4,7-epoxy-2H-isoindol-2-yl]-2-(trifluoromethyl)benzonitrile;
(3aα,4β,5β,6β,7β,7aα)-4-(Octahydro-4,5,6,7-tetramethyl-1,3-dioxo-4,7-epoxy-2H-isoindol-2-yl)-2-(trifluoromethyl)benzonitrile;
(3aα,4β,7β,7aα)-4-[Octahydro-4-methyl-1,3-dioxo-7-[2-[4-(trifluoromethyl)phenoxy]ethyl]-4,7-epoxy-2H-isoindol-2-yl]-2-(trifluoromethyl)benzonitrile, faster eluting antipode;
(3aα,4β,7β,7aα)-4-[Octahydro-4-methyl-1,3-dioxo-7-[2-[4-(trifluoromethyl)phenoxy]ethyl]-4,7-epoxy-2H-isoindol-2-yl]-2-(trifluoromethyl)benzonitrile, slower eluting enantiomer;
(3aα,4β,5β,7β,7aα)-4-(Octahydro-5-hydroxy-4,7-dimethyl-1,3-dioxo-4,7-epoxy-2H-isoindol-2-yl)-2-(trifluoromethyl)benzonitrile;
(3aα,4β,5β,7β,7aα)-4-(Octahydro-5-hydroxy-4,7-dimethyl-1,3-dioxo-4,7-epoxy-2H-isoindol-2-yl)-2-(trifluoromethyl)benzonitrile;
((αR)-α-Methoxybenzeneacetic acid, 2-[(3aα,4β,7β,7aα)-2-(4-cyano-1-naphthalenyl)octahydro-7-methyl-1,3-dioxo-4,7-epoxy-4H-isoindol-4-y]ethyl ester;
(3aα,4β,7β,7aα)-2-(Methylthio)-4-(octahydro-4,7-dimethyl-1,3-dioxo-4,7-epoxy-2H-isoindol-2-yl)benzonitrile;
(3aα,4β,7β,7aα)-2-(Methylsulfinyl)-4-(octahydro-4,7-dimethyl-1,3-dioxo-4,7-epoxy-2H-isoindol-2-yl)benzonitrile;
(3aα,4β,7β,7aα)-2-(Methylsulfonyl)-4-(octahydro-4,7-dimethyl-1,3-dioxo-4,7-epoxy-2H-isoindol-2-yl)benzonitrile;
(3aα,4β,5β,7β,7aα)-7-[2-[[(1,1-Dimethylethyl)dimethylsilyl]oxy]ethyl]hexahydro-5-hydroxy-4-methyl-2-(4-nitro-1-naphthalenyl)-4,7-epoxy-1H-isoindole-1,3(2H)-dione;
(3aα,4β,5β,7β,7aα)-Hexahydro-5-hydroxy-7-(2-hydroxyethyl)-4-methyl-2-(4-nitro-1-naphthalenyl)-4,7-epoxy-1H-isoindole-1,3(2H)-dione;
(3aα,4β,5β,7β,7aα)-7-[2-(4-Fluorophenoxy)ethyl]hexahydro-5-hydroxy-4-methyl-2-(4-nitro-1-naphthalenyl)-4,7-epoxy-1H-isoindole-1,3(2H)-dione;
(3aα,4β,5β,6β,7β,7aα)-4-(Octahydro-5,6-dihydroxy-4,7-dimethyl-1,3-dioxo-4,7-epoxy-2H-isoindol-2-yl)-2-(trifluoromethyl)benzonitrile;
(3aα,4β,5α,6α,7β,7aα)-4-(Octahydro-5,6-dihydroxy-4,7-dimethyl-1,3-dioxo-4,7-epoxy-2H-isoindol-2-yl)-2-(trifluoromethyl)benzonitrile;
(3aα,4β,5β,6β,7β,7aα)-4-[Octahydro-5,6-dihydroxy-4-(hydroxyethyl)-7-methyl-1,3-dioxo-4,7-epoxy-2H-isoindol-2-yl]-1-naphthalenecarbonitrile;
(3aα,4β,5β,6β,7β,7aα)-4-[Octahydro-5,6-dihydroxy-4-methyl-1,3-dioxo-7-[2-[4-(trifluoromethyl)phenoxy]ethyl]-4,7-epoxy-2H-isoindol-2-yl]-2-(trifluoromethyl)benzonitrile;
(3aα,4β,5β,5aβ,8aβ,8bα)-4-(Decahydro-5-hydroxy-4-methyl-1,3-dioxo-4,8a-epoxy-2H-furo[3,2-e]isoindol-2-yl)-1-naphthalenecarbonitrile;
(3aα,4β,7β,7aα)-2-(4-Cyano-1-naphthalenyl)octahydro-7-methyl-1,3-dioxo-4,7-epoxy-4H-isoindole-4-acetic acid;
(3aα,4β,7β,7aα)-2-(4-Cyano-1-naphthalenyl)octahydro-7-methyl-1,3-dioxo-4,7-epoxy-4H-isoindole-4-acetic acid, methyl ester;
(3aα,4β,7β,7aα)-2-(4-Cyano-1-naphthalenyl)-N-[(4-fluorophenyl)methyl]octahydro-7-methyl-1,3-dioxo-4,7-epoxy-4H-isoindole-4-acetamide;
(3aα,4β,7β,7aα)-N-[2-[2-(4-Cyano-1-naphthalenyl)octahydro-7-methyl-1,3-dioxo-4,7-epoxy-4H-isoindol-4-yl]ethyl]-4-fluorobenzamide;
[3aR-(3aα,4β,7β,7aα)]-4-[Octahydro-4-(2-hydroxyethyl)-7-methyl-1,3-dioxo-4,7-epoxy-2H-isoindol-2-yl]-1-naphthalenecarbonitrile;
[3aS-(3aα,4β,7β,7aα)]-4-[Octahydro-4-(2-hydroxyethyl)-7-methyl-1,3-dioxo-4,7-epoxy-2H-isoindol-2-yl]-1-naphthalenecarbonitrile;
[3aR-(3aα,4β,7β,7aα)]-4-[4-[2-(3-Fluorophenoxy)ethyl]octahydro-7-methyl-1,3-dioxo-4,7-epoxy-2H-isoindol-2-yl]-1-naphthalenecarbonitrile;
[3aS-(-(3aα,4β,7β,7aα)]-4-[4-[2-(3-Fluorophenoxy)ethyl]octahydro-7-methyl-1,3-dioxo-4,7-epoxy-2H-isoindol-2-yl]-1-naphthalenecarbonitrile;
(4-Fluorophenyl)carbamic acid, 2-[(3aα,4 ,7β,7aα)-2-(4-cyano-1-naphthalenyl)octahydro-7-methyl-1,3-dioxo-4,7-epoxy-4H-isoindol-4-y]ethyl ester;
(3aα,4β,7β,7aα)-4-[Octahydro-4-(2-hydroxyethyl)-1,3-dioxo-4,7-epoxy-2H-isoindol-2-yl]-1-naphthalenecarbonitrile;
(3aα,4β,6β,7β,7aα)-4-[4-[2-(4-Cyanophenoxy)ethyl]octahydro-6-hydroxy-1,3-dioxo-4,7-epoxy-2H-isoindol-2-yl]-1-naphthalenecarbonitrile;
[3aS-(3aα,4β,5β,7β,7aα)]-4-[Octahydro-5-hydroxy-7-(2-hydroxyethyl)-4-methyl-1,3-dioxo-4,7-epoxy-2H-isoindol-2-yl]-1-naphthalenecarbonitrile;
[3aR-(3aα,4β,5β,7β,7aα)]-4-[Octahydro-5-hydroxy-7-(2-hydroxyethyl)-4-methyl-1,3-dioxo-4,7-epoxy-2H-isoindol-2-yl]-1-naphthalenecarbonitrile;
(3aα,4β,7β,7aα)-4-[4-[2-(4-Cyanophenoxy)ethyl]-7-ethyloctahydro-1,3-dioxo-4,7-epoxy-2H-isoindol-2-yl]-1-naphthalenecarbonitrile;
(3aα,4β,7β,7aα)-4-[2-(Acetyloxy)ethyl]-2-(4-cyano-1-naphthalenyl)hexahydro-7-methyl-4,7-epoxy-1H-isoindole-1,3(2H)-dione;
(3aα,4β,7β,7aα)-4-[Octahydro-4-methyl-1,3-dioxo-7-(2-oxoethyl)-4,7-epoxy-2H-isoindol-2-yl]-1-naphthalenecarbonitrile;
[3aα,4β(E),7β,7aα]-4-[4-[3-(4-Cyanophenyl)-2-propenyl]octahydro-7-methyl-1,3-dioxo-4,7-epoxy-2H-isoindol-2-yl]-1-naphthalenecarbonitrile;
[3aα,4β(Z),7β,7aα]-4-[4-[3-(4-Cyanophenyl)-2-propenyl]octahydro-7-methyl-1,3-dioxo-4,7-epoxy-2H-isoindol-2-yl]-1-naphthalenecarbonitrile;
(3aα,4β,7β,7aα)-4-[4-[3-(4-Cyanophenyl)propyl]octahydro-7-methyl-1,3-dioxo-4,7-epoxy-2H-isoindol-2-yl]-1-naphthalenecarbonitrile;
(3aα,4β,7β,7aα)-4-[4-[2-[(6-Chloro-1,2-benzisoxazol-3-yl)oxy]ethyl]octahydro-7-methyl-1,3-dioxo-4,7-epoxy-2H-isoindol-2-yl]-1-naphthalenecarbonitrile;
(3aα,4β,7β,7aα)-4-[Octahydro-4-methyl-7-[2-[(6-nitro-1H-indazol-3-yl)oxy]ethyl]-1,3-dioxo-4,7-epoxy-2H-isoindol-2-yl]-1-naphthalenecarbonitrile;
[3aS-(3aα,4β,5β,7β,7aα)]-4-[7-[2-(1,2-Benzisoxazol-3-yloxy)ethyl]octahydro-5-hydroxy-4-methyl-1,3-dioxo-4,7-epoxy-2H-isoindol-2-yl]-1-naphthalenecarbonitrile;
[3aR-(3aα,4β,5β,7β,7aα)]-4-[7-[2-(I,2-Benzisoxazol-3-yloxy)ethyl)octahydro-5-hydroxy-4-methyl-1,3-dioxo-4,7-epoxy-2H-isoindol-2-yl]-1-naphthalenecarbonitrile;
(3aα,4β,5β,7β,7aα)]-4-(Octahydro-5-hydroxy-4,7-dimethyl-1,3-dioxo-4,7-epoxy-2H-isoindol-2-yl)-2-(trifluoromethyl)benzonitrile;
(3aα,4β,5β,7β,7aα)]-4-(Octahydro-5-hydroxy-4,7-dimethyl-1,3-dioxo-4,7-epoxy-2H-isoindol-2-yl)-2-(trifluoromethyl)benzonitrile;
(3aα,4β,7β,7aα)-2-(4-Cyano-1-naphthalenyl)octahydro-1,3-dioxo-7-[2-(phenylmethoxy)ethyl]-4,7-epoxy-4H-isoindole-4-propanenitrile;
(3aα,4α,7α,7aα)-2-(4-Cyano-1-naphthalenyl)octahydro-1,3-dioxo-7-[2-(phenylmethoxy)ethyl]-4,7-epoxy-4H-isoindole-4-propanenitrile;
(3aα4β,7β,7aα)-2-(4-Cyano-1-naphthalenyl)octahydro-7-(2-hydroxyethyl)-1,3-dioxo-4,7-epoxy-4H-isoindole-4-propanenitrile;
(3aα,4α,7α,7aα)-2-(4-Cyano-1-naphthalenyl)octahydro-7-(2-hydroxyethyl)-1,3-dioxo-4,7-epoxy-4H-isoindole-4-propanenitrile;
(3aα,4β,7β,7aα)-2-(4-Cyano-1-naphthalenyl)-7-[2-(4-fluorophenoxy)ethyl]octahydro-1,3-dioxo-4,7-epoxy-4H-isoindole-4-propanenitrile;
(3aα,4β,7β,7aα)-2-(7-Chloro-2, 1 ,3-benzoxadiazol-4-yl)hexahydro-4,7-dimethyl-4,7-epoxy-1H-isoindole-1,3(2H)-dione;
(3aα,4β,7β,7aα)-2-(7-Chloro-2-methyl-4-benzofuranyl)hexahydro-4,7-dimethyl-4,7-epoxy-1H-isoindole-1,3(2H)-dione;
(3aα,4β,7β,7aα)-2-(7-Chloro-2-methylbenzo[b]thiophen-4-yl)hexahydro-4,7-dimethyl-4,7-epoxy-1H-isoindole-1,3(2H)-dione;
[3aα,4β(E),7β,7aα]-4-[2-(4-Cyano-1-naphthalenyl)octahydro-7-methyl-1,3-dioxo-4,7-epoxy-4H-isoindol-4-yl]-2-butenoic acid, phenylmethyl ester;
(3aα,4β,7β,7aα)-2-(4-Cyano-1-naphthalenyl)octahydro-7-methyl-1,3-dioxo-4,7-epoxy-4H-isoindole-4-butanoic acid;
(3aα,4β,7β,7aα)-2-(4-Cyano-1-naphthalenyl)-N-(4-fluorophenyl)octahydro-7-methyl-1,3-dioxo-4,7-epoxy-4H-isoindole-4-butanamide;
[3aS-(3aα,4β,5β,7β,7aα)]-4-[7-[2-(Acetyloxy)ethyl]octahydro-5-hydroxy-4-methyl-1,3-dioxo-4,7-epoxy-2H-isoindol-2-yl]-1-naphthalenecarbonitrile;
[3aR-(3aα,4β,5β,7β,7aα)]-4-[Octahydro-5-hydroxy-7-(2-hydroxyethyl)-4-methyl-1,3-dioxo-4,7-epoxy-2H-isoindol-2-yl]-1-naphthalenecarbonitrile;
(3aα,4β,7β,7aα(E)]-4-[Octahydro-4-methyl-1,3-dioxo-7-(4-oxo-4-phenyl-2-butenyl)-4,7-epoxy-2H-isoindol-2-yl]-1-naphthalenecarbonitrile;
(3aα,4β,7β,7aα(E)]-4-[Octahydro-4-methyl-1,3-dioxo-7-(4-oxo-4-phenyl-2-butenyl)-4,7-epoxy-2H-isoindol-2-yl]-1-naphthalenecarbonitrile;
(3aα,4β,7β,7aα)-(4-[7-[2-(4-Bromophenoxy)ethyl]octahydro-4-methyl-1,3-dioxo-4,7-epoxy-2H-isoindol-2-yl]-2-(trifluoromethyl)benzonitrile;
(3aα,4β,7β,7aα)-4-[Octahydro-7-[2-(4-iodophenoxy)ethyl]-4-methyl-1,3-dioxo-4,7-epoxy-2H-isoindol-2-yl]-2-(trifluoromethyl)benzonitrile;
(3aα,4β,7β,7aα)-4-[Octahydro-4-methyl-1,3-dioxo-7-[2-[4-(trifluoromethyl)phenoxy]ethyl]-4,7-epoxy-2H-isoindol-2-yl]-2-(trifluoromethyl)benzonitrile;
(3aα,4β,7β,7aα)-4-[Octahydro-7-[2-(4-methoxyphenoxy)ethyl]-4-methyl-1,3-dioxo-4,7-epoxy-2H-isoindol-2-yl]-2-(trifluoromethyl)benzonitrile;
(3aα,4β,7β,7aα)-4-[7-[2-(4-Ethoxyphenoxy)ethyl]octahydro-4-methyl-1,3-dioxo-4,7-epoxy-2H-isoindol-2-yl]-2-(trifluoromethyl)benzonitrile;
(3aα,4β,7β,7aα)-4-[7-[2-(4-Chlorophenoxy)ethyl]octahydro-4-methyl-1,3-dioxo-4,7-epoxy-2H-isoindol-2-yl]-2-(trifluoromethyl)benzonitrile;
(3aα,4β,7β,7aα)-4-[2-[2-[4-Cyano-3-(trifluoromethyl)phenyl]octahydro-7-methyl-1,3-dioxo-4,7-epoxy-4H-isoindol-4-yl]ethoxy]benzoic acid, methyl ester;
(3aα,4β,7β,7aα)-Hexahydro-4-(2-hydroxyethyl)-7-methyl-2-(3-methyl-4-nitrophenyl)-4,7-epoxy-1H-isoindole-1,3(2H)-dione;
(3aα,4β,7β,7aα)-4-[Octahydro-4-methyl-1,3-dioxo-7-[2-[4-(trifluoromethoxy)phenoxy]ethyl]-4,7-epoxy-2H-isoindol-2-yl]-2-(trifluoromethyl)benzonitrile;
(3aα,4β,7β,7aα)-2-(3,5-Dichlorophenyl)hexahydro-4,7-dimethyl-4,7-epoxy-1H-isoindole-1,3(2H)-dione;
(3aα,4β,7β,7aα)-Hexahydro-4,7-dimethyl-2-(4-nitro-1-naphthalenyl)-4,7-epoxy-1H-isoindole-1,3(2H)-dione;
(3aα,4β,7β,7aα)-4-[Octahydro-4-methyl-1,3-dioxo-7-[2-[4-(phenylmethoxy)phenoxy]ethyl]-4,7-epoxy-2H-isoindol-2-yl]-2-(trifluoromethyl)benzonitrile;
(3aα,4β,7β,7aα)-Hexahydro-4-(2-hydroxyethyl)-7-methyl-2-(4-nitro-1-naphthalenyl)-4,7-epoxy-1H-isoindole-1,3(2H)-dione;
(3aα,4β,7β,7aα)-4-[2-(4-Fluorophenoxy)ethyl]hexahydro-7-methyl-2-(3-methyl-4-nitrophenyl)-4,7-epoxy-1H-isoindole-1,3(2H)-dione;
(3aα,4β,7β,7aα)-4-[Octahydro-4-methyl-1,3-dioxo-7-[2-[4-[(trifluoromethyl)thio]phenoxy]ethyl]-4,7-epoxy-2H-isoindol-2-yl]-2-(trifluoromethyl)benzonitrile;
(3aα,4β,7β,7aα)-4-[Octahydro-4-methyl-7-[2-(4-nitrophenoxy)ethyl]-1,3-dioxo-5 4,7-epoxy-2H-isoindol-2-yl]-2-(trifluoromethyl)benzonitrile;
(3aα,4β,7β,7aα)-4-[2-(4-Fluorophenoxy)ethyl]hexahydro-7-methyl-2-(4-nitro-1-naphthalenyl)-4,7-epoxy-1H-isoindole-1,3(2H)-dione;
(3aα,4β,7β,7aα)-4-[Octahydro-7-methyl-1,3-dioxo-7-[2-[2-(trifluoromethyl)phenoxy]ethyl]-4,7-epoxy-2H-isoindol-2-yl]-2-(trifluoromethyl)benzonitrile;
(3aα,4β,7β,7aα)-4-[4-[2-(2-Bromophenoxy)ethyl]octahydro-7-methyl-1,3-dioxo-4,7-epoxy-2H-isoindol-2-yl]-2-(trifluoromethyl)benzonitrile;
(3aα,4β,7β,7aα)-4-[4-[2-(3-Fluorophenoxy)ethyl]octahydro-7-methyl-1,3-dioxo-4,7-epoxy-2H-isoindol-2-yl]-2-(trifluoromethyl)benzonitrile;
(3aα,4β,7β,7aα)-Hexahydro-2-[4-(1H-imidazol-1-yl)phenyl]-4-methyl-4,7-epoxy-1H-isoindole-1,3(2H)-dione;
(3aα,4β,7β,7aα)-2-[3-Chloro-4-(2-thiazolyl)phenyl]hexahydro-4-methyl-4,7-epoxy-1H-isoindole-1,3(2H)-dione;
(3aα,4β,7β,7aα)-Hexahydro-4,7-dimethyl-2-(3-methyl-4-nitrophenyl)-4,7-epoxy-1H-isoindole-1,3(2H)-dione;
(3aα,4β,7β,7aα)-Hexahydro-4,7-dimethyl-2-(2-methyl-4-nitrophenyl)-4,7-epoxy-1H-isoindole-1,3(2H)-dione;
(3aα,4β,7β,7aα)-2-(3,5-Dichlorophenyl)hexahydro-4-(2-hydroxyethyl)-7-methyl-4,7-epoxy-1H-isoindole-1,3(2H)-dione;
(3aα,4β,7β,7aα)-2-(3,5-Dichlorophenyl)-4-[2-(4-fluorophenoxy)ethyl]hexahydro-7-methyl-4,7-epoxy-1H-isoindole-1,3(2H)-dione;
(3aα,4β,7β,7aα)-4-[Octahydro-4-[2-(4-hydroxyphenoxy)ethyl]-7-methyl-1,3-dioxo-4,7-epoxy-2H-isoindol-2-yl]-2-(trifluoromethyl)benzonitrile;
(3aα,4β,7β,7aα)-4-[4-[2-(4-Cyanophenoxy)ethyl]octahydro-7-methyl-1,3-dioxo-4,7-epoxy-2H-isoindol-2-yl)-2-(trifluoromethyl)benzonitrile;
(3aα,4β,7β,7aα)-4-[Octahydro-4-methyl-1,3-dioxo-7-[2-[3-(trifluoromethyl)phenoxy]ethyl]-4,7-epoxy-2H-isoindol-2-yl]-2-(trifluoromethyl)benzonitrile;
(3aα,4β,7β,7aα)-4-[4-[2-(3-Bromophenoxy)ethyl]octahydro-7-methyl-1,3-dioxo-4,7-epoxy-2H-isoindol-2-yl]-2-(trifluoromethyl)benzonitrile;
(3aα,4β,7β,7aα)-4-[4-[(4-Fluorophenyl)methyl]octahydro-7-methyl-1,3-dioxo-4,7-epoxy-2H-isoindol-2-yl]-2-(trifluoromethyl)benzonitrile;
(3aα,4β,7β,7aα)-2-(1,6-Dihydro-1-methyl-6-oxo-3-pyridinyl)hexahydro-4,7-dimethyl-4,7-epoxy-1H-isoindole-1,3(2H)-dione;
(3aα,4β,7β,7aα)-Hexahydro-4,7-dimethyl-2-(1-methyl-6-oxo-3-piperidinyl)-4,7-epoxy-1H-isoindole-1,3(2H)-dione;
(3aα,4β,7β,7aα)-4-[4-[2-(3-Cyanophenoxy)ethyl]octahydro-7-methyl-1,3-dioxo-4,7-epoxy-2H-isoindol-2-yl]-2-(trifluoromethyl)benzonitrile;
(3aα,4β,7β,7aα)-4-[2-[4-Cyano-3-(trifluoromethyl)phenyl]octahydro-7-methyl-1,3-dioxo-4,7-epoxy-4H-isoindol-4-yl]ethoxy]benzoic acid, phenylmethyl ester;
(3aα,4β,7β,7aα)-4-[Octahydro-4-methyl-1,3-dioxo-7-(2-phenoxyethyl)-4,7-epoxy-2H-isoindol-2-yl]-2-(trifluoromethyl)benzonitrile;
(3aα,4β,7β,7aα)-2-(3,5-Dichloro-4-nitrophenyl)hexahydro-4,7-dimethyl-4,7-epoxy-1H-isoindole-1,3(2H)-dione;
(3aα,4β,7β,7aα)-2-(3,5-Dichloro-4-hydroxyphenyl)hexahydro-4,7-dimethyl-4,7-epoxy-1H-isoindole-1,3(2H)-dione;
(3aα,4β,7β,7aα)-2-(5-Fluoro-1-naphthalenyl)hexahydro-4,7-dimethyl-4,7-epoxy-1H-isoindole-1,3(2H)-dione;
(3aα,4β,7β,7aα)-Hexahydro-4,7-dimethyl-2-(1-naphthalenyl)-4,7-epoxy-1H-isoindole-1,3(2H)-dione;
(3aα,4β,7β,7aα)-Hexahydro-2-[3-methoxy-4-(5-oxazolyl)phenyl]-4,7-dimethyl-4,7-epoxy-1H-isoindole-1,3(2H)-dione;
(3aα,4β,7β,7aα)-Hexahydro-4-[2-(4-methoxyphenoxy)ethyl]-7-methyl-2-(4-nitro-1-naphthalenyl)-4,7-epoxy-1H-isoindole-1,3(2H)-dione;
(3aα,4β,7β,7aα)-Hexahydro-4-methyl-2-(4-nitro-1-naphthalenyl)-7-[2-[4-(trifluoromethyl)phenoxy]ethyl]-4,7-epoxy-1H-isoindole-1,3(2H)-dione;
(3aα,4β,7β,7aα)-Hexahydro-4-methyl-2-(4-nitro-1-naphthalenyl)-7-[2-(4-nitrophenoxy)ethyl]-4,7-epoxy-1H-isoindole-1,3(2H)-dione;
(3aα,4β,7β,7aα)-2-(1,6-Dihydro-1,4-dimethyl-6-oxo-3-pyridinyl)hexahydro-4,7-dimethyl-4,7-epoxy-1H-isoindole-1,3(2H)-dione;
(3aα,4β,7β,7aα)-4-[Octahydro-7-methyl-2-(4-nitro-1-naphthalenyl)-1,3-dioxo-4,7-epoxy-4H-isoindol-4-yl]ethoxy]benzonitrile;
(3aα,4β,7β,7aα)-4-(Octahydro-4,7-dimethyl-1,3-dioxo-4,7-epoxy-2H-isoindol-2-yl)-1,2-benzenedicarbonitrile;
(3aα,4β,7β,7aα)-4-(2-Bromoethyl)hexahydro-7-methyl-2-(4-nitro-1-naphthalenyl)-4,7-epoxy-1H-isoindole-1,3(2H)-dione;
(3aα,4β,7β,7aα)-4-[4-[2-(4-Cyanophenoxy)ethyl]octahydro-7-methyl-1,3-dioxo-4,7-epoxy-2H-isoindol-2-yl]-1-naphthalenecarbonitrile;
(3aα,4β,7β,7aα)-4-[Octahydro-4-[2-(4-methoxyphenoxy)ethyl]-7-methyl-1,3-dioxo-4,7-epoxy-2H-isoindol-2-yl]-1-naphthalenecarbonitrile;
(3aα,4β,7β,7aα)-4-[Octahydro-4-[2-(3-methoxyphenoxy)ethyl]-7-methyl-1,3-dioxo-4,7-epoxy-2H-isoindol-2-yl]-1-naphthalenecarbonitrile;
(3aα,4β,7β,7aα)-4-[4-[2-(3-Fluorophenoxy)ethyl]octahydro-7-methyl-1,3-dioxo-4,7-epoxy-2H-isoindol-2-yl]-1-naphthalenecarbonitrile;
(3aα,4β,7β,7aα)-4-[Octahydro-4-methyl-7-[2-[3-(4-morpholinyl)phenoxy]ethyl]-1,3-dioxo-4,7-epoxy-2H-isoindol-2-yl]-1-naphthalenecarbonitrile;
(3aα,4β,7β,7aα)-4-[Octahydro-4-methyl-7-[2-[4-nitro-3-(trifluoromethyl)phenoxy]ethyl]-1,3-dioxo-4,7-epoxy-2H-isoindol-2-yl]-1-naphthalenecarbonitrile;
(3aα,4β,7β,7aα)-4-[4-[2-(3-Cyanophenoxy)ethyl]octahydro-7-methyl-1,3-dioxo-4,7-epoxy-2H-isoindol-2-yl]-1-naphthalenecarbonitrile;
(3aα,4β,7β,7aα)-2-(2,3-Dihydro-3-methyl-2-oxo-6-benzothiazolyl)hexahydro-4,7-dimethyl-4,7-epoxy-1H-isoindole-1,3(2H)-dione;
(3aα,4β,7β,7aα)-2-(2,3-Dihydro-2-oxo-6-benzothiazolyl)hexahydro-4,7-dimethyl-4,7-epoxy-1H-isoindole-1,3(2H)-dione;
(3aα,4β,7β,7aα)-4-[4-[2-[3-(Dimethylamino)phenoxy]ethyl]octahydro-7-methyl-1,3-dioxo-4,7-epoxy-2H-isoindol-2-yl]-1-naphthalenecarbonitrile;
(3aα,4β,7β,7aα)-4-[2-[4-Cyano-3-(trifluoromethyl)phenyl]octahydro-7-methyl-1,3-dioxo-4,7-epoxy-4H-isoindol-4-yl[ethoxy]-1,2-benzenedicarbonitrile;
(3aα,4β,7β,7aα)-N-[2-Cyano-5-(octahydro-4,7-dimethyl-1,3-dioxo-4,7-epoxy-2H-isoindol-2-yl)phenyl]acetamide;
(3aα,4β,7β,7aα)-4-(Octahydro-4,7-dimethyl-1,3-dioxo-4,7-epoxy-2H-isoindol-2-yl)-2-(trifluoromethoxy)benzonitrile;
(3aα,4β,7β,7aα)-2-Methoxy-4-(octahydro-4,7-dimethyl-1,3-dioxo-4,7-epoxy-2H-isoindol-2-yl)benzonitrile;
(3aα,4β,7β,7aα)-2-[4-(4,5-Dichloro-1H-imidazol-1-yl)phenyl]hexahydro-4,7-dimethyl-4,7-epoxy-1H-isoindole-1,3(2H)-dione;
(3aα,4β,7β,7aα)-2-[4-(4-Bromo-1-methyl-1H-pyrazol-3-yl)phenyl]hexahydro-4,7-dimethyl-4,7-epoxy-1H-isoindole-1,3(2H)-dione;
(3aα,4β,7β,7aα)-4-[Octahydro-4-(2-hydroxyethyl)-7-methyl-1,3-dioxo-4,7-epoxy-2H-isoindol-2-yl]-1-naphthalenecarbonitrile;
(3aα,4β,7β,7aα)-2-lodo-4-(Octahydro-4,7-dimethyl-1,3-dioxo-4,7-epoxy-2H-isoindol-2-yl)benzonitrile;
(3aα,4β,7β,7aα)-4-[4-[2-(4-Fluorophenoxy)ethyl]octahydro-7-methyl-1,3-dioxo-4,7-epoxy-2H-isoindol-2-yl]-1-naphthalenecarbonitrile;
(3aα,4β,7β,7aα)-4-[Octahydro-4-methyl-1,3-dioxo-7-[2-[4-(trifluoromethyl)phenoxy]ethyl]-4,7-epoxy-2H-isoindol-2-yl]-1-naphthalenecarbonitrile;
(3aα,4β,7β,7aα)-4-[4-[2-(4-Cyano-3-fluorophenoxy)ethyl]octahydro-7-methyl-1,3-dioxo-4,7-epoxy-2H-isoindol-2-yl]-1-naphthalenecarbonitrile;
(3aα,4β,7β,7aα)-4-[Octahydro-4-methyl-1,3-dioxo-7-[2-[2,3,5,6-tetrafluoro-4-(trifluoromethyl)phenoxy]ethyl]-4,7-epoxy-2H-isoindol-2-yl]-1-naphthalenecarbonitrile;
(3aα,4β,7β,7aα)-Hexahydro-4,7-dimethyl-2-[4-(1H-1,2,4-triazol-3-yl)phenyl]-4,7-epoxy-1H-isoindole-1,3(2H)-dione;
(3aα,4β,7β,7aα)-2-[4-(4,5-Dihydro-5-oxo-1,2,4-oxadiazol-3-yl)phenyl]hexahydro-4,7-dimethyl-4,7-epoxy-1H-isoindole-1,3(2H)-dione;
(3aα,4β,7β,7aα)-Hexahydro-2-[3-methoxy-4-(2-oxazolyl)phenyl]-4,7-dimethyl-4,7-epoxy-1H-isoindole-1,3(2H)-dione;
(3aα,4β,7β,7aα)-Hexahydro-2-(4-hydroxy-1-naphthalenyl)-4,7-dimethyl-4,7-epoxy-1H-isoindole-1,3(2H)-dione;
(3aα,4β,7β,7aα)-Hexahydro-2-(8-hydroxy-5-quinolinyl)-4,7-dimethyl-4,7-epoxy-1H-isoindole-1,3(2H)-dione, trifluoroacetate;
(3aα,4β,7β,7aα)-4-[Octahydro-4-methyl-1,3-dioxo-7-[2-[methyl(phenylmethyl)amino]ethyl]-4,7-epoxy-2H-isoindol-2-yl]-2-(trifluoromethyl)benzonitrile;
(3aα,4β,7β,7aα)-Hexahydro-4,7-dimethyl-2-(5-quinolinyl)-4,7-epoxy-1H-isoindole-1,3(2H)-dione;
(3aα,4β,7β,7aα)-5-(Octahydro-4,7-dimethyl-1,3-dioxo-4,7-epoxy-2H-isoindol-2-yl)-2-pyridinecarbonitrile;
(3aα,4β,7β,7aα)-5-(Octahydro-4,7-dimethyl-1,3-dioxo-4,7-epoxy-2H-isoindol-2-yl)-8-quinolinecarbonitrile;
(3aα,4β,7β,7aα)-2-(5-Bromo-4-nitro-1-naphthalenyl)hexahydro-4,7-dimethyl-4,7-epoxy-1H-isoindole-1,3(2H)-dione;
(3aα,4β,7β,7aα)-2-(5-Bromo-1-naphthalenyl)hexahydro-4,7-dimethyl-4,7-epoxy-1H-isoindole-1,3(2H)-dione;
(3aα,4β,7β,7aα)-Hexahydro-4,7-dimethyl-2-[8-(trifluoromethyl)-4-quinolinyl]-4,7-epoxy-1H-isoindole-1,3(2H)-dione;
4-Fluorobenzoic acid, 2-[(3aα,4β,7β,7aα)-2-(4-cyano-1-naphthalenyl)octahydro-7-methyl-1,3-dioxo-4,7-epoxy-4H-isoindol-4-y]ethyl ester;
Benzeneacetic acid, 2-[(3aα,4β,7β,7aα)-2-(4-cyano-1-naphthalenyl)octahydro-7-methyl-1,3-dioxo-4,7-epoxy-4H-isoindol-4-y]ethyl ester;
4-Fluorobenzeneacetic acid, 2-[(3aα,4β,7β,7aα)-2-(4-cyano-1-naphthalenyl)octahydro-7-methyl-1,3-dioxo-4,7-epoxy-4H-isoindol-4-y]ethyl ester;
(3aα,4β,7β,7aα)-Hexahydro-4-methyl-7-[2-[4-(methylsulfonyl)phenoxy]ethyl]-2-(4-nitro-1-naphthalenyl)-4,7-epoxy-1H-isoindole-1,3(2H)-dione;
(3aα,4β,7β,7aα)-Hexahydro-2-(2-naphthalenyl)-4,7-dimethyl-4,7-epoxy-1H-isoindole-1,3(2H)-dione;
(3aα,4β,7β,7aα)-2-(4-Chloro-1-naphthalenyl)hexahydro-4,7-dimethyl-4,7-epoxy-1H-isoindole-1,3(2H)-dione;
(3aα,4β,7β,7aα)-N-[(4-Chlorophenyl)methyl]-2-(4-cyano-1-naphthalenyl)octahydro-7-methyl-1,3-dioxo-4,7-epoxy-4H-isoindole-4-acetamide;
4,7,7-Trimethyl-3-oxo-2-oxabicyclo[2.2.1]heptane-l1-carboxylic acid, 2-[(3aα,41,7β,7aα)-2-(4-cyano-1-naphthalenyl)octahydro-7-methyl-1,3-dioxo-4,7-epoxy-4H-isoindol-4-y]ethyl ester;
(αS)-α-Methoxy-α-(trifluoromethyl)benzeneacetic acid, 2-[(3aα,4β,7β,7aα)-2-(4-cyano-1-naphthalenyl)octahydro-7-methyl-1,3-dioxo-4,7-epoxy-4H-isoindol-4-y]ethyl ester;
(αR)-α-Methoxy-α-(trifluoromethyl)benzeneacetic acid, 2-[(3aα,4β,7β,7aα)-2-(4-cyano-1-naphthalenyl)octahydro-7-methyl-1,3-dioxo-4,7-epoxy-4H-isoindol-4-y]ethyl ester;
(3aα,4β,7β,7aα)-4-[Octahydro-4-methyl-7-[2-[(7-methyl-1,2-benzisoxazol-3-yl)oxy]ethyl]-1,3-dioxo-4,7-epoxy-2H-isoindol-2-yl]-1-naphthalenecarbonitrile;
(3aα,4β,7β,7aα)-4-[4-[2-(1,2-Benzisoxazol-3-yloxy)ethyl]octahydro-7-methyl-1,3-dioxo-4,7-epoxy-2H-isoindol-2-yl]-1-naphthalenecarbonitrile;
(3aα,4β,7β,7aα)-4-[2-(Benzoyloxy)ethyl]-2-(4-cyano-1-naphthalenyl)hexahydro-7-methyl-4,7-epoxy-1H-isoindole-1,3(2H)-dione;
(3aα,4β,7β,7aα)-2-(4-Cyano-1-naphthalenyl)-4-[2-[(4-nitrobenzoyl)oxy]ethyl]hexahydro-7-methyl-4,7-epoxy-1H-isoindole-1,3(2H)-dione;
4-Chlorobenzoic acid, 2-[(3aα,4β,7β,7aα)-2-(4-cyano-1-naphthalenyl)octahydro-7-methyl-1,3-dioxo-4,7-epoxy-4H-isoindol-4-y]ethyl ester;
[3aα,4β,7β,7aα(E)]-4-[Octahydro-4-methyl-7-[3-(1-naphthalenyl)-2-propenyl]-1,3-dioxo-4,7-epoxy-2H-isoindol-2-yl]-1-naphthalenecarbonitrile;
(3aα,4β,7β,7aα)-4-[Octahydro-4-methyl-7-[3-(1-naphthalenyl)propyl]-1,3-dioxo-4,7-epoxy-2H-isoindol-2-yl]-1-naphthalenecarbonitrile;
(3aα,4β,7β,7aα)-Hexahydro-4,7-dimethyl-2-(2-methyl-6-quinolinyl)-4,7-epoxy-1H-isoindole-1,3(2H)-dione;
(3aα,4β,7β,7aα)-Hexahydro-2-(5-isoquinolinyl)-4,7-dimethyl-4,7-epoxy-1H-isoindole-1,3(2H)-dione;
(3aα,4β,7β,7aα)-2-(6-Benzothiazolyl)hexahydro-4,7-dimethyl-4,7-epoxy-1H-isoindole-1,3(2H)-dione;
[3aα,4β,7β,7aα(E)]-4-[Octahydro-4-methyl-1,3-dioxo-7-(4-oxo-4-phenyl-2-butenyl)-4,7-epoxy-2H-isoindol-2-yl]-1-naphthalenecarbonitrile;
(3aα,4β,7β,7aα)-2-(4-Cyano-1-naphthalenyl)octahydro-N-(2-hydroxyphenyl)-7-methyl-1,3-dioxo-4,7-epoxy-4H-isoindole-4-acetamide;
[3aα,4β(E),7β,7aα]-4-[Octahydro-4-methyl-7-[3-(6-methyl-2-pyridinyl)-2-propenyl]-1,3-dioxo-4,7-epoxy-2H-isoindol-2-yl]-1-naphthalenecarbonitrile;
(3aα,4β,7β,7aα)-4-[Octahydro-4-methyl-7-[3-(6-methyl-2-pyridinyl)propyl]-1,3-dioxo-4,7-epoxy-2H-isoindol-2-yl]-1-naphthalenecarbonitrile;
[3aR-(3aα,4β,7β,7aα)]-4-[Octahydro-4-[2-(3-methoxyphenoxy)ethyl]-7-methyl-1,3-dioxo-4,7-epoxy-2H-isoindol-2-yl]-1-naphthalenecarbonitrile;
[3aS-(3aα,4β,7β,7aα)]-4-[Octahydro-4-[2-(3-methoxyphenoxy)ethyl]-7-methyl-1,3-dioxo-4,7-epoxy-2H-isoindol-2-yl]-1-naphthalenecarbonitrile;
[3aR-(3aα,4β,7β,7aα)]-4-[4-[2-(4-Cyanophenoxy)ethyl]octahydro-7-methyl-1,3-dioxo-4,7-epoxy-2H-isoindol-2-yl]-1-naphthalenecarbonitrile;
[3aS-(3aα,4β,7β,7aα)]-4-[4-[2-(4-Cyanophenoxy)ethyl]octahydro-7-methyl-1,3-dioxo-4,7-epoxy-2H-isoindol-2-yl]-1-naphthalenecarbonitrile;
(3aα,4α,7α,7aα)-4-[4-[(4-Fluorophenyl)methyl]octahydro-7-methyl-1,3-dioxo-4,7-epoxy-2H-isoindol-2-yl]-2-(trifluoromethyl)benzonitrile;
(3aα,4α,7α,7aα)-Hexahydro-4,7-dimethyl-2-(1-methyl-6-oxo-3-piperidinyl)-4,7-epoxy-1H-isoindole-1,3(2H)-dione;
(3aα,4α,7α,7aα)-2-(1,6-Dihydro-1,4-dimethyl-6-oxo-3-pyridinyl)hexahydro-4,7-dimethyl-4,7-epoxy-1H-isoindole-1,3(2H)-dione;
(3aα,4β,7β,7aα)-2-[4-Cyano-3-(trifluoromethyl)phenyl]octahydro-1,3-dioxo-7-[2-(phenylmethoxy)ethyl]-4,7-epoxy-4H-isoindole-4-propanenitrile;
(3aα,4β,7β,7aα)-2-[4-Cyano-3-(trifluoromethyl)phenyl]octahydro-1,3-dioxo-7-[2-(phenylmethoxy)ethyl]-4,7-epoxy-4H-isoindole-4-propanenitrile;
(3aα,4β,7β,7aα)-4-[7-[2-(4-Cyanophenoxy)ethyl]octahydro-5-hydroxy-4-methyl-1,3-dioxo-4,7-epoxy-2H-isoindol-2-yl]-1-naphthalenecarbonitrile;
[3aS-(3aα,4β,7β,7aα)]-4-[7-[2-(1,3-Benzodioxol-5-yloxy)ethyl]octahydro-5-hydroxy-4-methyl-1,3-dioxo-4,7-epoxy-2H-isoindol-2-yl]-1-naphthalenecarbonitrile;
[3aR-(3aα,4β,7β,7aα)]-4-[7-[2-(1,3-Benzodioxol-5-yloxy)ethyl]octahydro-5-hydroxy-4-methyl-1,3-dioxo-4,7-epoxy-2H-isoindol-2-yl]-1-naphthalenecarbonitrile;
[3aS-(3aα,4β,7β,7aα)]-4-[7-[2-[(5-Chloro-2-pyridinyl)oxy]ethyl]octahydro-5-hydroxy-4-methyl-1,3-dioxo-4,7-epoxy-2H-isoindol-2-yl]-1-naphthalenecarbonitrile;
[3aR-(3aα,4β,7β,7aα)]-4-[7-[2-[(5-Chloro-2-pyridinyl)oxy]ethyl]octahydro-5-hydroxy-4-methyl-1,3-dioxo-4,7-epoxy-2H-isoindol-2-yl]-1-naphthalenecarbonitrile;
[3aS-(3aα,4β,7β,7aα)]-4-[7-[2-(4-Chlorophenoxy)ethyl]octahydro-5-hydroxy-4-methyl-1,3-dioxo-4,7-epoxy-2H-isoindol-2-yl]-1-naphthalenecarbonitrile;
[3aR-(3aα,4β,7β,7aα)]-4-[7-[2-(4-Chlorophenoxy)ethyl]octahydro-5-hydroxy-4-methyl-1,3-dioxo-4,7-epoxy-2H-isoindol-2-yl]-1-naphthalenecarbonitrile;
[3aS-(3aα,4β,7β,7aα)]-4-[7-[2-(4-Acetylphenoxy)ethyl]octahydro-5-hydroxy-4-methyl-1,3-dioxo-4,7-epoxy-2H-isoindol-2-yl]-1-naphthalenecarbonitrile;
[3aR-(3aα,4β,7β,7aα)]-4-[7-[2-(4-Acetylphenoxy)ethyl]octahydro-5-hydroxy-4-methyl-1,3-dioxo-4,7-epoxy-2H-isoindol-2-yl]-1-naphthalenecarbonitrile;
[3aS-(3aα,4β,7β,7aα)]-4-[7-[2-(3-Cyanophenoxy)ethyl]octahydro-5-hydroxy-4-methyl-1,3-dioxo-4,7-epoxy-2H-isoindol-2-yl]-1-naphthalenecarbonitrile;
[3aR-(3aα,4β,7β,7aα)]-4-[7-[2-(3-Cyanophenoxy)ethyl]octahydro-5-hydroxy-4-methyl-1,3-dioxo-4,7-epoxy-2H-isoindol-2-yl]-1-naphthalenecarbonitrile;
[3aS-(3aα,4β,7β,7aα)]-4-[Octahydro-5-hydroxy-4-methyl-1,3-dioxo-7-[2-[(5,6,7,8-tetrahydro-1-naphthalenyl)oxy]ethyl]-4,7-epoxy-2H-isoindol-2-yl]-1-naphthalenecarbonitrile;
[3aR-(3aα,4β,7β,7aα)]-4-[Octahydro-5-hydroxy-4-methyl-1,3-dioxo-7-[2-[(5,6,7,8-tetrahydro-1-naphthalenyl)oxy]ethyl]-4,7-epoxy-2H-isoindol-2-yl]-1-naphthalenecarbonitrile;
[3aS-(3aα,4β,7β,7aα)]-4-[Octahydro-5-hydroxy-4-methyl-1,3-dioxo-7-[2-[(5,6,7,8-tetrahydro-5-oxo-1-naphthalenyl)oxy]ethyl]-4,7-epoxy-2H-isoindol-2-yl]-1-naphthalenecarbonitrile;
[3aR-(3aα,4β,7β,7aα)]-4-[Octahydro-5-hydroxy-4-methyl-1,3-dioxo-7-[2-[(5,6,7,8-tetrahydro-5-oxo-1-naphthalenyl)oxy]ethyl]-4,7-epoxy-2H-isoindol-2-yl]-1-naphthalenecarbonitrile;
[3aS-(3aα,4β,7β,7aα)]-4-[7-[2-(4-Fluorophenoxy)ethyl]octahydro-5-hydroxy-4-methyl-1,3-dioxo-4,7-epoxy-2H-isoindol-2-yl]-1-naphthalenecarbonitrile;
[3aR-(3aα,4β,7β,7aα)]-4-[7-[2-(4-Fluorophenoxy)ethyl]octahydro-5-hydroxy-4-methyl-1,3-dioxo-4,7-epoxy-2H-isoindol-2-yl]-1-naphthalenecarbonitrile;
[3aS-(3aα,4β,7β,7aα)]-4-[Octahydro-5-hydroxy-4-methyl-7-[2-[(4-methyl-2-oxo-2H-1-benzopyran-7-yl)oxy]ethyl]-1,3-dioxo-4,7-epoxy-2H-isoindol-2-yl]-1-naphthalenecarbonitrile;
[3aR-(3aα,4β,7β,7aα)]-4-[Octahydro-5-hydroxy-4-methyl-7-[2-[(4-methyl-2-oxo-2H-1-benzopyran-7-yl)oxy]ethyl]-1,3-dioxo-4,7-epoxy-2H-isoindol-2-yl]-1-naphthalenecarbonitrile;
[3aS-(3aα,4β,7β,7aα)]-4-[7-[2-(3,5-Dimethoxyphenoxy)ethyl]octahydro-5-hydroxy-4-methyl-1,3-dioxo-4,7-epoxy-2H-isoindol-2-yl]-1-naphthalenecarbonitrile;
[3aR-(3aα,4β,7β,7aα)]-4-[7-[2-(3,5-Dimethoxyphenoxy)ethyl]octahydro-5-hydroxy-4-methyl-1,3-dioxo-4,7-epoxy-2H-isoindol-2-yl]-1-naphthalenecarbonitrile;
[3aR-(3aα,4β,7β,7aα)]-]4-[7-[2-(4-Chloro-3-methylphenoxy)ethyl]octahydro-5-hydroxy-4-methyl-1,3-dioxo-4,7-epoxy-2H-isoindol-2-yl]-1-naphthalenecarbonitrile;
[3aR-(3aα,4β,7β,7aα)]-4-[7-[2-(4-Cyano-2,3-difluorophenoxy)ethyl]octahydro-5-hydroxy-4-methyl-1,3-dioxo-4,7-epoxy-2H-isoindol-2-yl]-1-naphthalenecarbonitrile;
[3aS-(3aα,4β,7β,7aα)]-4-[7-[2-[(5-Chloro-1,2-benzisoxazol-3-yl)oxy]ethyl]octahydro-5-hydroxy-4-methyl-1,3-dioxo-4,7-epoxy-2H-isoindol-2-yl]-1-naphthalenecarbonitrile;
[3aR-(3aα,4β,7β,7aα)]-4-[7-[2-[(5-Chloro-1,2-benzisoxazol-3-yl)oxy]ethyl]octahydro-5-hydroxy-4-methyl-1,3-dioxo-4,7-epoxy-2H-isoindol-2-yl]-1-naphthalenecarbonitrile;
[3aR-(3aα,4β,7β,7aα)]-3-[2-[2-(4-Cyano-1-naphthalenyl)octahydro-6-hydroxy-7-methyl-1,3-dioxo-4,7-epoxy-4H-isoindol-4-yl]ethoxy]-5-isoxazolecarboxylic acid, methyl ester;
[3aR-(3aα,4β,7β,7aα)]-4-[Octahydro-5-hydroxy-4-methyl-1,3-dioxo-7-[2-[4-(1H-1,2,4-triazol-1-yl)phenoxy]ethyl]-4,7-epoxy-2H-isoindol-2-yl]-1-naphthalenecarbonitrile;
[3aS-(3aα,4β,7β,7aα)]-4-[7-[2-[(7-Chloro-4-quinolinyl)oxy]ethyl]octahydro-5-hydroxy-4-methyl-1,3-dioxo-4,7-epoxy-2H-isoindol-2-yl]-1-naphthalenecarbonitrile, trifluoroacetate;
[3aR-(3aα,4β,7β,7aα)]-4-[7-[2-[(7-Chloro-4-quinolinyl)oxy]ethyl]octahydro-5-hydroxy-4-methyl-1,3-dioxo-4,7-epoxy-2H-isoindol-2-yl]-1-naphthalenecarbonitrile, trifluoroacetate;
(1aα,2β,2aα,5aα,6βb,6aα)-4-[2-[2-[[(1,1-dimethylethyl)-dimethylsilyl]oxy]ethyl]octahydro-6-methyl-3,5-dioxo-2,6-epoxy-4H-oxireno[f]isoindol-4-yl]-1-naphthalenecarbonitrile;
[3aR-(3aα,4β,7β,7aα)]-4-[4-Ethyloctahydro-7-(2-hydroxyethyl)-1,3-dioxo-4,7-epoxy-2H-isoindol-2-yl]-1-naphthalenecarbonitrile;
[3aS-(3aα,4β,7β,7aα)]-4-[4-Ethyloctahydro-7-(2-hydroxyethyl)-1,3-dioxo-4,7-epoxy-2H-isoindol-2-yl]-1-naphthalenecarbonitril;
[3aR-(3aα,4β,7β,7aα)]-4-[4-[2-(4-Cyanophenoxy)ethyl]-7-ethyloctahydro-1,3-dioxo-4,7-epoxy-2H-isoindol-2-yl]-1-naphthalenecarbonitrile; and
[3aS-(3aα,4β,7β,7aα)]-4-[4-[2-(4-Cyanophenoxy)ethyl]-7-ethyloctahydro-1,3-dioxo-4,7-epoxy-2H-isoindol-2-yl]-1-naphthalenecarbonitrile.

5. A pharmaceutical composition capable of treating a NHR—associated condition, comprising a compound of the following formula I or a pharmaceutically acceptable salt thereof, and a pharmaceutically acceptable carrier: 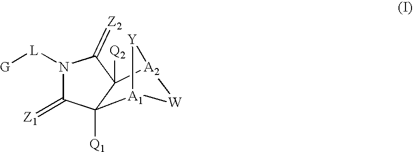
wherein the symbols have the following meanings and are, for each occurrence, independently selected:
G is an aryl or heterocyclo group, where said group is mono- or polycyclic, and which is optionally substituted at one or more positions;
Z₁ is O, S, NH, or NR⁶;
Z₂ is O, S, NH, or NR⁶;
A₁ is CR⁷ or N;
A₂ is CR⁷ or N;
Y is J—J′—J″ where J is (CR⁷R⁷′)n and n=0-3, J′ is a bond or O, S, S═O, SO₂, NH, NR⁷, C═O, OC═O, NR¹C═O, CR⁷R⁷′, C═CR⁸R⁸′, R²P═O, R²P═S, R²OP═O, R²NHP═O, OP═OOR², OP═ONHR², OP═OR², OSO₂, C═NR⁷, NHNH, NHNR⁶, NR⁶NH, N═N, cycloalkyl or substituted cycloalkyl, cycloalkenyl or substituted cycloalkenyl, heterocyclo or substituted heterocyclo or aryl or substituted aryl, and J″ is (CR⁷R⁷′)n and n=0-3, where Y is not a bond;
W is CR⁷R⁷′—CR⁷R⁷′, CR⁸═CR⁸′, CR⁷R⁷′—C═O, NR⁹—CR⁷R⁷′, N═CR⁸, N═N, NR⁹—NR⁹′, S—CR⁷R⁷′, SO—CR⁷R⁷′, SO₂—CR⁷R⁷′, cycloalkyl or substituted cycloalkyl, cycloalkenyl or substituted cycloalkenyl, heterocyclo or substituted heterocyclo, or aryl or substituted aryl, wherein when W is not NR⁹—CR⁷R⁷′, N═CR⁸, N═N, NR⁹—NR⁹′, S—CR⁷R⁷′, SO—CR⁷R⁷′, SO₂—CR⁷R⁷′, or heterocyclo or substituted heterocyclo, then J′ must be O, S, S═O, SO₂, NH, NR⁷, OC═O, NR¹C═O, OP═OOR², OP═ONHR², OSO₂, NHNH, NHNR⁶, NR⁶NH, or N═N;
Q₁ is H, alkyl or substituted alkyl, alkenyl or substituted alkenyl, cycloalkyl or substituted cycloalkyl, cycloalkenyl or substituted cycloalkenyl, heterocycloalkyl or substituted heterocycloalkyl, arylalkyl or substituted arylalkyl, alkynyl or substituted alkynyl, aryl or substituted aryl, heterocyclo or substituted heterocyclo, halo, CN, R¹OC═O, R⁴C═O, R⁵R⁶NC═O, HOCR⁷R⁷′, nitro, R¹OCH₂, R¹O, NH₂, C═OSR¹, SO₂R¹ or NR⁴R⁵;
Q₂ is H, alkyl or substituted alkyl, alkenyl or substituted alkenyl, cycloalkyl or substituted cycloalkyl, cycloalkenyl or substituted cycloalkenyl, heterocycloalkyl or substituted heterocycloalkyl, arylalkyl or substituted arylalkyl, alkynyl or substituted alkynyl, aryl or substituted aryl, heterocyclo or substituted heterocyclo, halo, CN, R¹OC═O, R⁴C═O, R⁵R⁶NC═O, HOCR⁷R⁷′, nitro, R¹OCH₂, R¹O, NH₂, C═OSR¹, SO₂R¹ or NR⁴R⁵;
L is a bond, (CR⁷R⁷′)n, NH, NR⁵, NH(CR⁷R⁷′)n or NR⁵(CR⁷R⁷′)n, where n=0-3;
R¹ and R¹′ are each independently H, alkyl or substituted alkyl, cycloalkyl or substituted cycloalkyl, cycloalkenyl or substituted cycloalkenyl, heterocyclo or substituted heterocyclo, cycloalkylalkyl or substituted cycloalkyalkyl, cycloalkenylalkyl or substituted cycloalkenylalkyl, heterocycloalkyl or substituted heterocycloalkyl, aryl or substituted aryl, arylalkyl or substituted arylalkyl;
R² is alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl;
R³ and R³′ are each independently H, alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, halo, CN, hydroxylamine, hydroxamide, alkoxy or substituted alkoxy, amino, NR¹R², thiol, alkylthio or substituted alkylthio;
R⁴ is H, alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, R¹C═O, R¹NHC═O, SO₂OR¹, or SO₂NR¹R¹′;
R⁵ is alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, R¹C═O, R¹NHC═O, SO₂R¹, SO₂OR¹, or SO₂NR¹R¹′;
R⁶ is alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, CN, OH, OR¹, R¹C═O, R¹NHC═O, SO₂R¹, SO₂OR¹, or SO₂NR¹R¹′;
R⁷ and R⁷′ are each independently H, alkyl or substituted alkyl, alkenyl or substituted alkenyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, halo, CN, OR¹, nitro, hydroxylamine, hydroxylamide, amino, NHR⁴, NR²R⁵, NOR¹, thiol, alkylthio or substituted alkylthio, R¹C═O, R¹OC═O, R¹NHC═O, SO₂R¹, SOR¹, PO₃R¹R¹′, R¹R¹′NC═O, C═OSR¹, SO₂R¹, SO₂OR¹, or SO₂NR¹R¹′, or, wherein A₁, or A₂ contains a group R⁷ and W contains a group R⁷, said R⁷ groups of A₁ or A₂ and W together form a heterocyclic ring;
R⁸ and R⁸′ are each independently H, alkyl or substituted alkyl, alkenyl or substituted alkenyl, cycloalkyl or substituted cycloalkyl, cycloalkenyl or substituted cycloalkenyl, heterocyclo or substituted heterocyclo, cycloalkylalkyl or substituted cycloalkyalkyl, cycloalkenylalkyl or substituted cycloalkenylalkyl, heterocycloalkyl or substituted heterocycloalkyl, aryl or substituted aryl, arylalkyl or substituted arylalkyl, nitro, halo, CN, OR¹, amino, NHR⁴, NR²R⁵, NOR¹, alkylthio or substituted alkylthio, C═OSR¹, R¹OC═O, R¹C═O, R¹NHC═O, R¹R¹′NC═O, SO₂OR, S═OR¹, SO₂R¹, PO₃R¹R¹′, or SO₂NR¹R¹′; and
R⁹ and R⁹′ are each independently H, alkyl or substituted alkyl, alkenyl or substituted alkenyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, CN, OH, OR¹, R¹C═O, R¹OC═O, R¹NHC═O, SO₂R¹, SO₂OR¹, or SO₂NR¹R¹′.

6. A pharmaceutical composition of claim 5 further comprising another anti-cancer agent.

7. A method of modulating the function of a nuclear hormone receptor which comprises administering to a mammalian species in need thereof an effective nuclear hormone receptor modulating amount of a compound of the following formula I: 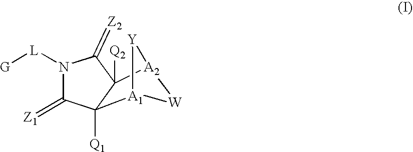
wherein the symbols have the following meanings and are, for each occurrence, independently selected:
G is an aryl or heterocyclo group, where said group is mono- or polycyclic, and which is optionally substituted at one or more positions;
Z₁ is O, S, NH, or NR⁶;
Z₂ is O, S, NH, or NR⁶;
A₁ is CR⁷ or N;
A₂ is CR⁷ or N;
Y is J—J′—J″ where J is (CR⁷R⁷′)n and n=0-3, J′ is a bond or O, S, S═O, SO₂, NH, NR⁷, C═O, OC═O, NR¹C═O, CR⁷R⁷′, C═CR⁸R⁸′, R²P═O, R²P═S, R²OP═O, R²NHP═O, OP═OOR², OP═ONHR², OP═OR², OSO₂, C═NR⁷, NHNH, NHNR⁶, NR⁶NH, N═N, cycloalkyl or substituted cycloalkyl, cycloalkenyl or substituted cycloalkenyl, heterocyclo or substituted heterocyclo or aryl or substituted aryl, and J″ is (CR⁷R⁷′)n and n=0-3, where Y is not a bond;
W is CR⁷R⁷′—CR⁷R⁷′, CR⁸═CR⁸′, CR⁷R⁷′—C═O, NR⁹—CR⁷R⁷′, N═CR⁸, N═N, NR⁹—NR⁹′, S—CR⁷R⁷′, SO—CR⁷R⁷′, SO₂—CR⁷R⁷′, cycloalkyl or substituted cycloalkyl, cycloalkenyl or substituted cycloalkenyl, heterocyclo or substituted heterocyclo, or aryl or substituted aryl, wherein when W is not NR⁹—CR⁷R⁷′, N═CR⁸, N═N, NR⁹—NR⁹′, S—CR⁷R⁷′, SO—R⁷R⁷′, SO₂—CR⁷R⁷′, or heterocyclo or substituted heterocyclo, then J′ must be O, S, S═O, SO₂, NH, NR⁷, OC═O, NR¹C═O, OP═OOR², OP═ONHR², OSO₂, NHNH, NHNR⁶, NR⁶NH, or N═N;
Q₁ is H, alkyl or substituted alkyl, alkenyl or substituted alkenyl, cycloalkyl or substituted cycloalkyl, cycloalkenyl or substituted cycloalkenyl, heterocycloalkyl or substituted heterocycloalkyl, arylalkyl or substituted arylalkyl, alkynyl or substituted alkynyl, aryl or substituted aryl, heterocyclo or substituted heterocyclo, halo, CN, R¹OC═O, R⁴C═O, R⁵R⁶NC═O, HOCR⁷R⁷′, nitro, R¹OCH₂, R¹O, NH₂, C═OSR¹, SO₂R¹ or NR⁴R⁵;
Q₂ is H, alkyl or substituted alkyl, alkenyl or substituted alkenyl, cycloalkyl or substituted cycloalkyl, cycloalkenyl or substituted cycloalkenyl, heterocycloalkyl or substituted heterocycloalkyl, arylalkyl or substituted arylalkyl, alkynyl or substituted alkynyl, aryl or substituted aryl, heterocyclo or substituted heterocyclo, halo, CN, R¹OC═O, R⁴C═O, R⁵R⁶NC═O, HOCR⁷R⁷′, nitro, R¹OCH₂, R¹O, NH₂, C═OSR¹, SO₂R¹ or NR⁴R⁵;
L is a bond, (CR⁷R⁷′)n, NH, NR⁵, NH(CR⁷R⁷′)n or NR⁵(CR⁷R⁷′)n, where n=0-3;
R¹ and R¹′ are each independently H, alkyl or substituted alkyl, cycloalkyl or substituted cycloalkyl, cycloalkenyl or substituted cycloalkenyl, heterocyclo or substituted heterocyclo, cycloalkylalkyl or substituted cycloalkyalkyl, cycloalkenylalkyl or substituted cycloalkenylalkyl, heterocycloalkyl or substituted heterocycloalkyl, aryl or substituted aryl, arylalkyl or substituted arylalkyl;
R² is alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl;
R³ and R³′ are each independently H, alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, halo, CN, hydroxylamine, hydroxamide, alkoxy or substituted alkoxy, amino, NR¹R², thiol, alkylthio or substituted alkylthio;
R⁴ is H, alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, R¹C═O, R¹NHC═O, SO₂OR¹, or SO₂NR¹R¹′;
R⁵ is alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, R¹C═O, R¹NHC═O, SO₂R¹, SO₂OR¹, or SO₂NR¹R¹′;
R⁶ is alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, CN, OH, OR¹, R¹C═O, R¹NHC═O, SO₂R¹, SO₂OR¹, or SO₂NR¹R¹′;
R⁷ and R⁷′ are each independently H, alkyl or substituted alkyl, alkenyl or substituted alkenyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, halo, CN, OR¹, nitro, hydroxylamine, hydroxylamide, amino, NHR⁴, NR²R⁵, NOR¹, thiol, alkylthio or substituted alkylthio, R¹C═O, R¹OC═O, R¹NHC═O, SO₂R¹, SOR¹, PO₃R¹R¹′, R¹R¹′NC═O, C═OSR¹, SO₂R¹, SO₂OR¹, or SO₂NR¹R¹′, or, wherein A₁ or A₂ contains a group R⁷ and W contains a group R⁷, said R⁷′ groups of A₁ or A₂ and W together form a heterocyclic ring;
R⁸ and R⁸′ are each independently H, alkyl or substituted alkyl, alkenyl or substituted alkenyl, cycloalkyl or substituted cycloalkyl, cycloalkenyl or substituted cycloalkenyl, heterocyclo or substituted heterocyclo, cycloalkylalkyl or substituted cycloalkyalkyl, cycloalkenylalkyl or substituted cycloalkenylalkyl, heterocycloalkyl or substituted heterocycloalkyl, aryl or substituted aryl, arylalkyl or substituted arylalkyl, nitro, halo, CN, OR¹, amino, NHR⁴, NR²R⁵, NOR¹, alkylthio or substituted alkylthio, C═OSR¹, R¹OC═O, R¹C═O, R¹NHC═O, R¹R¹′NCC═O, SO₂OR¹, S═OR¹, SO₂R¹, PO₃R¹R¹′, or SO₂NR¹R¹′; and
R⁹ and R⁹′ are each independently H, alkyl or substituted alkyl, alkenyl or substituted alkenyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, CN, OH, OR¹, R¹C═O, R¹OC═O, R¹NHC═O, SO₂R¹, SO₂OR¹, or SO₂NR¹R¹′.

8. The method of claim 7 wherein said nuclear hormone receptor is a steroid binding nuclear hormone receptor.

9. The method of claim 7 wherein said nuclear hormone receptor is the androgen receptor.

10. The method of claim 7 wherein said nuclear hormone receptor is the estrogen receptor.

11. The method of claim 7 wherein said nuclear hormone receptor is the progesterone receptor.

12. The method of claim 7 wherein said nuclear hormone receptor is the glucocorticoid receptor.

13. The method of claim 7 wherein said nuclear hormone receptor is the mineralocorticoid receptor.

14. The method of claim 7 wherein said nuclear hormone receptor is the aldosterone receptor.

15. A method for treating a condition or disorder comprising administering to a mammalian species in need thereof a therapeutically effective amount of a compound of the following formula: 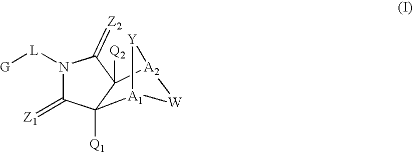
wherein the symbols have the following meanings and are, for each occurrence, independently selected:
G is an aryl or heterocyclo group, where said group is mono- or polycyclic, and which is optionally substituted at one or more positions;
Z₁ is O, S, NH, or NR⁶;
Z₂ is O, S, NH, or NR⁶;
A₁ is CR⁷ or N;
A₂ is CR⁷ or N;
Y is J—J′—J″ where J is (CR⁷R⁷′)n and n=0-3, J′ is a bond or O, S, S═O, SO₂, NH, NR⁷, C═O, OC═O, NR¹C═O, CR⁷R⁷′, C═CR⁸R⁸′, R²P═O, R²P═S, R²OP═O, R²NHP═O, OP═OOR², OP═ONHR², OP═OR², OSO₂, C═NR⁷, NHNH, NHNR⁶, NR⁶NH, N═N, cycloalkyl or substituted cycloalkyl, cycloalkenyl or substituted cycloalkenyl, heterocyclo or substituted heterocyclo or aryl or substituted aryl, and J″ is (CR⁷R⁷′)n and n=0-3, where Y is not a bond;
W is CR⁷R⁷′—CR⁷R⁷′, CR⁸═CR⁸′, CR⁷R⁷′—C═O, NR⁹—CR⁷R⁷′, N═CR⁸, N═N, NR⁹—NR⁹′, S—CR⁷R⁷′, SO—CR⁷R⁷′SO₂—CR⁷R⁷′, cycloalkyl or substituted cycloalkyl, cycloalkenyl or substituted cycloalkenyl, heterocyclo or substituted heterocyclo, or aryl or substituted aryl, wherein when W is not NR⁹—CR⁷R⁷′, N═CR⁸, N═N, NR⁹—NR⁹, S—CR⁷R⁷′, SO—CR⁷R⁷′, SO₂—CR⁷R⁷′, or heterocyclo or substituted heterocyclo, then J′ must be O, S, S═O, SO₂, NH, NR⁷, OC═O, NR¹C═O, OP═OOR², OP═ONHR², OSO₂, NHNH, NHNR⁶, NR⁶NH, or N═N;
Q₁ is H, alkyl or substituted alkyl, alkenyl or substituted alkenyl, cycloalkyl or substituted cycloalkyl, cycloalkenyl or substituted cycloalkenyl, heterocycloalkyl or substituted heterocycloalkyl, arylalkyl or substituted arylalkyl, alkynyl or substituted alkynyl, aryl or substituted aryl, heterocyclo or substituted heterocyclo, halo, CN, R¹OC═O, R⁴C═O, R⁵R⁶NC═O, HOCR⁷R⁷′, nitro, R¹OCH₂, R¹O, NH₂, C═OSR¹, SO₂R¹ or NR⁴R⁵;
Q₂ is H, alkyl or substituted alkyl, alkenyl or substituted alkenyl, cycloalkyl or substituted cycloalkyl, cycloalkenyl or substituted cycloalkenyl, heterocycloalkyl or substituted heterocycloalkyl, arylalkyl or substituted arylalkyl, alkynyl or substituted alkynyl, aryl or substituted aryl, heterocyclo or substituted heterocyclo, halo, CN, R¹OC═O, R⁴C═O, R⁵R⁶NC═O, HOCR⁷R⁷′, nitro, R¹OCH₂, R¹O, NH₂, C═OSR¹, SO₂R¹ or NR⁴R⁵;
L is a bond, (CR⁷R⁷′)n, NH, NR⁵, NH(CR⁷R⁷′)n or NR⁵(CR⁷R⁷′)n, where n=0-3;
R¹ and R¹′ are each independently H, alkyl or substituted alkyl, cycloalkyl or substituted cycloalkyl, cycloalkenyl or substituted cycloalkenyl, heterocyclo or substituted heterocyclo, cycloalkylalkyl or substituted cycloalkyalkyl, cycloalkenylalkyl or substituted cycloalkenylalkyl, heterocycloalkyl or substituted heterocycloalkyl, aryl or substituted aryl, arylalkyl or substituted arylalkyl;
R² is alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl;
R³ and R³′ are each independently H, alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, halo, CN, hydroxylamine, hydroxamide, alkoxy or substituted alkoxy, amino, NR¹R², thiol, alkylthio or substituted alkylthio;
R⁴ is H, alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, R¹C═O, R¹NHC═O, SO₂OR¹, or SO₂NR¹R¹′;
R⁵ is alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, R¹C═O, R¹NHC═O, SO₂R¹, SO₂OR¹, or SO₂NR¹R¹′;
R⁶ is alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, CN, OH, OR¹, R¹C═O, R¹NHC═O, SO₂R¹, SO₂OR¹, or SO₂NR¹R¹′;
R⁷ and R⁷′ are each independently H, alkyl or substituted alkyl, alkenyl or substituted alkenyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, halo, CN, OR¹, nitro, hydroxylamine, hydroxylamide, amino, NHR⁴, NR²R⁵, NOR¹, thiol, alkylthio or substituted alkylthio, R¹C═O, R¹OC═O, R¹NHC═O, SO₂R¹, SOR¹, PO₃R¹R¹′, R¹R¹′NC═O, C═OSR¹, SO₂R¹, SO₂OR¹, or SO₂NR¹R¹′, or, wherein A₁ or A₂ contains a group R⁷ and W contains a group R⁷, said R⁷ groups of A₁ or A₂ and W together form a heterocyclic ring;
R⁸ and R⁸′ are each independently H, alkyl or substituted alkyl, alkenyl or substituted alkenyl, cycloalkyl or substituted cycloalkyl, cycloalkenyl or substituted cycloalkenyl, heterocyclo or substituted heterocyclo, cycloalkylalkyl or substituted cycloalkyalkyl, cycloalkenylalkyl or substituted cycloalkenylalkyl, heterocycloalkyl or substituted heterocycloalkyl, aryl or substituted aryl, arylalkyl or substituted arylalkyl, nitro, halo, CN, OR¹, amino, NHR⁴, NR²R⁵, NOR¹, alkylthio or substituted alkylthio, C═OSR¹, R¹OC═O, R¹C═O, R¹NHC═O, R¹R¹′NC═O, SO₂OR¹, S═OR¹, SO₂R¹, PO₃R¹R¹′, or SO₂NR¹R¹′; and
R⁹ and R⁹′ are each independently H, alkyl or substituted alkyl, alkenyl or substituted alkenyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, CN, OH, OR¹, R¹C═O, R¹OC═O, R¹NHC═O, SO₂R¹, SO₂OR¹, or SO₂NR¹R¹′;
wherein said condition or disorder is selected from the group consisting of proliferate diseases, cancers, benign prostate hypertrophia, adenomas and neoplasies of the prostate, benign or malignant tumor cells containing the androgen receptor, heart disease, angiogenic conditions or disorders, hirsutism, acne, hyperpilosity, inflammation, immune modulation, seborrhea, endometriosis, polycystic ovary syndrome, androgenic alopecia, hypogonadism, osteoporosis, suppressing spermatogenisis, libido, cachexia, anorexia, inhibition of muscular atrophy in ambulatory patients, androgen supplementation for age related decreased testosterone levels in men, cancers expressing the estrogen receptor, prostate cancer, breast cancer, endometrial cancer, hot flushes, vaginal dryness, menopause, amennoreahea, dysmennoreahea, contraception, pregnancy termination, cancers containing the progesterone receptor, endometriosis, cachexia, menopause, cyclesynchrony, meniginoma, fibroids, labor induction, autoimmune diseases, Alzheimer's disease, psychotic disorders, drug dependence, non-insulin dependent Diabetes Mellitus, dopamine receptor mediated disorders, congestive heart failure, disregulation of cholesterol homeostasis, and attenuating the metabolism of a pharmaceutical agent.

16. A method for preparation of a compound of the following formula XVI, or salt thereof: 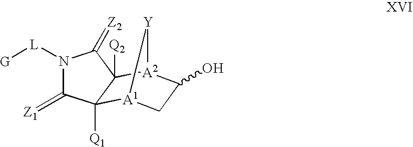
where
G is an aryl or heterocyclo group, where said group is mono- or polycyclic, and which is optionally substituted at one or more positions;
Z₁ is O, S, NH, or NR⁶;
Z₂ is O, S, NH, or NR⁶;
A₁ is CR⁷ or N;
A₂ is CR⁷ or N;
Y′ is J—J′—J″ where J is (CR⁷R⁷′)n and n=0-3, J′ is O, S, S═O, SO₂, NH, NR⁷, OP═OOR², OC═O, NR¹C═O, OP═ONHR², OSO₂, NHNH, NHNR⁶, NR⁶NH, or N═N, and J″ is (CR⁷R⁷′)n and n=0-3;
Q₁ is H, alkyl or substituted alkyl, alkenyl or substituted alkenyl, cycloalkyl or substituted cycloalkyl, cycloalkenyl or substituted cycloalkenyl, heterocycloalkyl or substituted heterocycloalkyl, arylalkyl or substituted arylalkyl, alkynyl or substituted alkynyl, aryl or substituted aryl, heterocyclo or substituted heterocyclo, halo, CN, R¹OC═O, R⁴C═O, R⁵R⁶NC═O, HOCR⁷R⁷′, nitro, R¹OCH₂, R¹O, NH₂, C═OSR¹, SO₂R¹ or NR⁴R₅;
Q₂ is H, alkyl or substituted alkyl, alkenyl or substituted alkenyl, cycloalkyl or substituted cycloalkyl, cycloalkenyl or substituted cycloalkenyl, heterocycloalkyl or substituted heterocycloalkyl, arylalkyl or substituted arylalkyl, alkynyl or substituted alkynyl, aryl or substituted aryl, heterocyclo or substituted heterocyclo, halo, CN, R¹OC═O, R⁴C═O, R⁵R⁶NC═O, HOCR⁷R⁷′, nitro, R¹OCH₂, R¹O, NH₂, C═OSR¹, SO₂R¹ or NR⁴R⁵;
L is a bond, (CR⁷R⁷′)n, NH, NR⁵ or NR⁵(CR⁷R⁷′)n, where n=0-3;
R¹ and R¹′ are each independently H, alkyl or substituted alkyl, cycloalkyl or substituted cycloalkyl, cycloalkenyl or substituted cycloalkenyl, heterocyclo or substituted heterocyclo, cycloalkylalkyl or substituted cycloalkyalkyl, cycloalkenylalkyl or substituted cycloalkenylalkyl, heterocycloalkyl or substituted heterocycloalkyl, aryl or substituted aryl, arylalkyl or substituted arylalkyl;
R² is alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl;
R⁴ is H, alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, R¹C═O, R¹NHC═O, SO₂OR¹, or SO₂NR¹R¹′;
R⁵ is alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, R¹C═O, R¹NHC═O, SO₂R¹, SO₂OR¹, or SO₂NR¹R¹′;
R⁶ is alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, CN, OH, OR¹, R¹C═O, R¹NHC═O, SO₂R¹, SO₂OR¹, or SO₂NR¹R¹′; and
R⁷ and R⁷′ are each independently H, alkyl or substituted alkyl, alkenyl or substituted alkenyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, halo, CN, OR¹, nitro, hydroxylamine, hydroxylamide, amino, NHR⁴, NR²R⁵, NOR¹, thiol, alkylthio or substituted alkylthio, R¹C═O, R¹OC═O, R¹NHC═O, SO₂R¹, SOR¹, PO₃R¹R¹′, R¹R¹′NC═O, C═OSR¹, SO₂R¹, SO₂OR¹, or SO₂NR¹R¹′;
comprising the steps of contacting a compound of the following formula XV, or salt thereof: 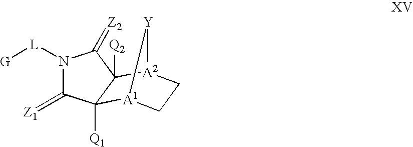
where the symbols are as defined above;
with an enzyme or microorganism capable of catalyzing the hydroxylation of said compound XV to said compound XVI, and effecting said hydroxylation.

17. A method for preparation of a compound of the following formula XVIII, or salt thereof: 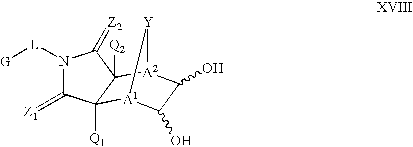
where
G is an aryl or heterocyclo group, where said group is mono- or polycyclic, and which is optionally substituted at one or more positions;
Z₁ is O, S, NH, or NR⁶;
Z₂ is O, S, NH, or NR⁶;
A₁ is CR⁷ or N;
A₂ is CR⁷ or N;
Y is J—J′—J″ where J is (CR⁷R⁷′)n and n=0-3, J′ is O, S, S═O, SO₂, NH, NR⁷, OP═OOR², OC═O, NR¹C═O, OP═ONHR², OSO₂, NHNH, NHNR⁶, NR⁶NH, or N═N, and J″ is (CR⁷R⁷′)n and n=0-3;
Q₁ is H, alkyl or substituted alkyl, alkenyl or substituted alkenyl, cycloalkyl or substituted cycloalkyl, cycloalkenyl or substituted cycloalkenyl, heterocycloalkyl or substituted heterocycloalkyl, arylalkyl or substituted arylalkyl, alkynyl or substituted alkynyl, aryl or substituted aryl, heterocyclo or substituted heterocyclo, halo, CN, R¹OC═O, R⁴C═O, R⁵R⁶NC═O, HOCR⁷R⁷′, nitro, R¹OCH₂, R¹O, NH₂, C═OSR¹, SO₂R¹ or NR⁴R⁵;
Q₂ is H, alkyl or substituted alkyl, alkenyl or substituted alkenyl, cycloalkyl or substituted cycloalkyl, cycloalkenyl or substituted cycloalkenyl, heterocycloalkyl or substituted heterocycloalkyl, arylalkyl or substituted arylalkyl, alkynyl or substituted alkynyl, aryl or substituted aryl, heterocyclo or substituted heterocyclo, halo, CN, R¹O C═O, R⁴C═O, R⁵R⁶N C═O, HOCR⁷R⁷′, nitro, R¹OCH₂, R¹O, NH₂, C═OSR¹, SO₂R¹ or NR⁴R⁵;
L is a bond, (CR⁷R⁷′)n, NH, NR⁵ or NR⁵(CR⁷R⁷′)n, where n=0-3;
R¹ and R¹′ are each independently H, alkyl or substituted alkyl, cycloalkyl or substituted cycloalkyl, cycloalkenyl or substituted cycloalkenyl, heterocyclo or substituted heterocyclo, cycloalkylalkyl or substituted cycloalkyalkyl, cycloalkenylalkyl or substituted cycloalkenylalkyl, heterocycloalkyl or substituted heterocycloalkyl, aryl or substituted aryl, arylalkyl or substituted arylalkyl;
R² is alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl;
R⁴ is H, alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, R¹C═O, R¹NHC═O, SO₂OR¹, or SO₂NR¹R¹′;
R⁵ is alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, R¹C═O, R¹NHC═O, SO₂R¹, SO₂OR¹, or SO₂NR¹R¹′;
R⁶ is alkyl or substituted alkyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, CN, OH, OR¹, R¹C═O, R¹NHC═O, SO₂R¹, SO₂OR¹, or SO₂NR¹R¹′; and
R⁷ and R⁷′ are each independently H, alkyl or substituted alkyl, alkenyl or substituted alkenyl, cycloalkyl or substituted cycloalkyl, cycloalkenyl or substituted cycloalkenyl, heterocyclo or substituted heterocyclo, cycloalkylalkyl or substituted cycloalkylalkyl, cycloalkenylalkyl or substituted cycloalkenylalkyl, heterocycloalkyl or substituted heterocycloalkyl, aryl or substituted aryl, arylalkyl or substituted arylalkyl, halo, CN, OR¹, nitro, hydroxylamine, hydroxylamide, amino, NHR⁴, NR²R⁵, NOR¹, thiol, alkylthio or substituted alkylthio, R¹C═O, R¹OC═O, R¹NHC═O, SO₂R¹, SOR¹, PO₃R¹R¹′, R¹R¹′NC═O, C═OSR¹, SO₂R¹, SO₂OR¹, or SO₂NR¹R¹′;
comprising the steps of contacting a compound of the following formula XVII, or salt thereof: 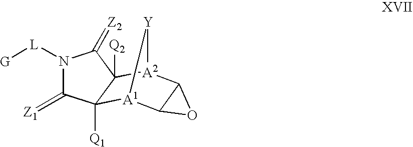
where the symbols are as defined above;
with an enzyme or microorganism capable of catalyzing the opening of the epoxide ring of compound XVII to form the diol of said compound XVIII, and effecting said ring opening and diol formation.

18. A compound of the following formula Ib: 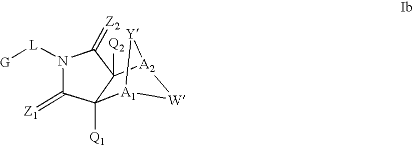
where G, Z₁, Z₂, Q₁ and Q₂ are as defined in claim 1;
Y′ is J—J′—J″ where J is (CR⁷R⁷′)n and n=0-3, J′ is a bond or O, S, S═O, SO₂, NH, NR⁷, CR⁷R⁷′, R²P═O, R²P═S, R²OP═O, R²NHP═O, OP═OOR², OP═ONHR², OSO₂, NHNH, NHNR⁶, NR⁶NH, N═N, cycloalkyl or substituted cycloalkyl, cycloalkenyl or substituted cycloalkenyl, or heterocyclo or substituted heterocyclo, and J″ is (CR⁷R⁷′)n and n=0-3, where Y is not a bond; and
W′ is CR⁷′R⁷—CR⁷R⁷′, CR⁷R⁷′—C═O, NR⁹—CR⁷R⁷′, N═CR⁸, N═N, NR⁹′, cycloalkyl or substituted cycloalkyl, cycloalkenyl or substituted cycloalkenyl, heterocyclo or substituted heterocyclo, or aryl or substituted aryl, wherein,
when W′ is not NR⁹—CR⁷R⁷′, N═CR⁸, N═N, NR⁹—NR⁹, or heterocyclo or substituted heterocyclo, then J′ must be O, S, S═O, SO₂, NH, NR⁷, OP═OOR², OP═ONHR², OSO₂, NHNH, NHNR⁶, NR⁶NH, or N═N; or alternatively,
Y′ is CR⁷R⁷′—C═O and W is NR⁹—CR⁷R⁷′;
L is a bond; and
A₁ and A₂ are as defined above with the proviso that, when Y′=O and W′=—CH₂—
CH₂—, then at least one of A₁ or A₂ is not CH;
with the further provisos (2), (3), (6), (7) and (8) of claim 1.
